(19) United States
(12) Patent Application Publication
(10) Pub. No.: US 2026/0297574 A1
(43) Pub. Date: Oct. 1, 2026

(54) EXTRAHEPATIC DELIVERY OF DOUBLE-STRANDED RNA AGENTS

(71) Applicant: ALNYLAM PHARMACEUTICALS, INC., Cambridge, MA (US)

(72) Inventors: Jayaprakash K. NAIR, Cambridge, MA (US); Maja JANAS, Cambridge, MA (US); Kirk BROWN, Cambridge, MA (US); Haiyan PENG, Cambridge, MA (US); Christopher S. THEILE, Cambridge, MA (US); Kevin DOOLEY, Cambridge, MA (US); Vasant R. JADHAY, Cambridge, MA (US); Martin A. MAIER, Cambridge, MA (US); Muthiah MANOHARAN, Cambridge, MA (US); Dhrubajyoti DATTA, Cambridge, MA (US); Masaaki NAKATA, Cambridge, MA (US); Mimouna MADAOUI, Cambridge, MA (US)

(21) Appl. No.: 19/474,339

(22) PCT Filed: Apr. 12, 2024
(86) PCT No.: PCT/US2024/024433

§ 371 Date: Oct. 10, 2025

Related U.S. Application Data
(60) Provisional application No. 63/458,788, filed on Apr. 12, 2023.
(60) Provisional application No. 63/524,117, filed on Jun. 29, 2023.
(60) Provisional application No. 63/566,106, filed on Mar. 15, 2024.

(51) Int. Cl. C12N 15/113

(52) U.S. Cl. CPC C12N 15/113; C12N 2310/14; C12N 2310/3515; C12N 2320/32

(57) ABSTRACT

One aspect of the invention relates to double-stranded RNA (dsRNA) agent for modulating the expression of a target gene in the central nervous system (CNS), comprising an antisense strand which is complementary to the target gene in the CNS; a sense strand which is complementary to the antisense strand; and one or more lipophilic moieties containing one or more saturated or unsaturated C₂₂ hydrocarbon chains conjugated to at least one strand, optionally via a linker or carrier. Another aspect of the invention relates to a pharmaceutical composition comprising the dsRNA agent. Other aspects of the invention relate to a method of modulating the expression of a target gene in a CNS cell gene and a method treating or preventing a CNS disorder in a subject, comprising administering to the cell or the subject in a therapeutically effective amount of the dsRNA agent.

### CROSS-REFERENCE TO RELATED APPLICATIONS

This application claims benefit of priority to U.S. Provisional Application No. 63/458,788, filed Apr. 12, 2023; U.S. Provisional Application No. 63/524,117, filed Jun. 29, 2023; and U.S. Provisional Application No. 63/566,106 filed Mar. 15, 2024; all of which are herein incorporated by reference in their entirety.

### SEQUENCE LISTING

The instant application contains a Sequence Listing which has been submitted in XML format and is hereby incorporated by reference in its entirety. Said XML copy, created on Apr. 11, 2024, is named 29520_1512-PCT__ALN-499-WO_SL.xml and is 2,102,861 bytes in size.

### BACKGROUND

Efficient delivery of a dsRNA agent to cells in vivo requires specific targeting and substantial protection from the extracellular environment, particularly serum proteins. RNAi-based therapeutics show promising clinical data for treatment of liver-associated disorders. However, siRNA delivery into extra-hepatic tissues remains an obstacle, limiting the use of siRNA-based therapies.

One of the factors that limit the experimental and therapeutic application of dsRNA agents in vivo is the ability to deliver intact siRNA efficiently.

Delivery of oligonucleotides to the central nervous system (CNS) poses particular problems due to the blood brain barrier (BBB) that free oligonucleotides cannot cross. One means to deliver oligonucleotides into the CNS is by intrathecal delivery. However, the oligonucleotides need also to be efficiently internalized into target cells of the CNS to achieve the desired therapeutic effect. Previous work has typically used delivery reagents such as liposomes, cationic lipids, and nanoparticles forming complexes to aid the intracellular internalization of oligonucleotides into cells of neuronal origin.

Thus, there is a continuing need for new and improved compositions and methods for delivering siRNA molecules in vivo, without the use of tissue delivery reagents, to achieve and enhance the therapeutic potential of dsRNA agents.

### SUMMARY

One aspect of the invention provides a compound (e.g., an oligonucleotide that can be either single-stranded or double-stranded) comprising one or more lipophilic monomers, containing one or more lipophilic moieties, conjugated to one or more positions on at least one strand of the oligonucleotide, optionally via a linker or carrier.

Some embodiments of the invention provide a compound (e.g., a double-stranded RNA (dsRNA) agent) for modulating the expression of a target gene in the central nervous system (CNS) comprising: an antisense strand which is complementary to a target gene in the CNS; a sense strand which is complementary to said antisense strand; and one or more lipophilic moieties containing one or more saturated or unsaturated C₂₂ hydrocarbon chains, conjugated to one or more positions on at least one strand, optionally via a linker or carrier.

In some embodiments, the target gene in the CNS is selected from the group consisting of APP, SOD1, SCN9A, HTT (HUNTINGTIN), APOE, LRRK2, PRNP, SCD5, GPR75, MAPT, SNCA, ABLIM3, ADRA2A, ATXN1, ATXN2, ATXN3, ELOVL1, FLNA, NOGO-L or NOGO-R, HIF-1α, RHO-A, NAV1.8, CD45, GSK-3, GSK3a, MIG-12, Mgat1, Mgat4, SLC35A1, SLC35A2, GNE, TMPRSS6, Complement Component C3, APCS, C9orf72, CHI3L1/YKL-40, EXT1, EXT2, NDST2, RPS25, ALK, and SCD5. In some embodiments, the target gene in the CNS is selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, TTR, SCN9A, LRRK2, GPR75, APOE, SCD5, ELOVL1, FLNA, ALK, CHI3L1(YKL-40), RPS25, α2-AR, and GSK3α.

In some embodiments, the lipophilicity of the lipophilic moiety, measured by octanol-water partition coefficient, log K_{ow}, exceeds 0. The lipophilic moiety may possess a log K_{ow} exceeding 1, exceeding 1.5, exceeding 2, exceeding 3, exceeding 4, exceeding 5, or exceeding 10.

In some embodiments, the hydrophobicity of the compound (e.g., a dsRNA agent), measured by the unbound fraction in the plasma protein binding assay of the compound, exceeds 0.2. In one embodiment, the plasma protein binding assay determined is an electrophoretic mobility shift assay (EMSA) using human serum albumin protein. The hydrophobicity of the compound, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

In some embodiments, one or more lipophilic moieties can be an aliphatic, cyclic such as alicyclic, or polycyclic such as polyalicyclic compound, such as a steroid (e.g., sterol) or a linear or branched aliphatic hydrocarbon. Exemplary lipophilic moieties are lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

Suitable lipophilic moieties also include those containing a saturated or unsaturated C₄-C₃₀ hydrocarbon chain (e.g., C₄-C₃₀ alkyl or alkenyl, linear or branched), and an optional functional group selected from the group consisting of hydroxyl, amine, carboxylic acid, carboxylic acid ester, sulfonate, ether, phosphate, thiol, azide, alkyne (e.g., a terminal alkyne), cycloalkyne (e.g., cyclooctyne, dibenzocyclooctyne, or aza-dibenzocyclooctyne), trans-cyclooctenyl, N-maleimidyl, and 1,2,4,5-tetrazin-3-yl. The functional groups are useful to attach the lipophilic moiety to the dsRNA agent.

In some embodiments, one or more lipophilic moieties can contain a saturated or unsaturated C₂₀-C₃₀ hydrocarbon chain (e.g., a linear C₂₀-C₃₀ alkyl or alkenyl). In one embodiment, one or more lipophilic moieties can contain a saturated or unsaturated C₂₀-C₂₈ hydrocarbon chain; saturated or unsaturated C₂₀-C₂₆ hydrocarbon chain; saturated or unsaturated C₂₀-C₂₄ hydrocarbon chain. In one embodiment, one or more lipophilic moieties can contain a saturated or unsaturated C₂₀ hydrocarbon chain; a saturated or unsaturated C₂₁ hydrocarbon chain; a saturated or unsaturated C₂₂ hydrocarbon chain; a saturated or unsaturated C₂₃ hydrocarbon chain; or a saturated or unsaturated C₂₄ hydrocarbon chain.

In other embodiments, the lipophilic moiety contains a C₂₀ alkyl chain, or a C₂₁ alkyl chain, or a C₂₂ alkyl chain, or a C₂₃ alkyl chain, or a C₂₄ alkyl chain. Each of the preceding, in another embodiment, may be a linear alkyl chain (e.g., n-eicosyl, or n-henicosanyl, n-docosanyl, or n-tricosanyl, or n-tetracosanyl).

In one embodiment, one or more lipophilic moieties can contain a saturated or unsaturated C₂₂ hydrocarbon chain (e.g., a linear or branched C₂₂ alkyl or alkenyl). In some embodiments, one or more lipophilic moieties can contain two or more carbon-carbon double bonds. In some embodiments, one or more lipophilic moieties can contain one or more carbon-carbon double bonds and one or more carbon-carbon triple bonds (e.g., an “enyne”, such as a conjugated enyne).

Examples of branched lipophilic moieties include, but are not limited to, docosan-2-yl, docosan-3-yl, docosan-4-yl, docosan-5-yl, docosan-6-yl, docosan-7-yl, docosan-8-yl, docosan-9-yl, docosan-10-yl, docosan-11-yl, 2-(decyl)dodecan-1-yl, 2-(nonyl)tridecan-1-yl, 2-(octyl)tetradecan-1-yl, 2-(heptyl)pentadecan-1-yl, 2-(hexyl)hexadecan-1-yl, 2-(pentyl)heptadecan-1-yl, 2-(butyl)octadecan-1-yl, 2-(propyl)nonadecan-1-yl, 2-(ethyl)eicosan-1-yl, 2-(methyl)henicosan-1-yl, 3-(nonyl)tridecan-1-yl, 3-(octyl)tetradecan-1-yl, 3-(heptyl)pentadecan-1-yl, 3-(hexyl)hexadecan-1-yl, 3-(pentyl)heptadecan-1-yl, 3-(butyl)octadecan-1-yl, 3-(propyl)nonadecan-1-yl, 3-(ethyl)eicosan-1-yl, 3-(methyl)henicosan-1-yl, 4-(octyl)tetradecan-1-yl, 4-(heptyl)pentadecan-1-yl, 4-(hexyl)hexadecan-1-yl, 4-(pentyl)heptadecan-1-yl, 4-(butyl)octadecan-1-yl, 4-(propyl)nonadecan-1-yl, 4-(ethyl)eicosan-1-yl, 4-(methyl)henicosan-1-yl, 5-(heptyl)pentadecan-1-yl, 5-(hexyl)hexadecan-1-yl, 5-(pentyl)heptadecan-1-yl, 5-(butyl)octadecan-1-yl, 5-(propyl)nonadecan-1-yl, 5-(ethyl)eicosan-1-yl, 5-(methyl)henicosan-1-yl, 6-(hexyl)hexadecan-1-yl, 6-(pentyl)heptadecan-1-yl, 6-(butyl)octadecan-1-yl, 6-(propyl)nonadecan-1-yl, 6-(ethyl)eicosan-1-yl, 6-(methyl)henicosan-1-yl, 7-(pentyl)heptadecan-1-yl, 7-(butyl)octadecan-1-yl, 7-(propyl)nonadecan-1-yl, 7-(ethyl)eicosan-1-yl, 7-(methyl)henicosan-1-yl, 8-(butyl)octadecan-1-yl, 8-(propyl)nonadecan-1-yl, 8-(ethyl)eicosan-1-yl, 8-(methyl)henicosan-1-yl, 9-(propyl)nonadecan-1-yl, 9-(ethyl)eicosan-1-yl, 9-(methyl)henicosan-1-yl, 10-(ethyl)eicosan-1-yl, 10-(methyl)henicosan-1-yl, and 11-(methyl)henicosan-1-yl.

In some embodiments, one or more lipophilic moieties can contain one or more hydrocarbon chains such that the total number of carbon atoms in the lipophilic moiety is 20-30 (e.g, 21 or 22). For example, the lipophilic moiety can be of the formula

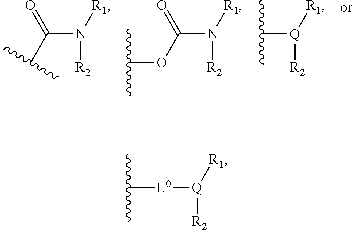

wherein Q is branch point (e.g., N or C(H)), L⁰ is a divalent linking group (L⁰ may be one of the linkers/tethers as defined herein), and R₁ and R₂ are each independently a C₁-C₂₀ hydrocarbon chain, such that the sum of the carbon atoms in R₁ and R₂ is 20 to 30 (e.g., 22, or 21 when Q is C(H)). In other embodiments, Q is of the formula, —N(R₃)—[CH₂]ₙ—N(R₄)—, wherein n is 2-10 and R₃ and R₄ are each independently a C₁-C₂₀ hydrocarbon chain, such that the sum of the carbon atoms in R₁, R₂, R₃, and R₄ is 20 to 30 (e.g., 22). In other embodiments, Q is of the formula,

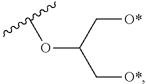

wherein the * are bonded to R₁ and R₂, wherein sum of carbon atoms in R₁ and R₂ is 20 to 30 (e.g., 22). In some embodiments, one or more of R₁, R₂, R₃, and R₄ can contain two or more carbon-carbon double bonds. In some embodiments, one or more of R₁, R₂, R₃, and R₄ can contain one or more carbon-carbon double bonds and one or more carbon-carbon triple bonds (e.g., an “enyne”, such as a conjugated enyne). In some embodiments, one or more of R₁, R₂, R₃, and R₄ can contain a functional group selected from the group consisting of hydroxyl, amine, carboxylic acid, carboxylic acid ester, sulfonate, ether, phosphate, thiol, azide, alkyne (e.g., a terminal alkyne), cycloalkyne (e.g., cyclooctyne, dibenzocyclooctyne, or aza-dibenzocyclooctyne), trans-cyclooctenyl, N-maleimidyl, and 1,2,4,5-tetrazin-3-yl.

In some embodiments, one or more lipophilic moieties can be a C₆-C₃₀ moiety having a free terminal carboxylic acid functionality (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexaenoic acid).

In some embodiments, one or more lipophilic moieties can be a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexaenoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.).

In some embodiments, two or more lipophilic moieties may be conjugated to the dsRNA agent.

In some embodiments, at least one lipophilic moiety containing one or more saturated or unsaturated C₂₂ hydrocarbon chains is conjugated to the dsRNA agent.

In some embodiment, at least one C₂₂ hydrocarbon chain is a saturated or unsaturated, linear or branched C₂₂ hydrocarbon chain. The one or more C₂₂ hydrocarbon chains may contain a functional group selected from the group consisting of hydroxyl, amine, carboxylic acid, carboxylic acid ester, sulfonate, ether, phosphate, thiol, azide, alkyne (e.g., a terminal alkyne), and cycloalkyne (e.g., cyclooctyne, dibenzocyclooctyne, or aza-dibenzocyclooctyne), trans-cyclooctenyl, N-maleimidyl, and 1,2,4,5-tetrazin-3-yl. The functional groups are useful to attach the lipophilic moiety to the dsRNA agent.

In some embodiments, one or more lipophilic moieties can have the formula, -G₂-R^{G}, wherein G₂ is a saturated or unsaturated C₂₀-C₃₀ hydrocarbon group (e.g., a saturated or unsaturated C₂₁ or C₂₂ hydrocarbon group) and R^{G} is selected from the group consisting of hydrogen, hydroxy, amino, —COOH, and —C(O)NH₂.

In some embodiments, one or more lipophilic moieties can have the formula, -G₂-R^{G}, wherein G₂ is a saturated or unsaturated C₂₁ hydrocarbon group and R^{G} is —COOH or —C(O)NH₂.

In some embodiments, one or more lipophilic moieties can have the formula, -G₂-R^{G}, wherein G₂ is a saturated or unsaturated C₂₂ hydrocarbon group and R^{G} is —COOH or —C(O)NH₂.

In some embodiments, one or more lipophilic moieties can have the formula, -G₂-R^{G}, wherein G₂ is a saturated or unsaturated C₂₂ hydrocarbon group and R^{G} is —OH.

In some embodiments, one or more lipophilic moieties can have the formula
-G₃-L^{K}-G₂-R^{G}, wherein:
G₃ is a saturated or unsaturated C₁₋₂₀ hydrocarbon group (e.g., C₁₋₆ alkylene; C₂₋₆ alkylene; or hexylene);
L^{K} is a linking group such as —O—, —N(H)—, —S—, —S—S—, —C(O)O—, —OC(O)—, —C(O)N(H)—, —N(H)C(O), —OC(O)N(H)—, —N(H)C(O)O—, —S(O)₂—, —S(O)₂O—, —S(O)₂N(H)—, —P(O)(OH)O—, —OP(O)(OH)—, —P(S)(OH)O—, —OP(S)(OH)—, —OP(O)(OH)O—, —OP(S)(OH)O—,
G₂ is a saturated or unsaturated C₂₁-C₂₂ hydrocarbon group; and
R^{G} is hydrogen, hydroxy, amino, —COOH, or —C(O)NH₂.
   For example, when L^{K} contains a carbonyl attached to G₂ (e.g., (—N(H)C(O)—, or —OC(O)—), then G₂ is a C₂₁ hydrocarbon group; when L^{K} does not contain a carbonyl attached to G₂, then G₂ is a C₂₂ hydrocarbon group. In one embodiment, R^{G} is hydrogen. In one embodiment, R^{G} is OH. In one embodiment, R^{G} is COOH. In one embodiment, R^{G} is CONH₂. In one embodiment, R_{G} is amino.

In some embodiments, one or more lipophilic moieties can have the formula
-L^{K2}-G₃-L^{K}-G₂-R^{G}, wherein:
G₂ is a saturated or unsaturated C₂₁-C₂₂ hydrocarbon group;
R^{G} is hydrogen, hydroxy, amino, —COOH, or —C(O)NH₂; G₃ is a saturated or unsaturated C₁₋₂₀ hydrocarbon group (e.g., C₁₋₆ alkylene; C₂₋₆ alkylene; or hexylene); and
L^{K} and L^{K2} are independently a linking group such as —O—, —N(H)—, —S—, —S—S—, —C(O)O—OC(O)—, —C(O)N(H)—, —N(H)C(O), —OC(O)N(H)—, —N(H)C(O)O—, —S(O)₂—, —S(O)₂O—, —S(O)₂N(H)—, —P(O)(OH)O—, —OP(O)(OH)—, —P(S)(OH)O—, —OP(S)(OH)—, —OP(O)(OH)O—, or —OP(S)(OH)O— (e.g., L^{K2} is —C(O)—, —S(O)₂—, —P(O)(OH)O—, —OP(O)(OH)—, —P(S)(OH)O—, —OP(S)(OH)—, —OP(O)(OH)O—, or —OP(S)(OH)O—).

For example, when L^{K} contains a carbonyl attached to G₂ (e.g., (—N(H)C(O)— or —OC(O)—), then G₂ is a C₂₁ hydrocarbon group; and when L^{K} does not contain a carbonyl attached to G₂, then G₂ is a C₂₂ hydrocarbon group. (e.g., (—N(H)C(O)— or —OC(O)—). In one embodiment, R^{G} is hydrogen. In one embodiment, R^{G} is OH. In one embodiment, R^{G} is COOH. In one embodiment, R^{G} is CONH₂. In one embodiment, R^{G} is amino. In one embodiment, L^{K2} is —P(O)(OH)O—, —OP(O)(OH)—, —P(S)(OH)O—, —OP(S)(OH)—, —OP(O)(OH)O—, or —OP(S)(OH)O—. In one embodiment, L^{K2} is —OP(O)(OH)O—, or —OP(S)(OH)O—. In one embodiment, L^{K2} is —OP(O)(OH)O. In one embodiment, L^{K2} is —OP(S)(OH)O—.

In some embodiments, at least one C₂₂ hydrocarbon chain comprises a C₂₂ acid. Exemplary C₂₂ acids include but are not limited to docosanoic acid, 6-octyltetradecanoic acid, 10-hexylhexadecanoic acid, all-cis-7,10,13,16,19-docosapentaenoic acid, all-cis-4,7,10,13,16,19-docosahexaenoic acid, all-cis-13,16-docosadienoic acid, all-cis-7,10,13,16-docosatetraenoic acid, all-cis-4,7,10,13,16-docosapentaenoic acid, cis-13-docosenoic acid,

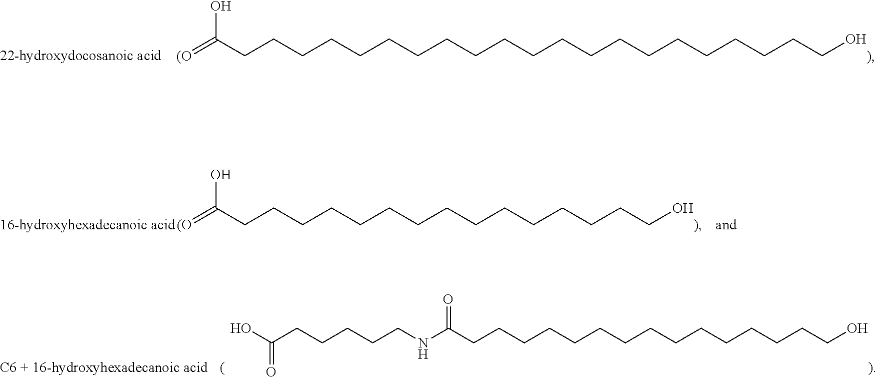

In some embodiments, one or more lipophilic moieties can have the formula, -G₂-R^{G}, wherein G₂ is a saturated or unsaturated C₂₂ hydrocarbon group and R^{G} is hydrogen or hydroxy.

In some embodiments, at least one C₂₂ hydrocarbon chain comprises a C₂₂ alcohol. Exemplary C₂₂ alcohol include but are not limited to 1-docosanol, 6-octyltetradecan-1-ol, 10-hexylhexadecan-1-ol, cis-13-docosen-1-ol, docosan-9-ol, docosan-2-ol, docosan-10-ol, docosan-11-ol, cis-4,7,10,13,16,19-docosahexanol, 22-hydroxydocosanoic acid, 16-hydroxyhexadecanoic acid, and C6+16-hydroxyhexadecanoic acid.

In some embodiments, at least one C₂₂ hydrocarbon chain comprises a C₂₂ amide. Exemplary C₂₂ amides include but are not limited to (E)-Docos-4-enamide, (E)-Docos-5-enamide, (Z)-Docos-9-enamide, (E)-Docos-11-enamide, 12-Docosenamide, (Z)-Docos-13-enamide, (Z)—N-Hydroxy-13-docoseneamide, (E)-Docos-14-enamide, 6-cis-Docosenamide, 14-Docosenamide Docos-11-enamide, (4E,13E)-Docosa-4,13-dienamide, and (5E,13E)-Docosa-5,13-dienamide.

The lipophilic moiety may be conjugated to any part of the dsRNA agent, e.g., a nucleobase, sugar moiety, or internucleosidic linkage. It is understood that when a lipophilic moiety is conjugated to a modified internucleotide linkage, then it is considered to be “at” a position in the oligonucleotide when forming part of the internucleotide linkage on the 3′-side of the referenced nucleotide.

The lipophilic moiety may be conjugated to the dsRNA agent via a direct attachment to the nucleobase, ribosugar, or internucleosidic linkage of the dsRNA agent. Alternatively, the lipophilic moiety may be conjugated to the dsRNA agent via a non-ribose replacement unit, such as a linker or carrier.

In some embodiments, the lipophilic moiety is conjugated to the ribosugar of the dsRNA agent, In one embodiment, the lipophilic moiety is conjugated to the 2′ position of the ribosugar of the dsRNA agent, In one embodiment, the lipophilic moiety is conjugated to the 3′ position of the ribosugar of the dsRNA agent, In one embodiment, the lipophilic moiety is conjugated to the 5′ position of the ribosugar of the dsRNA agent,

In some embodiments, the lipophilic moiety is conjugated to the 2′-O— position of the ribosugar of the dsRNA agent, optionally via one or more linkers. For instance, the lipophilic moiety is conjugated to the 2′ position of the ribosugar of the dsRNA as 2′-OCH₂C(O)N(H)-lipophilic moiety.

In some embodiments, the lipophilic moiety is conjugated to the 3′-position of the ribosugar of the dsRNA agent, optionally via one or more linkers. For instance, the lipophilic moiety is conjugated to the 3′ position of the ribosugar of the dsRNA as 3′-(N-lipophilic moiety) phosphoramidate (e.g., 3′-P(═O)(O)(O)N-lipophilic moiety.

In certain embodiments, the lipophilic moiety is conjugated to the dsRNA agent via one or more linkers (tethers).

In some embodiments, the lipophilic moiety is conjugated to a nucleobase, optionally via one or more linkers (tethers). In some embodiments, the lipophilic moiety is conjugated to a sugar moiety, optionally via one or more linkers (tethers). In some embodiments, the lipophilic moiety is conjugated to an internucleotide phosphate linker, optionally via one or more linkers (tethers).

In some embodiments, the lipophilic moiety is conjugated to the dsRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, mannose, and combinations thereof.

In certain embodiments, the lipophilic moiety is conjugated to the dsRNA agent via a non-ribose replacement unit, i.e., a carrier, that replaces one or more nucleotide(s) of the dsRNA agent. The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone, a glycerol backbone, or a diethanolamine backbone.

In some embodiments, the carrier replaces one or more nucleotide(s) in the dsRNA agent. In some embodiments, the carrier replaces one or more nucleotide(s) in the internal position(s) of the dsRNA agent. In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

In some embodiments, the lipophilic moiety conjugated via a carrier may be represented by one of the following formulae:

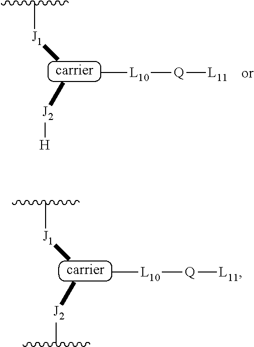

wherein.
J₁ and J₂ are each independently O, S, NR^{N}, optionally substituted alkyl, OC(O)NH, NHC(O)O, C(O)NH, NHC(O), OC(O), C(O)O, OC(O)O, NHC(O)NH, NHC(S)NH, OC(S)NH, OP(N(R^{P})₂)O, or OP(N(R^{P})₂);
[symbol] is a cyclic group or an acyclic group;
R^{N} is H, optionally substituted alkyl, optionally substituted alkenyl, optionally substituted alkynyl, optionally substituted aryl, optionally substituted cycloalkyl, optionally substituted aralkyl, optionally substituted heteroaryl, or an amino protecting group;
R^{P} is independently for each occurrence H, optionally substituted alkyl, optionally substituted alkenyl, optionally substituted alkynyl, optionally substituted aryl, optionally substituted cycloalkyl, or optionally substituted heteroaryl;
L₁₀ is substituted or unsubstituted, saturated or unsaturated C₃-C₈ hydrocarbon, (e.g., C₃-C₈ alkyl, alkenyl, or alkynyl, or C₃-C₈ hydrocarbon containing two or more double bonds); the substituted groups include those already described herein for “substituted” hydrocarbon, alkyl, alkenyl, or alkynyl;
L₁₁ is substituted or unsubstituted, saturated or unsaturated C₆-C₂₆ hydrocarbon, (e.g., C₆-C₂₆ alkyl, alkenyl, or alkynyl, or C₃-C₈ hydrocarbon containing two or more double bonds); the substituted groups include those already described herein for “substituted” hydrocarbon, alkyl, alkenyl, or alkynyl; and
Q is absent when there is no nucleobase on the carrier, or a cleavable group that will cleave L₁₀ from L₁₁ at least 10% in vivo. For instance, Q may be a cleavable group that can be cleaved in vivo to cleave L₁₁ off the lipophilic monomer by about 10-70%, about 15-50%, about 20-40%, or about 20-30%. Exemplary cleavable groups include —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)N(R⁵)—, —N(R⁵)C(O)—, —C(S)N(R⁵)—, —N(R⁵)C(S)—, —N(R⁵)C(O)N(R⁵)—, —N(R⁵)C(O)C(R³)(R⁴)OC(O)—, —C(O)OC(R³)(R⁴)C(O)N(R⁵)—, —OC(O)O—, —OSi(R⁵)₂O—, —C(O)(CR³R⁴)C(O)O—, —OC(O)(CR³R₄)C(O)—,

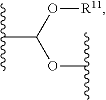

or combinations thereof, R¹¹ is a C₂-C₈ alkyl or alkenyl. For each occurrence, R³, R⁴, and R⁵ are each independently H or C₁-C₄ alkyl.

In one embodiment, the cleavability of Q is determined by the stability of ligands in cerebral spinal fluid (CSF), the stability of ligands in plasma, the stability of ligands in brain homogenate or tissue homogenate (e.g., liver, etc.).

The cyclic and acyclic groups include those already described herein.

In one embodiment, the acyclic group is a serinol, glycerol, or diethanolamine backbone.

In one embodiment, the cyclic group is selected from the group consisting of pyrrolidinyl, hydroxyprolinyl, cyclopentyl, cyclohexyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the cyclic group is a ribose or a ribose analog. Examples of ribose analogs include arabinose, 4′-thio ribose, 2′-O-methyl ribose, GNA, UNA, and LNA analogs.

In some embodiments, the lipophilic moiety containing the one or more saturated or unsaturated C₂₂ hydrocarbon chains is a lipophilic monomer selected from the group consisting of:

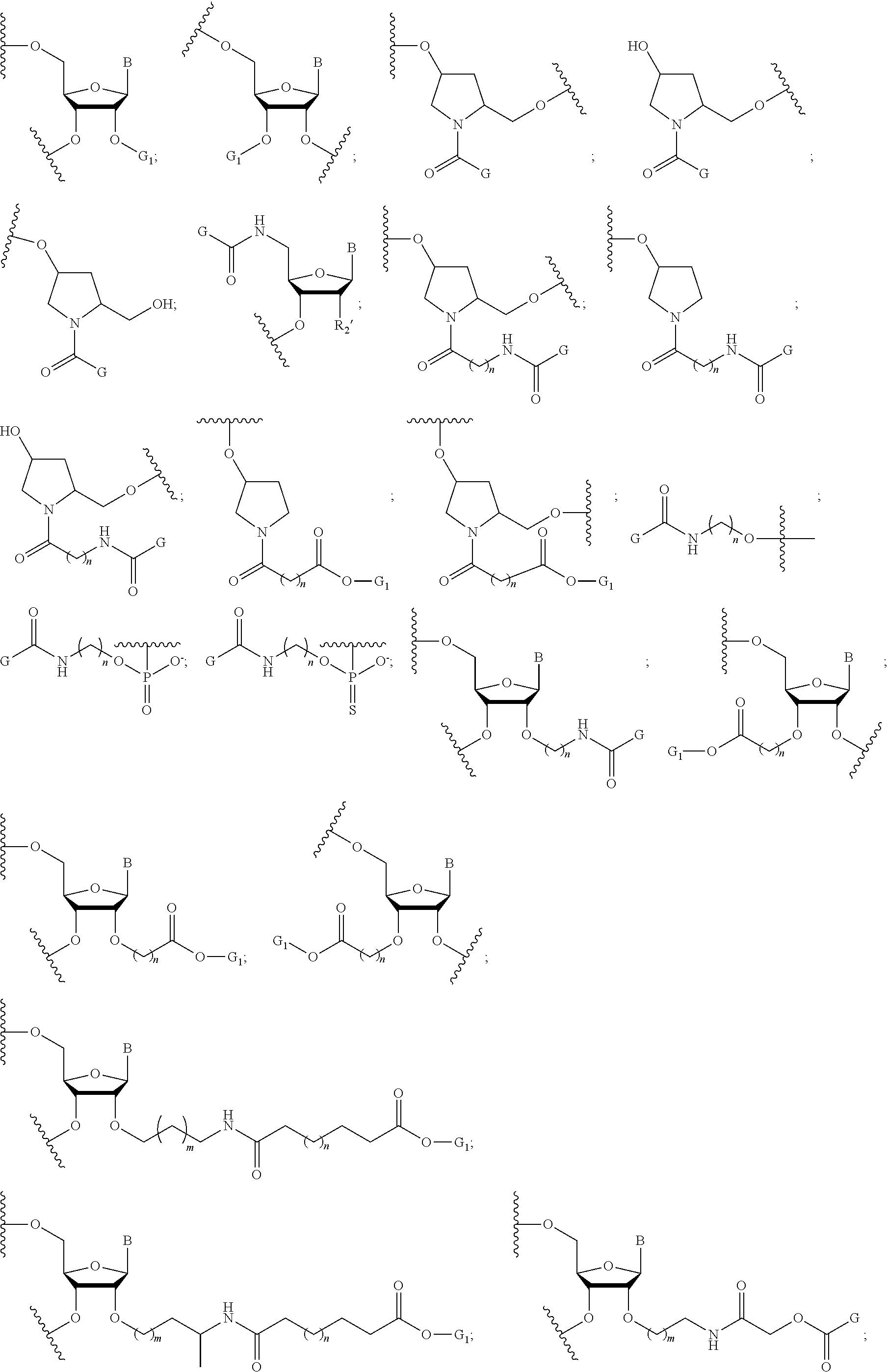

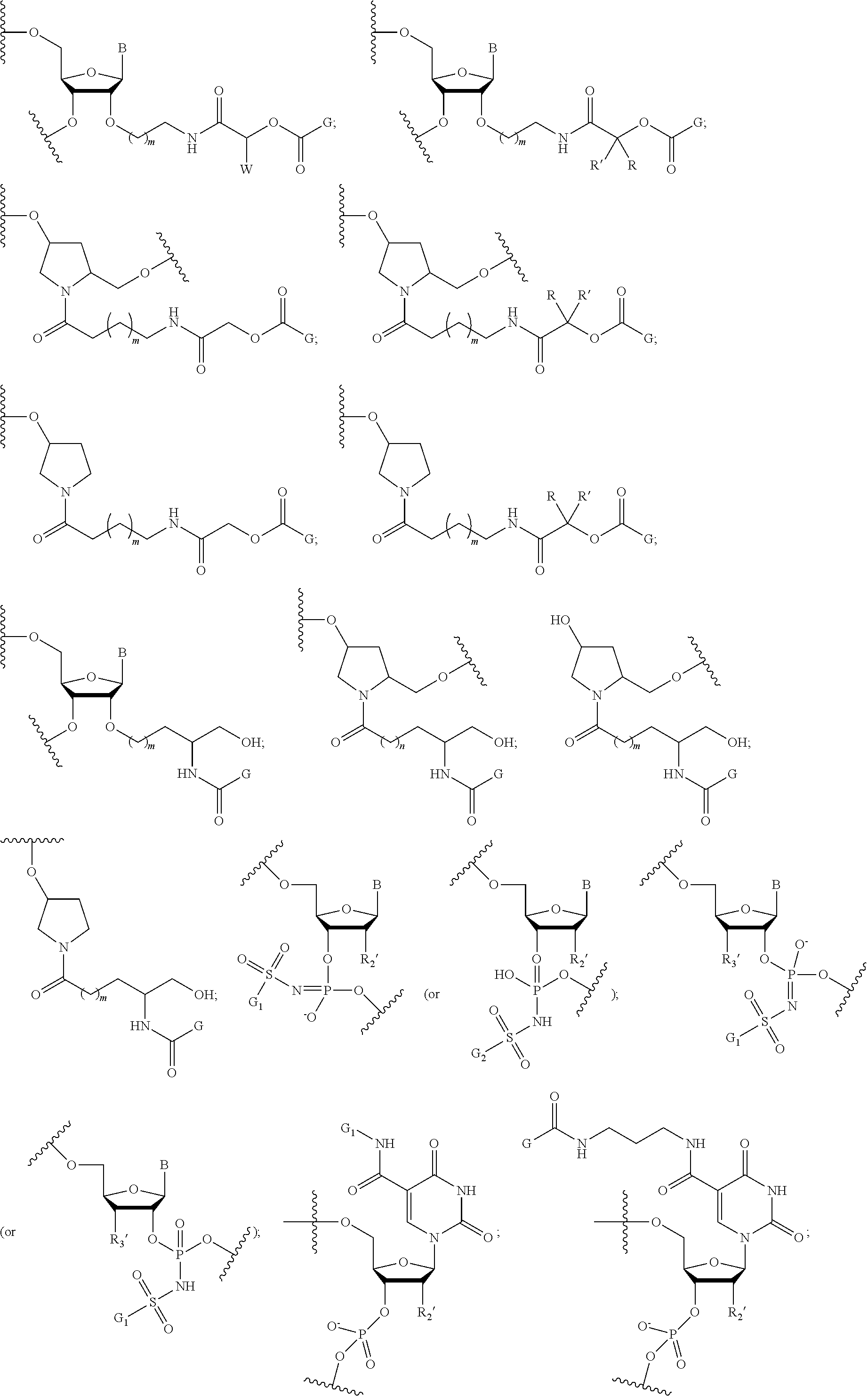

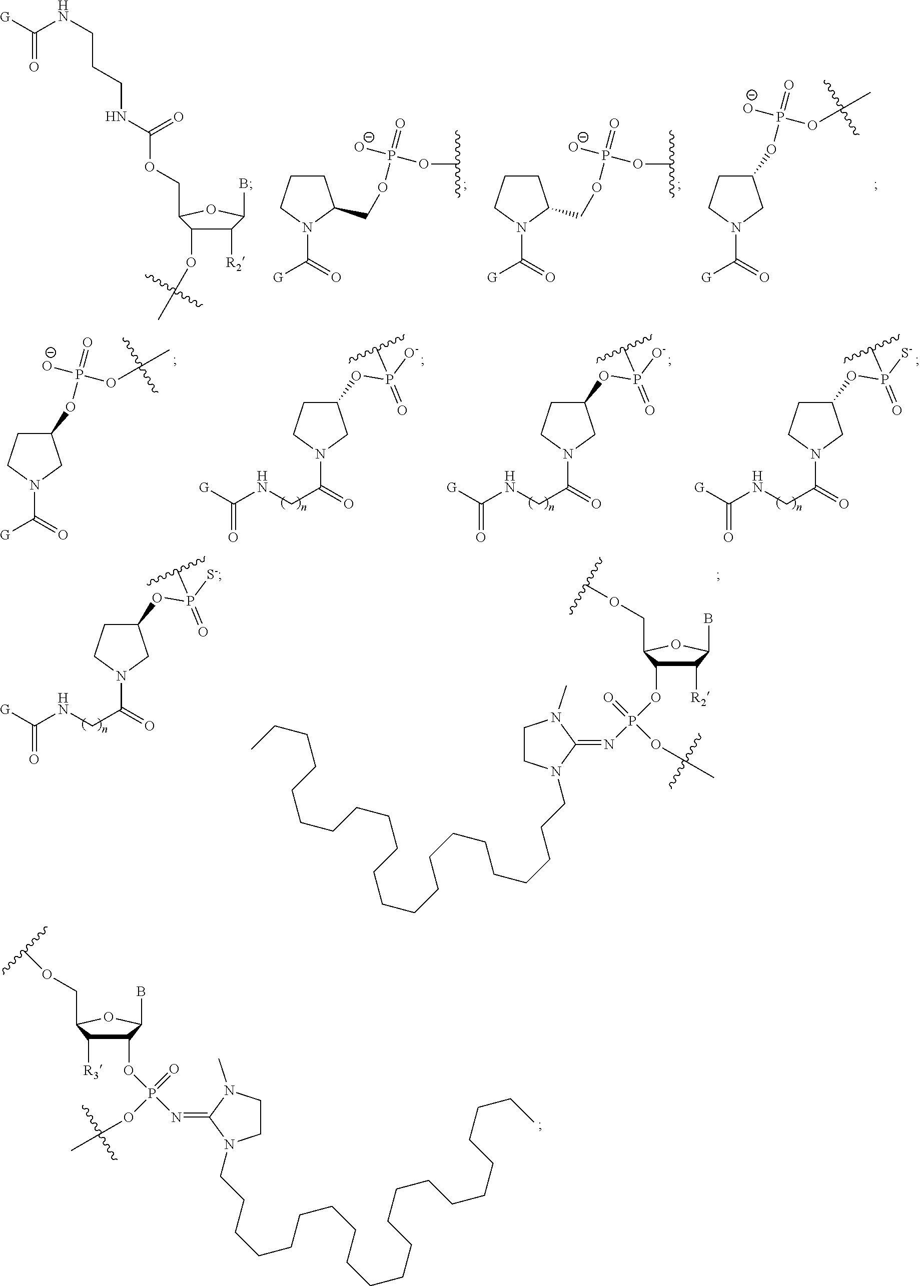

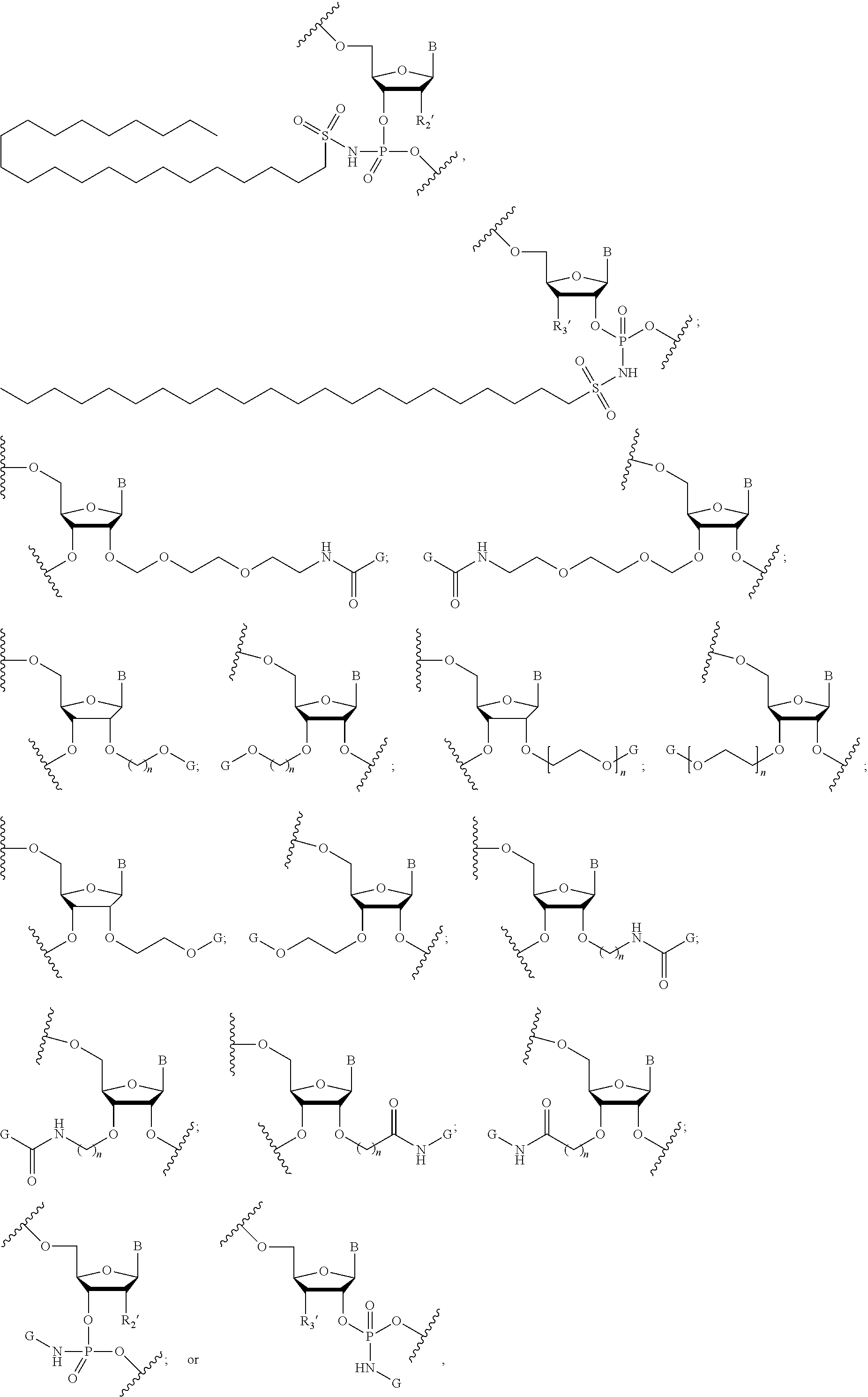

wherein:
B is an optionally modified nucleobase;
G is G₁ or a saturated or unsaturated C₂₁ hydrocarbon chain (i.e., G together with the carbonyl to which it is attached may form a group with 22 carbons) (for instance, G may be a linear or branched C₂₁ alkyl group), wherein G is optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl (for instance, G is optionally substituted with a —OR^{G}, —C(O)OR^{G}, or —N(R^{G})C(O)R^{G});
G₁ is a saturated or unsaturated C₂₂ hydrocarbon chain (for instance, G₁ may be a linear or branched C₂₂ alkyl group), wherein G¹ is optionally substituted with one or two groups selected from the group consisting of halogen, —OR^{G1}, —SR^{G1}, —N(R^{G1})₂, —C(O)OR^{G1}, —OC(O)R^{G1}, —C(O)N(R^{G1})₂, —N(R^{G1})C(O)R^{G1}, —N(R^{G1})C(O)OR^{G1}, —N(R^{G1})SO₂(R^{G1}), or —SO₂N(R^{G1})₂, wherein each R^{G1} is independently hydrogen or C₁-C₆ alkyl (for instance, G₁ is optionally substituted with a —OR^{G1}, —C(O)OR^{G1}, or —N(R^{G1})C(O)R^{G1});
integer m is 0-8 (for instance, m is 0; or m is 1-8; or m is 0-6; or m is 1; or 2; or 3; or 4; or 5; or 6; or 7; or 8);
integer n is 1-21 (for instance, 1-12, 1-10, 1-8, 1-6, 1-4, or 1-2; or 2 or 3 or 4 or 5 or 6);
W is an alkyl group such as a C₁-C₄ alkyl (e.g., methyl, ethyl, propyl, isopropyl, butyl, isobutyl, t-butyl);
R, R′, and R″ are each independently H or an alkyl group such as a C₁-C₄ alkyl (e.g., methyl, ethyl, propyl, isopropyl, t-butyl); and
R₂′ or R₃′ may be any functional group that is an acceptable 2′-modification for a ribose sugar. Examples of suitable R₂′ or R₃′groups include, but are not limited to, hydrogen, halogen (e.g., 2′-fluoro), hydroxy, 2′-O-alkyl (e.g., 2′-OMethyl), 2′-O-methoxyalkyl (e.g., 2′-O-methoxymethyl, 2′-O-methoxyethyl, or 2′-O-2-methoxypropanyl) modification, 2′-O-allyl modification, 2′-C-allyl modification, 2′-O—N-methylacetamido (2′-O-NMA, i.e. —OCH₂C(O)N(H)Me) modification, 2′-O-dimethylaminoethoxyethyl (2′-O-DMAEOE) modification, 2′-O-aminopropyl (2′-O-AP) modification, or 2′-ara-F modification. For instance, R₂′ or R₃′ may be H, OH, F, OMe, O-methoxyalkyl, O-allyl, O—N-methylacetamido, O-dimethylaminoethoxyethyl, or O-aminopropyl.

In some embodiments, the lipophilic monomer selected from the group consisting of

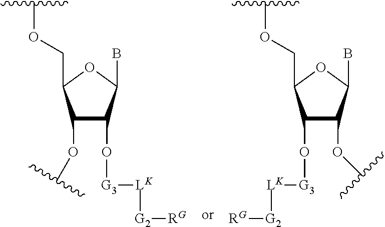

wherein.
B is an optionally modified nucleobase;
G₃ is a saturated or unsaturated C₁₋₂₀ hydrocarbon group (e.g., C₁₋₆ alkylene; C₂₋₆ alkylene; or hexylene);
L^{K} is a linking group such as —O—, —N(H)—, —S—, —S—S—, —C(O)O—, OC(O)—, —C(O)N(H)—, —N(H)C(O), —OC(O)N(H)—, —N(H)C(O)O—, —S(O)₂—, —S(O)₂O—, —S(O)₂N(H)—, —P(O)(OH)O—, —OP(O)(OH)—, —P(S)(OH)O—, —OP(S)(OH)—, —OP(O)(OH)O—, —OP(S)(OH)O—,
G₂ is a saturated or unsaturated C₂₁-C₂₂ hydrocarbon group; and
R^{G} is hydrogen, hydroxy, amino, —COOH, or —C(O)NH₂.
   For example, when L^{K} contains a carbonyl attached to G₂ (e.g., (—N(H)C(O)— or —OC(O)—), then G₂ is a C₂₁ hydrocarbon group; and when L^{K} does not contain a carbonyl attached to G₂, then G₂ is a C₂₂ hydrocarbon group. In one embodiment, R^{G} is hydrogen. In another embodiment, R^{G} is OH, In one embodiment, R^{G} is COOH. In another embodiment, R^{G} is CONH₂. In one embodiment, R^{G} is amino.

In the above structures for the lipophilic monomers, the monomers may also contain one or more asymmetric centers and thus occur as racemates and racemic mixtures, single enantiomers, individual diastereomers and diastereomeric mixtures. All such isomeric forms of the monomers are expressly included. Further, in the preceding and throughout the present application, where a modified internucleotide linkage is shown with substituent atoms fully described at the phosphorous atom, e.g.,

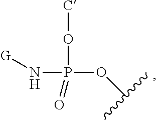

where C′ is the 2′-carbon or 3′-carbon atom of a ribose ring, it is understood that the oxygen having the broken bond is the 5′-oxygen of the subsequent nucleotide.

In the above structures for the lipophilic monomers, the alkylene chain can contain one or more unsaturated bonds.

Specific embodiments of the lipophilic monomers containing the saturated or unsaturated C₂₂ hydrocarbon chains include:

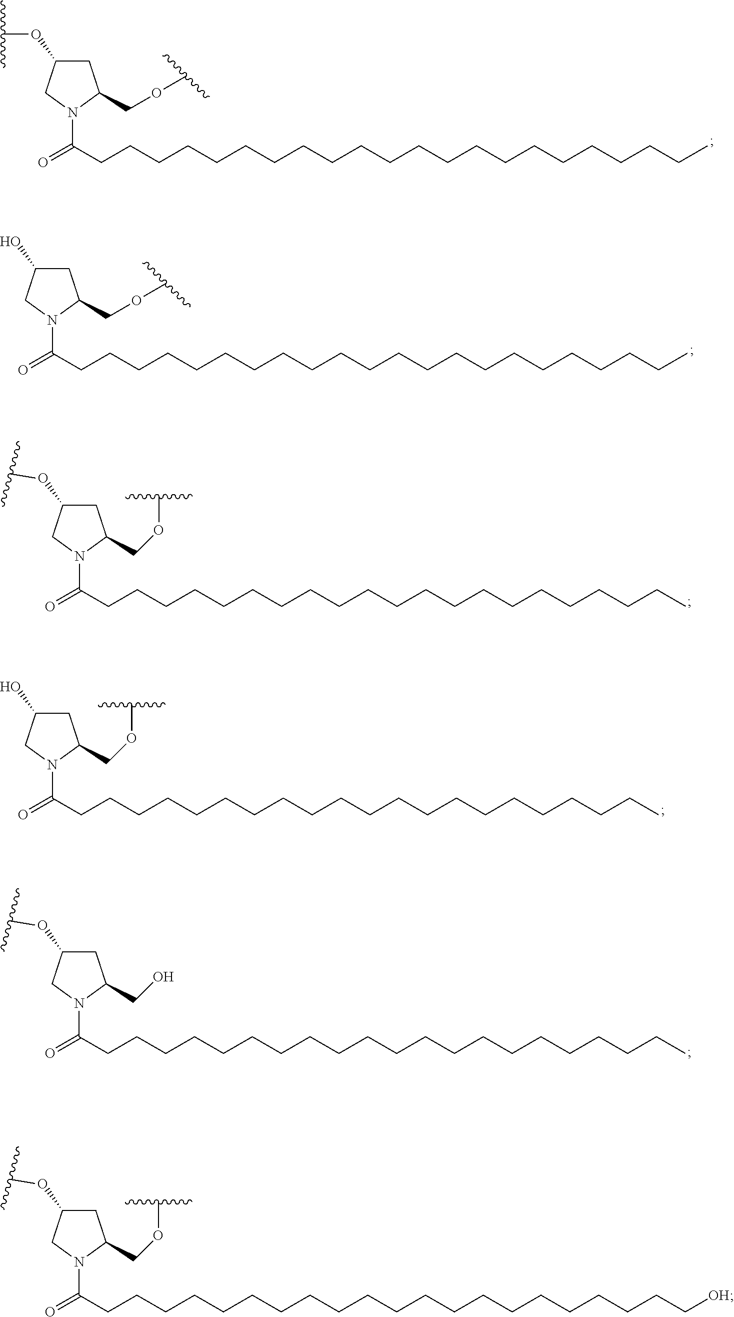 [SMILES: *OC[C@@H]1C[C@@H](O*)CN1C(=O)CCCCCCCCCCCCCCCCCCCCCC ; *OC[C@@H]1C[C@@H](O)CN1C(=O)CCCCCCCCCCCCCCCCCCCCCC ; *OC[C@@H]1C[C@@H](O*)CN1C(=O)CCCCCCCCCCCCCCCCCCCCC ; *OC[C@@H]1C[C@@H](O)CN1C(=O)CCCCCCCCCCCCCCCCCCCCC ; *O[C@@H]1C[C@@H](CO)N(C(=O)CCCCCCCCCCCCCCCCCCCCC)C1 ; *OC[C@@H]1C[C@@H](O*)CN1C(=O)CCCCCCCCCCCCCCCCCCCCCO]

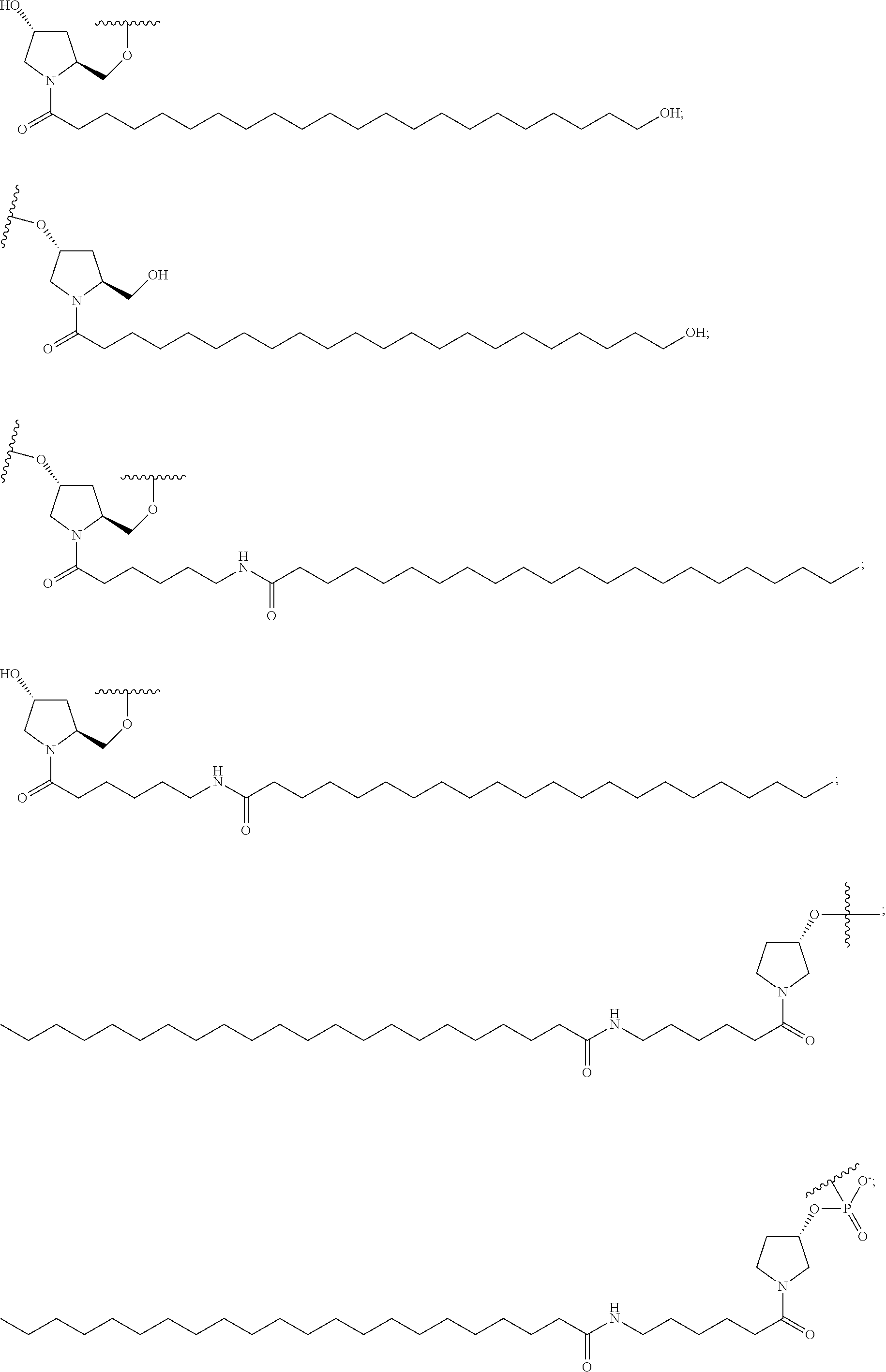

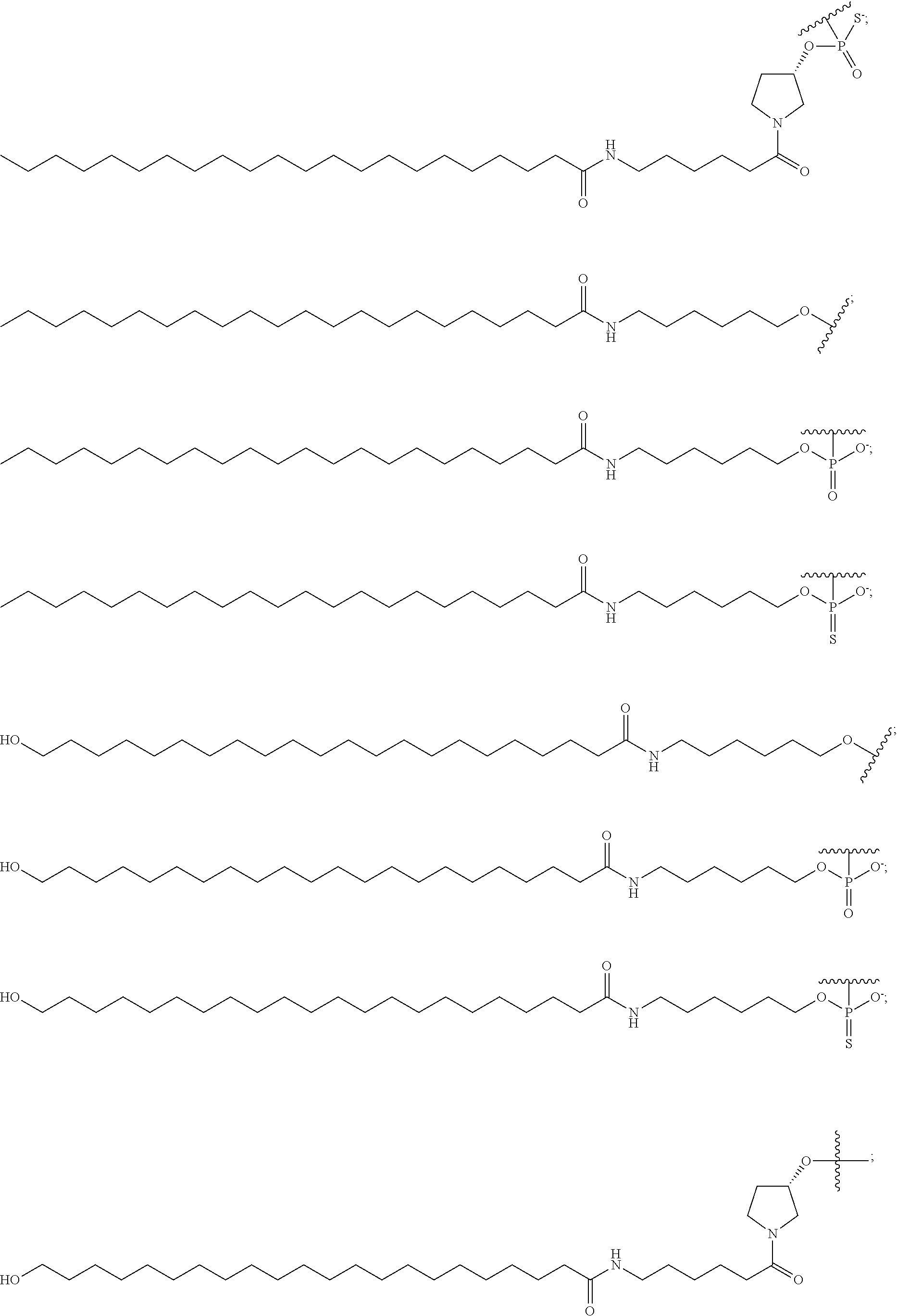

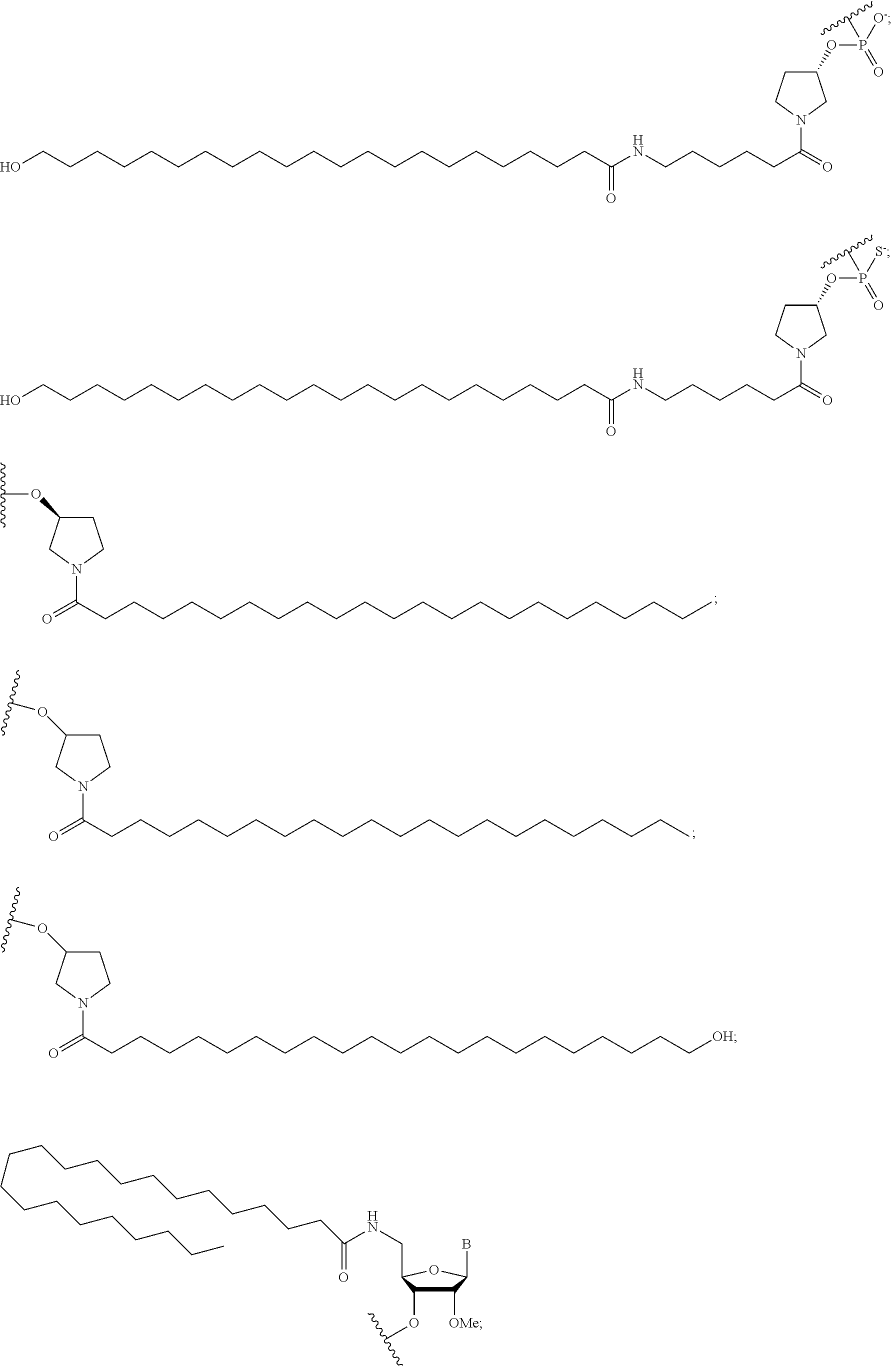

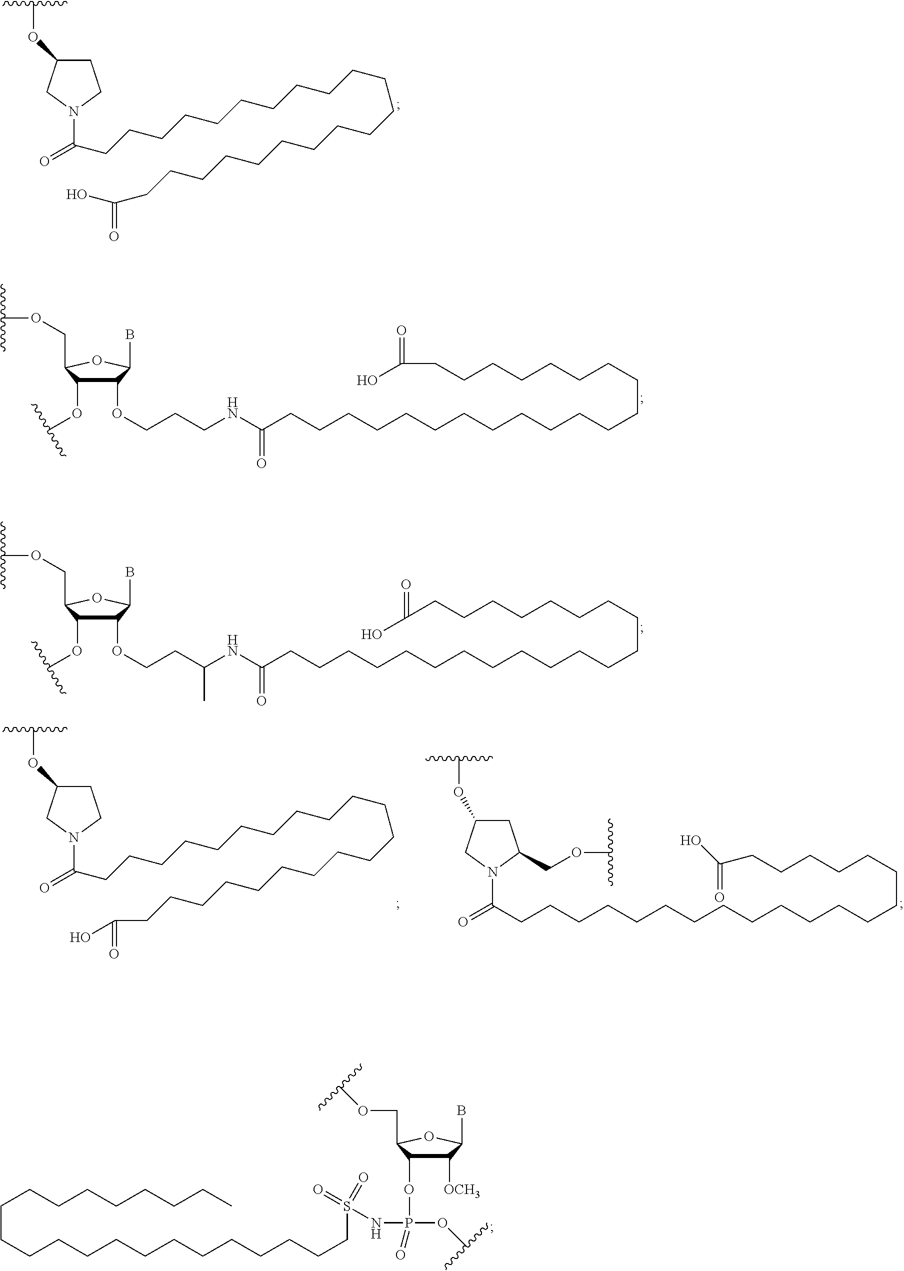

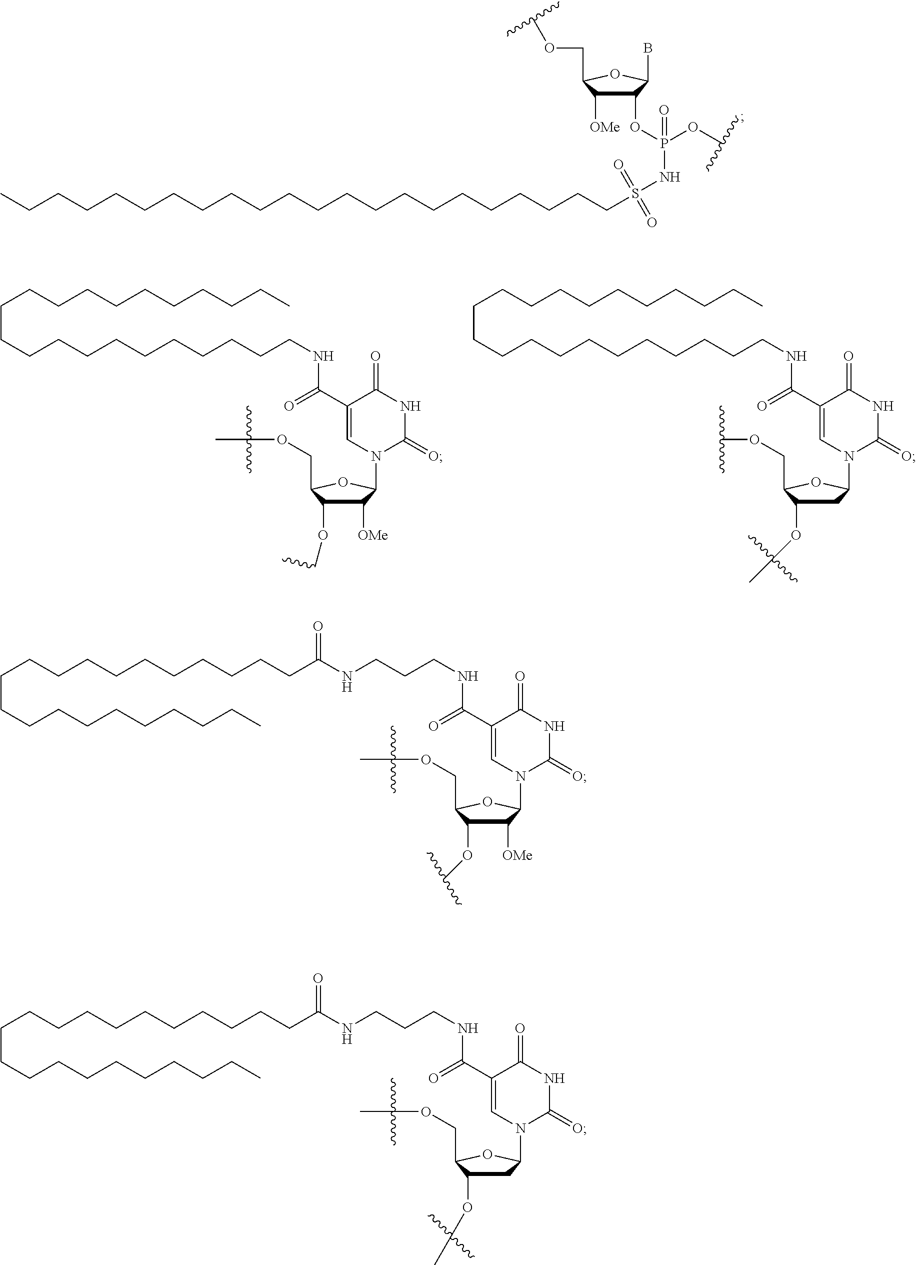

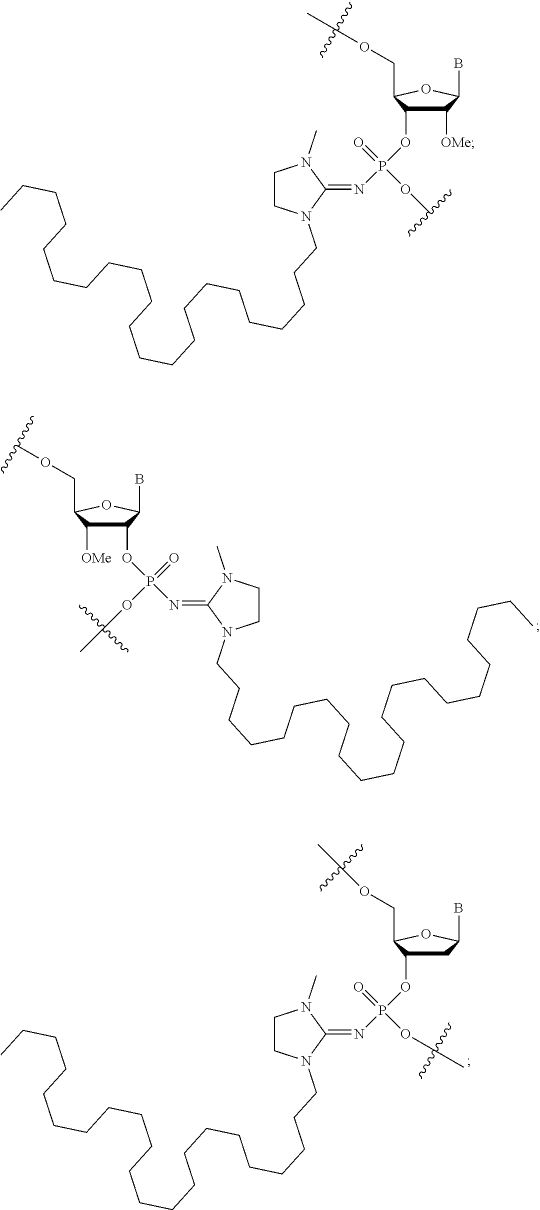

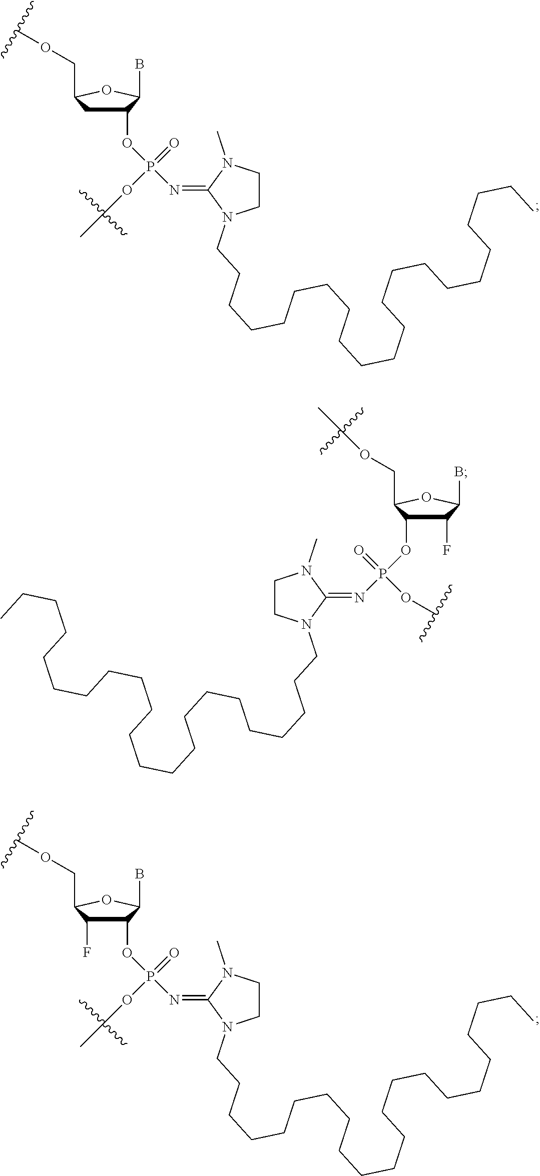

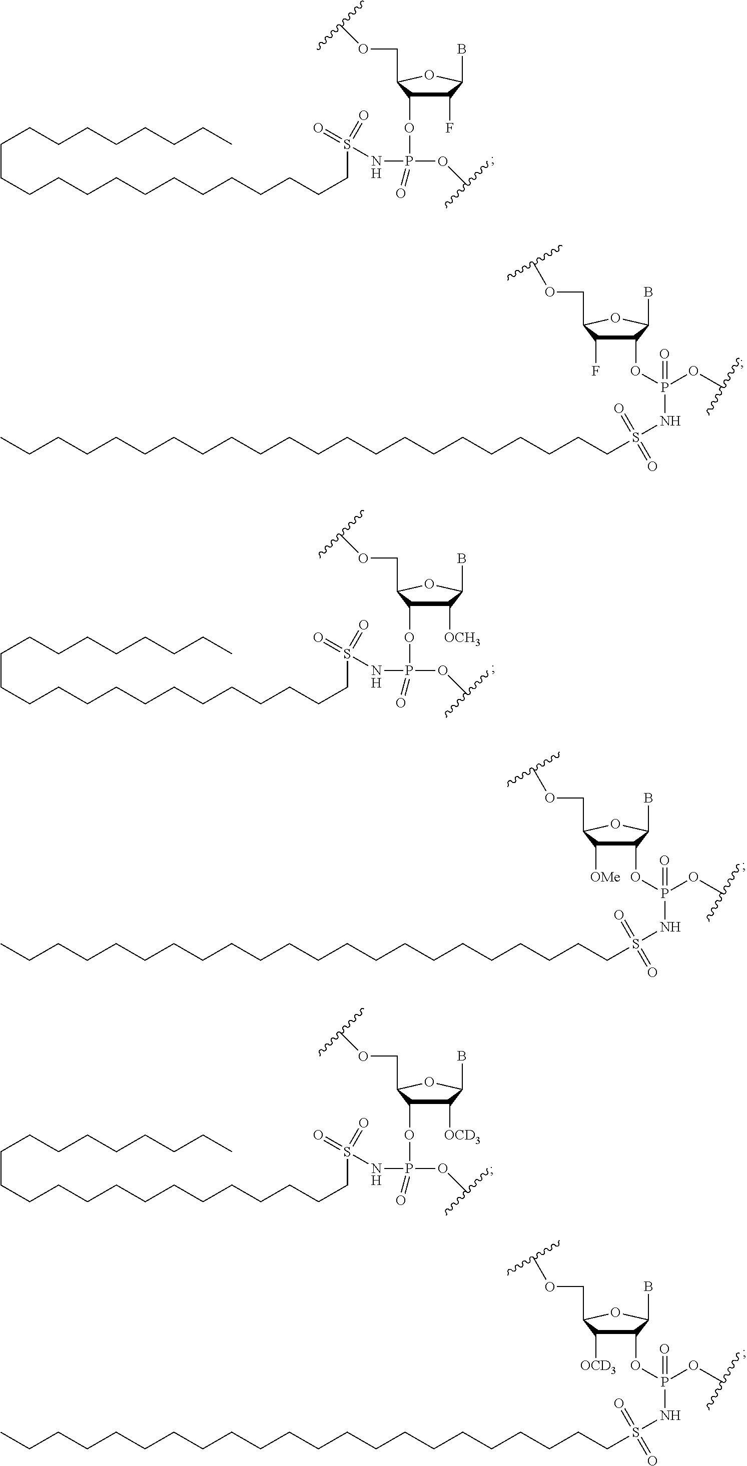

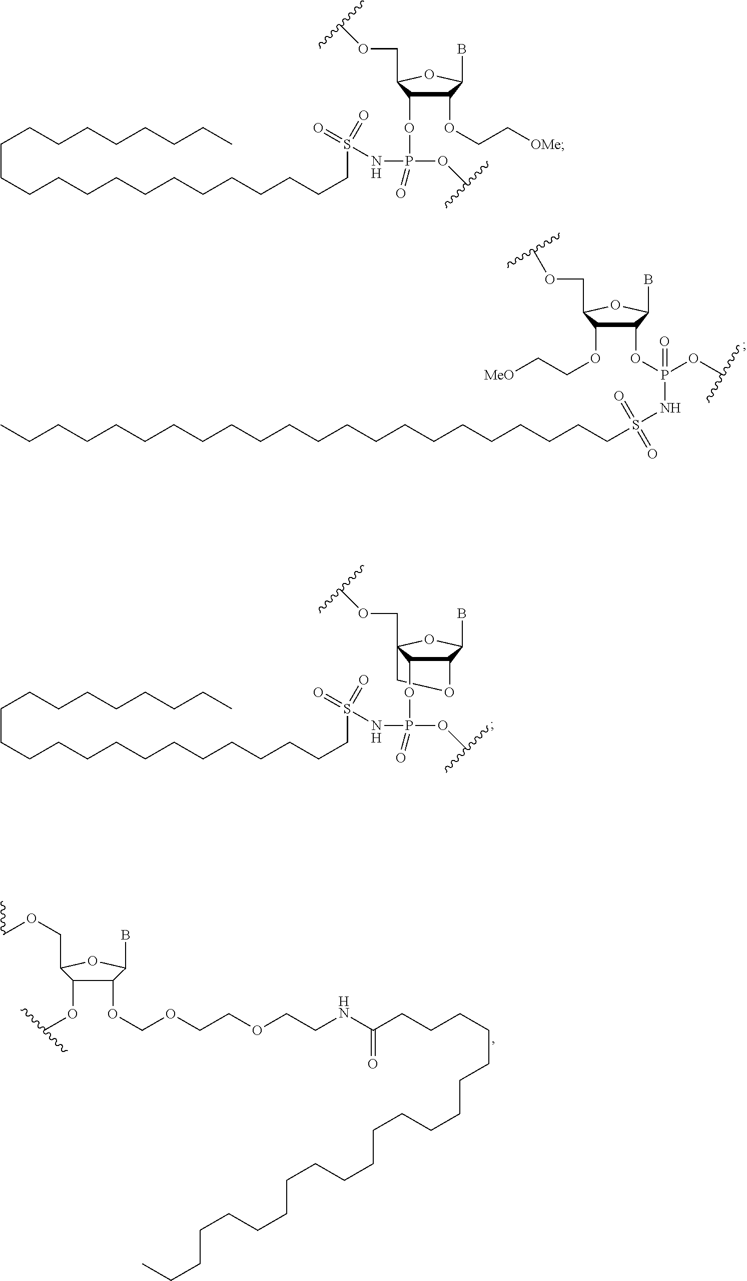

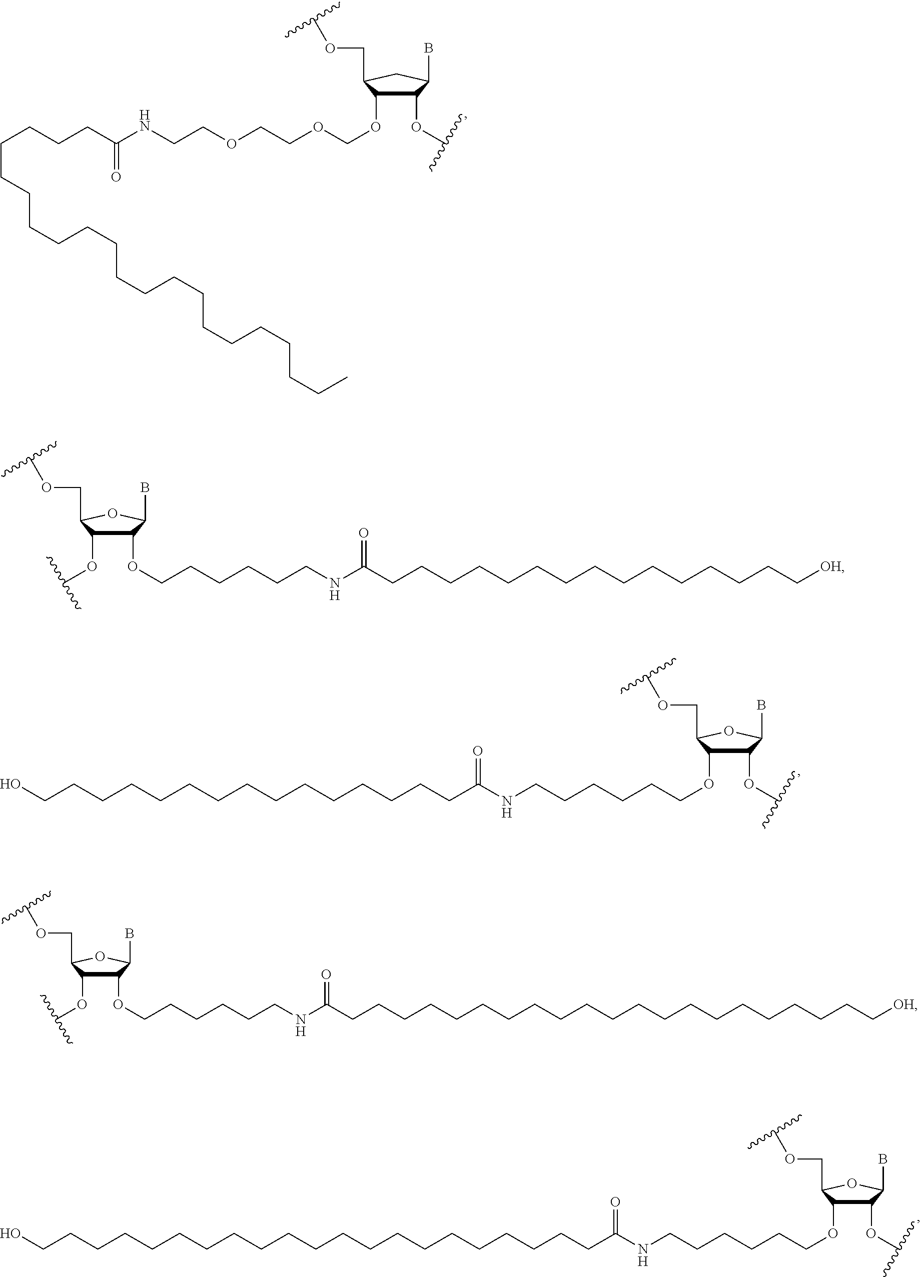

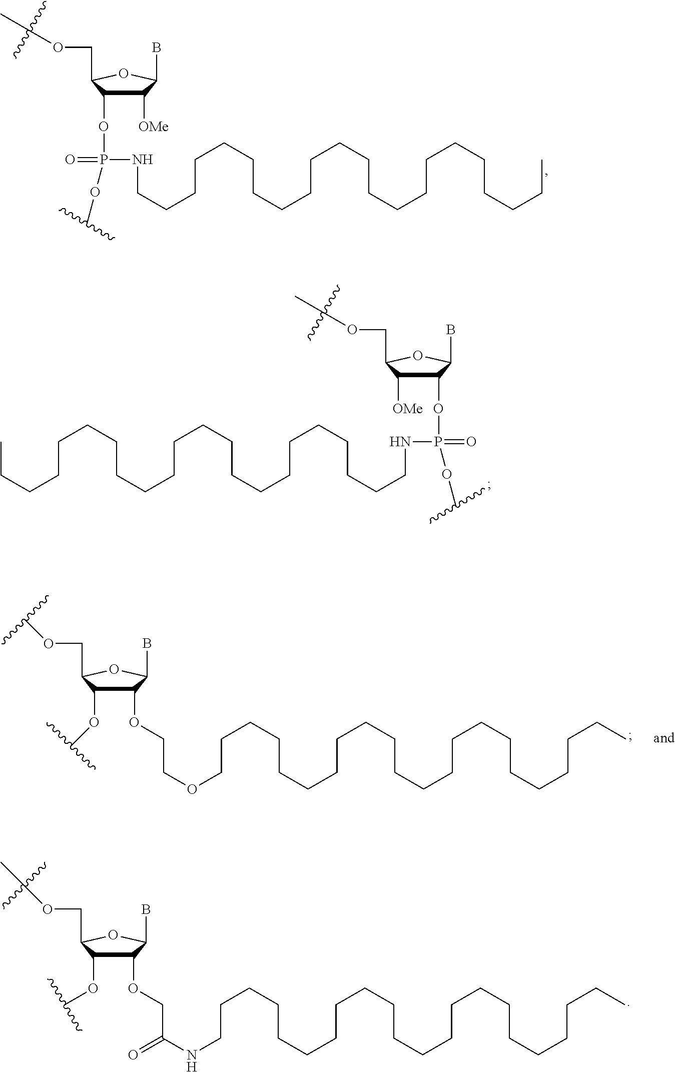

In these structures, B is a modified or unmodified nucleobase.

In one embodiment, the lipophilic monomer is:

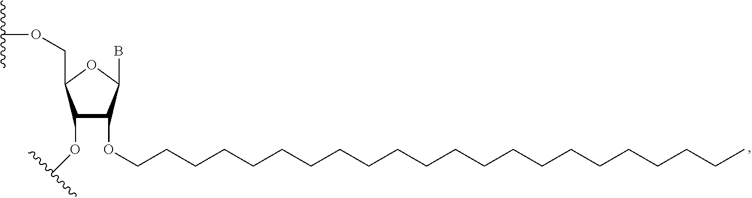

wherein B is a modified or unmodified nucleobase.

In one embodiment, the lipophilic monomer is:

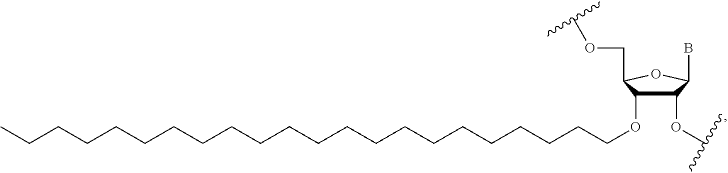

wherein B is a modified or unmodified nucleobase.

In one embodiment, the lipophilic monomer is:

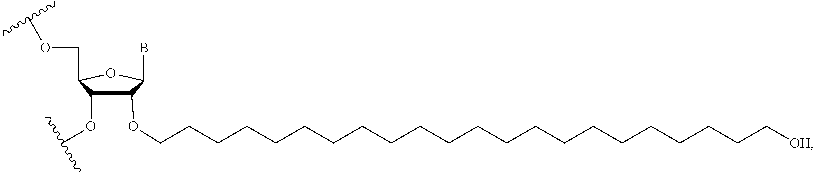

wherein B is a modified or unmodified nucleobase.

In one embodiment, the lipophilic monomer is:

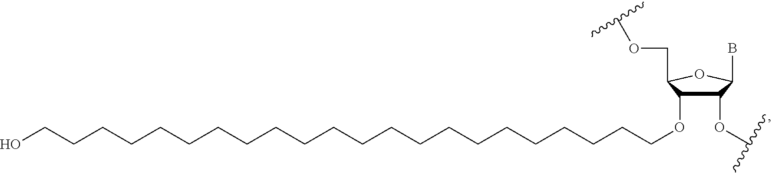

wherein B is a modified or unmodified nucleobase.

In some embodiments, the lipophilic monomer is:

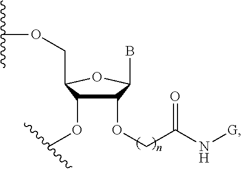

wherein n is an integer of 1-21, for instance, 1-12, 1-10, 1-8, 1-6, 1-4, or 1-2, or 2 or 3 or 4 or 5 or 6); G is a C₂₂ hydrocarbon chain, optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl; and nucleobase B is a modified or unmodified nucleobase. In one embodiment, n is 1. In one embodiment, n is 2-6. In one embodiment, n is 6. In one embodiment, G is C₂₂ alkyl chain.

In some embodiments, the lipophilic monomer is:

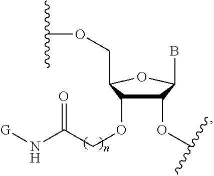

wherein n is an integer of 1-21, for instance, 1-12, 1-10, 1-8, 1-6, 1-4, or 1-2, or 2 or 3 or 4 or 5 or 6); G is a C₂₂ hydrocarbon chain, optionally substituted with one or two groups selected from the group consisting of halogen, —OR^{G}, —SR^{G}, —N(R^{G})₂, —C(O)OR^{G}, —OC(O)R^{G}, —C(O)N(R^{G}) 2, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl; and nucleobase B is a modified or unmodified nucleobase. In one embodiment, n is 1. In one embodiment, n is 2-6. In one embodiment, n is 6. In one embodiment, G is C₂₂ alkyl chain.

In some embodiments, the lipophilic monomer is:

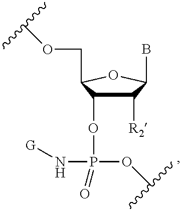

wherein R₂′ is H, OH, F, Ome, O-methoxyalkyl, O-allyl, O—N-methylacetamido; and nucleobase B is a modified or unmodified nucleobase. In one embodiment, R₂′ is H, OH, F, Ome, or O-methoxyalkyl. In one embodiment, G is C₂₂ alkyl chain.

In some embodiments, the lipophilic monomer is:

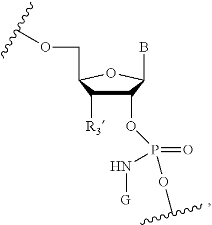

wherein R₃′ is H, OH, F, Ome, O-methoxyalkyl, O-allyl, O—N-methylacetamido; and nucleobase B is a modified or unmodified nucleobase. In one embodiment, R₃′ is H, OH, F, Ome, or O-methoxyalkyl. In one embodiment, G is C₂₂ alkyl chain.

In one embodiment, the lipophilic monomer is:

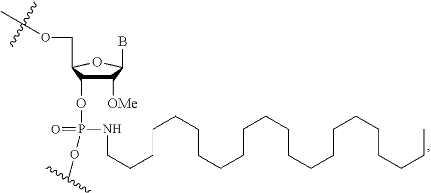

wherein B is a modified or unmodified nucleobase.

In one embodiment, the lipophilic monomer is:

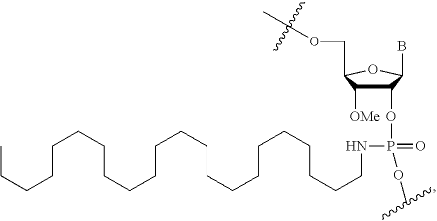

wherein B is a modified or unmodified nucleobase.

In one embodiment, the lipophilic monomer is:

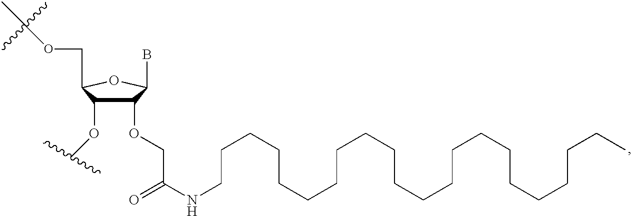

wherein B is a modified or unmodified nucleobase.

In one embodiment, the lipophilic monomer is:

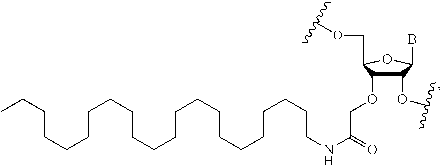

wherein B is a modified or unmodified nucleobase.

In one embodiment, the lipophilic monomer is:

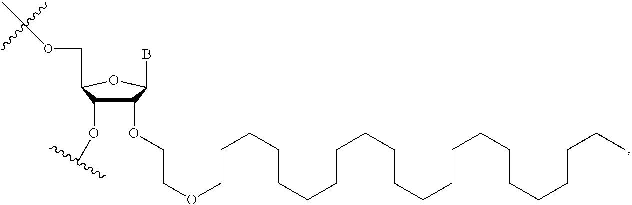

wherein B is a modified or unmodified nucleobase.

In one embodiment, the lipophilic monomer is:

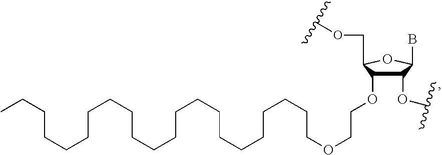

wherein B is a modified or unmodified nucleobase.

In one embodiment, the lipophilic monomer is:

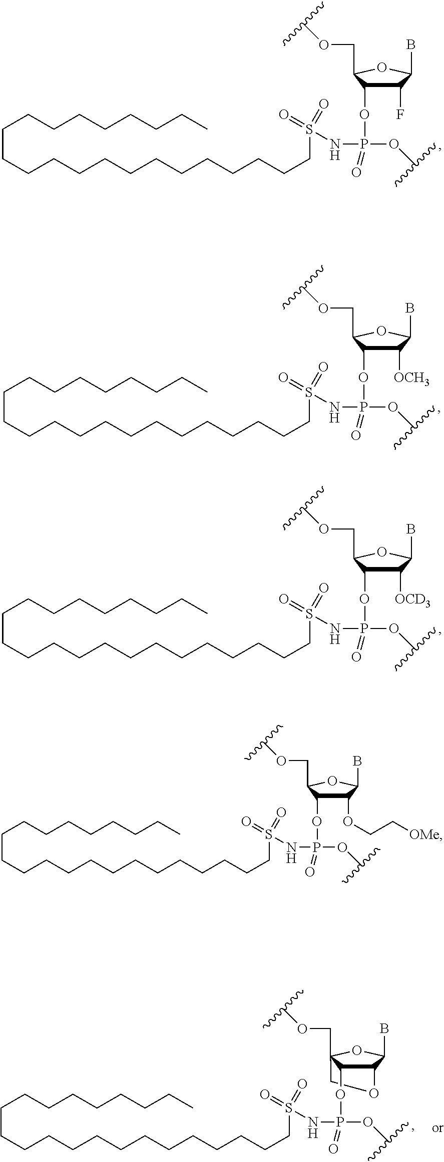

wherein B is a modified or unmodified nucleobase, wherein the phosphorous atom in the internucleotide linkage is optionally enriched in the Sp or Rp isomer, or is racemic.

In one embodiment, the lipophilic monomer is

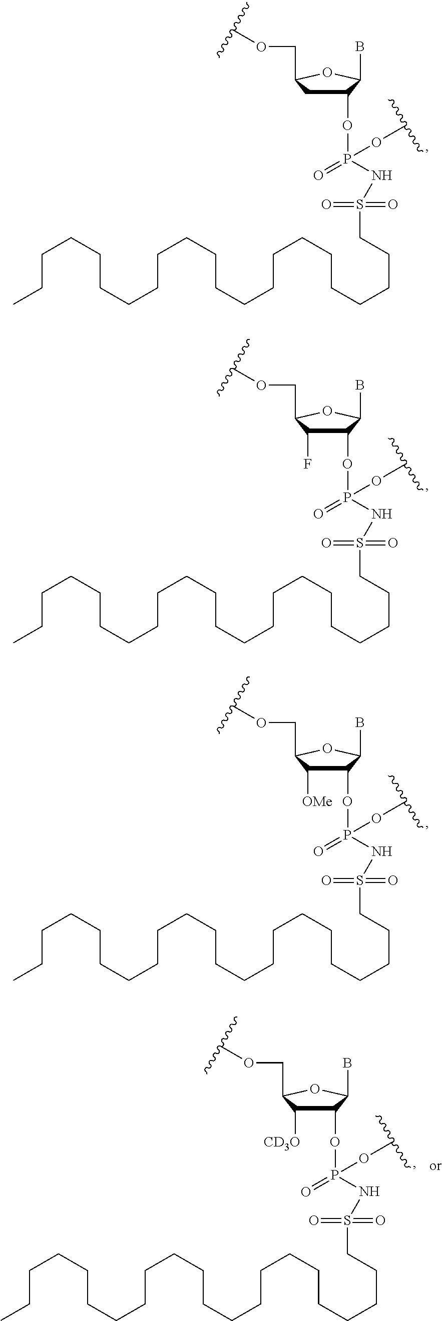

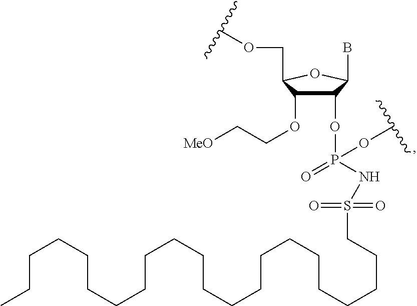

wherein B is a modified or unmodified nucleobase, wherein the phosphorous atom in the internucleotide linkage is optionally enriched in the Sp or Rp isomer, or is racemic.

In some embodiments, the lipophilic moiety containing one or more saturated or unsaturated C₂₂ hydrocarbon chains is a lipophilic monomer selected from one of the members of group (i), group (ii), and group (iii):

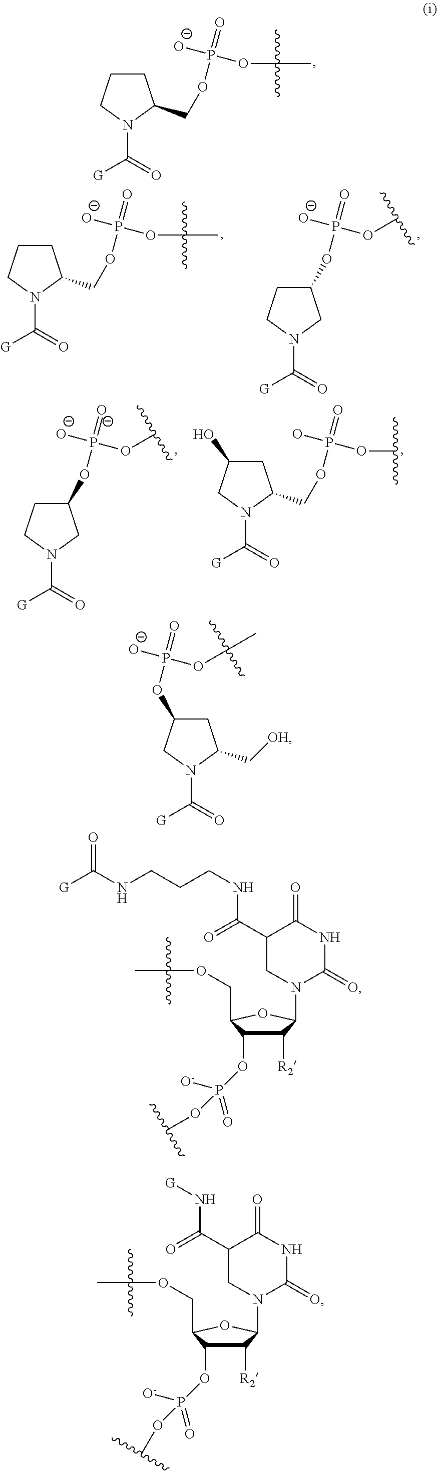

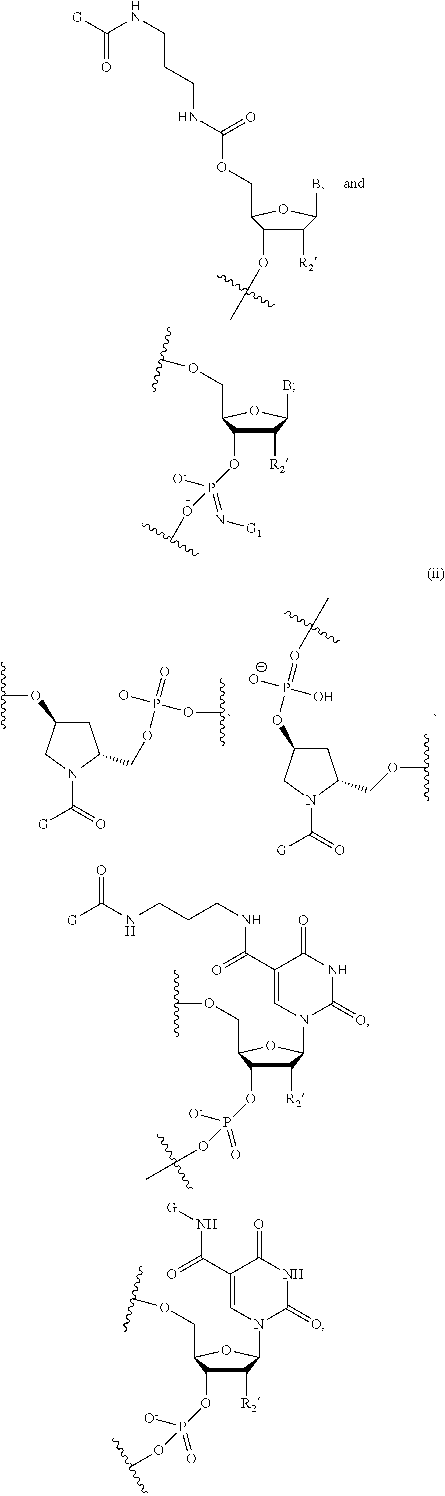

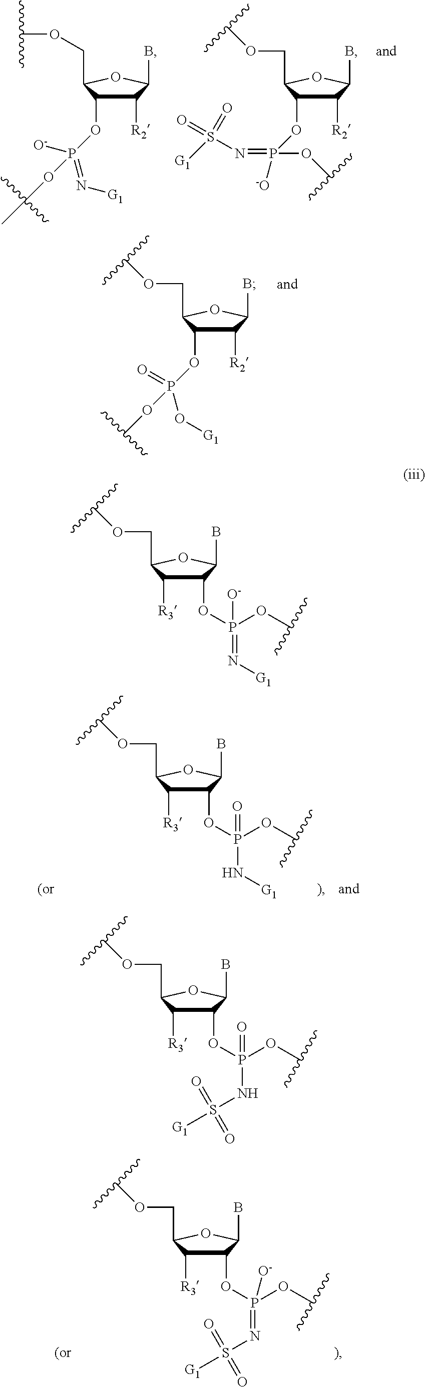

wherein:
B is an optionally modified nucleobase;
G is G₁ or a saturated or unsaturated C21 hydrocarbon chain (i.e., G together with the carbonyl to which it is attached may form a group with 22 carbons), wherein G is optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl (for instance, G is optionally substituted with a —OR^{G}, —C(O)OR^{G}, or —N(R^{G})C(O)R^{G});
G₁ is a saturated or unsaturated C22 hydrocarbon chain, wherein G¹ is optionally substituted with one or two groups selected from the group consisting of halogen, —OR^{G1}, —SR^{G1}, —N(R^{G1})₂, —C(O)OR^{G1}, —OC(O)R^{G1}, —C(O)N(R^{G1})₂, —N(R^{G1})C(O)R^{G1}, —N(R^{G1})C(O)OR^{G1}, —N(R^{G1})SO₂(R^{G1}), or —SO₂N(R^{G1})₂, wherein each R^{G1} is independently hydrogen or C₁-C₆ alkyl (for instance, G₁ is optionally substituted with a —OR^{G1}, —C(O)OR^{G1}, or —N(R^{G1})C(O)R^{G1}); and
R₂′ or R₃′ may be any functional group that is an acceptable 2′-modification for a ribose sugar. Examples of suitable R₂′ or R₃′ groups include, but are not limited to, hydrogen, halogen (e.g., 2′-fluoro), hydroxy, 2′-O-alkyl (e.g., 2′-OMethyl), 2′-O-methoxyalkyl (e.g., 2′-O-methoxymethyl, 2′-O-methoxyethyl, or 2′-O-2-methoxypropanyl) modification, 2′-O-allyl modification, 2′-C-allyl modification, 2′-O—N-methylacetamido (2′-O-NMA, i.e., —OCH₂C(O)N(H)Me) modification, 2′-O-dimethylaminoethoxyethyl (2′-O-DMAEOE) modification, 2′-O-aminopropyl (2′-O-AP) modification, or 2′-ara-F modification. For instance, R₂′ or R₃′ may be H, OH, F, OMe, O-methoxyalkyl, O-allyl, O—N-methylacetamido, O-dimethylaminoethoxyethyl, or O-aminopropyl.

In some embodiments, the lipophilic moiety containing one or more saturated or unsaturated C₂₂ hydrocarbon chains is a lipophilic monomer selected from one of the members of group (i′), group (ii′), and group (iii′):

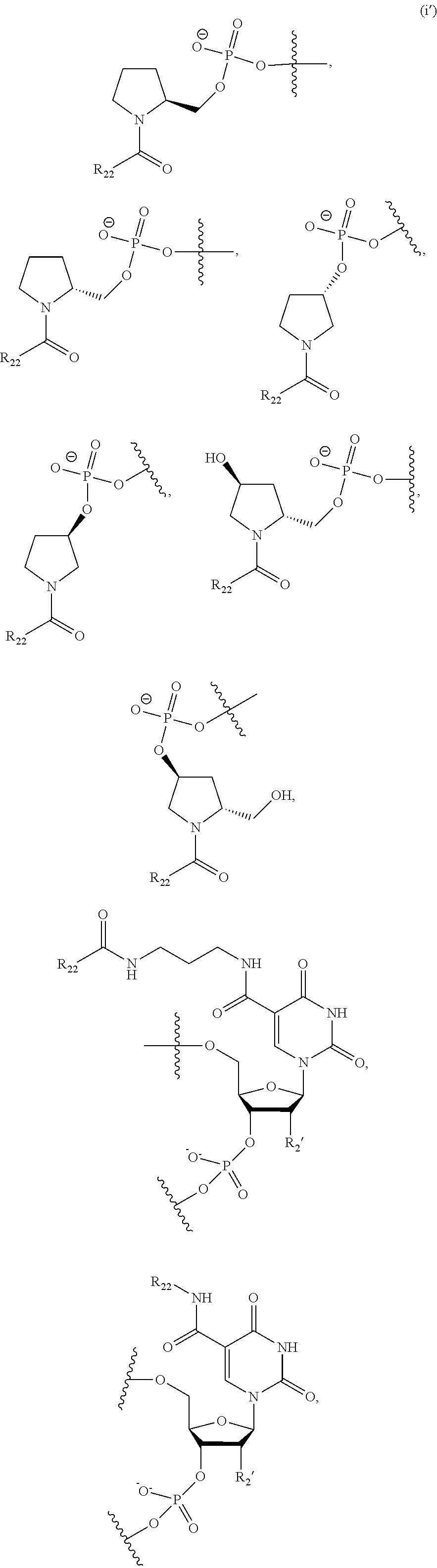

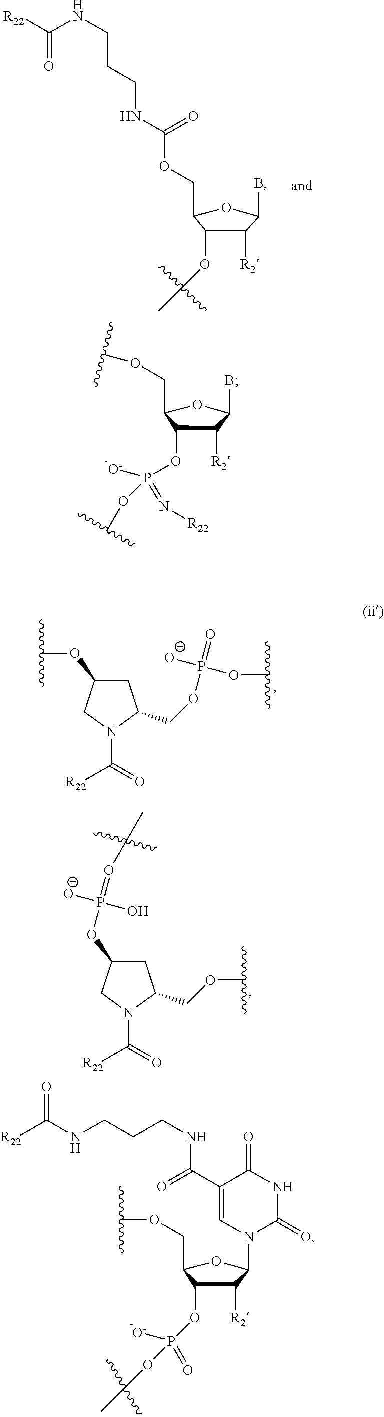

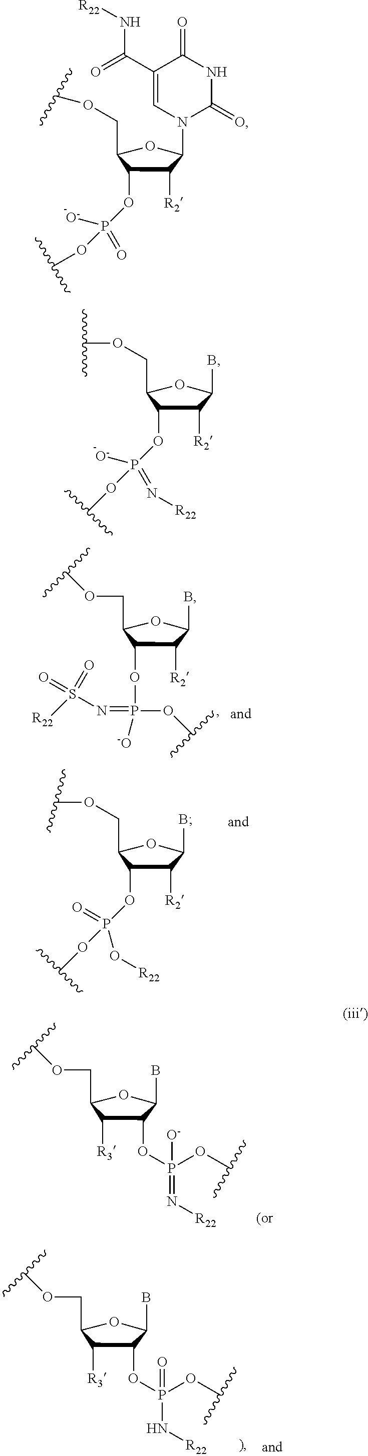

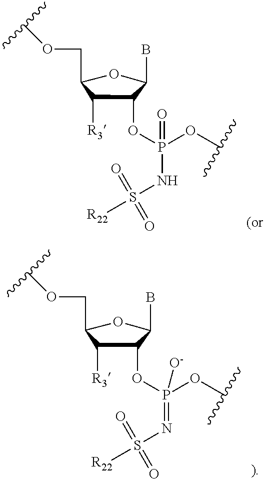

In these embodiments, B is an optionally modified nucleobase. R₂₂ is a saturated or unsaturated C₂₂ hydrocarbon chain. For instance, R₂₂ is a linear or branched C₂₂ alkyl group. R₂′ may be any functional group that is an acceptable 2′-modification for a ribose sugar. Examples of suitable R₂′ or R₃′ groups include, but are not limited to, hydrogen, halogen (e.g., 2′-fluoro), hydroxy, 2′-O-alkyl (e.g., 2′-OMethyl), 2′-O-methoxyalkyl (e.g., 2′-O-methoxymethyl, 2′-O-methoxyethyl, or 2′-O-2-methoxypropanyl) modification, 2′-O-allyl modification, 2′-C-allyl modification, 2′-O—N-methylacetamido (2′-O-NMA, i.e. —OCH₂C(O)N(H)Me) modification, 2′-O-dimethylaminoethoxyethyl (2′-O-DMAEOE) modification, 2′-O-aminopropyl (2′-O-AP) modification, or 2′-ara-F modification. For instance, R₂′ or R₃′ is H, OH, F, OMe, O-methoxyalkyl, O-allyl, O—N-methylacetamido, O-dimethylaminoethoxyethyl, or O-aminopropyl. B is a modified or unmodified nucleobase.

In some embodiments, the lipophilic moiety is conjugated to the 3′-end or 5′-end of one of the sense and antisense strands via a direct bond or through a carrier or linker. In some embodiments, the lipophilic moiety is conjugated to the 3′-end of the sense or antisense strand via a direct bond or through a carrier or linker. In some embodiments, the lipophilic moiety is conjugated to the 5′-end of the sense or antisense strand via a direct bond or through a carrier or linker.

In some embodiments, the lipophilic moiety is of the formula

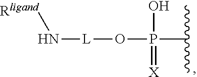

or a salt thereof, wherein X is O or S (e.g., S); L is a divalent linking group (e.g., C₁₋₂₀ alkyl or C₁₋₁₀ alkyl-S—S—C₁₋₁₀ alkyl). In one embodiment, the lipophilic moiety is of the formula

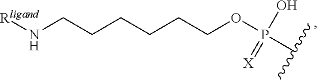

or a salt thereof, wherein X is O or S (e.g., S). In these embodiments, R^{ligand} is selected from the groups listed in Table R-1.

TABLE R-1
An exemplary list of groups for R^{ligand}

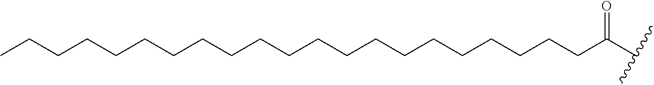 [SMILES: *C(=O)CCCCCCCCCCCCCCCCCCCCC]

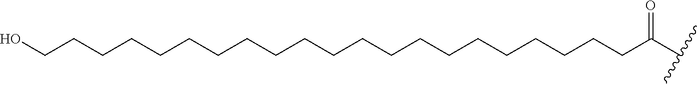 [SMILES: *C(=O)CCCCCCCCCCCCCCCCCCCCCO]

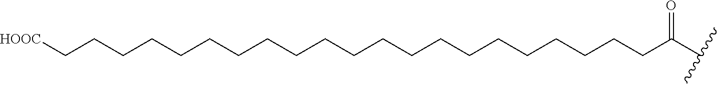 [SMILES: *C(=O)CCCCCCCCCCCCCCCCCCCCCC(=O)O]

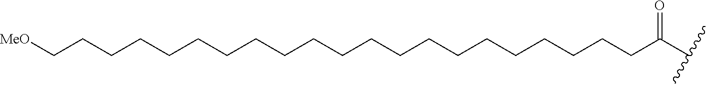 [SMILES: *C(=O)CCCCCCCCCCCCCCCCCCCCCOC]

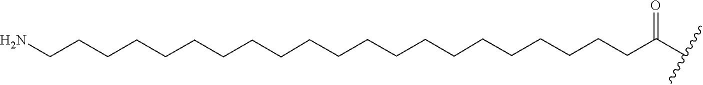 [SMILES: *C(=O)CCCCCCCCCCCCCCCCCCCCCN]

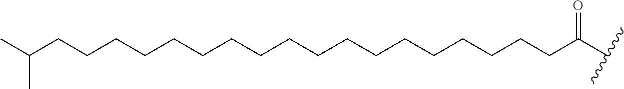 [SMILES: *C(=O)CCCCCCCCCCCCCCCCCCC(C)C]

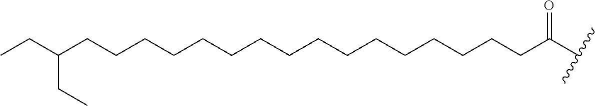 [SMILES: *C(=O)CCCCCCCCCCCCCCCCC(CC)CC]

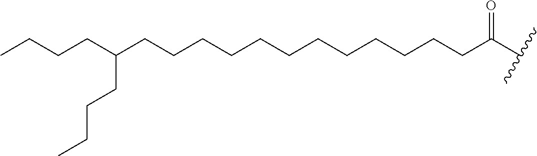 [SMILES: *C(=O)CCCCCCCCCCCCC(CCCC)CCCC]

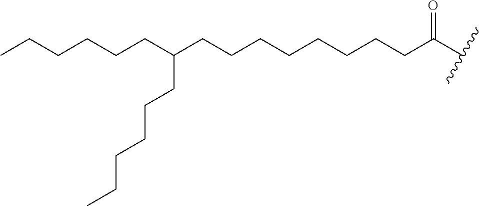 [SMILES: *C(=O)CCCCCCCCC(CCCCCC)CCCCCC]

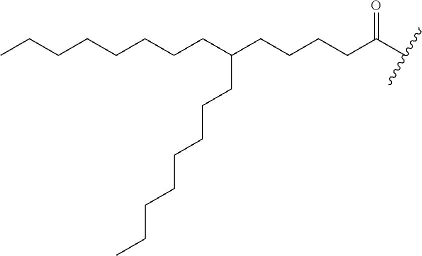 [SMILES: *C(=O)CCCCC(CCCCCCCC)CCCCCCCC]

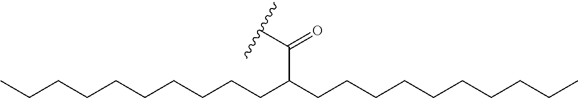 [SMILES: *C(=O)C(CCCCCCCCCC)CCCCCCCCCC]

In one embodiment, the lipophilic moiety is bonded to the 5′-oxygen of the 5′-terminal nucleotide or the 3′-oxygen of the 3′-terminal nucleotide, and is of the formula

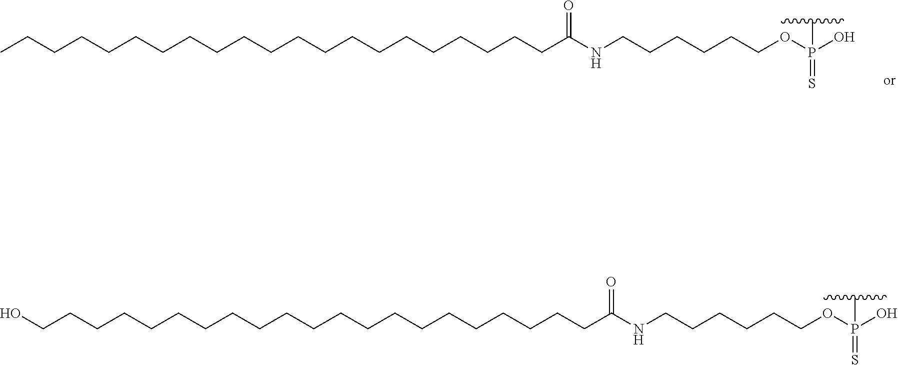 [SMILES: *P(O)(=S)OCCCCCCNC(=O)CCCCCCCCCCCCCCCCCCCCC ; *P(O)(=S)OCCCCCCNC(=O)CCCCCCCCCCCCCCCCCCCCCO]

In some embodiments, the lipophilic moiety is conjugated to the 3′-end or 5′-end of one of the sense and antisense strands via a carrier or linker, and the carrier or linker is an inverted abasic nucleotide, such as an inverted abasic deoxyribonucleotide or an inverted abasic ribonucleotide, each connected to the remainder of the oligonucleotide via a phosphodiester (PO) or phosphorothioate (PS) linkage. Examples include, but are not limited to,

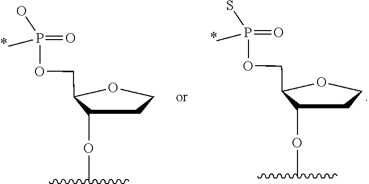

In some embodiments, the lipophilic moiety is bonded to the 5′-oxygen of the 5′-terminal nucleotide or the 3′-oxygen of the 3′-terminal nucleotide, and is of the formula

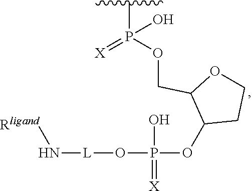

or a salt thereof, wherein each X is independently O or S (e.g., each is S); R^{ligand} is selected from the groups listed in Table R-1; and L is a divalent linking group (e.g., C₁₋₂₀ alkyl or C₁₋₁₀ alkyl-S—S—C₁₋₁₀ alkyl).

For example, the lipophilic moiety is bonded to the 5′-oxygen of the 5′-terminal nucleotide or the 3′-oxygen of the 3′-terminal nucleotide, and is of the formula

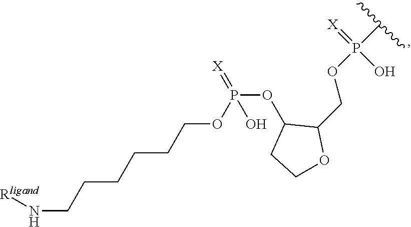

or a salt thereof, wherein each X is O or S (e.g., each is S) and R^{ligand} is selected from the groups listed in Table R-1. In some embodiments, R^{ligand} is selected from the groups listed in Table R-2.

TABLE R-2
An exemplary list of groups for R^{ligand}

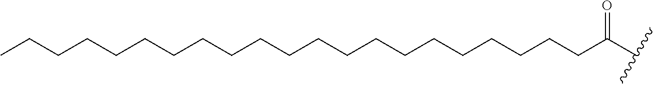 [SMILES: *C(=O)CCCCCCCCCCCCCCCCCCCCC]

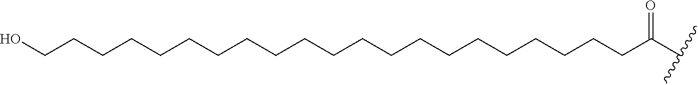 [SMILES: *C(=O)CCCCCCCCCCCCCCCCCCCCCO]

In some embodiments, the lipophilic moiety is bonded to the 5′-oxygen of the 5′-terminal nucleotide or the 3′-oxygen of the 3′-terminal nucleotide, and is of the formula

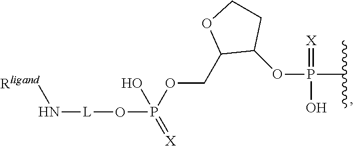

or a salt thereof, wherein each X is independently O or S (e.g., each is S) and R^{ligand} is selected from the groups listed in Table R-1, and L is a divalent linking group (e.g., C₁₋₂₀ alkyl or C₁₋₁₀ alkyl-S—S—C₁₋₁₀ alkyl.

In one embodiment, the lipophilic moiety is bonded to the 5′-oxygen of the 5′-terminal nucleotide or the 3′-oxygen of the 3′-terminal nucleotide, and is of the formula

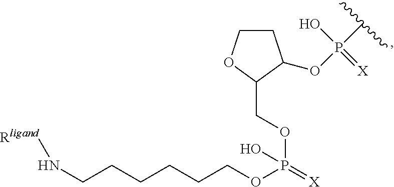

or a salt thereof, wherein each X is O or S (e.g., each is S) and R^{ligand} is selected from the groups listed in Table R-1. In some embodiments, R^{ligand} is selected from the groups listed in Table R-2.

In some embodiments, the dsRNA agent comprises a double-stranded region formed between the sense and antisense strands and optionally one or two single-stranded non-loop overhang, and wherein the one or more lipophilic moieties are conjugated to either the double-stranded region or the non-loop overhang. In some embodiments, the dsRNA agent does not contain a loop (e.g., stem loop) region. In some embodiments, the dsRNA agent contains a loop (e.g., stem loop) region, and the one or more lipophilic moieties are not conjugated to the loop (e.g., stem loop) region.

In some embodiments, the dsRNA agent comprises a sense strand of 10 to 53 nucleotides in length, in which the sense strand forms a duplex region with the antisense strand. For instance, the sense strand may be 10 to 49, 12 to 49, 12 to 45, 12 to 42, 12 to 40, 15 to 49, 15 to 45, 15 to 42, 15 to 40, 15 to 38, or 15 to 36 nucleotides in length. In some embodiments, the duplex region is at least 15, at least 16, at least 17, at least 18, at least 19, at least 20, or at least 21 nucleotides in length. In some embodiments, the region of complementarity to the target sequence is at least 19 contiguous nucleotides in length.

In some embodiments, the sense strand comprises at its 3′-end a stem-loop set forth as: S₁-L-S₂, in which S₁ is complementary to S₂, and in which L forms a loop between S₁ and S₂.

In some embodiments, the first 17 to 25 nucleotides counting from 5′ end of the sense strand forms a duplex region with the antisense strand, and the last 11 to 28 counting from 5′ end of the sense strand forms a 3′-end a stem-loop set forth as: S₁-L-S₂.

In some embodiments, the length of the stem loop S₁-L-S₂ is 11 to 28, 13 to 26, or 15 to 24 nucleotides in length. In one embodiment, the stem loop S₁-L-S₂ is 16 nucleotides in length. In some embodiments, the stem loop S₁-L-S₂ comprises a sequence of GCAGCCGAAAGGCUGC (SEQ ID NO: 1).

In some embodiments, L is at least 3, 4, or 5 nucleotides in length. In some embodiments, L comprises a sequence of GAAA.

In some embodiments, the sense strand is 36 nucleotides in length, the first 20 nucleotide counting from 5′ end of the sense strand forms a duplex region with the antisense strand, and the last 16 nucleotides forms a stem loop S₁-L-S₂. In one embodiment, the 16-nucleotide stem loop S₁-L-S₂ has the sequence of GCAGCCGAAAGGCUGC (SEQ ID NO: 2), wherein L is GAAA.

In some embodiments, the one or more lipophilic moieties are conjugated to a non-terminal position of the sense strand.

In some embodiments, the one or more lipophilic moieties are conjugated to one or more nucleotides of the stem loop S₁-L-S₂. In some embodiments, the one or more lipophilic moieties are conjugated to one or more nucleotides of the loop L.

In some embodiments, S₁ and S₂ are complementary and contain 4-10 nucleotides, e.g., S₁ and S₂ each contain 6 complementary nucleotides.

In some embodiments, S₁ and S₂ are complementary and contain 4-10 nucleotides and L is GAAA, e.g., S₁ and S₂ each contain 6 complementary nucleotides and L is GAAA.

In some embodiments, the one or more lipophilic moieties containing one or more saturated or unsaturated C₂₂ hydrocarbon chains are conjugated to one or more internal positions on at least one strand of the dsRNA agent.

In some embodiments, the internal positions include all positions except three terminal positions from each end of the at least one strand. In some embodiments, the internal positions include all positions except two terminal positions from each end of the at least one strand.

In some embodiments, the internal positions exclude a cleavage site region of the sense strand. In some embodiments, the internal positions exclude positions 9-11, positions 9-12, or positions 11-13, counting from the 5′-end of the sense strand.

In some embodiments, the internal positions exclude a cleavage site region of the antisense strand. In some embodiments, the internal positions exclude positions 12-14, counting from the 5′-end of the antisense strand. In some embodiments, the internal positions exclude positions 2-5, counting from the 5′-end of the antisense strand.

In some embodiments, the one or more lipophilic moieties containing one or more saturated or unsaturated C₂₂ hydrocarbon chains are conjugated to one or more of the following internal positions: positions 4-8 and 13-18 on the sense strand, and positions 6-10 and 15-18 on the antisense strand, counting from the 5′ end of each strand. In some embodiments, the one or more lipophilic moieties are conjugated to one or more of the following internal positions: positions 5, 6, 7, 15, 16, and 17 on the sense strand, and positions 15, 16, and 17 on the antisense strand, counting from the 5′-end of each strand.

In some embodiments, the one or more lipophilic moieties are conjugated to one or more of the following internal positions: positions 5, 6, 7, 15, 16, and 17 on the sense strand, and positions 6, 7, 8, 9, 10, 15, 16, and 17 on the antisense strand, counting from the 5′-end of each strand.

In some embodiments, the one or more lipophilic moieties containing one or more saturated or unsaturated C₂₂ hydrocarbon chains are conjugated to position 5 on the sense strand, counting from the 5′-end of the sense strand.

In some embodiments, the one or more lipophilic moieties containing one or more saturated or unsaturated C₂₂ hydrocarbon chains are conjugated to position 6 on the sense strand, counting from the 5′-end of the sense strand.

In some embodiments, the one or more lipophilic moieties containing one or more saturated or unsaturated C₂₂ hydrocarbon chains are conjugated to position 7 on the sense strand, counting from the 5′-end of the sense strand.

In some embodiments, the one or more lipophilic moieties containing one or more saturated or unsaturated C₂₂ hydrocarbon chains are conjugated to position 15 on the sense strand, counting from the 5′-end of the sense strand.

In some embodiments, the one or more lipophilic moieties containing one or more saturated or unsaturated C₂₂ hydrocarbon chains are conjugated to position 16 on the sense strand, counting from the 5′-end of the sense strand.

In some embodiments, the one or more lipophilic moieties containing one or more saturated or unsaturated C₂₂ hydrocarbon chains are conjugated to position 17 on the sense strand, counting from the 5′-end of the sense strand.

In some embodiments, the one or more lipophilic moieties containing one or more saturated or unsaturated C₂₂ hydrocarbon chains are conjugated to position 6 on the antisense strand, counting from the 5′-end of the antisense strand.

In some embodiments, the one or more lipophilic moieties containing one or more saturated or unsaturated C₂₂ hydrocarbon chains are conjugated to position 7 on the antisense strand, counting from the 5′-end of the antisense strand.

In some embodiments, the one or more lipophilic moieties containing one or more saturated or unsaturated C₂₂ hydrocarbon chains are conjugated to position 8 on the antisense strand, counting from the 5′-end of the antisense strand.

In some embodiments, the one or more lipophilic moieties containing one or more saturated or unsaturated C₂₂ hydrocarbon chains are conjugated to position 9 on the antisense strand, counting from the 5′-end of the antisense strand.

In some embodiments, the one or more lipophilic moieties containing one or more saturated or unsaturated C₂₂ hydrocarbon chains are conjugated to position 10 on the antisense strand, counting from the 5′-end of the antisense strand.

In some embodiments, the one or more lipophilic moieties containing one or more saturated or unsaturated C₂₂ hydrocarbon chains are conjugated to position 15 on the antisense strand, counting from the 5′-end of the antisense strand.

In some embodiments, the one or more lipophilic moieties containing one or more saturated or unsaturated C₂₂ hydrocarbon chains are conjugated to position 16 on the antisense strand, counting from the 5′-end of the antisense strand.

In some embodiments, the one or more lipophilic moieties containing one or more saturated or unsaturated C₂₂ hydrocarbon chains are conjugated to position 17 on the antisense strand, counting from the 5′-end of the antisense strand.

In some embodiments, the dsRNA agent comprises at least one lipophilic monomer

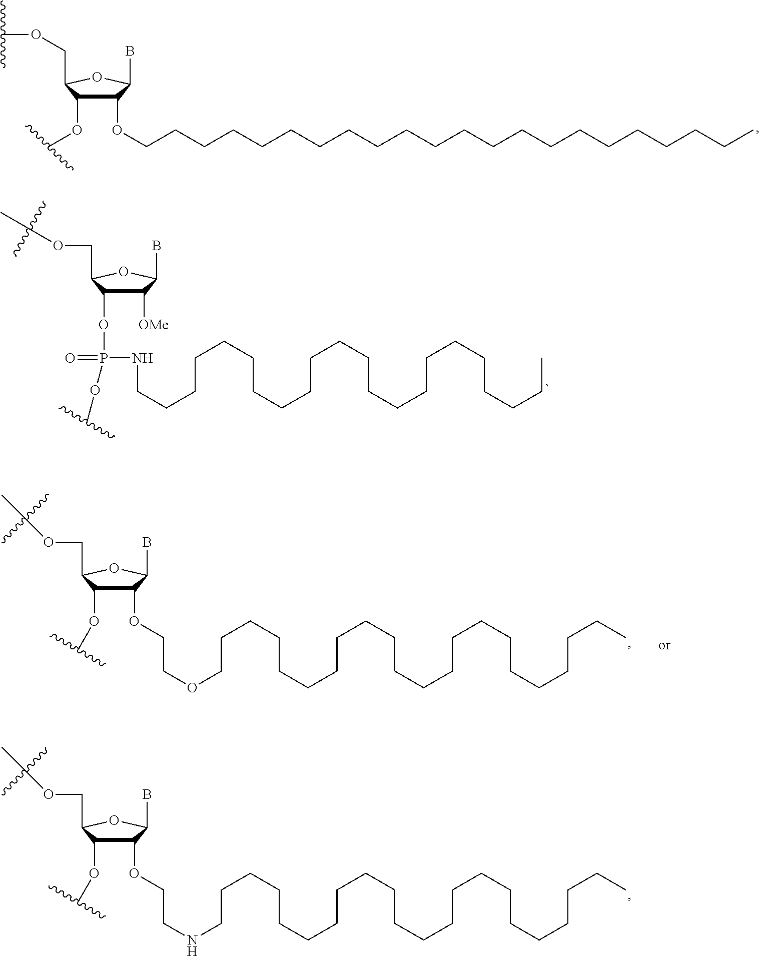

wherein B is a modified or unmodified nucleobase. In one embodiment, this lipophilic monomer is conjugated to position 5 on the sense strand, counting from the 5′-end of the sense strand. In one embodiment, this lipophilic monomer is conjugated to position 6 on the sense strand, counting from the 5′-end of the sense strand. In one embodiment, this lipophilic monomer is conjugated to position 7 on the sense strand, counting from the 5′-end of the sense strand. In one embodiment, this lipophilic monomer is conjugated to position 15 on the sense strand, counting from the 5′-end of the sense strand. In one embodiment, this lipophilic monomer is conjugated to position 16 on the sense strand, counting from the 5′-end of the sense strand. In one embodiment, this lipophilic monomer is conjugated to position 17 on the sense strand, counting from the 5′-end of the sense strand.

In some embodiments, the one or more lipophilic moieties containing one or more saturated or unsaturated C₂₂ hydrocarbon chains are conjugated to one or more terminal positions: position 1, 2, or 3 on the sense or antisense strand, counting from the 5′ end or 3′ end of each strand. In some embodiments, at least one lipophilic moieties containing one or more saturated or unsaturated C₂₂ hydrocarbon chains are conjugated to one or more terminal positions: position 1, 2, or 3 on the sense or antisense strand, counting from the 5′ end of each strand. In one embodiment, a lipophilic moiety containing a saturated or unsaturated C₂₂ hydrocarbon chain is conjugated to terminal position 1 on the sense or antisense strand, counting from the 5′ end of each strand. In some embodiments, at least one lipophilic moieties containing one or more saturated or unsaturated C₂₂ hydrocarbon chains are conjugated to one or more terminal positions: position 1, 2, or 3 on the sense or antisense strand, counting from the 3′ end of each strand. In one embodiment, a lipophilic moiety containing a saturated or unsaturated C₂₂ hydrocarbon chain is conjugated to terminal position 1 on the sense or antisense strand, counting from the 3′ end of each strand.

In some embodiments, at least one lipophilic moiety containing one or more saturated or unsaturated C₂₂ hydrocarbon chains is conjugated to position 1 on the sense or antisense strand, counting from the 5′ end of each strand. In certain embodiments, the conjugation to position 1 is by modification of the 2′-position on the sugar moiety of the nucleotide at position 1. In certain embodiments, the conjugation to position 1 is by modification of the 5′-position on the sugar moiety of the nucleotide at position 1. In certain embodiments, the conjugation to position 1 is by modification of the 4′-position on the sugar moiety of the nucleotide at position 1. In certain embodiments, the conjugation to position 1 is by modification of the nucleobase of the nucleotide at position 1.

In some embodiments, the sense and antisense strands of the dsRNA agent are each 15 to 30 nucleotides in length. In one embodiment, the sense and antisense strands of the dsRNA agent are each 19 to 25 nucleotides in length. In one embodiment, the sense and antisense strands of the dsRNA agent are each 21 to 23 nucleotides in length.

In some embodiments, the dsRNA agent comprises a single-stranded overhang on at least one of the termini, e.g., 3′ and/or 5′ overhang(s) of 1-10 nucleotides in length, for instance, an overhang of 1, 2, 3, 4, 5, or 6 nucleotides. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length. In some embodiments, the dsRNA agent may also have a blunt end, located at the 5′-end of the antisense strand (or the 3′-end of the sense strand), or vice versa. In one embodiment, the dsRNA agent comprises a 3′ overhang at the 3′-end of the antisense strand, and optionally a blunt end at the 5′-end of the antisense strand. In one embodiment, the dsRNA agent has a 5′ overhang at the 5′-end of the sense strand, and optionally a blunt end at the 5′-end of the antisense strand. In one embodiment, the dsRNA agent has two blunt ends at both ends of the dsRNA duplex.

In one embodiment, the sense strand of the dsRNA agent is 21-nucleotide in length, and the antisense strand is 23-nucleotide in length, wherein the strands form a double-stranded region of 21 consecutive base pairs having a 2-nucleotide long single stranded overhangs at the 3′-end.

In one embodiment, the dsRNA agent has two blunt ends at both ends of the dsRNA duplex such that the sense strand of the dsRNA agent is 19-nucleotide in length, and the antisense strand is 19-nucleotide in length, wherein the strands form a double-stranded region of 19 consecutive base pairs.

In one embodiment, the dsRNA agent has two blunt ends at both ends of the dsRNA duplex such that the sense strand of the dsRNA agent is 20-nucleotide in length, and the antisense strand is 20-nucleotide in length, wherein the strands form a double-stranded region of 20 consecutive base pairs.

In one embodiment, the dsRNA agent has two blunt ends at both ends of the dsRNA duplex such that the sense strand of the dsRNA agent is 21-nucleotide in length, and the antisense strand is 21-nucleotide in length, wherein the strands form a double-stranded region of 21 consecutive base pairs.

In one embodiment, the dsRNA agent has two blunt ends at both ends of the dsRNA duplex such that the sense strand of the dsRNA agent is 22-nucleotide in length, and the antisense strand is 22-nucleotide in length, wherein the strands form a double-stranded region of 22 consecutive base pairs.

In one embodiment, the dsRNA agent has two blunt ends at both ends of the dsRNA duplex such that the sense strand of the dsRNA agent is 23-nucleotide in length, and the antisense strand is 23-nucleotide in length, wherein the strands form a double-stranded region of 23 consecutive base pairs.

In some embodiments, the sense strand further comprises at least one phosphorothioate linkage at the 3′-end. In some embodiments, the sense strand further comprises at least two phosphorothioate linkages at the 3′-end. In some embodiments, one or more lipophilic monomers (e.g., including a lipophilic moiety containing one or more saturated or unsaturated C₂₂ hydrocarbon chains) are located on the 3′-end of the sense strand. In one embodiment, one of the phosphorothioate linkages is located between the lipophilic monomer and the first nucleotide from the 3′-end of the sense strand.

In some embodiments, the sense strand further comprises at least one phosphorothioate linkage at the 5′-end. In some embodiments, the sense strand further comprises at least two phosphorothioate linkages at the 5′-end. In some embodiments, one or more lipophilic monomers (e.g., including a lipophilic moiety containing one or more saturated or unsaturated C₂₂ hydrocarbon chains) are located on the 5′-end of the sense strand. In one embodiment, one of the phosphorothioate linkages is located between the lipophilic monomer and the first nucleotide from the 5′-end of the sense strand.

In some embodiments, the antisense strand further comprises at least one phosphorothioate linkage at the 3′-end. In some embodiments, the antisense strand further comprises at least two phosphorothioate linkages at the 3′-end. In some embodiments, one or more lipophilic monomers (e.g., including a lipophilic moiety containing one or more saturated or unsaturated C₂₂ hydrocarbon chains) are located on the 3′-end of the antisense strand. In one embodiment, one of the phosphorothioate linkages is located between the lipophilic monomer and the first nucleotide from the 3′-end of the antisense strand.

In some embodiments, the antisense strand further comprises at least one phosphorothioate linkage at the 5′-end. In some embodiments, the antisense strand further comprises at least two phosphorothioate linkages at the 5′-end. In some embodiments, one or more lipophilic monomers (e.g., including a lipophilic moiety containing one or more saturated or unsaturated C₂₂ hydrocarbon chains) are located on the 5′-end of the antisense strand. In one embodiment, one of the phosphorothioate linkages is located between the lipophilic monomer and the first nucleotide from the 5′-end of the antisense strand.

In some embodiments, the dsRNA agent further comprises a phosphate or phosphate mimic at the 5′-end of the sense or antisense strand. In one embodiment, there is a phosphate or phosphate mimic at the 5′-end of the sense strand. In one embodiment, there is a phosphate or phosphate mimic at the 5′-end of the antisense strand.

In some embodiments, the phosphate mimic is 5′-end phosphorothioate (5′-PS), 5′-end phosphorodithioate (5′-PS2), 5′ end vinylphosphonate (5′-VP), 5′-end methylphosphonate (MePhos), or 5′-deoxy-5′-C-malonyl

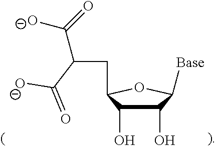

In one embodiment, the phosphate mimic is a 5′-vinyl phosphonate (VP). In one embodiment, the phosphate mimic is a 5′-(E)-vinyl phosphonate (VP) isomer (i.e., trans-vinylphosphate), 5′-(Z)-VP isomer (i.e., cis-vinylphosphate), or mixtures thereof.

In exemplary embodiments, a 5′-vinyl phosphonate modified nucleotide of the disclosure has the structure:

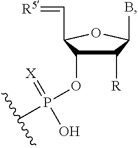

wherein.
X is O or S;
R is hydrogen, hydroxy, fluoro, or C₁₋₂₀alkoxy (e.g., methoxy or n-hexadecyloxy);
R^{5′} is ═C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

In one embodiment, R^{5′} is ═C(H)—P(O)(OH)₂ and the double bond between the C5′ carbon and R5′ is in the E orientation. In another embodiment, R is methoxy and R^{5′} is ═C(H)—P(O)(OH)₂ and the double bond between the C5′ carbon and R^{5′} is in the E orientation. In another embodiment, X is S, R is methoxy, and R^{5′} is ═C(H)—P(O)(OH)₂ and the double bond between the C5′ carbon and R5′ is in the E orientation.

In some embodiments, the —CH₂OH group at the 4′-position of the 5′-terminal nucleotide is replaced with a phosphate mimic of the formula —O—CH₂—P(O)(OR)₂, wherein each R is independently hydrogen or C₁₋₄ alkyl (e.g., one R group is hydrogen and one R group is methyl; or both R groups are hydrogen).

In one embodiment, the phosphate mimic is a 5′-cyclopropyl phosphonate (VP) (i.e., the CH₂OH group at the 4′-position of the 5′-terminal nucleotide is replaced with a group of the formula -Cy-P(O)(OR)₂, wherein Cy is a cyclopropyl ring and each R is independently hydrogen or C₁₋₄ alkyl (e.g., one R group is hydrogen or both R groups are hydrogen).

In some exemplary embodiments, the 5′-end phosphate mimic is

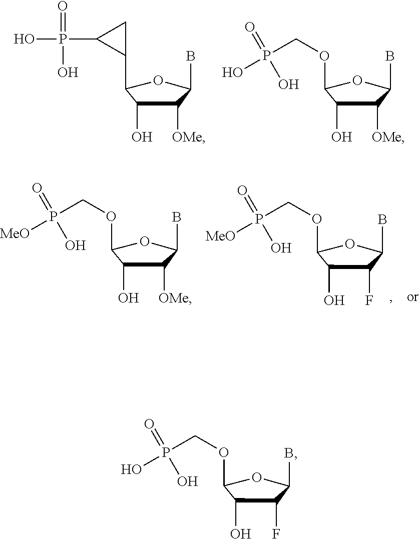

or a salt (e.g., sodium salt) thereof, wherein B is an optionally modified nucleobase (e.g., U).

In some embodiments, the 5′-end phosphate mimic is part of a modified 5′-terminal nucleotide. For example, the phosphate mimic may be part of a modified 5′-terminal nucleotide having the structure

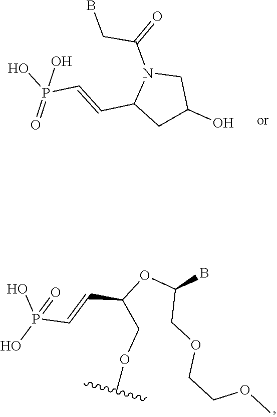

wherein B is an optionally modified nucleobase.

In some embodiments, the 5′-end phosphate mimic can also include a 5′-phosphate prodrug or 5′-phosphonate prodrug. In some embodiments, the 5′-phosphate prodrug or 5′-phosphonate prodrug has a structure of formulas disclosed in WO2022/147214, which is incorporated herein by reference. In some exemplary embodiments, the 5′-phosphate prodrug or 5′-phosphonate prodrug is:

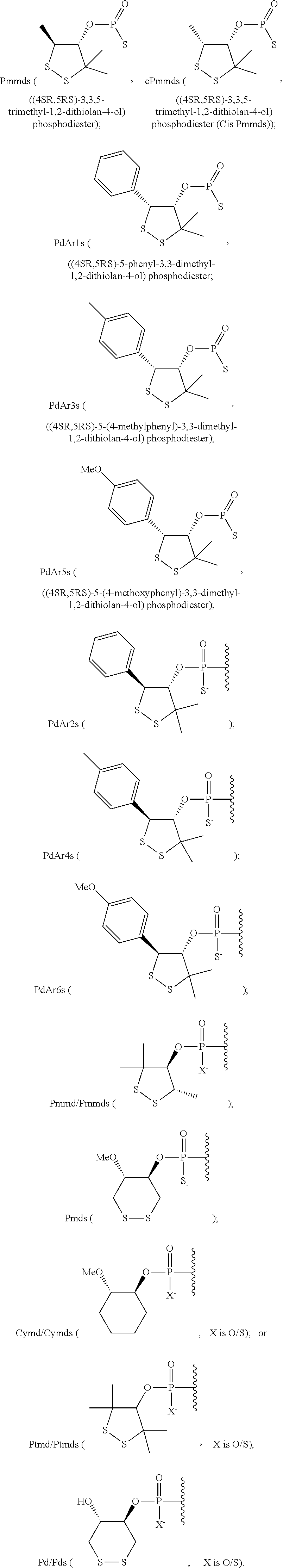

In some exemplary embodiments, the 5′-phosphate prodrug or 5′-phosphonate prodrug is:

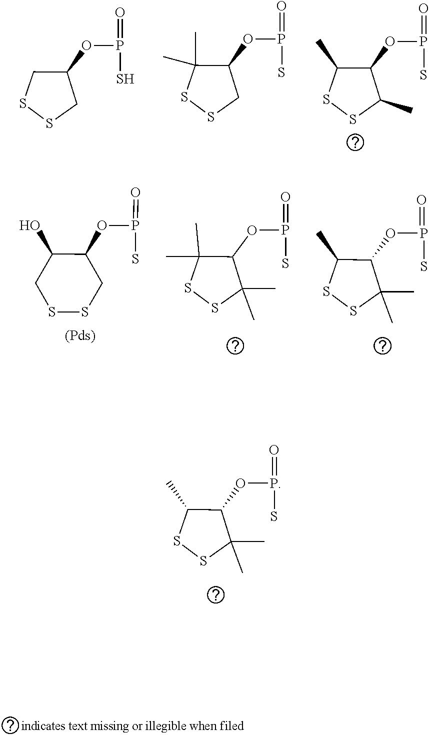

The siRNA containing one of the above list of 5′ modified phosphate prodrugs generally has an activity comparable to that of the siRNA containing 5′-VP. In some exemplary embodiments, the 5′-phosphate prodrug or 5′-phosphonate prodrug is.

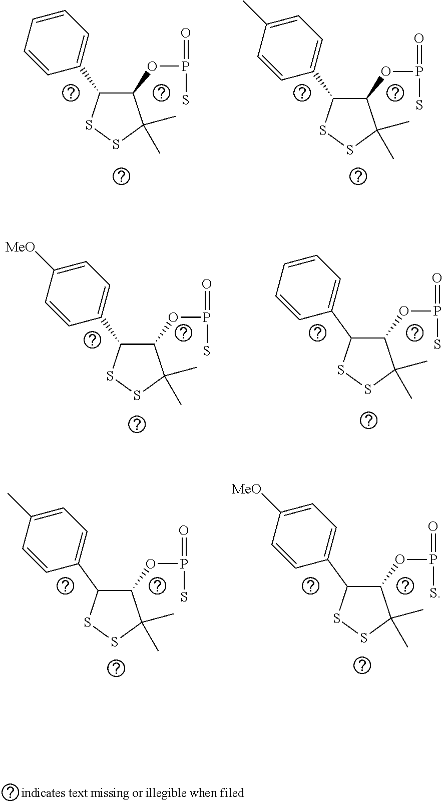

The siRNA containing one of the above list of 5′ modified phosphate prodrugs generally has an improved stability than that of the siRNA containing 5′-VP and has a better or comparable activity than that of the siRNA containing 5′-VP.

In some embodiments, the 5′-end of the antisense strand of the dsRNA agent does not contain a 5′-vinyl phosphonate (VP).

In some embodiments, the sense strand comprises at least two phosphorothioate linkages, one phosphorothioate linkage at the 3′-end and one phosphorothioate linkage at the 5′-end. In some embodiments, the sense strand comprises at least three phosphorothioate linkages, one phosphorothioate linkage at the 3′-end and a block of two phosphorothioate linkages at the 5′-end. In some embodiments, the sense strand comprises at least three phosphorothioate linkages, a block of two phosphorothioate linkages at the 3′-end and one phosphorothioate linkage at the 5′-end. In some embodiments, the sense strand comprises at least four phosphorothioate linkages, a block of two phosphorothioate linkages at the 3′-end and a block of two phosphorothioate linkages at the 5′-end. In one embodiment, one or more of these terminal phosphorothioate linkages are internucleotide linkages between the terminal nucleotides at the 3′ end and/or 5′ end of the sense strand, for instance, a phosphorothioate internucleotide linkage between the nucleotides at positions 1 and 2, and/or positions 2 or 3, counting from either the 3′ end or the 5′ end of the antisense strand. In one embodiment, one or more of these terminal phosphorothioate linkages connect an inverted abasic nucleotide to the terminal nucleotide at the 3′ end and/or the 5′ end of the sense strand. In one embodiment, at least one of these terminal phosphorothioate linkages is located between the lipophilic monomer (e.g., including a lipophilic moiety containing one or more saturated or unsaturated C₂₂ hydrocarbon chains as described herein) and the first nucleotide from the 3′ end and/or the 5′-end of the sense strand. In some embodiments, the sense strand further comprises a phosphate, phosphate mimic, or 5′-phosphate prodrug or 5′-phosphonate prodrug, as described herein, at the 5′-end.

In some embodiments, the antisense strand comprises at least two phosphorothioate linkages, one phosphorothioate linkage at the 3′-end and one phosphorothioate linkage at the 5′-end. In some embodiments, the antisense strand comprises at least three phosphorothioate linkages, one phosphorothioate linkage at the 3′-end and a block of two phosphorothioate linkages at the 5′-end. In some embodiments, the antisense strand comprises at least three phosphorothioate linkages, a block of two phosphorothioate linkages at the 3′-end and one phosphorothioate linkage at the 5′-end. In some embodiments, the antisense strand comprises at least four phosphorothioate linkages, a block of two phosphorothioate linkages at the 3′-end and a block of two phosphorothioate linkages at the 5′-end. In one embodiment, one or more of these terminal phosphorothioate linkages are internucleotide linkages between the terminal nucleotides at the 3′ end and/or the 5′ end of the antisense strand, for instance, a phosphorothioate internucleotide linkage between the nucleotides at positions 1 and 2, and/or positions 2 or 3, counting from either the 3′ end or the 5′ end of the antisense strand. In one embodiment, one or more of these terminal phosphorothioate linkages connect an inverted abasic nucleotide to the terminal nucleotide at the 3′ end and/or the 5′ end of the antisense strand. In one embodiment, at least one of these terminal phosphorothioate linkages is located between the lipophilic monomer (e.g., including a lipophilic moiety containing one or more saturated or unsaturated C₂₂ hydrocarbon chains as described herein) and the first nucleotide from the 3′ end and/or the 5′-end of the antisense strand. In some embodiments, the antisense strand further comprises a phosphate, phosphate mimic, or 5′-phosphate prodrug or 5′-phosphonate prodrug, as described herein, at the 5′-end.

In some embodiments, the dsRNA agent comprises at least four phosphorothioate linkages—two phosphorothioate linkages at the sense strand, and two phosphorothiate linkages at the antisense strand: for each strand, one phosphorothioate linkage at each of the 3′-end and the 5′-end of the strand. In some embodiments, the dsRNA agent comprises at least six phosphorothioate linkages—two phosphorothioate linkages at the sense strand, and four phosphorothiate linkages at the antisense strand: for sense strand, one phosphorothioate linkage at each of the 3′-end and the 5′-end; and for antisense strand, a block of two phosphorothioate linkages at each of the 3′-end and the 5′-end. In some embodiments, the dsRNA agent comprises at least six phosphorothioate linkages—four phosphorothioate linkages at the sense strand, and two phosphorothiate linkages at the antisense strand: for sense strand, a block of two phosphorothioate linkages at each of the 3′ end and the 5′-end; and for antisense strand, a phosphorothioate linkage at each of the 3′-end and the 5′-end. In some embodiments, the dsRNA agent comprises at least eight phosphorothioate linkages—four phosphorothioate linkages at the sense strand, and four phosphorothiate linkages at the antisense strand: for each strand, a block of two phosphorothioate linkages at each of the 3′-end and the 5′-end of the strand. In one embodiment, one or more of these terminal phosphorothioate linkages are internucleotide linkages between the terminal nucleotides at the 3′ end and/or the 5′ end of the strand, for instance, a phosphorothioate internucleotide linkage between the nucleotides at positions 1 and 2, and/or positions 2 or 3, counting from either the 3′ end or the 5′ end of the strand. In one embodiment, one or more of these terminal phosphorothioate linkages connect an inverted abasic nucleotide to the terminal nucleotide at the 3′ end and/or the 5′ end of the strand. In one embodiment, at least one of these terminal phosphorothioate linkages is located between the lipophilic monomer (e.g., including a lipophilic moiety containing one or more saturated or unsaturated C₂₂ hydrocarbon chains as described herein) and the first nucleotide from the 3′ end and/or the 5′-end of the strand. In some embodiments, the sense strand or the antisense strand further comprises a phosphate, phosphate mimic, or 5′-phosphate prodrug or 5′-phosphonate prodrug, as described herein, at the 5′-end.

In some embodiments, the dsRNA agent further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the dsRNA agent further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the dsRNA agent has at least two phosphorothioate internucleotide linkages at the first five, four, three, or two nucleotides on the antisense strand (counting from the 5′ end). In some embodiments, the dsRNA agent has at least two phosphorothioate internucleotide linkages at the first five, four, three, or two nucleotides on the antisense strand (counting from the 3′ end).

In some embodiments, the first two internucleotide linkages at the 5′-end of the antisense strand are phosphorothioate linkages. In some embodiments, the first three internucleotide linkages at the 5′-end of the antisense strand are phosphorothioate linkages.

In some embodiments, the first two internucleotide linkages at the 5′-end of the antisense strand are phosphorothioate linkages; and the last two internucleotide linkages (which are at the 3′-end) of the antisense strand are phosphorothioate linkages.

In some embodiments, the first three internucleotide linkages at the 5′-end of the antisense strand are phosphorothioate linkages; and the last two internucleotide linkages (which are at the 3′-end) of the antisense strand are phosphorothioate linkages.

In some embodiments, the first three internucleotide linkages at the 5′-end of the antisense strand are phosphorothioate linkages; and the last one internucleotide linkage (which is at the 3′-end) of the antisense strand is a phosphorothioate linkage.

In some embodiments, the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, 100%, at least 95%, at least 90%, at least 85%, at least 80%, at least 75%, at least 70%, at least 65%, at least 60%, at least 55%, at least 50%, at least 45%, at least 40%, at least 35% or at least 30% of the antisense and sense strand of the dsRNA agent is modified. For example, when 50% of the dsRNA agent is modified, 50% of all nucleotides present in the dsRNA agent contain a modification as described herein.

In one embodiment, at least 30%, at least 35%, at least 40%, at least 45%, at least 50%, at least 55%, at least 60%, at least 65%, at least 70%, at least 75%, at least 80%, at least 85%, at least 90%, at least 95%, or substantially 100% of the nucleotides of the dsRNA agent is independently modified with 2′O-methyl, 2′-O-allyl, 2′-deoxy, or 2′-fluoro.

In one embodiment, the oligonucleotide is an antisense, and at least 30%, at least 35%, at least 40%, at least 45%, at least 50%, at least 55%, at least 60%, at least 65%, at least 70%, at least 75%, at least 80%, at least 85%, at least 90%, at least 95%, or substantially 100% of the nucleotides of the antisense is independently modified with LNA, CeNA, 2′-methoxyethyl, or 2′-deoxy.

In some embodiments, the sense and antisense strands of the dsRNA agent comprise less than 12, less than 10, less than 8, less than 6, less than 4, less than 2, or no 2′-F modified nucleotides. In some embodiments, the dsRNA agent has less than 12, less than 10, less than 8, less than 6, less than 4, less than 2, or no 2′-F modifications on the sense strand. In some embodiments, the dsRNA agent has less than 12, less than 10, less than 8, less than 6, less than 4, less than 2, or no 2′-F modifications on the antisense strand.

In some embodiments, the dsRNA agent has one or more 2′-F modifications on any position of the sense strand or antisense strand.

In some embodiments, the dsRNA agent has less than 20%, less than 15%, less than 10%, less than 5% non-natural nucleotide, or substantially no non-natural nucleotide. Examples of non-natural nucleotide include acyclic nucleotides, LNA, HNA, CeNA, 2′O-methoxyalkyl (e.g., 2′-O-methoxymethyl, 2′-O-methoxyethyl, or 2′-O-2-methoxypropanyl), 2′-O-allyl, 2′-C-allyl, 2′-fluoro, 2′-O—N-methylacetamido (2′-O-NMA), a 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA, FHNA, abasic sugar, abasic cyclic and open-chain alkyl.

In some embodiments, the antisense and sense strands of the dsRNA agent comprise at least 30%, at least 35%, at least 40%, at least 45%, at least 50%, at least 55%, at least 60%, at least 65%, at least 70%, at least 75%, at least 80%, at least 85%, at least 90%, at least 95%, or virtually 100% 2′O-methyl modified nucleotides.

In some embodiments, the dsRNA agent has greater than 80%, greater than 85%, greater than 90%, greater than 95%, or virtually 100% natural nucleotides. For the purpose of these embodiments, natural nucleotides can include those having 2′-OH, 2′-deoxy, and 2′-OMe.

In some embodiments, the antisense strand contains at least one unlocked nucleic acids (UNA) modification, e.g., at the seed region of the antisense strand. In some embodiments, the antisense strand contains at least one glycerol nucleic acid (GNA) modification, e.g., at the seed region of the antisense strand. In one embodiment, the seed region is at positions 2-8 (e.g., positions 5-7) of the 5′-end of the antisense strand.

In one embodiment, the dsRNA agent comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agent has less than 20%, less than 15%, less than 10%, less than 5% non-natural nucleotide, or substantially no non-natural nucleotide.

In one embodiment, the dsRNA agent comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agent has greater than 80%, greater than 85%, greater than 95%, or virtually 100% natural nucleotides, such as those having 2′-OH, 2′-deoxy, or 2′-OMe.

In some embodiments, the dsRNA agent further comprises a targeting ligand that targets a receptor which mediates delivery to a specific CNS tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

Some embodiments of the invention provides a dsRNA agent comprising a sense strand and an antisense strand, each strand independently having a length of 15 to 35 nucleotides; at least two phosphorothioate internucleotide linkages between the first five nucleotides counting from the 5′ end of the antisense strand; at least three, four, five, or six 2′-deoxy modifications on the sense and/or antisense strands; wherein the dsRNA agent has a double stranded (duplex) region of between 19 to 25 base pairs; wherein the dsRNA agent comprises a ligand; and wherein the sense strand does not comprise a glycol nucleic acid (GNA).

It is understood that the antisense strand has sufficient complementarity to a target sequence to mediate RNA interference. In other words, the dsRNA agent is capable of inhibiting the expression of a target gene in the central nervous system (CNS).

In one embodiment, the dsRNA agent comprises at least three 2′-deoxy modifications. The 2′-deoxy modifications are at positions 2 and 14 of the antisense strand, counting from 5′-end of the antisense strand, and at position 11 of the sense strand, counting from 5′-end of the sense strand.

In one embodiment, the dsRNA agent comprises at least five 2′-deoxy modifications. The 2′-deoxy modifications are at positions 2, 12 and 14 of the antisense strand, counting from 5′-end of the antisense strand, and at positions 9 and 11 of the sense strand, counting from 5′-end of the sense strand.

In one embodiment, the dsRNA agent comprises at least seven 2′-deoxy modifications. The 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In one embodiment, the antisense strand comprises at least five 2′-deoxy modifications at positions 2, 5, 7, 12 and 14, counting from 5′-end of the antisense strand. The antisense strand has a length of 18-25 nucleotides, or a length of 18-23 nucleotides.

In one embodiment, the dsRNA agent can comprise one or more non-natural nucleotides. For example, the dsRNA agent can comprise less than 20%, e.g., less than 15%, less than 10%, or less than 5% non-natural nucleotides, or the dsRNA agent comprises no non-natural nucleotides. For example, the dsRNA agent comprises all natural nucleotides. Some exemplary non-natural nucleotides include, but are not limited to, acyclic nucleotides, locked nucleic acid (LNA), HNA, CeNA, 2′-methoxyethyl, 2′-O-allyl, 2′-C-allyl, 2′-fluoro, 2′-O—N-methylacetamido (2′-O-NMA), a 2′-O-dimethylaminoethoxyethyl (2′-O-DMAEOE), 2′-O-aminopropyl (2′-O-AP), and 2′-ara-F.

In one embodiment, the dsRNA agent comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the dsRNA agent has a duplex region of between 19 to 25 base pairs; wherein the dsRNA agent optionally comprises a ligand; wherein the sense strand does not comprise a glycol nucleic acid (GNA); and wherein the dsRNA agent comprises less than 20%, e.g., less than 15%, less than 10%, or less than 5% non-natural nucleotides or the dsRNA agent comprises all natural nucleotides.

In one embodiment, at least one the sense and antisense strands comprises at least one, e.g., at least two, at least three, at least four, at least five, at least six, or at least seven or more, 2′-deoxy modifications in a central region of the sense or antisense strand. Accordingly, in one embodiment, the dsRNA agent comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the dsRNA agent has a duplex region of between 19 to 25 base pairs; wherein the dsRNA agent optionally comprises a ligand; and wherein the sense strand and/or the antisense strand comprises at least one, e.g., at least two, at least three, at least four, at least five, at least six, or at least seven or more, 2′-deoxy modifications in a central region of the sense strand and/or the antisense strand.

In some embodiment, the sense strand has a length of 18 to 30 nucleotides and comprises at least two 2′-deoxy modifications in the central region of the sense strand. For example, the sense strand has a length of 18 to 30 nucleotides and comprises at least two 2′-deoxy modifications within positions 7, 8, 9, 10, 11, 12, and 13, counting from 5′-end of the sense strand.

In one embodiment, the antisense strand has a length of 18 to 30 nucleotides and comprises at least two 2′-deoxy modifications in the central region of the antisense strand. For example, the antisense strand has length of 18 to 30 nucleotides and comprises at least two 2′-deoxy modifications within positions 10, 11, 12, 13, 14, 15 and 16, counting from 5′-end of the antisense strand.

In one embodiment, the dsRNA agent comprises a sense strand and an antisense strand; wherein the sense strand has a length of 17-30 nucleotide and comprises at least one 2′-deoxy modification in the central region of the sense strand; and wherein the antisense strand independently has a length of 17-30 nucleotides and comprises at least two 2′-deoxy modifications in the central region of the antisense strand.

In one embodiment, the dsRNA agent comprises a sense strand and an antisense strand; wherein the sense strand has a length of 17-30 nucleotide and comprises at least two 2′-deoxy modifications in the central region of the sense strand; and wherein the antisense strand independently has a length of 17-30 nucleotides and comprises at least one 2′-deoxy modification in the central region of the antisense strand.

In one embodiment, the dsRNA agent comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the dsRNA agent has a duplex region of between 19 to 25 base pairs; wherein the dsRNA agent optionally comprises a ligand; and wherein the sense strand comprises at least one, e.g., at least two, at least three, at least four, at least five, at least six, at least seven or more, 2′-deoxy modifications in a central region of the sense strand.

In one embodiment, the dsRNA agent comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the dsRNA agent has a duplex region of between 19 to 25 base pairs; wherein the dsRNA agent optionally comprises a ligand; and wherein the antisense strand comprises at least one, e.g., at least two, at least three, at least four, at least five, at least six, at least seven or more, 2′-deoxy modifications in a central region of the antisense strand.

In one embodiment, the dsRNA agent comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the dsRNA agent has a duplex region of between 19 to 25 base pairs; wherein the dsRNA agent optionally comprises a ligand; wherein the dsRNA agent comprises less than 20%, e.g., less than 15%, less than 10%, or less than 5% non-natural nucleotides or the dsRNA agent comprises all natural nucleotides; and wherein the sense strand and/or the antisense strand comprises at least one, e.g., at least two, at least three, at least four, at least five, at least six, at least seven or more, 2′-deoxy modifications in a central region of the sense strand and/or the antisense strand.

In one embodiment, the dsRNA agent comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the dsRNA agent has a duplex region of between 19 to 25 base pairs; wherein the dsRNA agent optionally comprises a ligand; wherein the dsRNA agent comprises less than 20%, e.g., less than 15%, less than 10%, or less than 5% non-natural nucleotides or the dsRNA agent comprises all natural nucleotides; and wherein the sense strand comprises at least one, e.g., at least two, at least three, at least four, at least five, at least six, at least seven or more, 2′-deoxy modifications in a central region of the sense strand.

In one embodiment, the dsRNA agent comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the dsRNA agent has a duplex region of between 19 to 25 base pairs; wherein the dsRNA agent comprises a ligand; wherein the dsRNA agent comprises less than 20%, e.g., less than 15%, less than 10%, or less than 5% non-natural nucleotides or the dsRNA agent comprises all natural nucleotides; and wherein the antisense strand comprises at least one, e.g., at least two, at least three, at least four, at least five, at least six, at least seven or more, 2′-deoxy modifications in a central region of the antisense strand.

In one embodiment, when the dsRNA agent comprises less than 8 non-2′OMe nucleotides, the antisense stand comprises at least one DNA. For example, in any embodiment when the dsRNA agent comprises less than 8 non-2′OMe nucleotides, the antisense stand may comprise at least one DNA.

In one embodiment, when the antisense comprises two deoxy nucleotides and said nucleotides are at positions 2 and 14, counting from the 5′-end of the antisense strand, the dsRNA agent comprises 8 or less (e.g., 8, 7, 6, 5, 4, 3, 2, 1 or 0) non-2′OMe nucleotides. For example, in any one of the embodiments of the invention when the antisense comprises two deoxy nucleotides and said nucleotides are at positions 2 and 14, counting from the 5′-end of the antisense strand, the dsRNA agent comprises 0, 1, 2, 3, 4, 5, 6, 7 or 8 non 2′-OMe nucleotides.

Another aspect of the invention provides a cell comprising the dsRNA agents as described herein.

Another aspect of the invention provides a pharmaceutical composition comprising the dsRNA agents as described herein.

All the above embodiments relating to the lipophilic monomers, the lipophilic moieties, the saturated or unsaturated C₂₂ hydrocarbon chains, and their conjugation to the dsRNA agent in the first aspect of the invention relating to the dsRNA agent are suitable in these aspects of the invention relating to the cells and pharmaceutical compositions.

In another aspect, the invention further provides a method for delivering the dsRNA agent of the invention to a specific target gene in the central nervous system (CNS) of a subject by subcutaneous or intravenous administration. The invention further provides the dsRNA agent described herein for use in a method for delivering said agents to a specific target in a subject by subcutaneous, intravenous, intrathecal, or intracerebroventricular administration.

Another aspect of the invention relates to a method of modulating the expression of a target gene in a CNS cell, comprising administering to said cell the dsRNA agent as described herein.

Another aspect of the invention relates to a method of treating or preventing a CNS disorder in a subject, comprising administering to the subject a therapeutically effective amount of a dsRNA agent as described herein, thereby treating the subject by modulating the expression of the target gene in the CNS of the subject.

In one embodiment, the cell is within a subject. In one embodiment, the subject is a mammal. In one embodiment, the subject is a human.

Exemplary CNS disorders that can be treated by the method of the invention include Alzheimer, amyotrophic lateral sclerosis (ALS), frontotemporal dementia, Huntington, Parkinson, spinocerebellar, prion, and lafora.

All the above embodiments relating to the lipophilic monomers, the lipophilic moieties, the saturated or unsaturated C₂₂ hydrocarbon chains, and their conjugation to the dsRNA agent in the first aspect of the invention relating to the dsRNA agent are suitable in these aspects of the invention relating to a method for delivering the dsRNA agent, a method of modulating the expression of a target gene in a cell, and a method of treating or preventing a CNS disorder in a subject.

In some embodiments, the dsRNA agent is administered extrahepatically.

In one embodiment, the dsRNA agent is administered intrathecally or intracerebroventricularly. By intrathecal or intracerebroventricular administration of the dsRNA agent, the method can reduce the expression of a target gene in a brain or spine tissue, for instance, cortex, cerebellum, cervical spine, lumbar spine, and thoracic spine.

In some embodiments, the target gene in the CNS is selected from the group consisting of APP, SOD1, SCN9A, HTT (HUNTINGTIN), APOE, LRRK2, PRNP, SCD5, GPR75, MAPT, SNCA, ABLIM3, ADRA2A, ATXN1, ATXN2, ATXN3, ELOVL1, FLNA, NOGO-L or NOGO-R, HIF-1α, RHO-A, NAV1.8, CD45, GSK-3, GSK3a, MIG-12, Mgat1, Mgat4, SLC35A1, SLC35A2, GNE, TMPRSS6, Complement Component C3, APCS, C9orf72, CHI3L1/YKL-40, EXT1, EXT2, NDST2, RPS25, ALK, and SCD5. In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, TTR, SCN9A, LRRK2, GPR75, APOE, SCD5, ELOVL1, FLNA, ALK, CHI3L1(YKL-40), RPS25, α2-AR, and GSK3a.

In some embodiments, the dsRNA agent is administered at a dosage level of no more than 80%, no more than 75%, no more than 70%, no more than 65%, no more than 60%, no more than 55%, no more than 50%, no more than 45%, no more than 40%, no more than 35%, no more than ⅓, no more than 30%, no more than 25% of Dose 1, which indicates the dosage level of a comparative dsRNA agent, and the method achieves the same expression reduction of the target gene as administering Dose 1 of the comparative dsRNA agent. The comparative dsRNA agent has the same sense and antisense strands and a same conjugation with a same lipophilic moiety except containing a different hydrocarbon chain having fewer carbon atoms than the C₂₂ hydrocarbon chains (e.g., the lipophilic moiety of the comparative dsRNA agent contains a C16 hydrocarbon chain). In certain embodiments, the dsRNA agent contains a 2′-O-docosanyl modification and the comparative dsRNA agent contains a 2′-O-hexadecyl modification at the same position.

### BRIEF DESCRIPTION OF THE DRAWINGS

FIG. 1 shows the results of the inhibition of APP gene expression (sAPPα and sAPPβ, respectively) in NHP CSF following IT administration of an siRNA duplex with C22 conjugate (dosed at 6.7 mg and 20 mg) (on the right), as compared to an siRNA duplex with C16 conjugate (dosed at 20 mg and 60 mg) (on the left), at various time points over 113 days.

FIG. 2A compares the results of the inhibition of APP gene expression (sAPPα and sAPPβ, respectively) in NHP CSF following IT administration of an siRNA duplex with C22 conjugate (dosed at 20 mg) to the results of an siRNA duplex with C16 conjugate (dosed at 60 mg), at various time points over 113 days. FIG. 2B compares the results of the inhibition of APP gene expression (sAPPα and sAPPβ, respectively) in NHP CSF following IT administration of the siRNA duplex with C22 conjugate (dosed at 6.7 mg) to the results of an siRNA duplex with C16 conjugate (dosed at 20 mg), at various time points over 113 days.

FIG. 3A compares the overall results of the inhibition of APP gene expression (sAPPα and sAPPβ, respectively) in NHP CSF following IT administration of an siRNA duplex with C22 conjugate (dosed at 6.7 mg and 20 mg) to the results of an siRNA duplex with C16 conjugate (dosed at 20 mg and 60 mg), at various time points over 113 days. FIG. 3B compares the results of the inhibition of APP gene expression (sAPPα) in NHP CSF following IT administration of an siRNA duplex with C22 conjugate (dosed at 20 mg) to the results of an siRNA duplex with C16 conjugate (dosed at 20 mg and 60 mg), at various time points over 29 days.

FIG. 4 shows the results of the inhibition of APP gene expression (sAPPα and sAPPβ, respectively) in NHP CSF following IT administration (dosed at 20 mg) of an siRNA duplex with various C16 conjugates: C16 conjugated to N6 of the sense strand (C16, on the left), C16 conjugated to N1 of the sense strand (SS1 C16, in the middle), and C16 conjugated to N6 of the sense strand with a modified phosphate backbone where C₁₆ was conjugated (backbone, on the right), at various time points over 29 days.

FIG. 5A shows the results of the inhibition of SOD1 gene expression in mouse CNS tissues following ICV administration (dosed at 11 μg, 33 μg, 100 μg, or 150 μg) of an siRNA duplex with a C22 conjugate at N6 of the sense strand (AD-1427062) as compared to those of a C16 conjugate at N6 of the sense strand (AD-401824) on D15. FIG. 5B shows the results of the inhibition of SOD1 gene expression in rat CNS tissues following IT administration (dosed at 0.03 mg, 0.1 mg, 0.3 mg, and 0.9 mg) of an siRNA duplex with a C22 conjugate at N6 of the sense strand (AD-1427062) as compared to those of a C16 conjugate at N6 of the sense strand (AD-401824) on D15.

FIG. 6A shows the concentrations of the C22-conjugated siRNA duplex (AD-1427062) as compared to that of the C16-conjugated siRNA duplex (AD-401824) in rat frontal cortex tissue, following IT administration (dosed at 0.03 mg, 0.1 mg, 0.3 mg, and 0.9 mg) of the siRNA duplex on D15. FIG. 6B shows the concentrations of the C22-conjugated siRNA duplex (AD-1427062) as compared to that of the C16-conjugated siRNA duplex (AD-401824) in rat spine tissue, following IT administration (dosed at 0.03 mg, 0.1 mg, 0.3 mg, and 0.9 mg) of the siRNA duplex on D15. FIG. 6C shows the results of concentration ratios of the C22-conjugated siRNA duplex (AD-1427062) to the C16-conjugated siRNA duplex (AD-401824) in rat frontal cortex tissue and spine tissue, following IT administration (dosed at 0.03 mg, 0.1 mg, 0.3 mg, and 0.9 mg) of the siRNA duplex on D15.

FIG. 7A shows the results of the inhibition of SOD1 gene expression in rat CNS tissues following IT administration of a C22-conjugated siRNA duplex (AD-1427062) (dosed at 0.1 mg, 0.3 mg, and 0.6 mg) as compared to those of a C16-conjugated siRNA duplex (AD-401824) (dosed at 0.3 mg, 0.6 mg, and 0.9 mg) on D30. FIG. 7B shows the results of the inhibition of SOD1 gene expression in rat CNS tissues following IT administration of a C22-conjugated siRNA duplex (AD-1427062) (dosed at 0.3 mg and 0.6 mg) as compared to those of a C16-conjugated siRNA duplex (AD-401824) (dosed at 0.3 mg, 0.6 mg, and 0.9 mg) on D90.

FIGS. 8A-8D show the results of the inhibition of mRNA expression for rMAP2 (FIG. 8A), rMBP (FIG. 8B), rGFAP (FIG. 8C), and rAif1 (FIG. 8D) in rat CNS tissues following IT administration of a C22-conjugated siRNA duplex (dosed at 0.6 mg) as compared to those of a C16-conjugated siRNA (dosed at 0.6 mg) on D15. See Table 4 for corresponding siRNA duplex IDs for C22-conjugated siRNA duplex and C16-conjugated siRNA for each target referenced in the figures.

FIGS. 9A-9C show the results of the inhibition of CSF APP gene expression after intrathecal (IT) injection of the C22-conjugated siRNA duplexes (dosed at 20 mg and 60 mg at DO; and redosed at D29) in NHP CNS tissues (prefrontal cortex, putamen in FIG. 9A; hippocampus and cerebellum in FIG. 9B; and caudate and lumbar spine in FIG. 9C) at D106.

FIG. 10 shows the results of the 24-hour CSF PK data in NHP CSF following IT administration of a C22-conjugated siRNA duplex (dosed at 20 mg and 60 mg).

FIG. 11 shows the results of PK/PD correlation in NHP for all tissues following IT administration of a C22-conjugated siRNA duplex. The X axis shows the concentrations of the C22-conjugated siRNA duplexes in NHP CNS (all tissues) following IT administration of the siRNA duplex D85-D105. The Y axis shows the % cyno APP remaining in NHP CNS (all tissues) following IT administration of the siRNA duplex D85-D105.

FIG. 12A show the levels of CSF soluble APP protein (sAPPβ) after intrathecal (IT) injection of the C22-conjugated siRNA duplex (AD-2034768) (dosed at 20 mg and 60 mg) in NHP CSF at various time points over 78 days, as compared to the levels of CSF soluble APP protein (sAPPα and sAPPβ, respectively) after intrathecal (IT) injection of the C16-conjugated siRNA (AD-454843) (dosed at 72 mg) in NHP CSF at various time points over 85 days. FIG. 12B show the levels of CSF soluble APP protein (sAPPβ) after intrathecal (IT) injection of the C22-conjugated siRNA duplex (AD-1956470) (dosed at 20 mg and 60 mg) in NHP CSF at various time points over 78 days, as compared to the levels of CSF soluble APP protein (sAPPα and sAPPβ, respectively) after intrathecal (IT) injection of the C16-conjugated siRNA (AD-454842) (dosed at 72 mg) in NHP CSF at various time points over 85 days. FIG. 12C show the levels of CSF soluble APP protein (sAPPβ) after intrathecal (IT) injection of the C22-conjugated siRNA duplex (AD-2034769) (dosed at 20 mg and 60 mg) in NHP CSF at various time points over 78 days, as compared to the levels of CSF soluble APP protein (sAPPα and sAPPβ, respectively) after intrathecal (IT) injection of the C16-conjugated siRNA (AD-454972) (dosed at 72 mg) in NHP CSF at various time points over 85 days.

FIGS. 13A-13B show the inhibition of SOD1 gene expression in mouse CNS tissue (right hemisphere) and periphery tissues (heart, liver) following ICV administration (dosed at 100 μg) of an siRNA duplex, with a lipophilic moiety containing a C₂₂ hydrocarbon chain, with various chemical modifications on the lipophilic moiety, on D15. In FIG. 13A, the data for each tissue, from left to right, represent PBS, AS-401824, AD-1427062, AD-1623136, AD-1962193, AD-1962191, and AD-1962192, respectively. In FIG. 13B, the data for each tissue, from left to right, represent PBS, AS-401824, AD-1427062, AD-1623136, AD-1962193, AD-1949272, and AD-1949273, respectively.

FIG. 14A is a chart showing the positional impact of C22-conjugation across the siRNA sequence on the sense strand evaluated in rodent Neuro2a cells using exemplary siRNAs, targeting SOD1, comprising a C₂₂ hydrocarbon chain at various dosages (0.1 nM, 1 nM, and 10 nM, respectively). FIG. 14B is a chart showing the positional impact of C22-conjugation across the siRNA sequence on the sense strand evaluated in human Be2C cells using exemplary siRNAs, targeting SOD1, comprising a C₂₂ hydrocarbon chain at various dosages (0.1 nM, 1 nM, and 10 nM, respectively). See Table 7 for corresponding siRNA duplex IDs for various positions referenced in the figures.

FIG. 15A is a chart showing the positional impact of C22-conjugation across the siRNA sequence on the sense strand evaluated in rodent Neuro2a cells using exemplary siRNAs, targeting APP, comprising a C₂₂ hydrocarbon chain at various dosages (0.1 nM, 1 nM, and 10 nM, respectively). FIG. 15B is a chart showing the positional impact of C22-conjugation across the siRNA sequence on the sense strand evaluated in human Be2C cells using exemplary siRNAs, targeting SOD1, comprising a C₂₂ hydrocarbon chain at various dosages (0.1 nM, 1 nM, and 10 nM, respectively). See Table 8 for corresponding siRNA duplex IDs for various positions referenced in the figures.

FIG. 16A is a chart showing the positional impact of C22-conjugation across the siRNA sequence on the antisense strand evaluated in rodent Neuro2a cells using exemplary siRNAs, targeting SOD1, comprising a C₂₂ hydrocarbon chain at various dosages (0.1 nM, 1 nM, and 10 nM, respectively). FIG. 16B is a chart showing the positional impact of C22-conjugation across the siRNA sequence on the antisense strand evaluated in human Be2C cells using exemplary siRNAs, targeting SOD1, comprising a C₂₂ hydrocarbon chain at various dosages (0.1 nM, 1 nM, and 10 nM, respectively).

FIG. 17A is a chart showing the positional impact of C22-conjugation across the siRNA sequence on the antisense strand evaluated in rodent Neuro2a cells using exemplary siRNAs, targeting APP, comprising a C₂₂ hydrocarbon chain at various dosages (0.1 nM, 1 nM, and 10 nM, respectively). FIG. 17B is a chart showing the positional impact of C22-conjugation across the siRNA sequence on the antisense strand evaluated in human Be2C cells using exemplary siRNAs, targeting SOD1, comprising a C₂₂ hydrocarbon chain at various dosages (0.1 nM, 1 nM, and 10 nM, respectively).

FIG. 18 shows the results of the inhibition of SOD1 gene expression in mouse CNS tissue (right brain hemisphere) following ICV administration (dosed at 150 μg) of the siRNA duplexes, targeting SOD1, comprising a C22 hydrocarbon chain conjugated at various positions of the sense strand or antisense strand on D7. Controls included aCSF without siRNAs, an siRNA duplex without a lipophilic conjugation (AD-1964624, uncong.), and an siRNA duplex comprising a C16 hydrocarbon chain conjugated at N6 of the sense strand (AD-890098, SS6-C16). See Table 11 for corresponding positions associated with the siRNA duplex IDs referenced in the figure.

FIG. 19A shows the inhibition of SOD1 gene expression in rat CNS: right hemisphere brain tissues (striatum, frontal cortex, cerebellum, and hippocampus) and spine tissue (thoracic cord) following IT administration (dosed at 0.6 mg in 30 ul aCSF) of various siRNA duplexes, targeting SOD1, containing an internal conjugation (position 6 of the sense strand) of a lipophilic moiety containing a C22 hydrocarbon chain, on D14. FIG. 19B shows the inhibition of SOD1 gene expression in rat periphery tissues (liver, heart) following IT administration (dosed at 0.6 mg in 30 ul aCSF) of various siRNA duplexes, targeting SOD1, containing an internal conjugation (position 6 of the sense strand) of a lipophilic moiety containing a C₂₂ hydrocarbon chain, on D14. Controls included aCSF without siRNAs, an siRNA duplex comprising a C16 hydrocarbon chain conjugated at N6 of the sense strand, and an siRNA duplex comprising a C6-C16-OH moiety conjugated at N6 of the sense strand. In both figures, the data for each tissue, from left to right, represent aCSF, SS6 C16 (AD-401824), SS6 C22 (AD-1427062), SS6 C6-C16-OH (AD-2700143), respectively.

FIG. 20A shows the results of the inhibition of SOD1 gene expression in rat CNS: right hemisphere brain tissues (striatum, frontal cortex, cerebellum, and hippocampus) and spine tissue (thoracic cord) following IT administration (dosed at 0.6 mg in 30 ul aCSF) of various siRNA duplexes, targeting SOD1, containing 3′-terminal or 5′-terminal conjugation of a lipophilic moiety (C16 or C22), on D14. FIG. 20B shows the results of the inhibition of SOD1 gene expression in rat periphery tissues (liver, heart) following IT administration (dosed at 0.6 mg in 30 ul aCSF) of various siRNA duplexes, targeting SOD1, containing 3′-terminal or 5′-terminal conjugation of a lipophilic moiety (C16 or C22), on D14. In FIG. 20B, the various lipid conjugations include L54, L321, Q447, Q448, Q466, Q478, Q483, and internal C16 control, respectively.

### DETAILED DESCRIPTION

The present invention is based, at least in part, on the surprising discovery that conjugating a C₂₂ lipophilic moiety to at least one strand of a dsRNA agent (e.g., position 6 on the sense strand counting from the 5′-end) provides surprisingly high in vivo silencing potency to a CNS target gene at a relatively low dosage level, compared to a dsRNA agent having the same sense and antisense strands and a same conjugation with a same lipophilic moiety except containing a different hydrocarbon chain having fewer carbon atoms than the C₂₂ hydrocarbon chains (e.g., a C₁₆ lipophilic moiety). Thus, disclosed herein is an siRNA duplex comprising a C₂₂ conjugate for modulating the expression of a target gene in the central nervous system (CNS), which is capable of providing the desired silencing activity in the CNS at a significantly reduced dosage level.

One aspect of the invention provides double-stranded RNA (dsRNA) agent for modulating the expression of a target gene in the central nervous system (CNS) comprising: an antisense strand which is complementary to a target gene in the CNS; a sense strand which is complementary to said antisense strand; and one or more lipophilic moieties containing one or more saturated or unsaturated C₂₂ hydrocarbon chains, conjugated to one or more positions on at least one strand, optionally via a linker or carrier.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the compound (e.g., the dsRNA agent), conjugated to one or more lipophilic moieties, can be measured by its protein binding characteristics. For instance, the unbound fraction in the plasma protein binding assay of the compound can be determined to positively correlate to the relative hydrophobicity of the dsRNA agent, which can positively correlate to the silencing activity of the dsRNA agent.

In one embodiment, the plasma protein binding assay determined is an electrophoretic mobility shift assay (EMSA) using human serum albumin protein. The hydrophobicity of the dsRNA agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of dsRNA.

Accordingly, conjugating the lipophilic moieties to the dsRNA agent provides optimal hydrophobicity for the enhanced in vivo delivery of dsRNA.

In certain embodiments, one or more lipophilic moieties can be an aliphatic, cyclic such as alicyclic, or polycyclic such as polyalicyclic compound, such as a steroid (e.g., sterol) or a linear or branched aliphatic hydrocarbon. One or more lipophilic moieties may generally comprise a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, one or more lipophilic moieties may contain a C₄-C₃₀ hydrocarbon chain (e.g., C₄-C₃₀ alkyl or alkenyl). In some embodiment, one or more lipophilic moieties can contain a saturated or unsaturated C₆-C₁₈ hydrocarbon chain (e.g., a linear C₆-C₁₈ alkyl or alkenyl).

In some embodiments, two or more lipophilic moieties may be conjugated to the dsRNA agent. In some embodiments, only one lipophilic moiety is conjugated to the dsRNA agent.

In one embodiment, at least one lipophilic moiety contains a saturated or unsaturated C₂₂ hydrocarbon chain (e.g., a linear or branched C₂₂ alkyl or alkenyl).

The lipophilic moiety may be attached to the dsRNA agent by any method known in the art, including via a functional grouping already present in the lipophilic monomer or introduced into the dsRNA agent, such as a hydroxy group (e.g., CO—CH₂—OH). The functional groups already present in the lipophilic monomer or introduced into the dsRNA agent include, but are not limited to, hydroxyl, amine, carboxylic acid, sulfonate, phosphate, thiol, azide, and alkyne.

Conjugation of the dsRNA agent and the lipophilic moiety may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl, eicosanyl, docosanyl group, or the like.

In some embodiments, the lipophilic moiety is conjugated to the dsRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, one of the lipophilic moieties may be a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In some embodiments, one of the lipophilic moieties may be an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, one of the lipophilic moieties may be an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglobulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are hereby incorporated by reference in their entirety. Naproxen has the chemical name (S)-6-Methoxy-α-methyl-2-naphthaleneacetic acid and the structure is

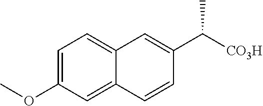

In certain embodiments, the ligand is ibuprofen or a structural derivative of ibuprofen. Procedures for the synthesis of ibuprofen can be found in U.S. Pat. No. 3,228,831, which are hereby incorporated by reference in their entirety. The structure of ibuprofen is

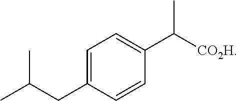

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In some embodiments, one of the lipophilic moieties may be a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexaenoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.).

In certain embodiments, more than one lipophilic moiety can be incorporated into the dsRNA agent, particularly when the lipophilic moiety has a low lipophilicity or hydrophobicity. In one embodiment, two or more lipophilic moieties are incorporated into the same strand of the dsRNA agent. In one embodiment, each strand of the dsRNA agent has one or more lipophilic moieties incorporated. In one embodiment, two or more lipophilic moieties are incorporated into the same position (i.e., the same nucleobase, same sugar moiety, or same internucleosidic linkage) of the dsRNA agent. This can be achieved by, e.g., a using a lipophilic monomer containing a carrier, and/or a branched linker, and/or one or more linkers that can link the two or more lipophilic moieties.

The lipophilic moiety may be conjugated to the dsRNA agent via a direct attachment to the nucleobase, ribosugar, or internucleosidic linkage of the dsRNA agent. Alternatively, the lipophilic moiety may be conjugated to the dsRNA agent via a non-ribose replacement unit, such as a linker or carrier.

In certain embodiments, the lipophilic moiety is conjugated to the dsRNA agent via one or more linkers (tethers).

In one embodiment, the lipophilic moiety is conjugated to the dsRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### Linkers Tethers

Linkers/Tethers are connected to the lipophilic moiety at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C1, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the lipophilic moiety. Non-limited examples of linkers/tethers (underlined) include TAP-(CH₂)ₙNH—; TAP-C(O)(CH₂)ₙNH—; TAP-NR″″(CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙ NH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ (LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙ SH, TAP-(CH₂)ₙ—(CH═CH₂), or TAP-C(O)(CH₂)ₙ (CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)~C(O)ONHS; TAP-C(O)(CH₂)~C(O)ONHS; or TAP-NR″″(CH₂)~C(O)ONHS, in which n is 1-6 and R″″ is C₁-C₆ alkyl; TAP-(CH₂)~C(O)OC₆F₅; TAP-C(O)(CH₂)~C(O) OC₆F₅; or TAP-NR″″(CH₂)ₙC(O) OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether

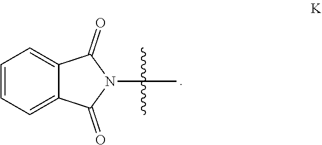 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the dsRNA agent from a ligand (e.g., a targeting or cell-permeable ligand, such as cholesterol) inside the cell, or into the desired compartment of the cell.

A chemical junction (e.g., a linking group) that links a ligand to a dsRNA agent can include a disulfide bond. When the dsRNA agent/ligand complex is taken up into the cell by endocytosis, the acidic environment of the endosome will cause the disulfide bond to be cleaved, thereby releasing the dsRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the dsRNA agent.

A tether can include a linking group that is cleavable by a particular enzyme. The type of linking group incorporated into a tether can depend on the cell to be targeted by the dsRNA agent. For example, a dsRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the dsRNA agent from a ligand that is attached to the distal end of the tether, thereby potentially enhancing silencing activity of the dsRNA agent. Other cell-types rich in esterases include cells of the lung, renal cortex, and testis.

Tethers that contain peptide bonds can be conjugated to dsRNA agents target to cell types rich in peptidases, such as liver cells and synoviocytes. For example, a dsRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the dsRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### Biocleavable Linkers Tethers

The linkers can also include biocleavable linkers that are nucleotide and non-nucleotide linkers or combinations thereof that connect two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include, without limitation, the following endosomal cleavable linkers as well as phosphoramidites:

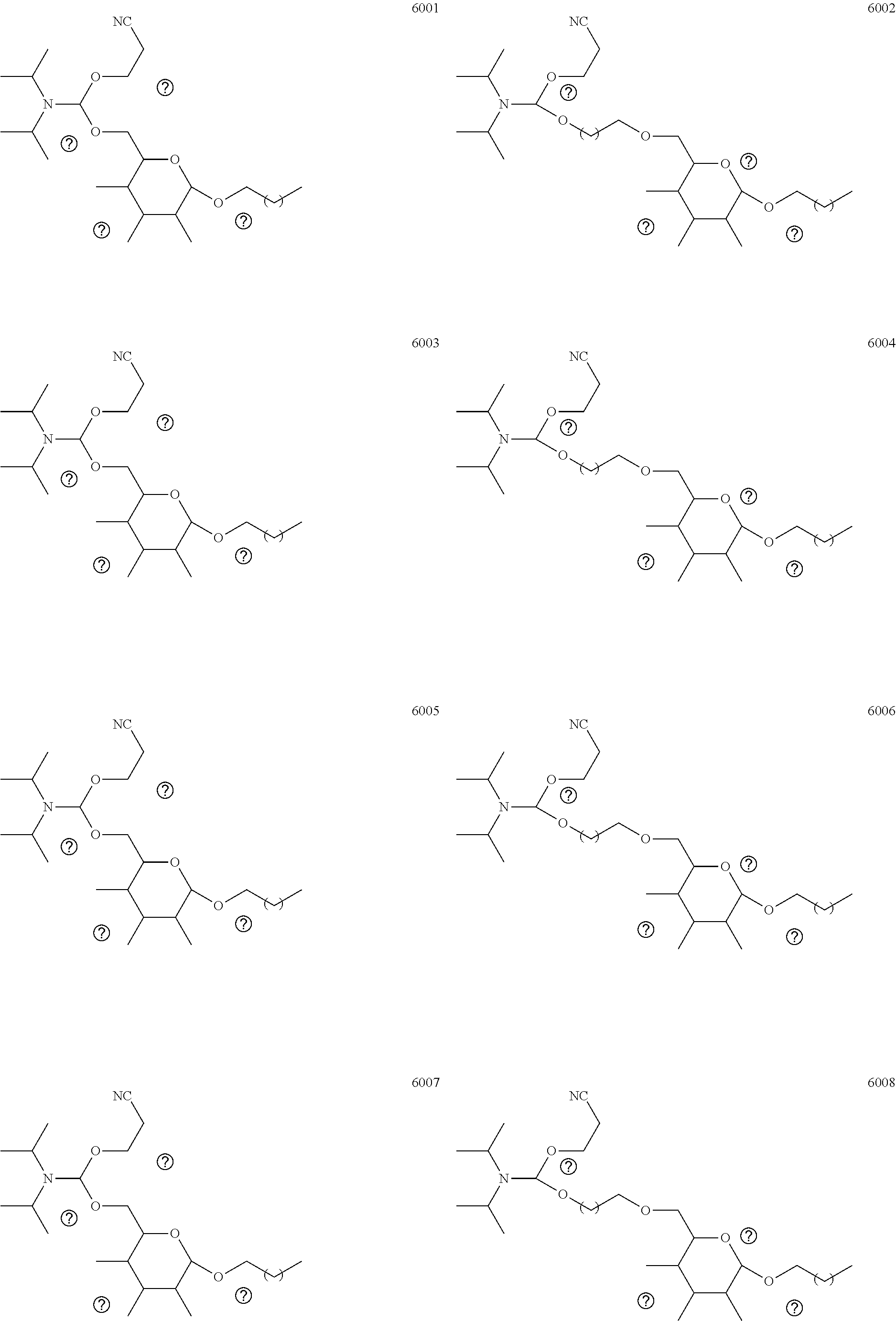

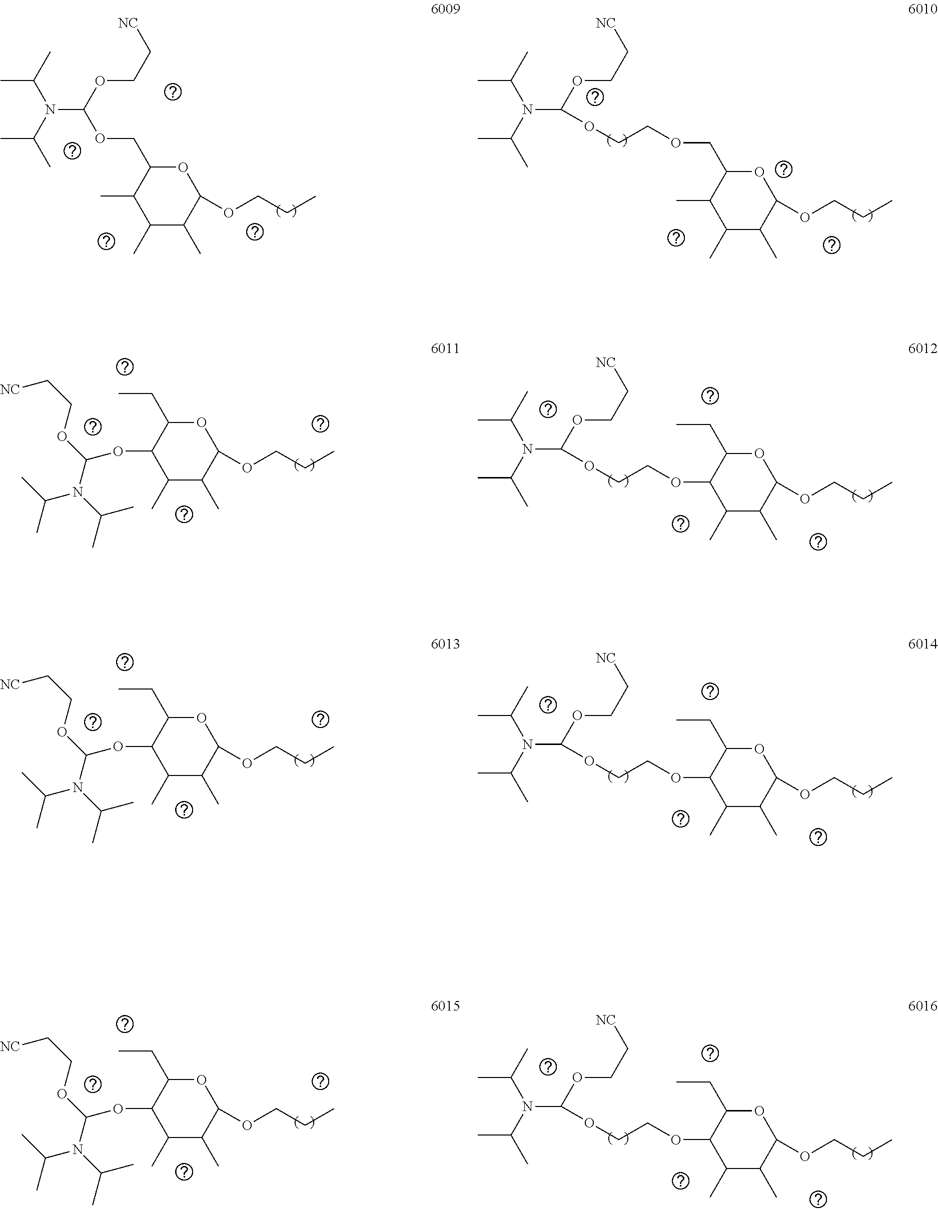

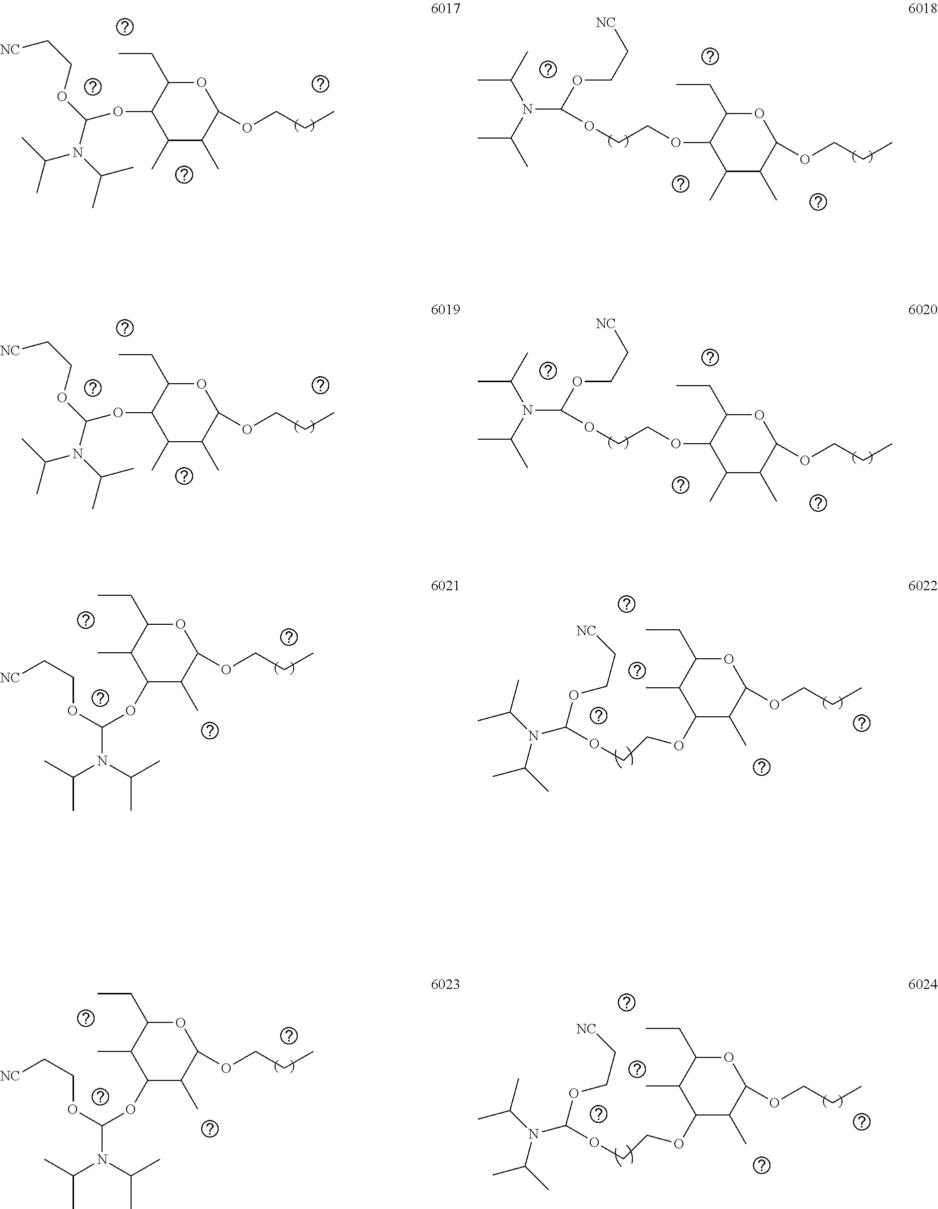

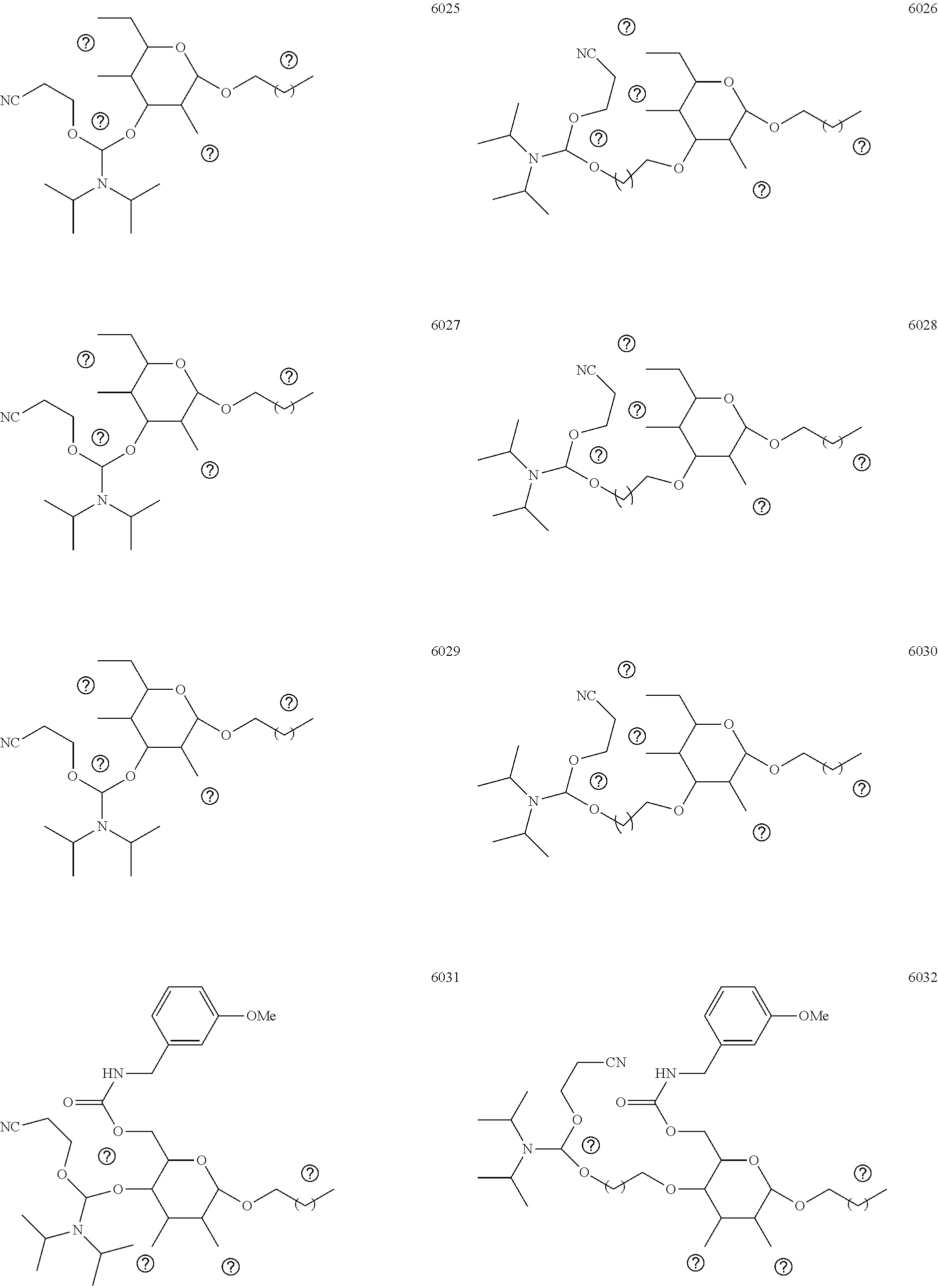

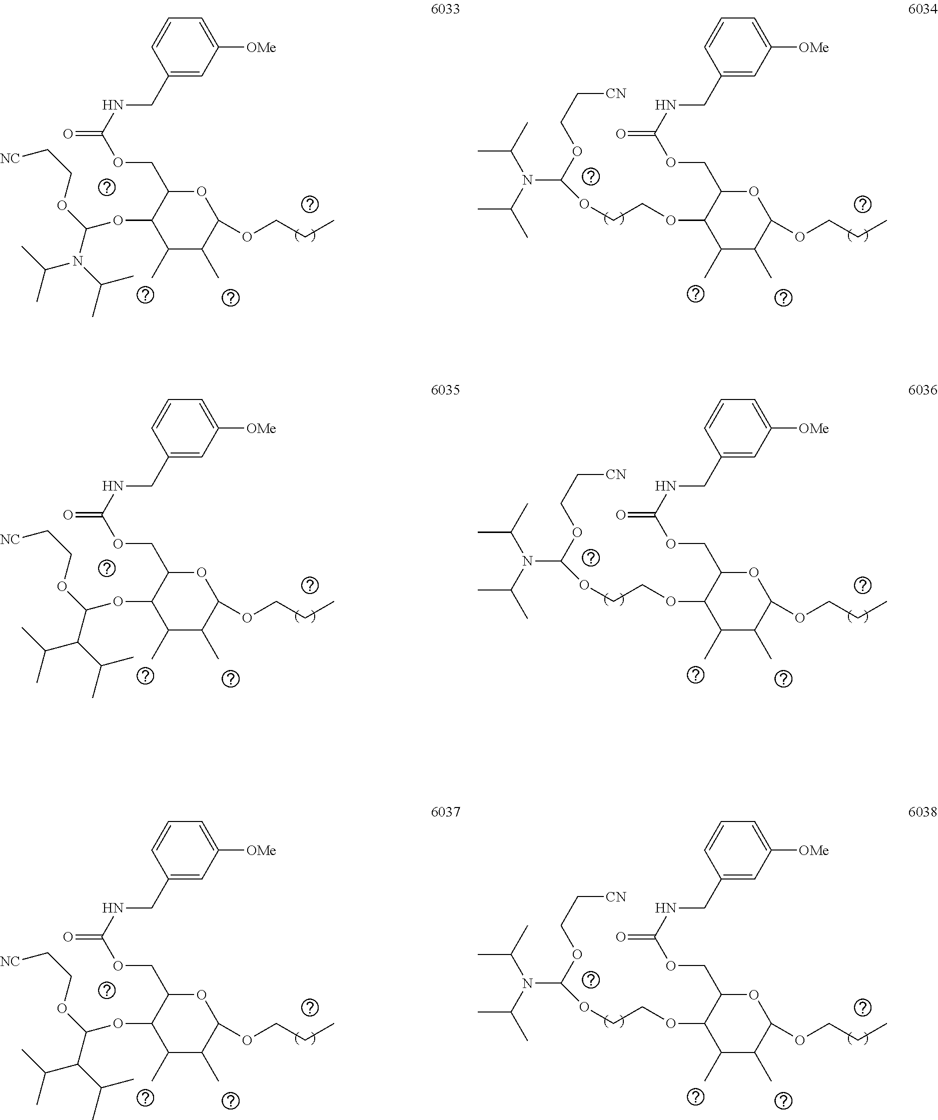

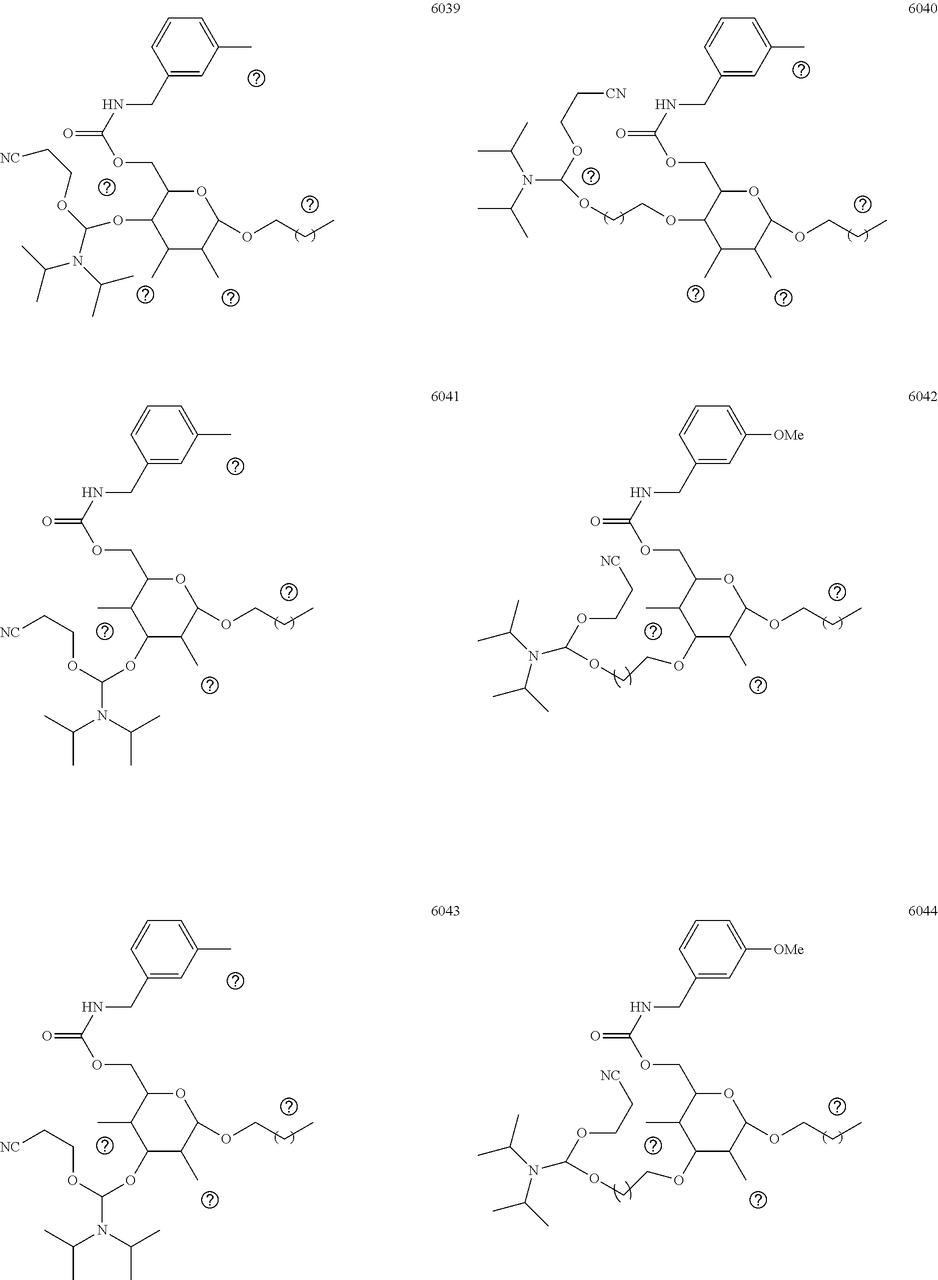

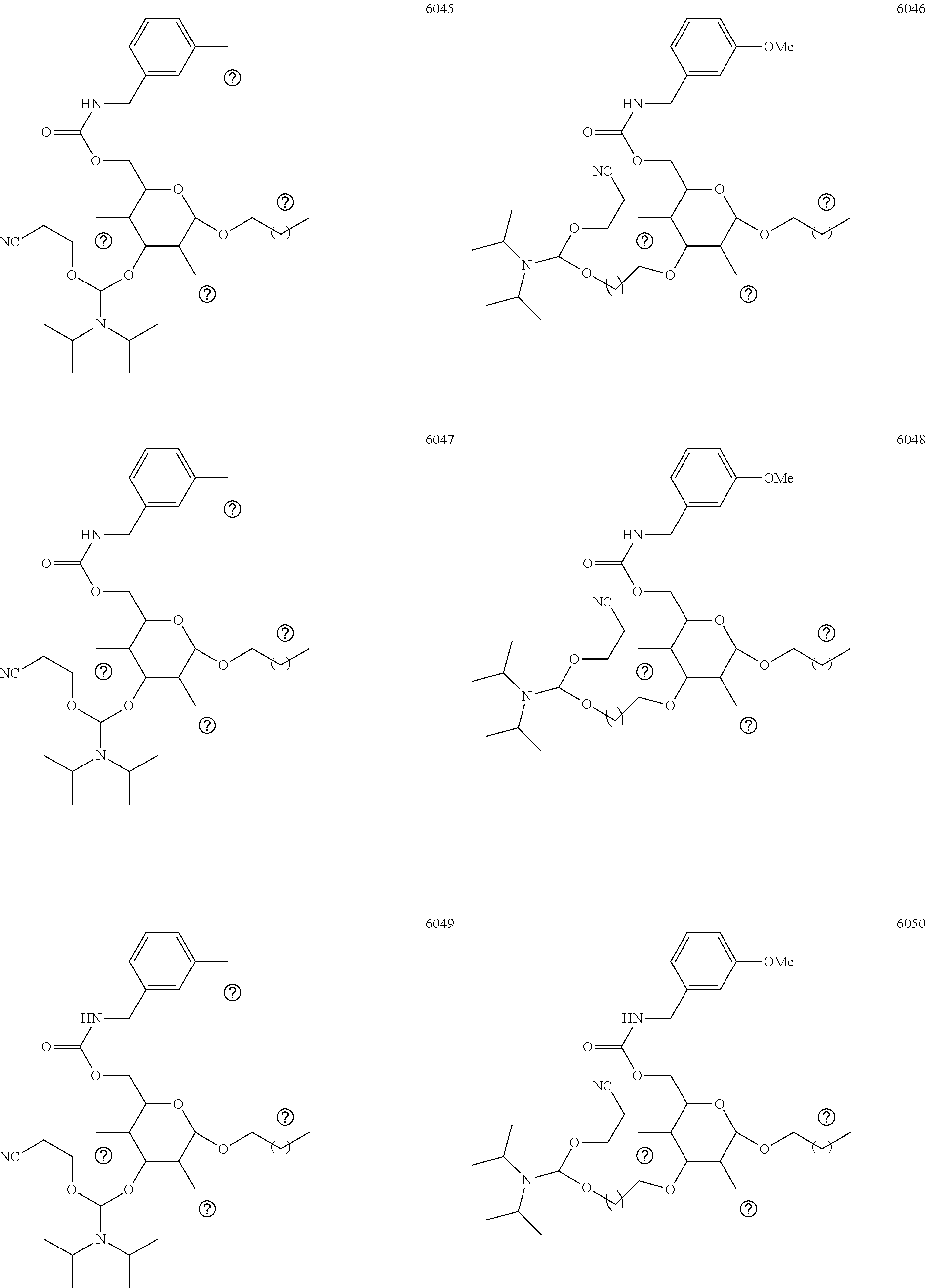

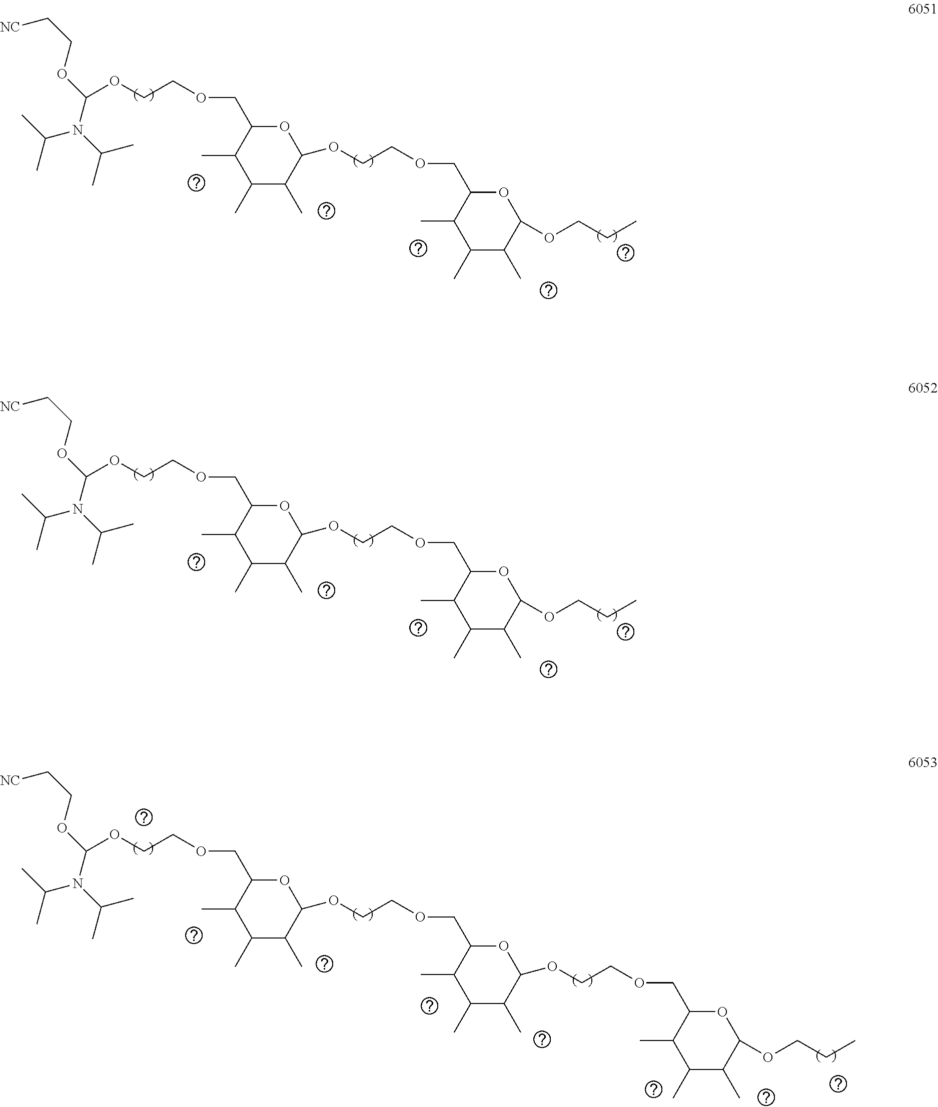

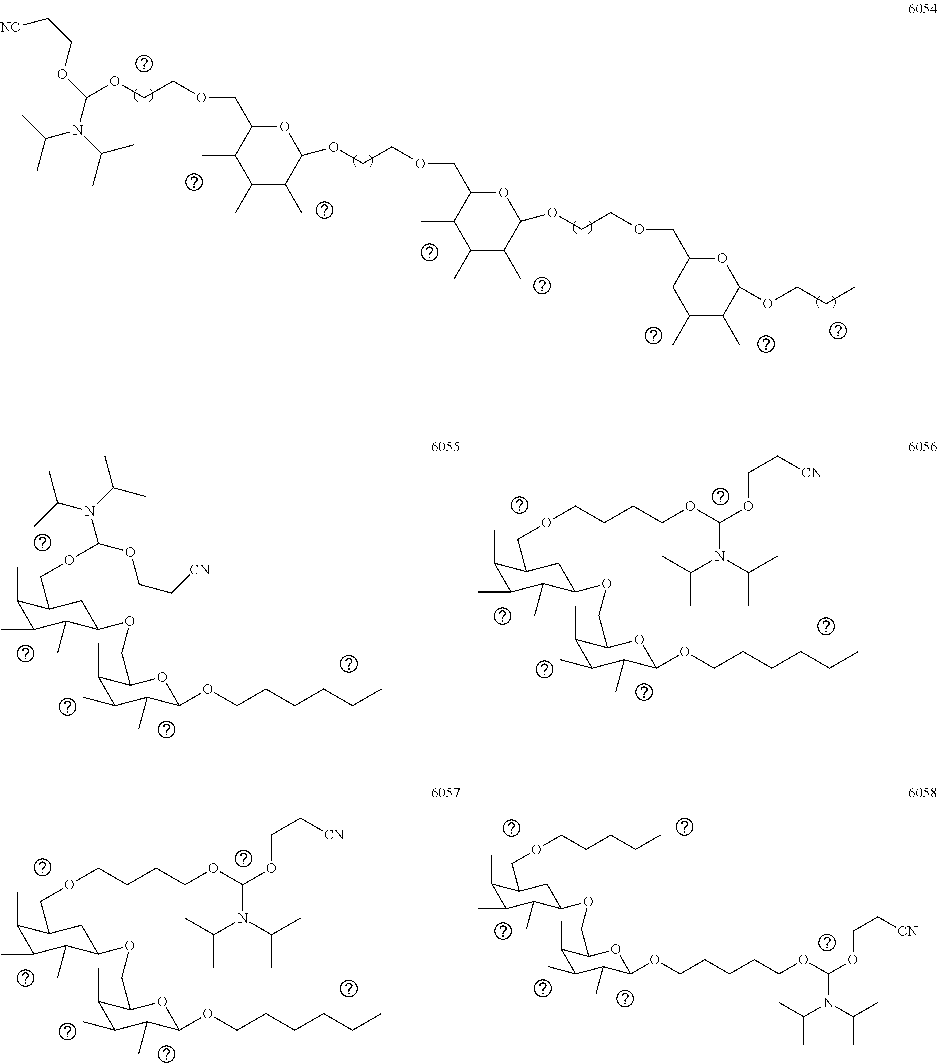

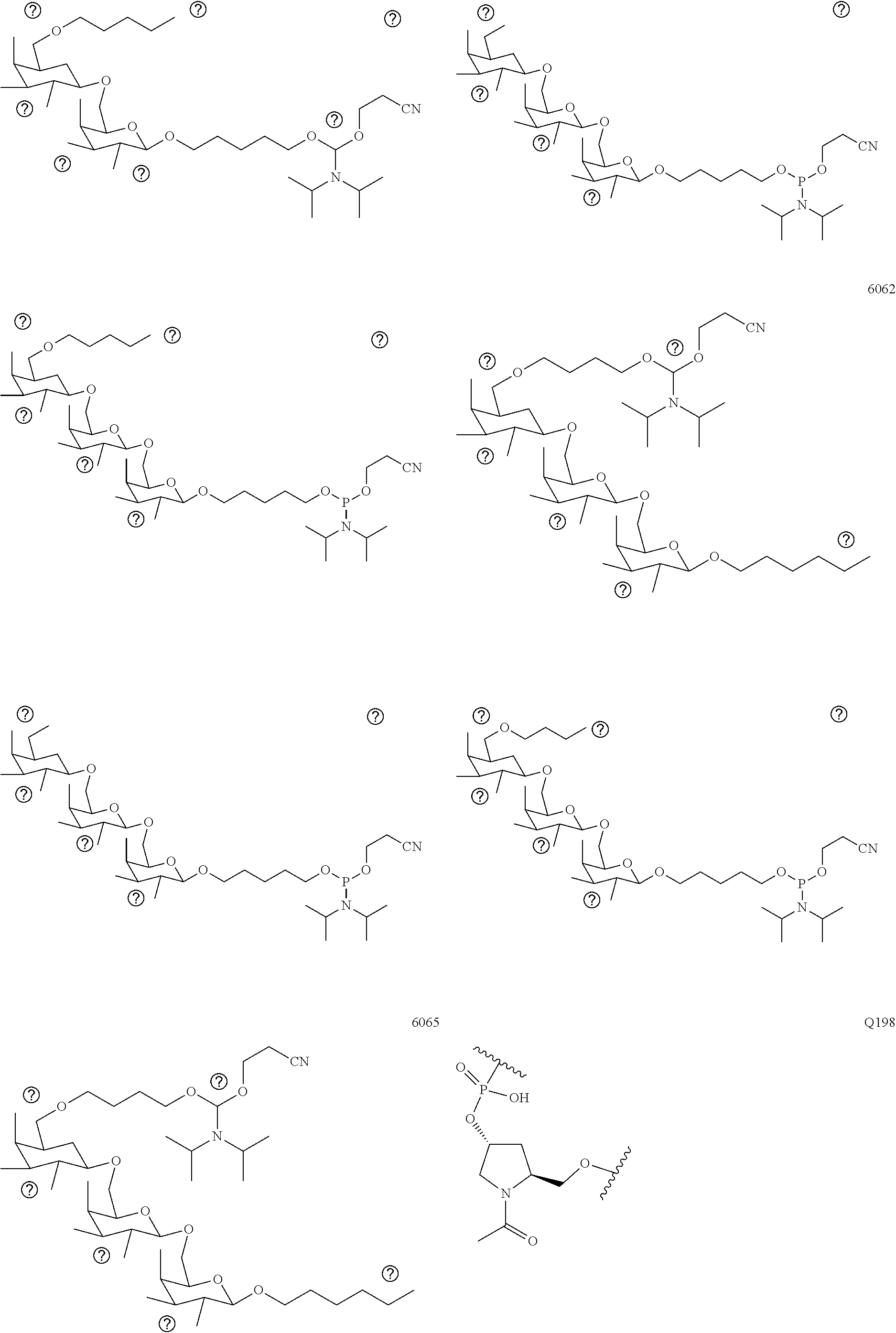

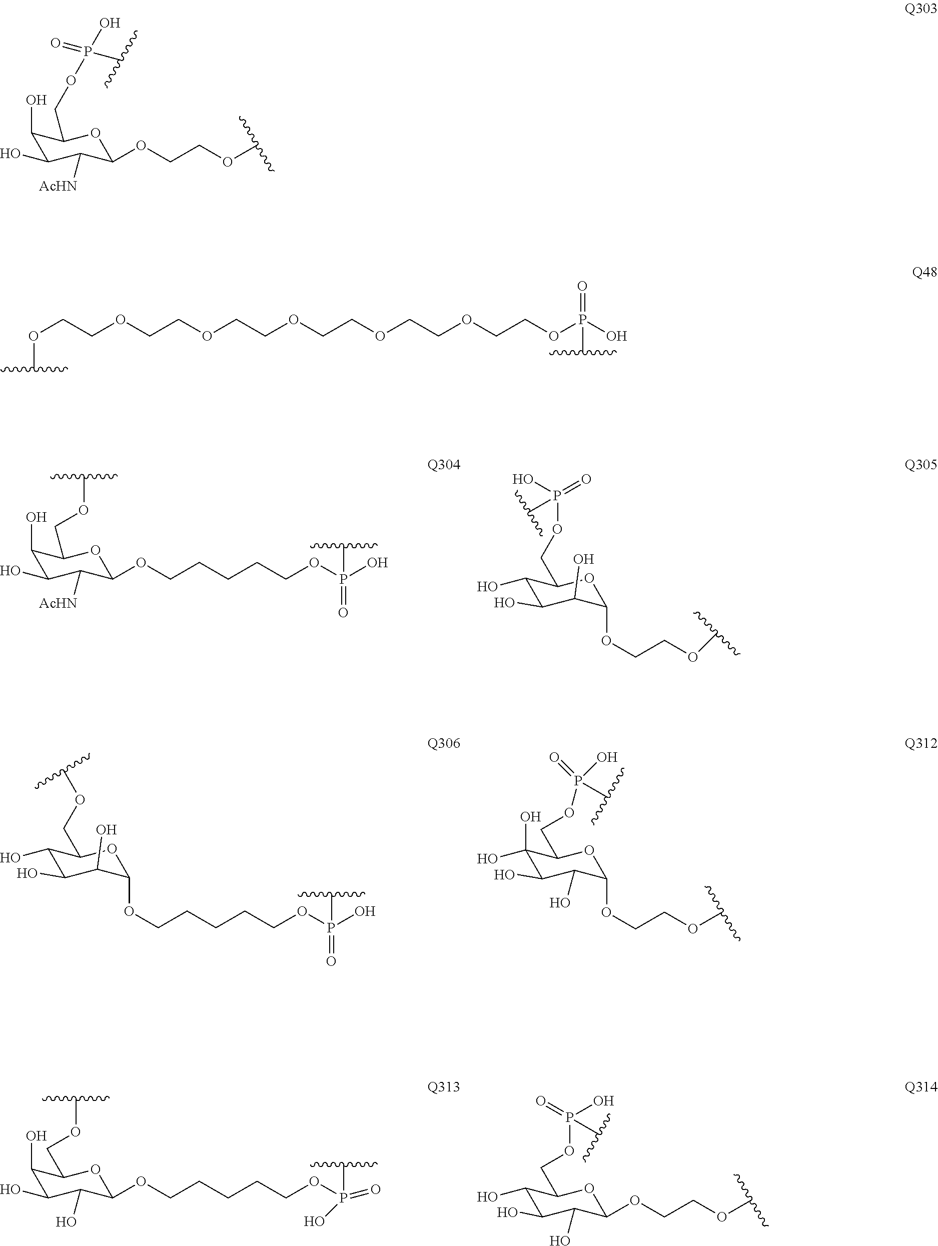

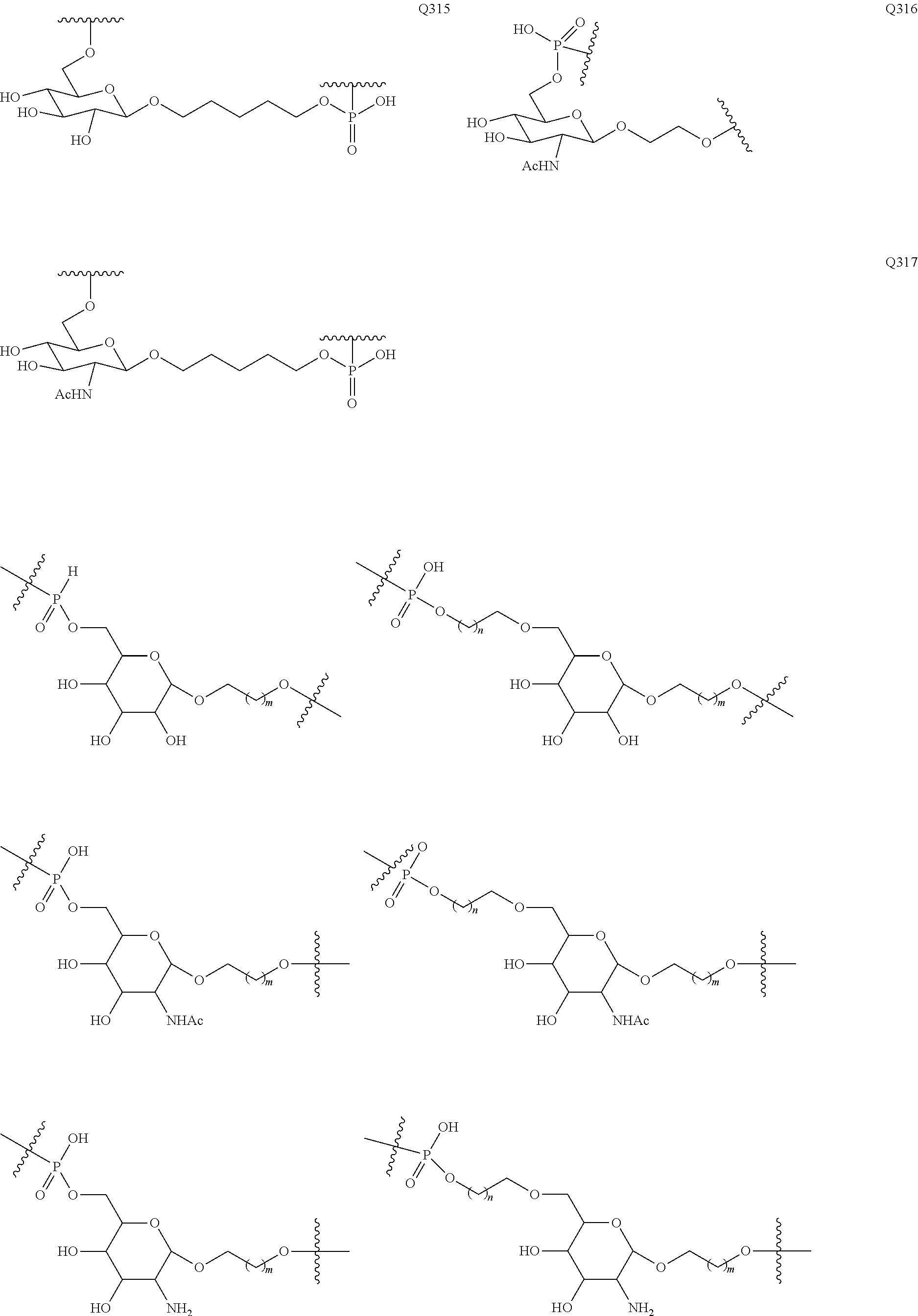

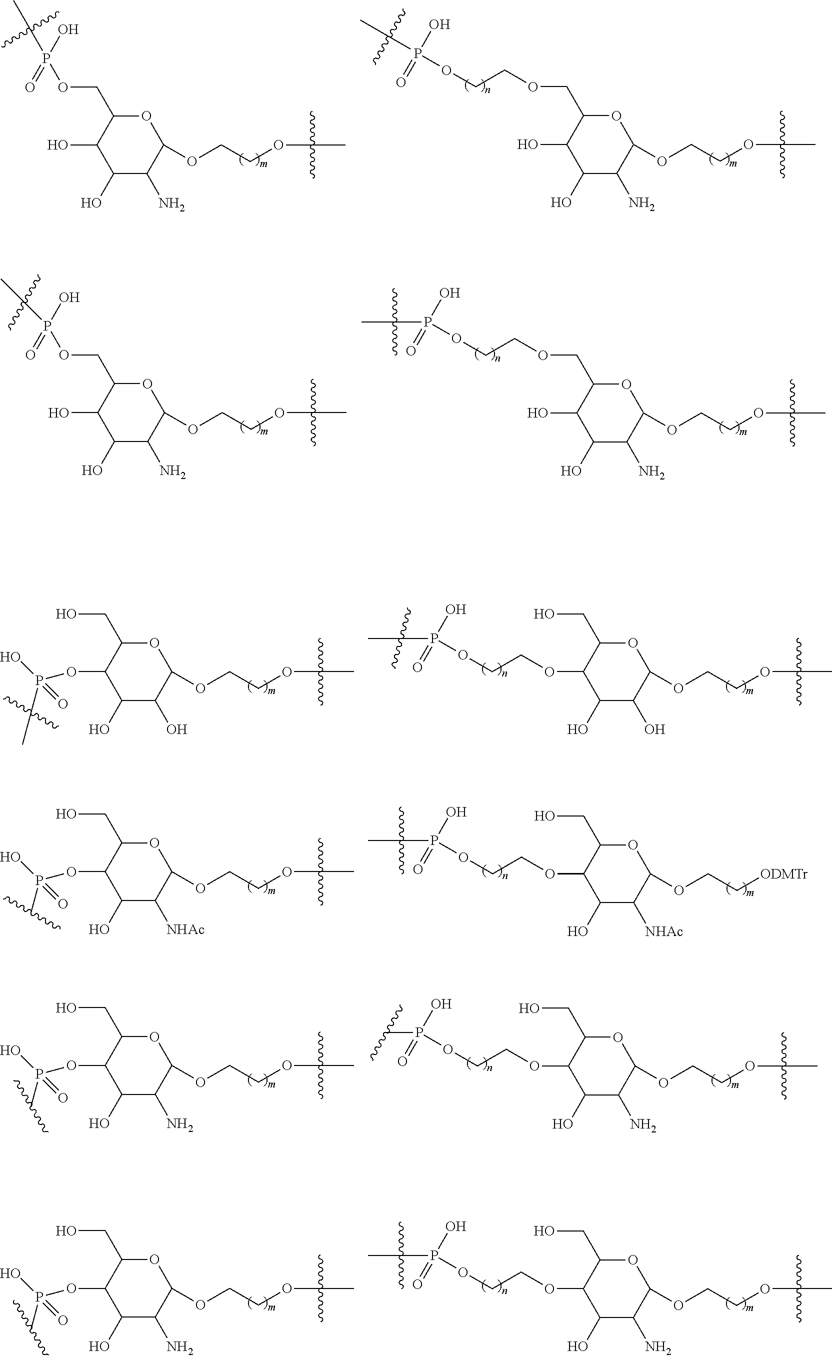

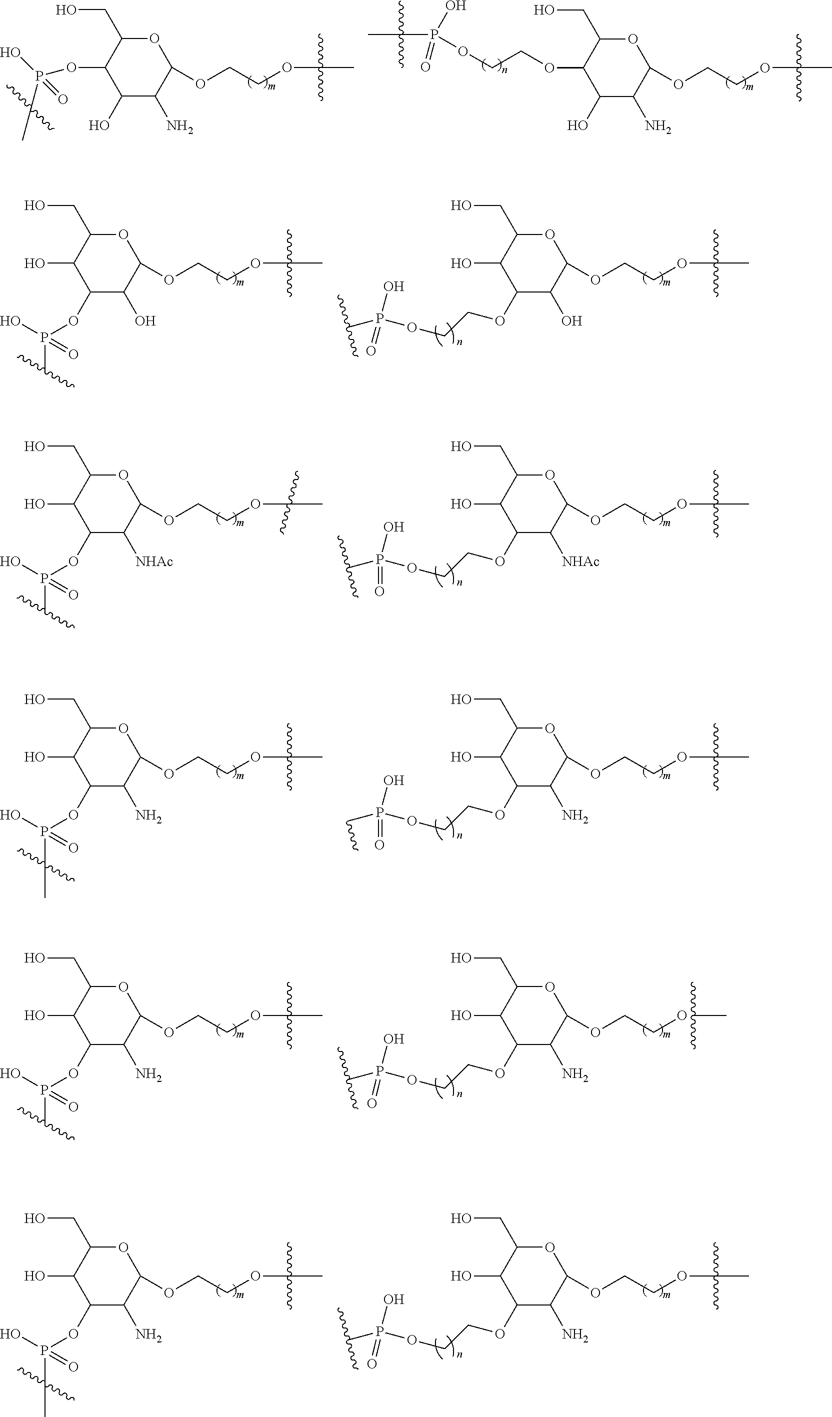

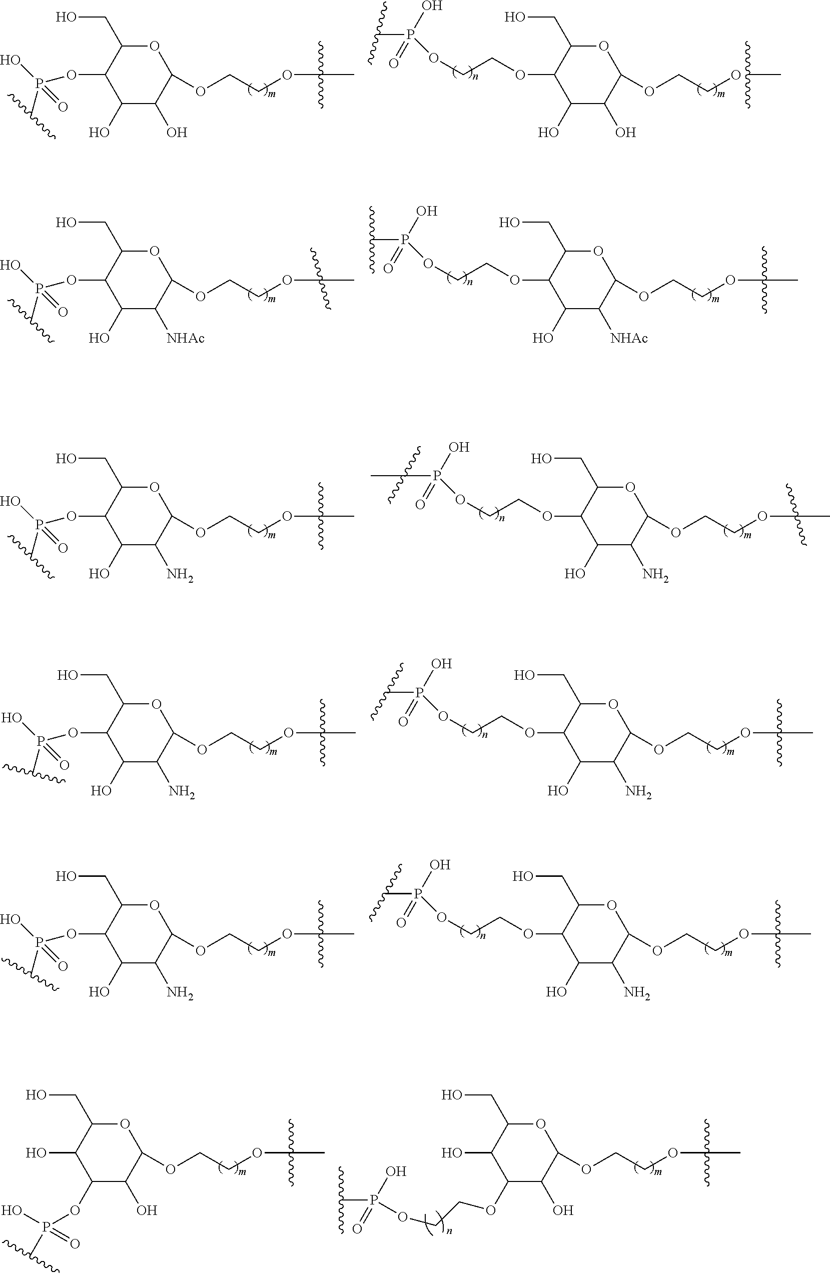

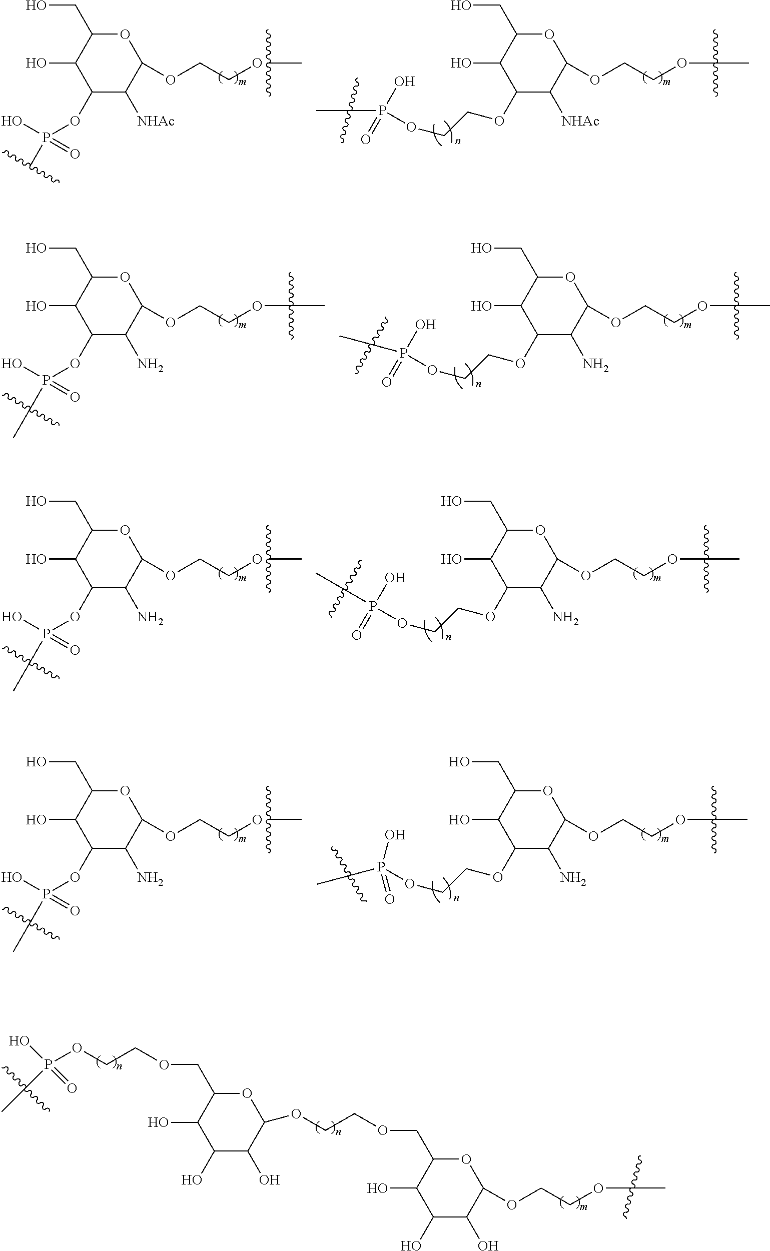

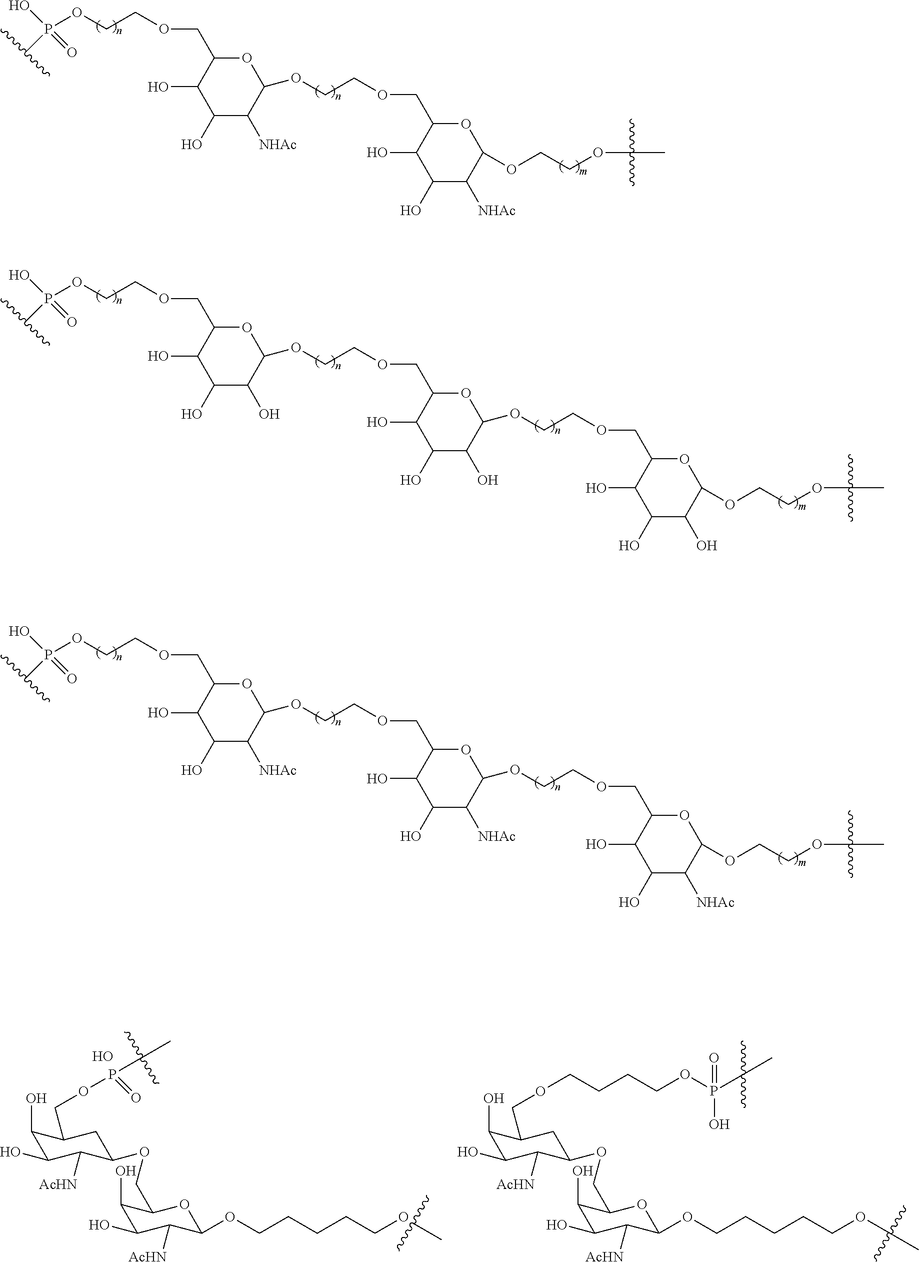

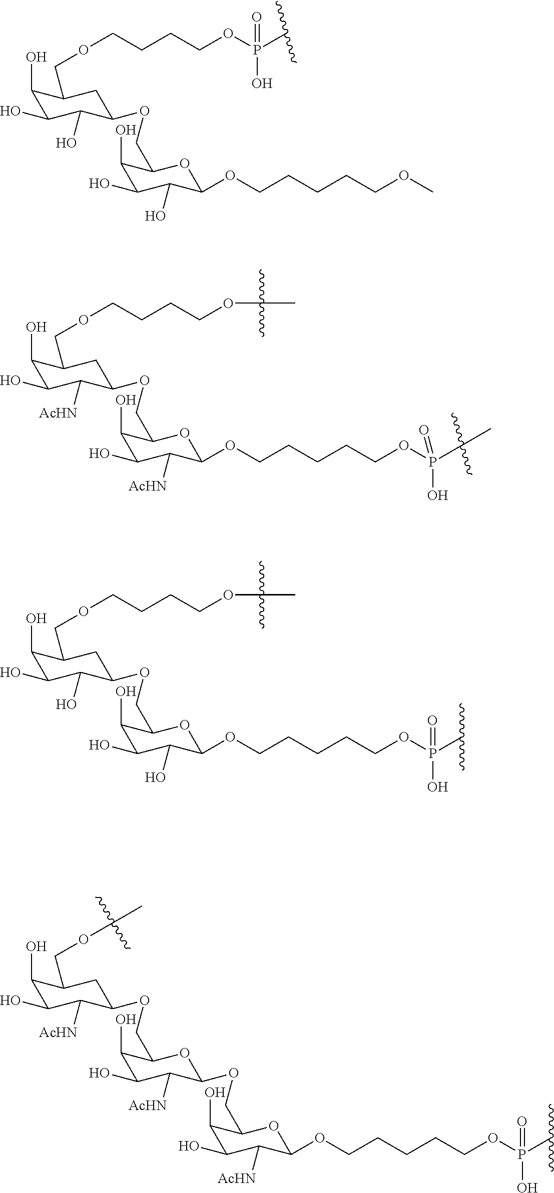 [SMILES: *P(=O)(O)OCCCCOCC1CC(OCC2OC(OCCCCCOC)C(O)C(O)C2O)C(O)C(O)C1O ; *OCCCCOCC1CC(OCC2OC(OCCCCCOP(*)(=O)O)C(NC(C)=O)C(O)C2O)C(NC(C)=O)C(O)C1O ; *OCCCCOCC1CC(OCC2OC(OCCCCCOP(*)(=O)O)C(O)C(O)C2O)C(O)C(O)C1O ; *OCC1CC(OCC2OC(OCC3OC(OCCCCCOP(*)(=O)O)C(NC(C)=O)C(O)C3O)C(NC(C)=O)C(O)C2O)C(NC(C)=O)C(O)C1O]

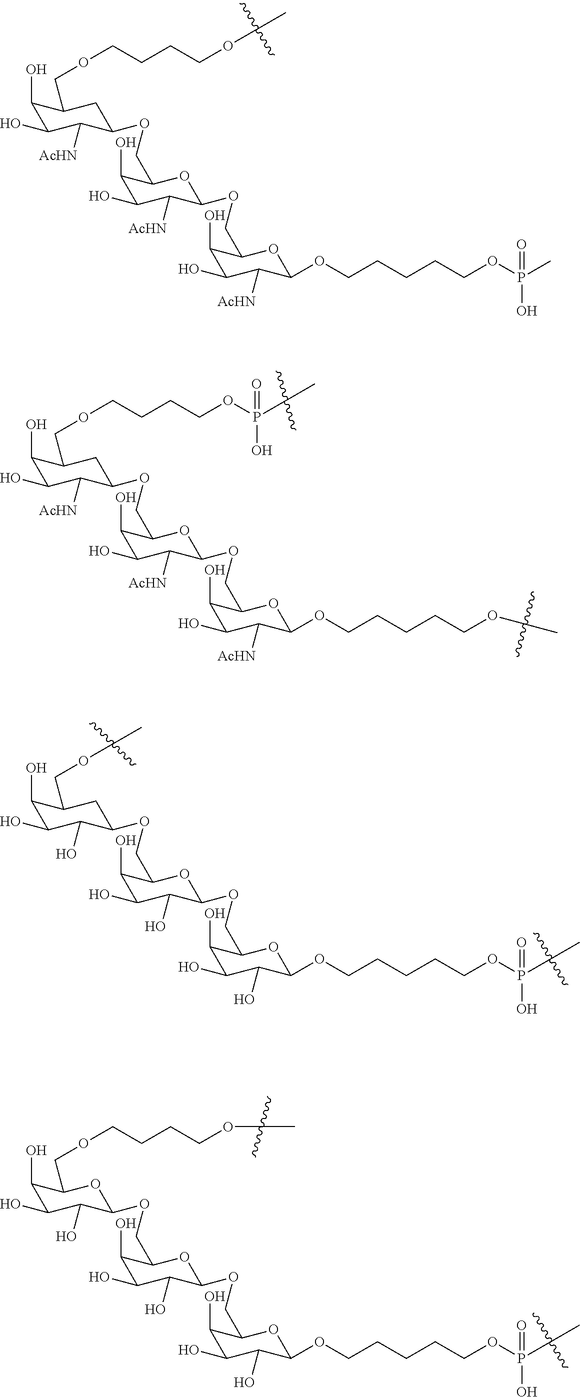 [SMILES: *OCCCCOCC1CC(OCC2OC(OCC3OC(OCCCCCOP(C)(=O)O)C(NC(C)=O)C(O)C3O)C(NC(C)=O)C(O)C2O)C(NC(C)=O)C(O)C1O ; *OCCCCCOC1OC(COC2OC(COC3CC(COCCCCOP(*)(=O)O)C(O)C(O)C3NC(C)=O)C(O)C(O)C2NC(C)=O)C(O)C(O)C1NC(C)=O ; *OCC1CC(OCC2OC(OCC3OC(OCCCCCOP(*)(=O)O)C(O)C(O)C3O)C(O)C(O)C2O)C(O)C(O)C1O ; *OCCCCOCC1OC(OCC2OC(OCC3OC(OCCCCCOP(*)(=O)O)C(O)C(O)C3O)C(O)C(O)C2O)C(O)C(O)C1O]

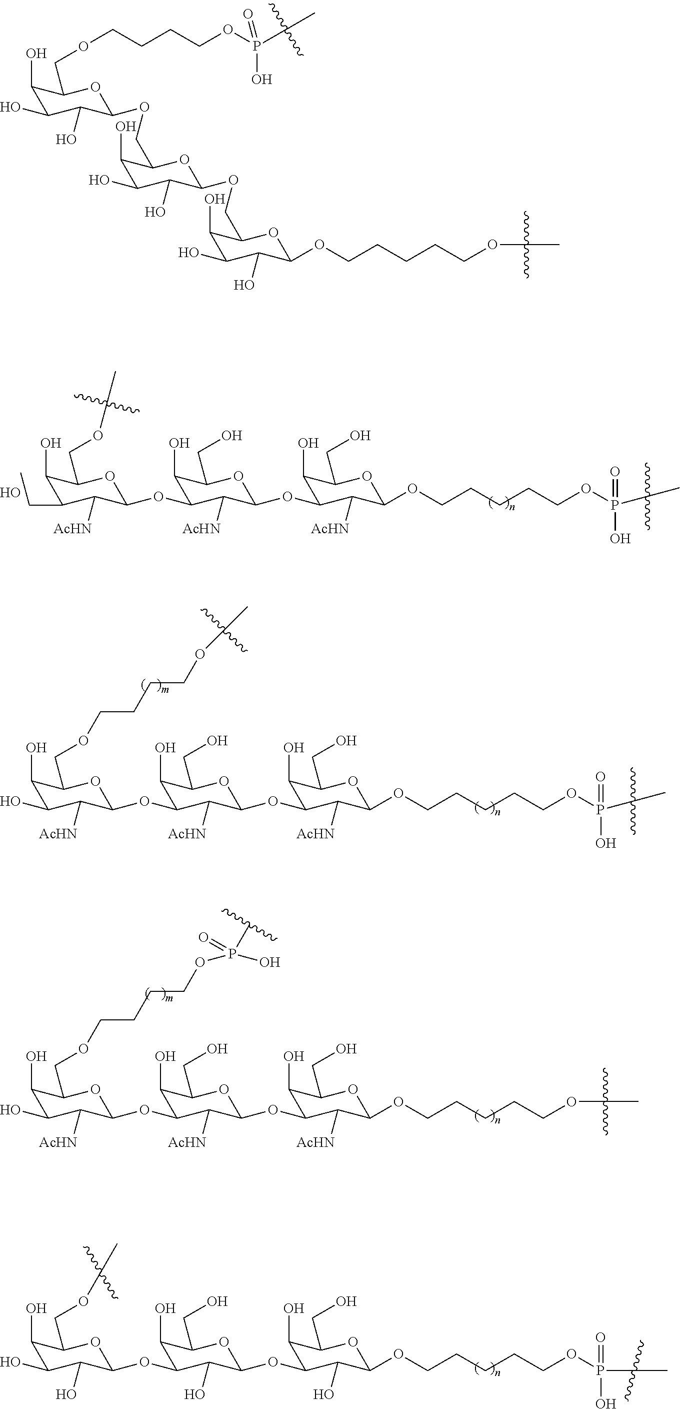

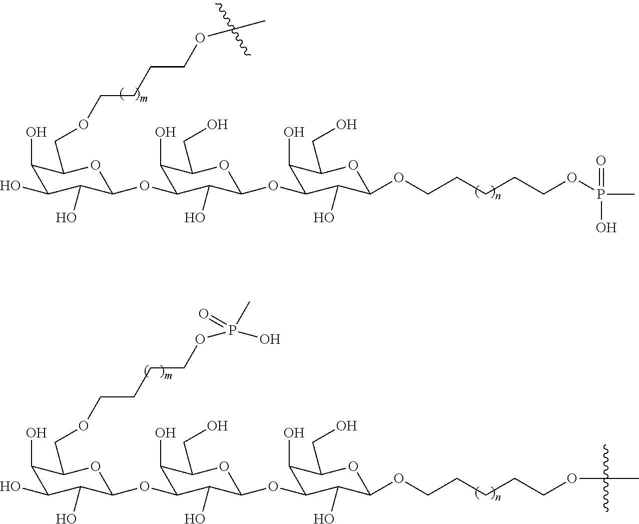

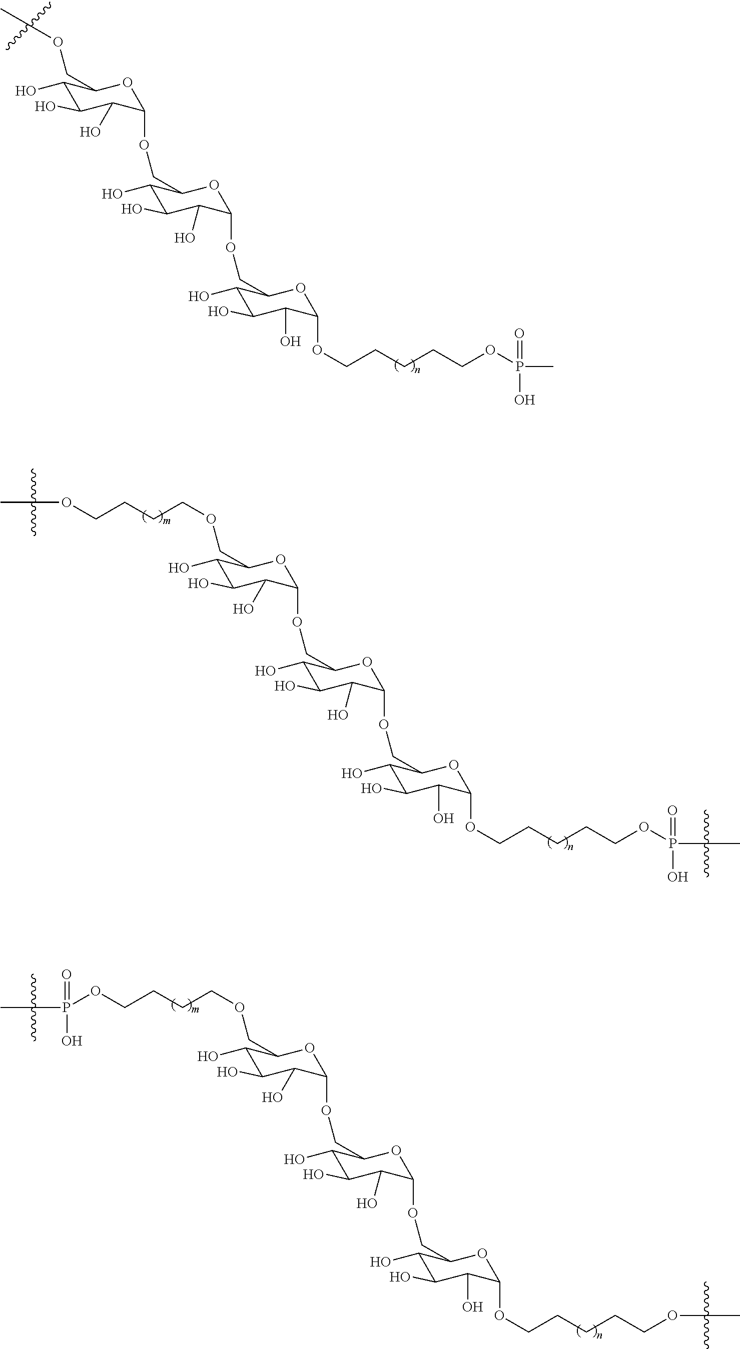

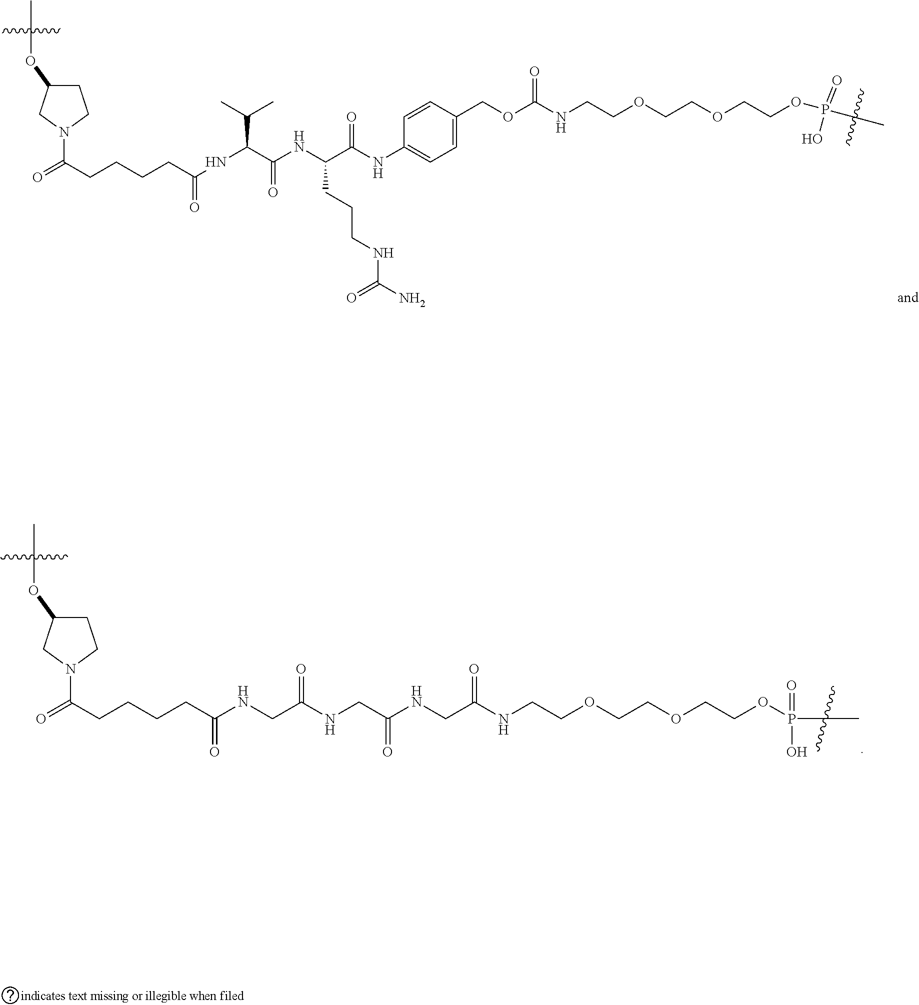 [SMILES: *O[C@H]1CCN(C(=O)CCCCC(=O)N[C@H](C(=O)N[C@@H](CCCNC(N)=O)C(=O)Nc2ccc(COC(=O)NCCOCCOCCOP(*)(=O)O)cc2)C(C)C)C1 ; *O[C@H]1CCN(C(=O)CCCCC(=O)NCC(=O)NCC(=O)NCC(=O)NCCOCCOCCOP(*)(=O)O)C1]

More discussion about the biocleavable linkers may be found in PCT application No. PCT/US18/14213, entitled “Endosomal Cleavable Linkers,” filed on Jan. 18, 2018, the content of which is incorporated herein by reference in its entirety.

### Carriers

In certain embodiments, the lipophilic moiety is conjugated to the dsRNA agent via a non-ribose replacement unit, i.e., a carrier that replaces one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

The carrier can replace one or more nucleotide(s) of the dsRNA agent.

In some embodiments, the carrier replaces one or more nucleotide(s) in the internal position(s) of the dsRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand (e.g., the lipophilic moiety). The lipophilic moiety can be directly attached to the carrier or indirectly attached to the carrier by an intervening linker/tether, as described above.

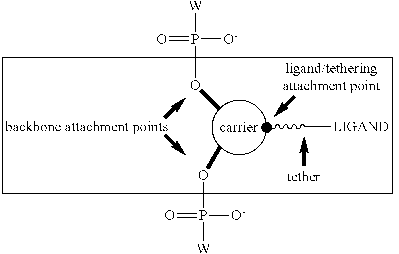

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in a dsRNA agent.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

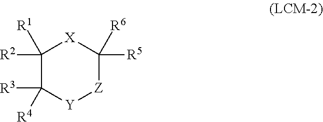

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b}; Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or
Si(X^{5′})(X^{5″})(X^{5′″}) in which (X^{5′}), (X^{5″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy; n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

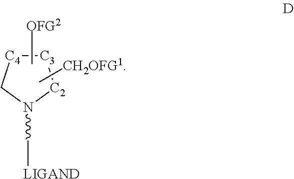

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

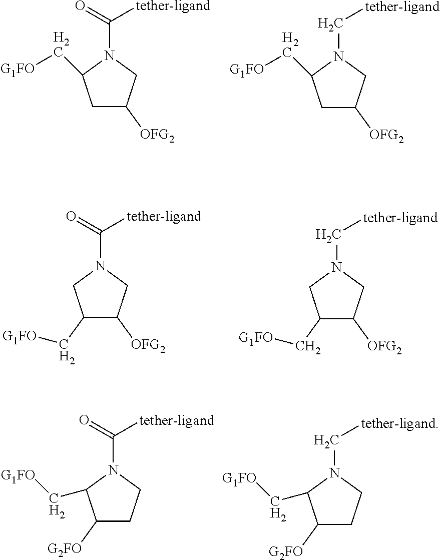

In certain embodiments, the carrier may be based on the piperdine ring system (E), e.g., X is N(CO)R⁷ or NR⁷, Y is CR⁹R¹⁰, and Z is CR¹¹R¹².

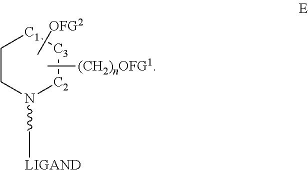

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFGⁿ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or
—(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹²

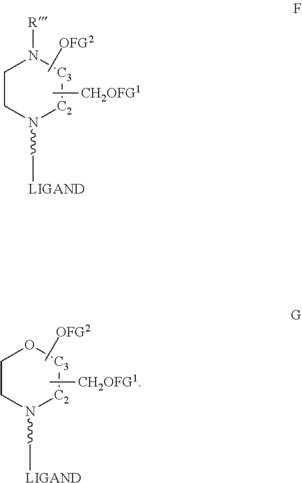

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

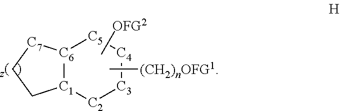

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

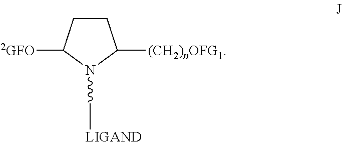

Thus, —(CH₂)~OFG¹ and OFG² may be cis or trans with respect to one another. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

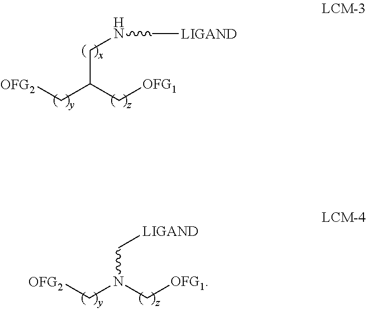

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the dsRNA agent comprises one or more lipophilic moieties conjugated to the 5′ end of the sense strand or the 5′ end of the antisense strand.

In certain embodiments, the lipophilic moiety is conjugated to the 5′-end of a strand via a carrier and/or linker. In one embodiment, the lipophilic moiety is conjugated to the 5′-end of a strand via a carrier of a formula:

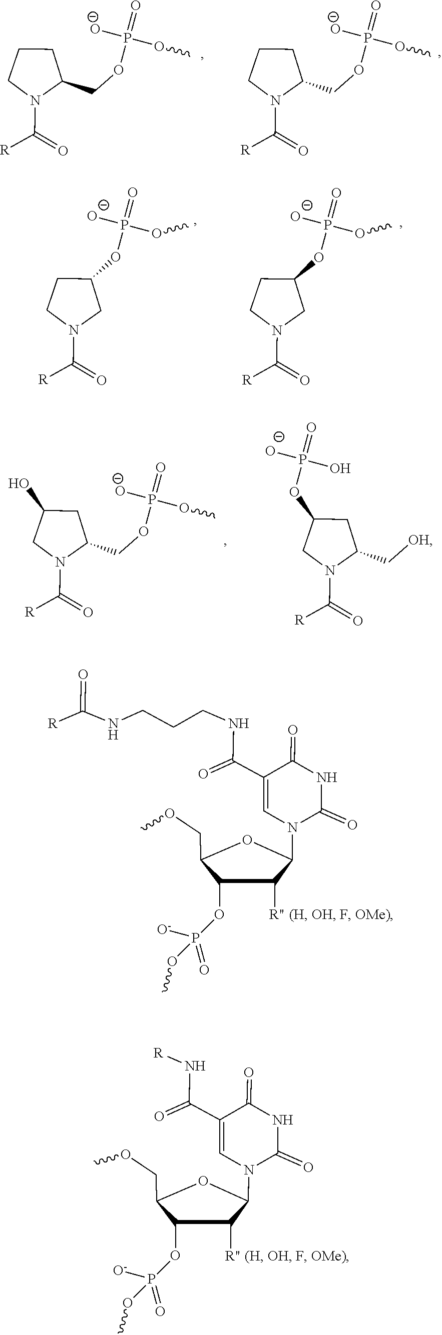

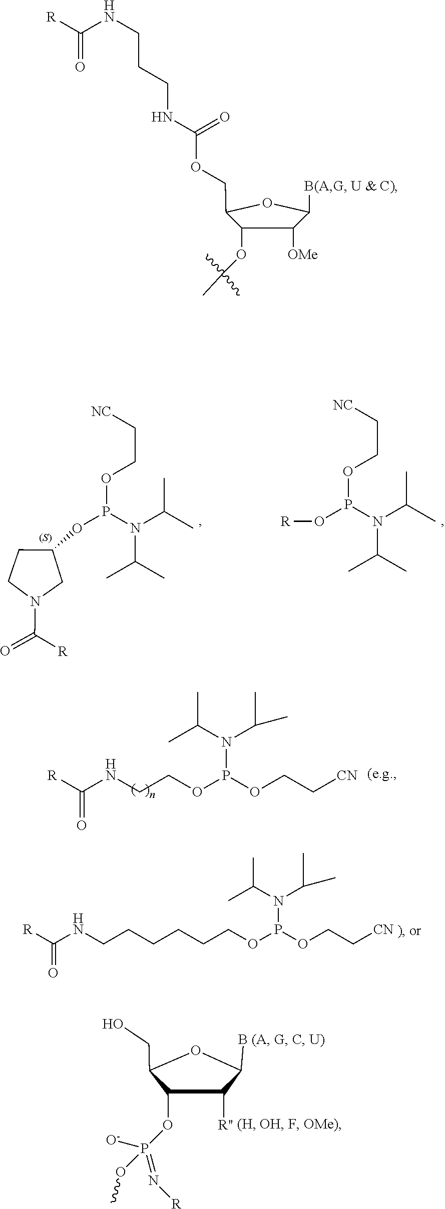

wherein R is a ligand such as the lipophilic moiety. In one embodiment, R is the saturated or unsaturated C₂₂ hydrocarbon chains as described above, optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl (for instance, R is optionally substituted with a —OR^{G}, —C(O)OR^{G}, or —N(R^{G})C(O)R^{G}). In one embodiment, R together with the carbonyl to which it is attached may form a group with 22 carbons (for instance, R may be a saturated or unsaturated C₂₁ hydrocarbon chain, such as a linear or branched C₂₁ alkyl group), optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl (for instance, R is optionally substituted with a —OR^{G}, —C(O)OR^{G}, or —N(R^{G})C(O)R^{G}). In some embodiments, R is substituted with OH or COOH

In some embodiments, the dsRNA agent comprises one or more lipophilic moieties conjugated to the 3′ end of the sense strand or the 3′ end of the antisense strand.

In certain embodiments, the lipophilic moiety is conjugated to the 3′-end of a strand via a carrier and/or linker. In one embodiment, the lipophilic moiety is conjugated to the 3′-end of a strand via a carrier of a formula:

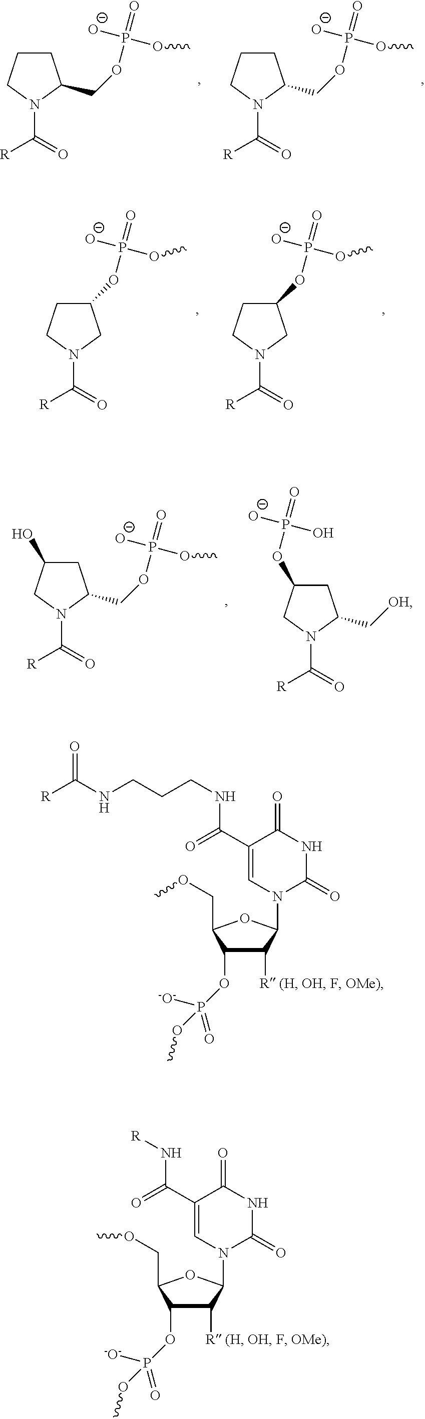

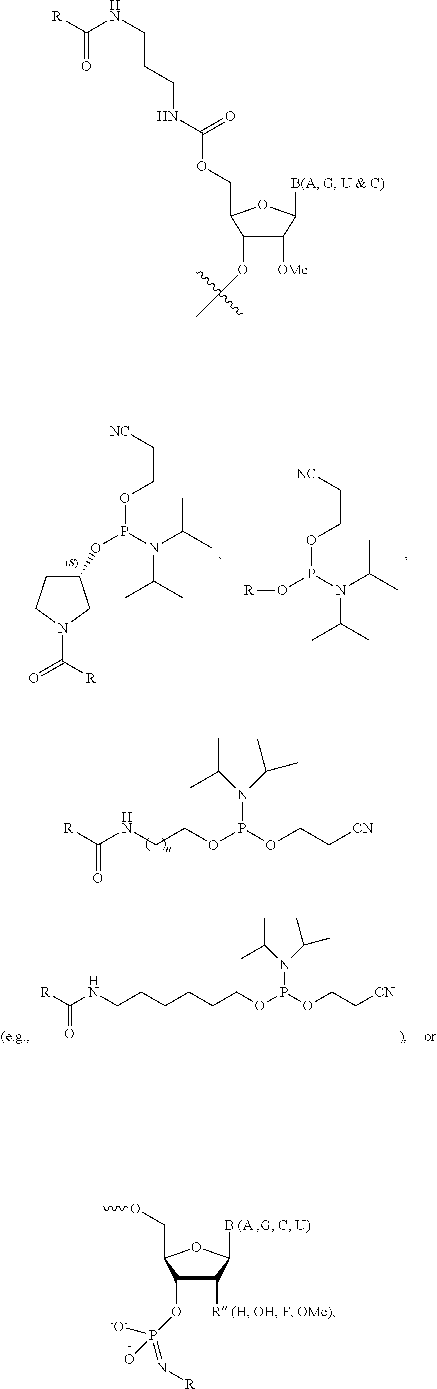

wherein R is a ligand such as the lipophilic moiety. In one embodiment, is the saturated or unsaturated C₂₂ hydrocarbon chains as described above, optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl (for instance, R is optionally substituted with a —OR^{G}, —C(O)OR^{G}, or —N(R^{G})C(O)R^{G}). In one embodiment, R together with the carbonyl to which it is attached may form a group with 22 carbons (for instance, R may be a saturated or unsaturated C₂₁ hydrocarbon chain, such as a linear or branched C₂₁ alkyl group), optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl (for instance, R is optionally substituted with a —OR^{G}, —C(O)OR^{G}, or —N(R^{G})C(O)R^{G}). In some embodiments, R is substituted with OH or COOH.

In certain embodiments, the lipophilic moiety is conjugated to the internal position of a strand via a carrier and/or linker. In one embodiment, the lipophilic moiety is conjugated to the internal position of a strand via a carrier of a formula:

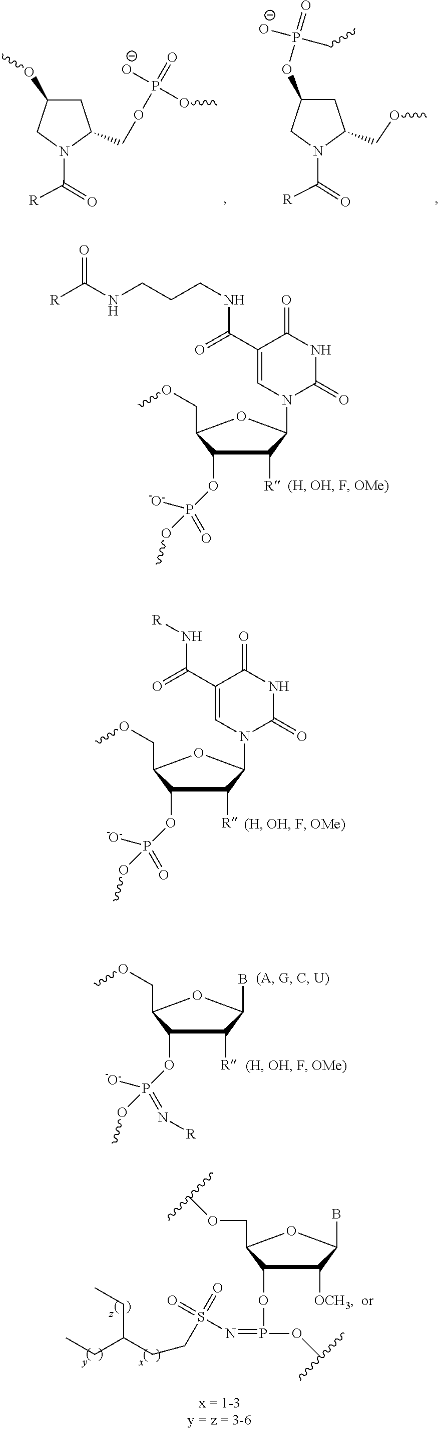

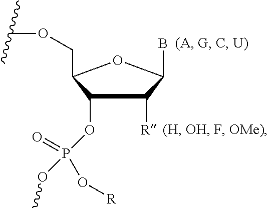

wherein R is a ligand such as the lipophilic moiety. In one embodiment, R is the saturated or unsaturated C₂₂ hydrocarbon chains as described above.

In some embodiments, the dsRNA agent comprises one or more lipophilic moieties conjugated to both ends of the sense strand.

In some embodiments, the dsRNA agent comprises one or more lipophilic moieties conjugated to both ends of the antisense strand.

In some embodiments, the dsRNA agent comprises one or more lipophilic moieties conjugated to internal position of the sense or antisense strand. In some embodiments, one or more lipophilic moieties are conjugated to the ribose, nucleobase, and/or at the internucleotide linkages. In some embodiments, one or more lipophilic moieties are conjugated to the ribose at the 2′ position, 3′ position, 4′ position, and/or 5′ position of the ribose. In some embodiments, one or more lipophilic moieties are conjugated at the nucleobase of natural (such as A, T, G, C, or U) or modified as defined herein. In some embodiments, one or more lipophilic moieties are conjugated at the phosphate or modified phosphate groups as defined herein.

In some embodiments, the dsRNA agent comprises one or more lipophilic moieties conjugated to the 5′ end or 3′ end of the sense strand, and one or more lipophilic moieties conjugated to the 5′ end or 3′ end of the antisense strand,

In some embodiments, the dsRNA agent comprises a lipophilic moiety conjugated to the terminal end of a strand via one or more linkers (tethers) and/or a carrier.

In one embodiment, the dsRNA agent comprises a lipophilic moiety conjugated to the terminal end of a strand via one or more linkers (tethers).

In one embodiment, the dsRNA agent comprises a lipophilic moiety conjugated to the 5′ end of the sense strand or antisense strand via a cyclic carrier, optionally via one or more intervening linkers (tethers).

In some embodiments, at least one lipophilic moiety is located on one or more terminal positions of the sense strand or antisense strand. In one embodiment, at least one lipophilic moiety is located on the 3′ end or 5′ end of the sense strand. In one embodiment, at least one lipophilic moiety is located on the 3′ end or 5′ end of the antisense strand.

In some embodiments, at least one lipophilic moiety is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, at least one lipophilic moiety is located on one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, at least one lipophilic moiety is located on one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, at least one lipophilic moiety is located on one or more positions of at least one end of the duplex region, which include all positions within the duplex region, but not include the overhang region or the carrier that replaces the terminal nucleotide on the 3′ end of the sense strand.

In one embodiment, at least one lipophilic moiety is located on the sense strand within the first five, four, three, two, or first base pairs at the 5′-end of the antisense strand of the duplex region.

In one embodiment, at least one lipophilic moiety is located on one or more internal positions on at least one strand, except the cleavage site region of the sense strand, for instance, the lipophilic moiety is not located on positions 9-12 counting from the 5′-end of the sense strand, for example, the lipophilic moiety is not located on positions 9-11 counting from the 5′-end of the sense strand. Alternatively, the internal positions exclude positions 11-13 counting from the 3′-end of the sense strand.

In one embodiment, at least one lipophilic moiety is located on one or more internal positions on at least one strand, which exclude the cleavage site region of the antisense strand. For instance, the internal positions exclude positions 12-14 counting from the 5′-end of the antisense strand.

In one embodiment, at least one lipophilic moiety is located on one or more internal positions on at least one strand, which exclude positions 11-13 on the sense strand, counting from the 3′-end, and positions 12-14 on the antisense strand, counting from the 5′-end.

In one embodiment, one or more lipophilic moieties are located on one or more of the following internal positions: positions 4-8 and 13-18 on the sense strand, and positions 6-10 and 15-18 on the antisense strand, counting from the 5′end of each strand.

In one embodiment, one or more lipophilic moieties are located on one or more of the following internal positions: positions 5, 6, 7, 15, and 17 on the sense strand, and positions 15 and 17 on the antisense strand, counting from the 5′end of each strand.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state, e.g., a CNS disorder or disease state.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The dsRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the overall double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand RNA agent” as used herein, is an RNA agent which is made up of a single molecule. It may include a duplexed region, formed by intra-strand pairing, e.g., it may be, or include, a hairpin or pan-handle structure. Single strand RNA agents may be antisense with regard to the target molecule. A single strand RNA agent may be sufficiently long that it can enter the RISC and participate in RISC mediated cleavage of a target mRNA. A single strand RNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an RNA strand that is unpaired with the opposing nucleotide in the duplex when a section of the RNA strand forms base pairs with another strand or with another section of the same strand.

Hairpin RNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) RNA agent” as used herein, is an RNA agent which includes more than one, and in some cases two, strands in which interchain hybridization can form a region of duplex structure.

As used herein, the terms “siRNA activity” and “RNAi activity” refer to gene silencing by an siRNA.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA, e.g., RNAi agent. The % and/or fold difference can be calculated relative to the control or the non-control, for example,

$$\% difference = \frac{[ expression with siRNA - expression without siRNA ]}{expression without siRNA}$$

$$or$$

$$\% difference = \frac{[ expression with siRNA - expression without siRNA ]}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The double-stranded RNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer dsRNAs of between 25 and 30 base pairs in length are preferred. In some embodiments, shorter dsRNAs of between 10 and 15 base pairs in length are preferred. In another embodiment, the dsRNA is at least 21 nucleotides long.

In some embodiments, the double-stranded iRNA comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The term “compound” as used herein, refers to an oligomeric compound that can be an oligonucleotide, an antisense, or an iRNA agent such as an siRNA.

The phrase “antisense strand” as used herein, refers to an oligomeric compound that is substantially or 100% complementary to a target sequence of interest. The phrase “antisense strand” includes the antisense region of both oligomeric compounds that are formed from two separate strands, as well as unimolecular oligomeric compounds that are capable of forming hairpin or dumbbell type structures. The terms “antisense strand” and “guide strand” are used interchangeably herein.

The phrase “sense strand” refers to an oligomeric compound that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, I. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region of a dsRNA is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a dsRNA is equal to or at least 14, 15, 16, 17, 18, 19, 20, 21, 22, 23, 23, 24, 25, 26, 27, 28, 29, or 30 nucleotides in length.

In some embodiments, the sense strand of a dsRNA is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the dsRNA are each 15 to 30 nucleotides in length.

In one embodiment, the sense and antisense strands of the dsRNA are each 19 to 25 nucleotides in length.

In one embodiment, the sense and antisense strands of the dsRNA are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded RNAs of the first strand and vice versa (e.g., a single-stranded loop). In some embodiments, the single-stranded nucleotides are present within 8 nucleotides from either end, for example 8, 7, 6, 5, 4, 3, or 2 nucleotide from either the 5′ or 3′ end of the region of complementarity between the two strands.

In one embodiment, the dsRNA comprises a single-stranded overhang on at least one of the termini. In one embodiment, the single-stranded overhang is 1, 2, or 3 nucleotides in length.

In one embodiment, the sense strand of the dsRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the dsRNA has a ZXY structure, such as is described in PCT Publication No. 2004080406, which is hereby incorporated by reference in its entirety.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

In certain embodiments, two oligomeric strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

siRNA Design

In one embodiment, the dsRNA agent is a double ended bluntmer of 19 nt in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In one embodiment, the dsRNA agent is a double ended bluntmer of 20 nt in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In one embodiment, the dsRNA agent is a double ended bluntmer of 21 nt in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the dsRNA agent comprises a 21 nucleotides (nt) sense strand and a 23 nucleotides (nt) antisense, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the dsRNA is blunt, while the other end is comprises a 2 nt overhang. Preferably, the 2 nt overhang is at the 3′-end of the antisense.

In one embodiment, the dsRNA agent comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the dsRNA agent comprises a sense and antisense strands, wherein said dsRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region which is at least 25 nucleotides in length, and said second strand is sufficiently complementary to a target mRNA along at least 19 nt of said second strand length to reduce target gene expression when said dsRNA agent is introduced into a mammalian cell, and wherein dicer cleavage of said dsRNA preferentially results in an siRNA comprising said 3′ end of said second strand, thereby reducing expression of the target gene in the mammal.

In one embodiment, the sense strand of the dsRNA agent contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′ end.

In one embodiment, the antisense strand of the dsRNA agent can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For dsRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the dsRNA from the 5′-end.

In some embodiments, the dsRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the dsRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the dsRNA agent comprises a sense strand and antisense strand each having 14 to 30 nucleotides, wherein the sense strand contains at least one motif of three 2′-F modifications on three consecutive nucleotides at positions 9, 10, 11 from the 5′end, and wherein the antisense strand contains at least one motif of three 2′-O-methyl modifications on three consecutive nucleotides at positions 11, 12, 13 from the 5′ end.

In one embodiment, the dsRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the dsRNA agent comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In one aspect, the invention relates to a double-stranded RNA (dsRNA) agent for inhibiting the expression of a target gene. The dsRNA agent comprises a sense strand and an antisense strand, each strand having 14 to 40 nucleotides. The dsRNA agent is represented by formula (I):

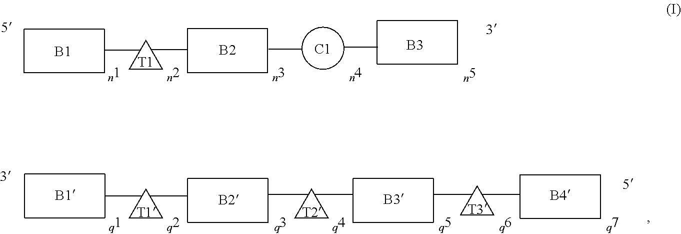

In formula (I), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

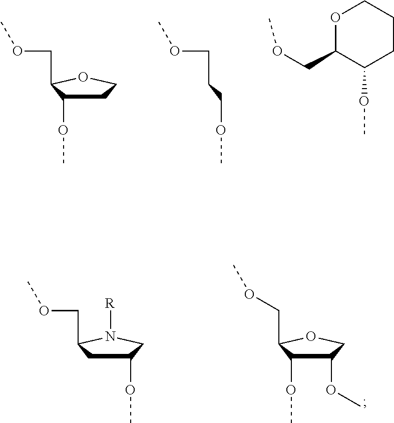

modification selected from the group consisting of:

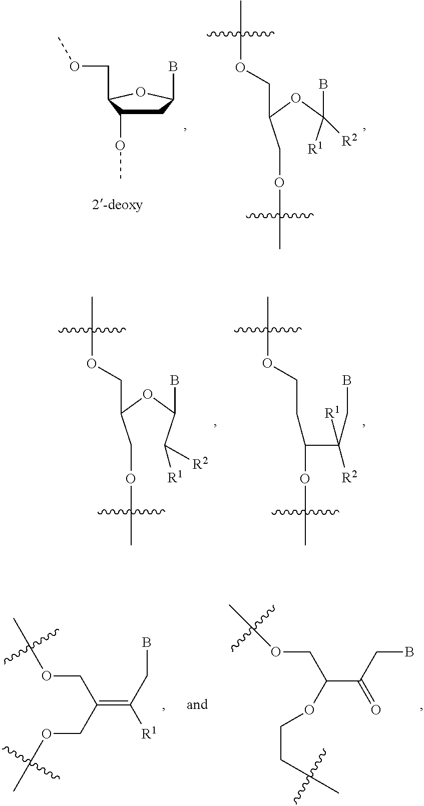

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

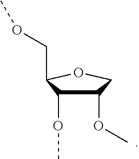

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.

n¹, n³, and q¹ are independently 4 to 15 nucleotides in length.

n⁵, q³, and q⁷ are independently 1-6 nucleotide(s) in length.

n⁴, q², and q⁶ are independently 1-3 nucleotide(s) in length; alternatively, n⁴ is 0. q⁵ is independently 0-10 nucleotide(s) in length.

n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The dsRNA agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

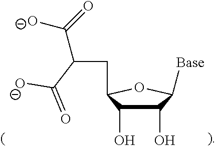

When the 5′-end phosphorus-containing group is 5′-end vinylphosphonate (5′-VP), the 5′-VP can be either 5′-E-VP isomer (i.e., trans-vinylphosphate,

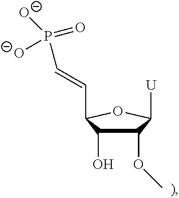

5′-Z-VP isomer (i.e., cis-vinylphosphate,

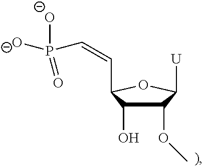

or mixtures thereof.

In one embodiment, the dsRNA agent comprises a phosphorus-containing group at the 5′-end of the sense strand. In one embodiment, the dsRNA agent comprises a phosphorus-containing group at the 5′-end of the antisense strand.

In one embodiment, the dsRNA agent comprises a 5′-P. In one embodiment, the dsRNA agent comprises a 5′-P in the antisense strand.

In one embodiment, the dsRNA agent comprises a 5′-PS. In one embodiment, the dsRNA agent comprises a 5′-PS in the antisense strand.

In one embodiment, the dsRNA agent comprises a 5′-VP. In one embodiment, the dsRNA agent comprises a 5′-VP in the antisense strand. In one embodiment, the dsRNA agent comprises a 5′-E-VP in the antisense strand. In one embodiment, the dsRNA agent comprises a 5′-Z-VP in the antisense strand.

In one embodiment, the dsRNA agent comprises a 5′-PS₂. In one embodiment, the dsRNA agent comprises a 5′-PS₂ in the antisense strand.

In one embodiment, the dsRNA agent comprises a 5′-PS₂. In one embodiment, the dsRNA agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a
5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a
5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, 100%, 95%, 90%, 85%, 80%, 75%, 70%, 65%, 60%, 55%, 50%, 45%, 40%, 35% or 30% of the dsRNA agent is modified. For example, when 50% of the dsRNA agent is modified, 50% of all nucleotides present in the dsRNA agent contain a modification as described herein.

In one embodiment, each of the sense and antisense strands is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent contains at least two different modifications.

In one embodiment, the dsRNA agent of Formula (I) further comprises 3′ and/or 5′ overhang(s) of 1-10 nucleotides in length. In one example, dsRNA agent of formula (I) comprises a 3′ overhang at the 3′-end of the antisense strand and a blunt end at the 5′-end of the antisense strand. In another example, the dsRNA agent has a 5′ overhang at the 5′-end of the sense strand.

In one embodiment, the dsRNA agent does not contain any 2′-F modification.

In one embodiment, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands of the dsRNA agent has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent is 100% complementary to a target RNA to hybridize thereto and inhibits its expression through RNA interference. In another embodiment, the antisense strand of the dsRNA agent is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand). Each of the embodiments and aspects described in this specification relating to the dsRNA represented by formula (I) can also apply to the dsRNA containing the thermally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end of the antisense strand.

In one embodiment, the dsRNA agents of comprise:
(a) a sense strand having:
   (i) a length of 18-23 nucleotides;
   (ii) three consecutive 2′-F modifications at positions 7-15; and
(b) an antisense strand having:
   (i) a length of 18-23 nucleotides;
   (ii) at least 2′-F modifications anywhere on the strand; and
   (iii) at least two phosphorothioate internucleotide linkages at the first five nucleotides (counting from the 5′ end);
wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise:
(a) a sense strand having:
   (i) a length of 18-23 nucleotides;
   (ii) less than four 2′-F modifications;
(b) an antisense strand having:
   (i) a length of 18-23 nucleotides;
   (ii) at less than twelve 2′-F modification; and
   (iii) at least two phosphorothioate internucleotide linkages at the first five nucleotides (counting from the 5′ end);
wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise:
(a) a sense strand having:
   (i) a length of 19-35 nucleotides;
   (ii) less than four 2′-F modifications;
(b) an antisense strand having:
   (i) a length of 19-35 nucleotides;
   (ii) at less than twelve 2′-F modification; and
   (iii) at least two phosphorothioate internucleotide linkages at the first five nucleotides (counting from the 5′ end);
wherein the duplex region is between 19 to 25 base pairs (preferably 19, 20, 21 or 22); and wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have less than 20%, less than 15% and less than 10% non-natural nucleotide.

Examples of non-natural nucleotide includes acyclic nucleotides, LNA, HNA, CeNA, 2′-methoxyethyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F, and others.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have greater than 80%, greater than 85% and greater than 90% natural nucleotide, such as 2′-OH, 2′-deoxy and 2′-OMe are natural nucleotides.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have 100% natural nucleotide, such as 2′-OH, 2′-deoxy and 2′-OMe are natural nucleotides.

In some embodiments, the dsRNA agent comprises a sense strand and an antisense strand, each strand independently having a length of 15 to 35 nucleotides, wherein the sense strand comprises a 2′-fluoro nucleotide at position 10, counting from 5′-end of the sense strand.

In some embodiments, the sense strand further comprises one or more, e.g., 1, 2, 3, 4 or 5 additional 2′-fluoro nucleotides. The additional 2′-fluoro nucleotides can be located anywhere in the sense strand.

In some embodiments, the sense strand further comprises a 2′-fluoro nucleotide at position 10, counting from 5′-end of the sense strand. In some embodiments, the sense strand further comprises a 2′-fluoro nucleotide at one or more of positions 8, 9, 11, and 12, counting from 5′-end of the sense strand. For example, the sense strand further comprises a 2′-fluoro nucleotide at position 9, counting from 5′-end of the sense strand; in other words, the sense strand comprises a 2′-fluoro nucleotide at positions 9 and 10, counting from 5′-end of the sense strand. In another example, the sense strand further comprises a 2′-fluoro nucleotide at position 11, counting from 5′-end of the sense strand; that is, the sense strand comprises a 2′-fluoro nucleotide at positions 10 and 11, counting from the 5′-end of the sense strand.

In some embodiments, the sense strand comprises a 2′-fluoro nucleotide at positions 9, 10 and 11, counting from the 5′-end of the sense strand. In some embodiments, the sense strand comprises a 2′-fluoro nucleotide at positions 7, 8, and 9, counting from 5′-end of the sense strand. In some embodiments, the sense strand comprises a 2′-fluoro nucleotide at positions 8, 9, and 10, counting from the 5′-end of the sense strand. In some embodiments, the sense strand comprises a 2′-fluoro nucleotide at positions 10, 11 and 12, counting from 5′-end of the sense strand.

In some embodiments, the sense strand comprises a 2′-fluoro nucleotide at one or more of positions that are opposite to positions 11, 12, and 13 of the antisense strand, counting from the 5′-end of the antisense strand. By “opposite to,” it is meant that the sense strand and the antisense strand form a duplex, and the position of a particular nucleotide on the sense strand is counted based on the position of the nucleotide on the antisense strand that has base pairing with the particular nucleotide on the sense strand.

In some embodiments, the sense strand does not comprise a 2′-fluoro nucleotide at position 7, counting from the 5′-end of the sense strand. For example, the sense strand comprises a 2′-OMe nucleotide at position 7, counting from the 5′-end of the sense strand.

In some embodiments, any of the nucleotides in the sense strand that is not a 2′-fluoro nucleotide is a 2′-OMe nucleotide.

In some embodiments, the antisense strand comprises one or more 2′-deoxy, e.g., 2′-H nucleotides. For example, the antisense strand comprises 1, 2, 3, 4, 5, 6 or more 2′-deoxy nucleotides. In some embodiments, the antisense strand comprises 2, 3, 4, 5 or 6 2′-deoxy nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from the 5′-end of the antisense strand. In some embodiments, the antisense comprises a 2′-deoxy nucleotide at positions 2 and 12, counting from the 5′-end of the antisense strand. In some embodiments, the antisense comprises a 2′-deoxy nucleotide at positions 5 and 7, counting from the 5′-end of the antisense strand. In some embodiments, the antisense comprises a 2′-deoxy nucleotide at positions 2, 5, 7 and 12, counting from the 5′-end of the antisense strand.

In some embodiments, the antisense strand comprises one or more, e.g., 1, 2, 3, 4, 5 or more of 2′-fluoro nucleotides. For example, the antisense strand comprises a 2′-fluoro nucleotide at position 14, counting from the 5′-end of the antisense strand.

In some embodiments, the antisense strand comprises a 2′-fluoro nucleotide at position 14 and a nucleotide other than a 2′-deoxy or 2′-fluoro at position 16, counting from the 5′-end of the antisense strand. For example, the antisense strand comprises a 2′-fluoro nucleotide at position 14 and a 2′-OMe at position 16, counting from the 5′-end of the antisense strand.

In some embodiments, the antisense strand comprises a 2′-deoxy nucleotide at positions 2 and 12 and 2′-fluoro nucleotide at position 14, counting from the 5′-end of the antisense strand. In some embodiments, the antisense strand comprises a 2′-deoxy nucleotide at positions 2 and 12, a 2′-fluoro nucleotide at position 14, and a nucleotide other than a 2′-deoxy or 2′-fluoro at position 16, counting from the 5′-end of the antisense strand. For example, the antisense strand comprises a 2′-deoxy nucleotide at positions 2 and 12, a 2′-fluoro nucleotide at position 14, and a 2′-OMe nucleotide at position 16, counting from the 5′-end of the antisense strand.

In some embodiments, the antisense strand comprises a 2′-deoxy nucleotide at position 14, counting from the 5′-end of the antisense stand, and the sense strand comprises a nucleotide other than a 2′-fluoro at position 7, counting from 5′-end of the sense strand. For example, the antisense strand comprises a 2′-deoxy nucleotide at positions 2, 12 and 14, counting from the 5′-end of the antisense stand, and the sense strand comprises a 2′-fluoro nucleotide at position 10 and a nucleotide other than a 2′-fluoro at position 7, counting from 5′-end of the sense strand.

In some embodiments, the sense strand comprises a 2′-fluoro nucleotide at position 10, counting from 5′-end of the sense strand, and the antisense strand comprises a 2′-deoxy nucleotide at positions 2, 5, 7 and 12, counting from 5′-end of the antisense strand, and (i) the antisense strand comprises a 2′-fluoro nucleotide at position 14 and a nucleotide other than a 2′-deoxy or 2′-fluoro nucleotide at position 16, counting from the 5′-end of the antisense strand; or (ii) the antisense strand comprises a 2′-deoxy nucleotide at position 14 or 16, counting from the 5′-end of the antisense strand, and the sense strand comprises a nucleotide other than a 2′-fluoro nucleotide at position 7, counting from the 5′-end of the sense strand.

In some embodiments, any of the nucleotides in the antisense strand that is not a 2′-fluoro nucleotide or not a 2′-deoxy nucleotide is a 2′-OMe nucleotide.

In one embodiment, the dsRNA agents a sense strand and an antisense strand, each strand having 14 to 30 nucleotides, wherein the sense strand sequence is represented by formula (I):

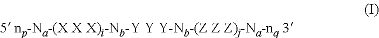

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 1, 2, 3, 4, 5, or 6 modified nucleotides;
each nₚ and n_{q} independently represent an overhang nucleotide;
wherein N_{b} and Y do not have the same modification;
wherein XXX, YYY and ZZZ each independently represent one motif of three identical modifications on three consecutive nucleotides;
wherein the dsRNA agents have one or more lipophilic monomers containing one or more lipophilic moieties conjugated to one or more positions on at least one strand; and
wherein the antisense strand of the dsRNA comprises two blocks of one, two or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, the dsRNA agent comprises an antisense strand sequence represented by formula (II):

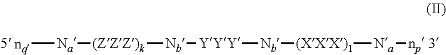

wherein:
k and 1 are each independently 0 or 1;
p and q are each independently 0-6;
each Nₐ′ independently represents an oligonucleotide sequence comprising 0-25 modified nucleotides, each sequence comprising at least two differently modified nucleotides;
each N_{b}′ independently represents an oligonucleotide sequence comprising 0-10 modified nucleotides;
each nₚ′ and n_{q}′ independently represent an overhang nucleotide comprising 0-6 nucleotides;
wherein N_{b}′ and Y′ do not have the same modification; and
X′X′X′, Y′Y′Y′ and Z′Z′Z′ each independently represent one motif of three identical modifications on three consecutive nucleotides.

Additional details about the motifs represented by formula (I) and formula (II) above may be found in WO 2013/074947, which is incorporated herein by reference in its entirety.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, 100%, 95%, 90%, 85%, 80%, 75%, 70%, 65%, 60%, 55%, 50%, 45%, 40%, 35% or 30% of the dsRNA agent is modified with 2′-OMe.

In some embodiments, each of the sense and antisense strands of the dsRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In some embodiments, each of the sense and antisense strands of the dsRNA agent contains at least two different modifications.

In some embodiments, the dsRNA agent of the invention does not contain any 2′-F modification.

In some embodiments, the dsRNA agent of the invention contains one, two, three, four, five, six, seven, eight, nine, ten, eleven or twelve 2′-F modification(s). In one example, the dsRNA agent of the invention contains nine or ten 2′-F modifications.

The dsRNA agent may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the dsRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In some embodiments, the antisense strand of the dsRNA agent is 100% complementary to a target RNA to hybridize thereto and inhibits its expression through RNA interference. In another embodiment, the antisense strand of the dsRNA agent is at least 95%, at least 90%, at least 85%, at least 80%, at least 75%, at least 70%, at least 65%, at least 60%, at least 55%, or at least 50% complementary to a target RNA.

### Nucleic Acid Modifications

In some embodiments, the dsRNA agent comprises at least one nucleic acid modification described herein. For example, at least one modification selected from the group consisting of modified internucleoside linkage, modified nucleobase, modified sugar, and any combinations thereof. Without limitations, such a modification can be present anywhere in the dsRNA agent. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide dsRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)pseudouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the dsRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic includes more complicated structures, such as for example a tricyclic phenoxazine nucleobase mimetic. Methods for preparation of the above noted modified nucleobases are well known to those skilled in the art.

### Nucleic Acid Modifications (Sugar)

Compound of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furanosyl is connected to the 4′ position by a linker selected independently from —[C(R1)(R2)]ₙ-, —[C(R1)(R2)]ₙ-O—, —[C(R1)(R2)]ₙ-N(R1)-, —[C(R1)(R2)]ₙ-N(R1)-O—, [C(R1R2)]ₙ-O—N(R1), —C(R1)=C(R2)-O—, —C(R1)=N—, —C(R1)=N—O—, C(═NR1)-, C(═NR1)-O—, —C(═O)—, —C(═O)O—, —C(═S)—, —C(═S)O—, —C(═S)S—, —O—, —Si(R1)2-, —S(═O)ₓ— and —N(R1)-;

wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂-O-2′, 4′-CH₂—O—N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)₁CH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and 0-CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)₁CH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Compound of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. dsRNA agent of the inventions can also contain one or more sugars that are the L isomer, e.g. L-nucleosides. Modification to the sugar group can also include replacement of the 4′-0 with a sulfur, optionally substituted nitrogen or CH₂ group. In some embodiments, linkage between C1′ and nucleobase is in a configuration.

Sugar modifications can also include a “acyclic nucleotide,” which refers to any nucleotide having an acyclic ribose sugar, e.g., wherein a C—C bonds between ribose carbons (e.g., C1′-C2′, C2′-C3′, C3′-C4′, C4′-04′, C1′-04′) is absent and/or at least one of ribose carbons or oxygen (e.g., C1′, C2′, C3′, C4′ or 04′) are independently or in combination absent from the nucleotide. In some embodiments, acyclic nucleotide is

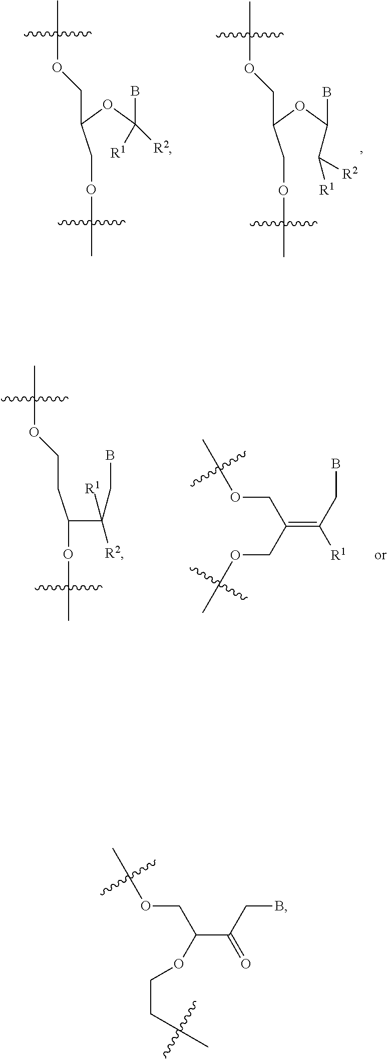

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl](2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

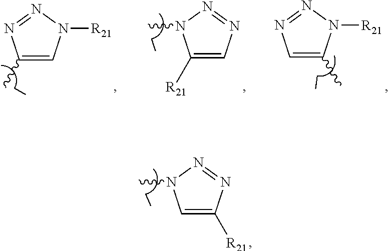

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alkali metal or transition metal with an overall charge of +1; and Y is O, S, or NR′, where R′ is hydrogen, optionally substituted aliphatic. Preferably this modification is at the 5′ terminal of the dsRNA.

In certain embodiments, the dsRNA agent of the invention comprises at least two regions of at least two contiguous monomers of the above formula. In certain embodiments, the dsRNA agent of the invention comprises a gapped motif. In certain embodiments, the dsRNA agent of the invention comprises at least one region of from about 8 to about 14 contiguous β-D-2′-deoxyribofuranosyl nucleosides. In certain embodiments, the dsRNA agent of the invention comprises at least one region of from about 9 to about 12 contiguous β-D-2′-deoxyribofuranosyl nucleosides.

In certain embodiments, the dsRNA agent of the invention comprises at least one (e.g., 1, 2, 3, 4, 5, 6, 7, 8, 9, 10, 11, 12, 13, 14, 15 or more) comprises at least one (S)-cEt monomer of the formula:

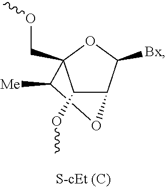

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH2-), thiodiester (—O—C(O)—S—), thionocarbamate (—O—C(O)(NH)—S—); siloxane (—O—Si(H)₂—O—); and N,N′-dimethylhydrazine (CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phosphotriesters, aminoalkylphosphotriesters, alkyl-phosphonaters (e.g., methyl-phosphonate), selenophosphates, phosphoramidates (e.g., N-alkylphosphoramidate), and boranophosphonates.

In some embodiments, the dsRNA agent of the invention comprises at least one (e.g., 1, 2, 3, 4, 5, 6, 7, 8, 9, 10, 11, 12, 13, 14, 15 or more and up to including all) modified or nonphosphodiester linkages. In some embodiments, the dsRNA agent of the invention comprises at least one (e.g., 1, 2, 3, 4, 5, 6, 7, 8, 9, 10, 11, 12, 13, 14, 15 or more and up to including all) phosphorothioate linkages.

The dsRNA agent of the inventions can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backbone-extended pyrrolidine PNA (bepPNA) nucleoside surrogates. A preferred surrogate is a PNA surrogate.

The dsRNA agent of the inventions described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the dsRNA agent of the inventions provided herein are all such possible isomers, as well as their racemic and optically pure forms.

### Nucleic Acid Modifications (Terminal Modifications)

In some embodiments, the dsRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the dsRNA agent does not contain a 5′-vinyl phosphonate (VP).

Ends of the dsRNA agent of the invention can be modified. Such modifications can be at one end or both ends. For example, the 3′ and/or 5′ ends of an dsRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type oligomeric compound.

Terminal modifications useful for modulating activity include modification of the 5′ end of dsRNAs with phosphate or phosphate analogs. In certain embodiments, the 5′end of an dsRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

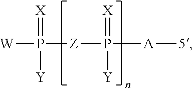

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently 0, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)2(X)P—O[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The dsRNA agents of the invention, such as iRNAs or dsRNA agents, can be optimized for RNA interference by increasing the propensity of the dsRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified abasic modifications are:

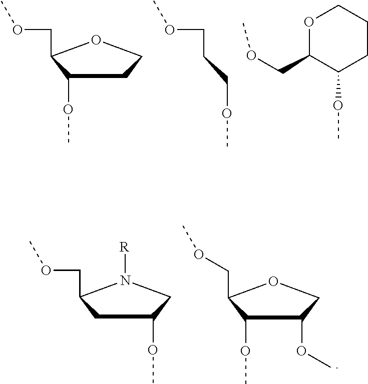

Exemplified sugar modifications are:

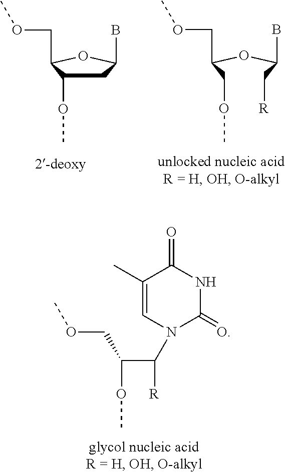

The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

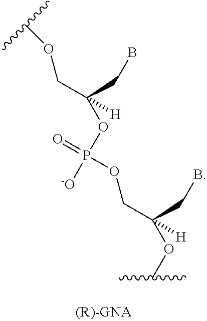

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA agents of the invention, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

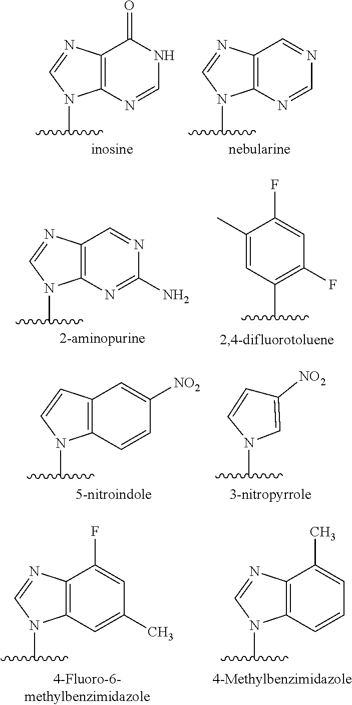

Exemplary phosphate modifications known to decrease the thermal stability of dsRNA duplexes compared to natural phosphodiester linkages are:

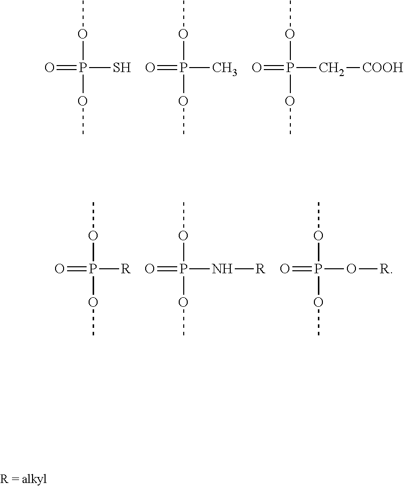

In some embodiments, the dsRNA agents can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the dsRNA agents can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In one embodiment, the dsRNA agent is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

In some embodiments, at least one strand of the dsRNA agent disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH2-), ethoxymethyl, etc., e.g. RP(OH)(O)—O-5′-).

### Target Genes

Without limitations, target genes for siRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the siRNAs include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; INK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLI1 fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in diploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides a dsRNA agent of the invention that modulates a micro-RNA.

### Targeting CNS

In some embodiments, the invention provides a dsRNA agent that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, the invention provides a dsRNA agent that targets TARDBP for ALS, MAPT (Tau) for Frontotemporal Dementia, and HTT for Huntington Disease.

In some embodiments, the invention provides a dsRNA agent that targets SNCA for Parkinson Disease, FUS for ALS, ATXN3 for Spinocerebellar Ataxia 3, ATXN1 for SCA1, genes for SCA7 and SCA8, ATN1 for DRPLA, MeCP2 for XLMR, PRNP for Prion Diseases, recessive CNS disorders: Lafora Disease, DMPK for DM1, and TTR for hATTR.

Spinocerebellar ataxia is an inherited brain-function disorder. Dominantly inherited forms of spinocerebellar ataxias, such as SCA1-8, are devastating disorders with no disease-modifying therapy. Exemplary targets include SCA2, SCA3, and SCA1.

Additional exemplary target genes are SCN9A, LRRK2, GPR75, APOE, SCB5, and GSK3α.

### Targeting ATXN2 for SCA2

Spinocerebellar Ataxia 2 (SCA2), a progressive ataxia, is the second most common SCA. Another disease associated with this target is amyotrophic lateral sclerosis (ALS). These diseases are debilitating and ultimately lethal diseases with no disease-modifying therapy. The prevalence of SCA is 2-6 per 100,000 people; ATXN2 causes 15% of SCA population worldwide and much more SCA populations in some countries, especially in Cuba (40 per 100,000 people). Targeting ATXN2 can be excellent via human molecular genetics, e.g., coding CAG repeat expansion in ATXN2 was discovered in familial and sporadic SCA and ALS, in tissues such as spinal cord, brainstem, or cerebellum. The mechanism of this targeting may be because autosomal dominant coding CAG expansion of ATXN2 causes expression of toxic, misfolded protein and Purkinje cell and neuronal death. The efficacy has been shown by 70% knockdown (KD) of ATXN2 mRNA; and mATXN2 mice KD POC has been demonstrated. With respect to safety, mATXN2 knockout (KO) mice have been reported healthy. Possible diagnosis includes family history; genetic testing; or early symptoms. Biomarkers that can be used include, e.g., CSF CAG mRNA and peptide repeat proteins.

### Targeting ATXN3 for SCA3

Spinocerebellar Ataxia 3 (SCA3), a progressive ataxia, is the most common SCA worldwide. This disease is debilitating and ultimately lethal disease with no disease-modifying therapy. It is the most common cause of SCA and the prevalence of SCA is 2-6 per 100,000 people; ATXN3 causes 21% of SCA population in US and much more in Europe, especially in Portugal. Targeting ATXN3 can be excellent via human molecular genetics, e.g., coding CAG repeat expansion in ATXN3 was discovered in familial and sporadic SCA, in tissues such as spinal cord, brainstem, or cerebellum. The mechanism of this targeting may be because autosomal dominant coding CAG expansion of ATXN3 causes expression of toxic, misfolded protein, Purkinje cell and neuron death. The efficacy has been shown by 70% KD of ATXN3 mRNA; and mATXN3 KD mice POC has been demonstrated. With respect to safety, mATXN3 KO mice have been reported healthy. Possible diagnosis includes family history; genetic testing; or early symptoms. Biomarkers that can be used include, e.g., CSF CAG mRNA and peptide repeat proteins.

### Targeting ATXN1 for SCA1

Spinocerebellar Ataxia 1 (SCA1), a progressive ataxia, is the first SCA gene discovered in 1993. This disease is debilitating and ultimately lethal disease with no disease-modifying therapy. The prevalence of SCA is 2-6 per 100,000 people; ATXN1 causes 6% of SCA population in US and worldwide, and much more in some countries (25% in Japan), especially in Poland (64%) and Siberia (100%). Targeting ATXN1 can be excellent via human molecular genetics, e.g., coding CAG repeat expansion in ATXN1 was discovered in familial and sporadic SCA, in tissues such as spinal cord, brainstem, or cerebellum. The mechanism of this targeting may be because autosomal dominant coding CAG expansion of ATXN1 causes expression of toxic, misfolded protein, Purkinje cell and neuronal death. The efficacy has been shown by 70% KD of ATXN1 mRNA; and mATXN1 mice POC has been demonstrated. With respect to safety, mATXN1 KO mice have been reported healthy. Possible diagnosis includes family history; genetic testing; or early symptoms. Biomarkers that can be used include, e.g., CSF CAG mRNA and peptide repeat proteins.

### Targeting ATXN7 for SCA7

Spinocerebellar Ataxia 7 (SCA7) causes progressive ataxia. This disease is debilitating and ultimately lethal cerebellar disorder with no disease-modifying therapy. The prevalence of SCA is 2-6 per 100,000 people; ATXN7 causes 5% of SCA population worldwide, and much more in some countries, especially in South Africa. Targeting ATXN7 can be excellent via human molecular genetics, e.g., coding CAG repeat expansion in ATXN7 discovered in familial and sporadic SCA, in tissues such as spinal cord, brainstem, or cerebellum. The mechanism of this targeting may be because autosomal dominant coding CAG expansion of ATXN1 causes expression of toxic, misfolded protein, inciting cone and rod dystrophy, Purkinje cell and neuronal lethality. The efficacy has been shown by 70% KD of ATXN1 mRNA, via intrathecal (IT) administration. Possible diagnosis includes family history; genetic testing; or early symptoms. Biomarkers that can be used include, e.g., CSF CAG mRNA and peptide repeat proteins.

### Targeting ATXN8 for SCA8

Spinocerebellar Ataxia 8 (SCA8), a progressive neurodegenerative ataxia is caused by CTG repeat expansion in ATXN8. This disease is debilitating and ultimately lethal disease with no disease-modifying therapy. The prevalence: SCA is 2-6 per 100,000 people; ATXN8 causes 3% of SCA population worldwide, and much more in some countries, especially in Finland. Targeting ATXN8 can be excellent via human molecular genetics, e.g., coding CTG repeat expansion in ATXN8 was discovered in familial and sporadic SCA, in tissues such as spinal cord, brainstem, or cerebellum. The mechanism of this targeting may be because autosomal dominant coding CTG expansion of ATXN8 causes expression of toxic, misfolded protein, inciting Purkinje cell and neuronal lethality. The efficacy has been shown by 70% KD of ATXN8 mRNA. Possible diagnosis includes family history; genetic testing; or early symptoms. Biomarkers that can be used include, e.g., CSF CTG mRNA and peptide repeat proteins.

### Targeting CACNA1A for SCA6

Spinocerebellar ataxia 6 (SCA6) is a progressive ataxia. This disease is debilitating and ultimately lethal disease with no disease-modifying therapy. The prevalence of SCA is 2-6 per 100,000 people; and CACNA1A causes 15% of SCA population worldwide. Targeting CACNA1A can be excellent via human molecular genetics, e.g., coding CAG repeat expansion in CACNA1A was discovered in familial and sporadic SCA, in tissues such as spinal cord, brainstem, or cerebellum. The mechanism of this targeting may be because autosomal dominant coding CAG expansion of CACNA1A causes expression of toxic, misfolded protein and Purkinje cell and neuronal death. The efficacy has been shown by 70% KD of CACNA1A CAG expansion. Possible diagnosis includes family history; genetic testing; or early symptoms. Biomarkers that can be used include, e.g., CSF CAG mRNA and peptide repeat proteins.

Exemplary target for inherited polyglutamine disorders includes Huntington disease (HD).

### Targeting HTT for Huntington Disease

Huntington mutations causes HD, a progressive CNS degenerative disease. This disease is debilitating and ultimately lethal disease with no disease-modifying therapy. The prevalence of HD is 5-10 per 100,000 people worldwide, and much more common in certain countries, especially in Venezuela. Targeting HTT can be excellent via human molecular genetics, e.g., coding CAG repeat expansion in HTT discovered in familial and sporadic HD, in tissues such as striatum, or cortex. The mechanism of this targeting may be because autosomal dominant coding CAG expansion of HTT causes expression of toxic, misfolded protein and neuronal death. The efficacy has been shown by 70% KD of HTT CAG expansion only; and murine POC has been demonstrated. With respect to safety, KO of HTT in mice can be lethal; KD in humans has been demonstrated. Possible diagnosis includes family history; genetic testing; early symptoms. Biomarkers that can be used include, e.g., CSF mRNA and peptide repeat proteins.

### Targeting ATN1 for DRPLA

Atrophin 1 mutations causes dentatorubral-pallidoluysian atrophy (DRPLA), which is a progressive spinocerebellar disorder similar to HD. This disease is debilitating and ultimately lethal disease with no disease-modifying therapy. The prevalence of DRPLA is 2-7 per 1,000,000 people in Japan. Targeting ATN1 can be excellent via human molecular genetics, e.g., coding CAG repeat expansion in ATN1 was discovered in familial and sporadic SCA, in tissues such as spinal cord, brainstem, cerebellum, or cortex. The mechanism of this targeting may be because autosomal dominant coding CAG expansion of ATN1 causes expression of toxic, misfolded protein and neuronal death. The efficacy has been shown by 70% KD of ATN1. With respect to safety, ATN1 KO mice have been reported healthy. Possible diagnosis includes family history; genetic testing; or early symptoms. Biomarkers that can be used include, e.g., CSF CAG mRNA and peptide repeat proteins.

### Targeting AR for Spinal and Bulbar Muscular Atrophy

Androgen receptor mutations causes spinal and bulbar muscular atrophy (SBMA, Kennedy disease), a progressive muscle wasting disease, and other diseases. This disease is debilitating and ultimately lethal disease with no disease-modifying therapy. The prevalence of SBMA is 2 per 100,000 males; females have a mild phenotype. Targeting AR can be excellent via human molecular genetics, e.g., coding CAG repeat expansion in AR discovered in familial SBMA, in tissues such as spinal cord, or brainstem. The mechanism of this targeting may be because X-linked coding CAG expansion of AR causes toxic gain-or-function and motor neuron lethality. The efficacy has been shown by 70% KD of AR. Possible diagnosis includes family history; genetic testing; or early symptoms. Biomarkers that can be used include, e.g., CSF CAG mRNA and peptide repeat proteins.

### Targeting FXN for Friedrich Ataxia

Recessive loss of function GAA expansion of FXN causes friedrich ataxia (FA), a progressive degenerative ataxia. This disease is debilitating and ultimately lethal disease with no disease-modifying therapy. The prevalence of FA is 2 per 100,000 people worldwide. Targeting FXN can be excellent via human molecular genetics, e.g., intron GAA repeat expansion in FXN was discovered in familial FA, in tissues such as spinal cord or cerebellum. The mechanism of this targeting may be because autosomal recessive non-coding FAA expansion of FXN causes deceased expression of FXN, an important mitochondrial protein. The efficacy has been shown by 70% KD of FXN intron GAS expansion. With respect to safety, KD of intron GAA is safe and effective in mice. Possible diagnosis includes family history; genetic testing; or early symptoms. Biomarkers that can be used include, e.g., CSF mRNA and peptide repeat proteins.

### Targeting FMR1 for FXTAS

Fragile X-associated tremor/ataxia syndrome (FXTAS), a progressive disorder of ataxia and cognitive loss in adults caused by FMR1 overexpression. This disease is debilitating disease with no disease-modifying therapy. The prevalence of FMR1 permutation is 1 in 500 males. Targeting FMR1 can be excellent via human molecular genetics, e.g., coding CCG repeat expansion pre-mutations in FMR1 was discovered in FXTAS, in tissues such as spinal cord, cerebellum, or cortex. The mechanism of this targeting may be because X-linked coding CCG expansion of FMR1 causes toxic mRNA. The efficacy has been shown by 70% KD of toxic mRNA. Possible diagnosis includes family history; genetic testing; or early symptoms. Biomarkers that can be used include, e.g., CSF mRNA and peptide repeat proteins.

### Targeting Upstream of FMR1 for Fragile X Syndrome

Fragile X syndrome (FRAXA), a progressive disorder of mental retardation, may be treated by targeting upstream mRNA of FMR1. This disease is debilitating disease with no disease-modifying therapy. The prevalence of FRAXA is 1 per 4,000 males and 1 per 8,000 females. Targeting FMR1 can be excellent via human molecular genetics, e.g., coding CCG repeat expansion in FMR1 was discovered in FRAXA, in tissues such as CNS. The mechanism of this targeting may be because X-linked coding CCG expansion of FMR1 causes LOF; and normal FMR1 functions to transport specific mRNAs from nucleus. The efficacy has been shown by 70% KD of toxic mRNA. Possible diagnosis includes family history; genetic testing; or early symptoms. Biomarkers that can be used include, e.g., CSF mRNA and peptide repeat proteins.

Dominant Inherited Amyotrophic Lateral Sclerosis is a devastating disorders with no disease-modifying therapy. Exemplary targets include C9orf72, ATXN2 (also causes SCA2), and MAPT.

### Targeting C9orf72 for ALS

C9orf72 is the most common cause of Amyotrophic Lateral Sclerosis (ALS) and Frontotemporal Dementia (FTD). These diseases are lethal disorders of motor neurons with no disease-modifying therapy. The prevalence of ALS is 2-5 per 100,000 people (10% is familial); C9orf72 causes 39% of familial ALS in US and Europe and 7% of sporadic ALS. Targeting C9orf72 can be excellent via human molecular genetics, e.g., hexa-nucleotide expansion was discovered in familial and sporadic ALS, in tissues such as upper and lower motor neurons (for ALS); or cortex (for FTD). The mechanism of this targeting may be because autosomal dominant hexa-nucleotide expansion causes repeat-associated non-AUG-dependent translation of toxic dipeptide repeat proteins and neuron lethality. The efficacy has been shown by 70% KD of C9orf72. With respect to safety, heterozygous LOF mutations of C9orf72 appear to be safe in humans and mice. Possible diagnosis includes family history; genetic testing; or early symptoms. Biomarkers that can be used include, e.g., CSF hexa-nucleotide repeat mRNAs and dipeptide repeat proteins.

### Targeting TARDBP for ALS

TARDBP mutations causes ALS and Frontotemporal Dementia (FTD). These diseases are lethal disorders of motor neurons with no disease-modifying therapy. The prevalence of ALS is 2-5 per 100,000 people (10% is familial); TARDBP causes 5% of familial ALS and 1.5% of sporadic ALS. Targeting TARDBP can be excellent via human molecular genetics, e.g., mutations were discovered in familial and sporadic ALS, in tissues such as upper and lower motor neurons (for ALS); or cortex (for FTD). The mechanism of this targeting may be because autosomal dominant TRDBP mutations cause toxic TRDBP protein and neuron lethality. The efficacy has been shown by 70% KD of TARDBP mutant alleles. Possible diagnosis includes family history; genetic testing; or early symptoms. Biomarkers that can be used include, e.g., CSF proteins.

### Targeting FUS for ALS

FUS mutations causes ALS and FTD. These diseases are lethal disorder of motor neurons with no disease-modifying therapy. The prevalence of ALS is 2-5 per 100,000 people (10% is familial); FUS causes 5% of familial ALS; FUS inclusions are often found in sporadic ALS. Targeting FUS can be excellent via human molecular genetics, e.g., mutations were discovered in familial ALS, in tissues such as upper and lower motor neurons for ALS. The mechanism of this targeting may be because autosomal dominant FUS mutations cause abnormal protein folding and neuron lethality. The efficacy has been shown by 70% KD of FUS mutant alleles. With respect to safety, KO mice struggle but survive and have an ADHD phenotype. Possible diagnosis includes family history; genetic testing; or early symptoms. Biomarkers that can be used include, e.g., CSF proteins.

### Targeting SOD1 for ALS

Dominant and recessive mutations of SOD1 cause ALS. This disease is lethal disorder of motor neurons with no disease-modifying therapy. The prevalence of ALS is 2-5 per 100,000 people (10% is familial); SOD1 causes 5-20% of familial ALS. Target SOD1 can be excellent via human molecular genetics, e.g., many SOD1 mutations associate with AD and AR ALS in families, in tissues such as upper and lower motor neurons for ALS. The efficacy of this targeting may need mutation-specific KD. Possible diagnosis includes family history; genetic testing; or early symptoms. Biomarkers may be mutation-specific.

Dominant Inherited Frontotemporal Dementia and Progressive Supra-nuclear Palsy. The targets include MAPT because it may be important for AD, or C9orf72.

### Targeting Microtubule-Associated Protein Tau for FTD-17 and PSP

Familial Frontotemporal Dementia 17 (FTD-17), a familial form of FTD lined to chromosome 17, and Familial Progressive Supra-nuclear Palsy may be caused by MAPT mutations, which may also cause rare forms of Progressive Supra-nuclear Palsy, Corticobasal Degeneration, Tauopathy with Respiratory Failure, Dementia with Seizures. These diseases are lethal neurodegenerative disorders with no disease-modifying therapy. The prevalence of FTD is 15-22 per 100,000 people; the prevalence of FTD-17 in Netherlands is 1 in 1,000,000 population. Targeting MAPT can be excellent via human molecular genetics, e.g., GOF point and splice site mutations of MAPT were discovered in familial and sporadic FTD, in tissues such as frontal or temporal cortex. The mechanism of this targeting may be because autosomal dominant GOF mutations of MAPT lead to toxic Tau peptides and neuronal death. The efficacy has been shown by 70% KD of MAPT. With respect to safety, MAPT KO mice have been reported healthy. Possible diagnosis includes family history; genetic testing; early symptoms. Biomarkers that can be used include, e.g., CSF Tau mRNAs and proteins.

### Targeting Sequestosome 1 for FTD and ALS

Sporadic FTD/ALS associate with dominant SQSTM1 mutations. This disease is lethal neurodegenerative disorder with no disease-modifying therapy. This is a very rare disease. Targeting Sequestosome 1 is reasonable via human molecular genetic association in sporadic cases, in tissues such as frontal and temporal cortex, or cerebellum and spinal cord. Possible diagnosis includes genetic testing; early symptoms.

Dominant Inherited Parkinson Disease is a devastating disorders with no disease-modifying therapy. The targets include SNCA.

### Targeting SNCA for Parkinson Disease

Alpha Synuclein mutations causes familial Parkinson disease (PD) and Lewy body dementia. These diseases are lethal neurodegenerative disorders with no disease-modifying therapy. The prevalence of PD is 4 million worldwide; ⅓ of PD is familial; 1% of fPD is caused by SNCA. Targeting SNCA can be excellent via human molecular genetics, e.g., SNCA point mutations and duplications cause familial PD, in tissues such as medulla oblongata; or substantia nigra of the midbrain. The mechanism of this targeting may be because overexpression or expression of abnormal SNCA protein leads to toxic peptides and neuronal death. The efficacy has been shown by 70% KD of SNCA. With respect to safety, SNCA KO mice are healthy. Possible diagnosis includes family history; genetic testing; or early symptoms. Biomarkers that can be used include, e.g., CSF SNCA mRNAs and proteins.

### Targeting LRRK2 for Parkinson Disease

Leucine-rich repeat kinase 2 mutations cause familial Parkinson disease. This disease is lethal neurodegenerative disorder with no disease-modifying therapy. The prevalence of PD is 4 million worldwide; ⅓ of PD is familial; 3-7% of fPD is caused by LRRK2. Targeting LRRK2 can be excellent via human molecular genetics, e.g., LRRK2 point mutations cause familial PD, in tissues such as medulla oblongata; or substantia nigra of the midbrain. Possible diagnosis includes family history; genetic testing; early symptoms. Biomarkers that can be used include, e.g., CSF mRNAs and proteins.

### Targeting GARS for Spinal Muscular Atrophy V

Autosomal dominant Glycyl-tRNA Synthetase mutations cause spinal muscular atrophy V (SMAV) or distal hereditary motor neuropathy Va. These diseases are neurodegenerative disorders with no disease-modifying therapy. These are very rare diseases. Targeting GARs can be good via human molecular genetics, e.g., GARS point mutations cause familial SMA, in tissues such as spinal cord. Possible diagnosis includes family history; genetic testing; early symptoms.

### Targeting Seiipin for Spinal Muscular Atrophy

Autosomal dominant Seipin mutations causes spinal muscular atrophy (SMA) or distal hereditary motor neuropathy. These diseases are neurodegenerative disorders with no disease-modifying therapy. These are very rare diseases. Targeting Seipin can be good via human molecular genetics, e.g., Seipin point mutations cause familial SMA, in tissues such as spinal cord. The mechanism of this targeting is probably GOF and toxic peptides. The efficacy has been shown by 50% KD. With respect to safety, recessive LOF mutations cause progressive encephalopathy with or without lipodystrophy. Possible diagnosis includes family history; genetic testing; or early symptoms.

Dominant Inherited Alzheimer Disease is a devastating disorders with no disease-modifying therapy. The targets include APP because of central mechanistic role in familial disease and possible role in common AD.

### Targeting APP for Alzheimer Disease

Amyloid precursor protein mutations causes early onset familial Alzheimer disease (EOFAD); AD in down syndrome; or AD. These diseases are lethal neurodegenerative disorders with no disease-modifying therapy. The prevalence of EOFAD-APP is 1% AD; the prevalence of Trisomy 21 is 1% AD; and the prevalence of AD is about 2.5-5 million in US. Targeting APP can be excellent via human molecular genetics, e.g., APP duplications and point mutations cause EOFAD, in tissues such as cerebral cortex or hippocampus. The mechanism of this targeting may be because APP overexpression or expression of toxic metabolites cause progressive neuronal death. The efficacy has been shown by 70% KD of APP. With respect to safety, KD mice have been reported healthy with some behavioral abnormalities; KD mice have been reported healthy with some spatial memory effects. Possible diagnosis includes family history; genetic testing; early symptoms; or MRI. Biomarkers that can be used include, e.g., CSF APP mRNA and peptides.

Sequential cleavage of APP occurs by two pathways. In one pathway, APP is cleaved by a-secretase followed by g-secretase in performing nonamyloidogenic processing of APP. In a second pathway, amyloidogenic processing of APP involves BACE1 cleavage followed by g-secretase. Both processes generate soluble ectodomains (sAPPα and sAPPβ) and identical intracellular C-terminal fragments (See Thinakaran and Koo. J. Biol. Chem. 283: 29615-19; Reinhard et al. The EMBO Journal, 24: 3996-4006; Walsh et al. Biochemical Society Transactions, 35: 416-420; O'Brien and Wong. Annu Rev Neurosci. 34: 185-204, all of which are incorporated herein by reference in their entirety). Available assays can also be used to detect soluble APP levels in human CSF samples. In particular, sAPPα and sAPPβ are soluble forms of APP and have been identified as serving as PD (pharmacodynamic) biomarkers. Analytes have also been detected in non-human primate (NHP) CSF samples, and such assays can enable efficacy studies in NHPs.

### Targeting PSEN1 for Alzheimer Disease

Presenilin 1 mutations causes early onset familial Alzheimer disease (EOFAD); or AD. These diseases are lethal neurodegenerative disorder with no disease-modifying therapy. Targeting PSEN1 can be excellent via human molecular genetics, e.g., PSEN1 point mutations cause EOFAD, in tissues such as cerebral cortex; or hippocampus. The mechanism of this targeting may be because autosomal dominant mutations of PSEN1 cause abnormal APP metabolism and toxic peptides cause progressive neuronal death. The efficacy has been shown by APP KD may obviate need for PSEN1-specific therapy. Possible diagnosis includes family history; genetic testing; early symptoms; or MRI. Biomarkers that can be used include, e.g., CSF PSEN1 and APP peptides.

### Targeting PSEN2 for Alzheimer Disease

Presenilin 2 mutations causes early onset familial Alzheimer disease (EOFAD); or AD. These diseases are lethal neurodegenerative disorder with no disease-modifying therapy. Targeting PSEN2 can be excellent via human molecular genetics, e.g., PSEN2 point mutations cause EOFAD, in tissues such as cerebral cortex or hippocampus. The mechanism of this targeting may be because autosomal dominant mutations of PSEN2 cause abnormal APP metabolism and toxic peptides cause progressive neuronal death. Possible diagnosis includes family history; genetic testing; early symptoms; or MRI. Biomarkers that can be used include, e.g., CSF PSEN2 and APP peptides.

### Targeting Apo E for Alzheimer Disease

Apolipoprotein E4 is associated with sporadic AD in the elderly. This disease is lethal neurodegenerative disorder with no disease-modifying therapy. The prevalence of AD is 2.5-5 million in US. Targeting Apo E may be effective because genomic evidence supporting the association between ApoE4 and AD is excellent in many populations. The target tissue may be cerebral cortex. It is not yet clear if Apo E4 contributes to the pathogenesis of AD despite the strong association in many populations. Thus far, data indicate that Apo E4 homozygosity indicates increased risk of AD in the elderly but is not sufficient for causing AD, even in the elderly. With respect to safety, KD of Apo E in CNS may be safe as human LOF mutations in Apo E are not associated with obvious neurologic defects, although systemic exposure may cause hyperlipoproteinemia type III. Possible diagnosis includes clinical diagnosis of AD; exclusion of EOFAD mutation; genetic testing for the Apo E4 genotype. Biomarkers that can be used include, e.g., CSF APP, Tau mRNA and peptides.

CNS Gene Duplication Disorders. Consistent KD by half may ameliorate these disorders. The targets include MeCP2.

### Targeting MeCP2 for X-Linked Mental Retardation

Methyl CpG Binding Protein 2 gene duplication causes X-linked Mental Retardation (XLMR). This disease is lethal cognitive disorder with no disease-modifying therapy. 1-15% of X-linked MR is caused by MeCP2 duplication; 2-3% of population has MR. Targeting MeCP2 can be excellent via human molecular genetics, e.g., MeCP2 duplication causes XLMR, in tissues such as cerebral cortex. The mechanism of this targeting may be because MeCP2 over-expression cause dysregulation of other gene and neurodegeneration. The efficacy has been shown by 50% KD of MeCP2; and ASO KD in mouse models reverse phenotype. With respect to safety, MeCP2 LOF mutations may cause Rett syndrome. Possible diagnosis includes family history; genetic testing; or early symptoms. Biomarkers that can be used include, e.g., CSF MeCP2 mRNA and peptides.

Dominant Inherited Cerebral Amyloid Angiopathy is a devastating disorder with no disease-modifying therapy. The targets include TTR.

Targeting TTR for hATTR CAA

This targeting may be a low risk introduction to CNS siRNA. Cerebral Amyloid Angiopathy (CAA) and Meningeal Amyloid are lethal disorders with no disease-modifying therapy. Targeting TTR can be excellent via human genetics and pharmacology. The target tissues can be CNS vascular system, or CNS. The mechanism of this targeting may be because Mutant protein accumulates in vascular adventitia, causing CNS bleeds. The efficacy has been shown by 70% KD of TTR. Possible diagnosis includes family history; genetic testing; or early symptoms. Biomarkers that can be used include, e.g., CSF mRNA and protein.

### Targeting ITM2B for CAA

Integral Membrane Protein 2B mutations causes Cerebral Amyloid Angiopathy (CAA), British Type or Familial British Dementia (FBD). This disease is lethal disorder with no disease-modifying therapy. This is a rare disease. Targeting ITM2B can be excellent via human molecular genetics. The target tissues can be CNS vascular system, or CNS. The mechanism of this targeting probably involves GOF mutations. The efficacy has been shown by 70% KD of ITM2B mutant allele. Possible diagnosis includes family history; genetic testing; or early symptoms. Biomarkers that can be used include, e.g., CSF mRNA and protein possible.

### Targeting CST3 for CAA

Cystatin C mutations causes familial cerebral amyloid angiopathy, Icelandic type. This disease is lethal disorder with no disease-modifying therapy. This is a rare disease, except in Iceland and Denmark. Targeting CST3 can be excellent via human genetics. The target tissue can be CNS vascular system. The mechanism of this targeting may be because mutant protein accumulates in vascular adventitia, causing CNS bleeds. The efficacy has been shown by possibly 70% KD of mutant allele. With respect to safety, CST3 KO mice may have risk of arthritis. Possible diagnosis includes family history; genetic testing; or early symptoms. Biomarkers that can be used include, e.g., CSF mRNA and protein possible.

### Targeting SPAST for Spastic Paraplegia

SPASTIN mutations causes Spastic Paraplegia (SP) 4 with cognitive loss. This disease is lower motor neurodegenerative disorder with no disease-modifying therapy. The prevalence of SP is 5 per 100,000 population; SP4 is 45% of dominant SP. Targeting SPAST can be excellent via human molecular genetics, e.g., SPAST trinucleotide mutations causes familial SP, in tissues such as spinal cord; or CNS. The mechanism of this targeting may be because nonsense and probable dominant-negative mutations cause abnormal microtubule metabolism and neurodegeneration. Possible diagnosis includes family history; genetic testing; or early symptoms. Biomarkers that can be used include, e.g., CSF SPAST mRNAs and proteins possible.

### Targeting KIF5A for Spastic Paraplegia

Kinesin Family Member 5A mutations causes Spastic Paraplegia (SP) 10 with peripheral neuropathy and other disorders. This disease is lower motor neurodegenerative disorder with no disease-modifying therapy. The prevalence of SP is 5 per 100,000 people; SP10 is 1 per 1,000,000 people. Targeting KIF5A can be excellent via human molecular genetics, e.g., KIF5A amino terminal missense mutations cause SP10; and KIF5A is expressed in the CNS and encodes a microtubule motor protein. The target tissue may be spinal cord. The mechanism of this targeting may be because autosomal dominant missense mutations cause SP10 possibly affect microtubule binding to the motor. The efficacy may be provided by possibly KD of mutant alleles. With respect to safety, KIF5A frameshift mutations cause Neonatal intractable myoclonus and splice site mutations are associated with familial ALS, possibly through LOF mechanisms. Possible diagnosis includes family history; genetic testing; or early symptoms. Biomarkers that can be used include, e.g., CSF mRNAs and proteins possible.

### Targeting ATL1 for Spastic Paraplegia

Atlastin mutations causes Spastic Paraplegia 3A and Sensory Neuropathy 1D, Hereditary Sensory Neuropathy (HSN). This disease is a lower motor neurodegenerative disorder with no disease-modifying therapy. The prevalence of SP is 5 per 100,000 people; SP3A is a rare dominant form. Targeting ATL1 can be excellent via human molecular genetics, e.g., ATL1 point mutations cause familial SP. The target tissue may be spinal cord. The mechanism of this targeting may be because autosomal dominant expression of dominant-negative ATL1 protein causes SP3A; however, LOF mutations causes Sensory Neuropathy 1D. The efficacy has been shown by 70% KD of specific ATL1 allele. With respect to safety, ATL1 heterozygous LOF mutations causes HSN1D. Possible diagnosis includes family history; genetic testing; or early symptoms. Biomarkers that can be used include, e.g., CSF ATL1 mRNAs and proteins.

### Targeting NIPA1 for Spastic Paraplegia

LOF NIPA1 mutations cause Spastic Paraplegia 6 with epilepsy and seizures. This disease is lower motor neurodegenerative disorder with no disease-modifying therapy. The prevalence of SP is 5 per 100,000 people; SP6 is a rare dominant form. Targeting NIPA1 can be excellent via human molecular genetics, e.g., NIPA1 point mutations cause familial SP. The target tissues can be spinal cord; or CNS. The mechanism of this targeting may be because autosomal dominant expression of defective membrane protein causes SP3A; and possibly LOF. Possible diagnosis includes family history; genetic testing; or early symptoms. Biomarkers that can be used include, e.g., CSF mRNAs and proteins possible.

Dominant Inherited Myotonic Dystrophy is a disorder of CNS, Skeletal Muscle and Cardiac Muscle Requiring CNS and Systemic Therapy. The targets include MPK for DM1.

### Targeting DMPK for Myotonic Dystrophy 1

CNS and systemic therapy needed for effective therapy targeting dystrophia Myotonica Protein Kinase. Myotonic dystrophy 1 (DM1) is a degenerative disorder of muscle and CNS. It is a lethal disorder with no disease-modifying therapy. The prevalence of DM1 is 1 per 8,000 people worldwide. Targeting DMPK can be excellent via human molecular genetics, e.g., DMPK CTG repeat expansion causes familial DM1. The target tissues may be skeletal muscle, cardiac muscle, or CNS. The mechanism of this targeting may be because autosomal dominant non-coding CTG repeat causes abnormal RNA processing and dominant negative effect; anticipation from extreme expansion causes early onset disease. The efficacy has been shown by 70% of DMPK; and ASO efficacy have been demonstrated in mice. The safety has been demonstrated in mice with KO and ASO KD. Possible diagnosis includes family history; genetic testing; or early symptoms. Biomarkers that can be used include, e.g., Blood and CSF mRNAs and proteins.

### Targeting ZNF9 for Myotonic Dystrophy 2

Zinc Finger Protein 9 mutations causes Myotonic dystrophy 2 (DM2), a degenerative disorder of skeletal muscle. This is a serious disorder with no disease-modifying therapy. The prevalence of DM2 is 1 per 8,000 people worldwide; it is the most common muscular dystrophy in adults. Targeting ZNF9 can be excellent via human molecular genetics, e.g., ZNF9 CTTG repeat expansion in intron 1 causes familial DM2. The target tissues can be skeletal muscle, or cardiac muscle. The mechanism of this targeting may be because autosomal dominant CTTG repeat expansion in intron 1 causes abnormal RNA metabolism and dominant negative effects. The efficacy has been shown by 70% of ZNF9. Safe KD in mice has been demonstrated. Possible diagnosis includes family history; genetic testing; or early symptoms. Biomarkers that can be used include, e.g., Blood mRNAs and proteins.

Dominant Inherited Prion Diseases are inherited, sporadic and transmissible PRNP disorders. The targets include PRNP.

### Targeting PRNP for Myotonic Prion Diseases

Myotonic prion diseases are dominant inherited Prion diseases, including PRNP-Related Cerebral Amyloid Angiopathy, Gerstmann-Straussler Disease (GSD), Creutzfeldt-Jakob Disease (CJD), Fatal Familial Insomnia (FFI), Huntington Disease-Like 1 (HDL1), and Kuru susceptibility. These diseases are lethal neurodegenerative disorders with no disease-modifying therapy. The prevalence of this type of diseases is 1 per 1,000,000 people. Targeting PRNP can be excellent via human molecular genetics, e.g., PRNP mutations cause familial and sporadic Prion disease. The target tissue can be CNS. The mechanism of this targeting may be because autosomal dominant protein mid-folding causes neurotoxicity. The efficacy has been shown by 70% of PRNP KD; and PRNP polymorphisms appear protective for Kuru. With respect to safety, PRNP KO mice have been reported healthy. Possible diagnosis includes family history; genetic testing; or early symptoms. Biomarkers that can be used include, e.g., CSF mRNAs and proteins.

### Targeting Glycogen Synthase for Myoclonic Epilepsy of Lafora

Laforin (EPM2A) gene mutations causes AR Myoclonic Epilepsy, an inherited progressive seizure disorder. This disease is a lethal disorder of seizures and cognitive decline with no disease-modifying therapy. The prevalence of this disease is 4 per 1,000,000 people. Targeting Glycogen Synthase can be excellent via human molecular genetics, e.g., mutations causes AR familial Myoclonic Epilepsy of Lafora. The target tissue may be CNS. The mechanism of this targeting may be because autosomal recessive dysfunction of Laforin causes misfolding of glycogen and foci for seizures. The efficacy has been shown by 70% KD of Glycogen synthase GYS1. With respect to safety, GYS1 deficiency causes skeletal and cardiac muscle glycogen deficiency; GYS1 mice that survive have muscle defects. Possible diagnosis includes family history; genetic testing; or early symptoms. Biomarkers that can be used include, e.g., CSF mRNAs and protein.

### Ligands

In certain embodiments, the dsRNA agent of the invention is further modified by covalent attachment of one or more conjugate groups. In general, conjugate groups modify one or more properties of the attached dsRNA agent of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent dsRNA agent such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the dsRNA agent further comprises a targeting ligand that targets a receptor which mediates delivery to a specific CNS tissue. These targeting ligands can be conjugated in combination with the lipophilic moiety to enable specific intrathecal and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly Laspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA) (SEQ ID NO: 3); AALAEALAEALAEALAEALAEALAAAAGGC (EALA) (SEQ ID NO: 4); ALEALAEALEALAEA (SEQ ID NO: 5); GLFEAIEGFIENGWEGMIWDYG (INF-7) (SEQ ID NO: 6); GLFGAIAGFIENGWEGMIDGWYG (Inf HA-2) (SEQ ID NO: 7); GLFEAIEGFIENGWEGMIDGWYGCGLFEAIEGFIENGWEGMID GWYGC (diINF-7) (SEQ ID NO: 8); GLFEAIEGFIENGWEGMIDGGCGLFEAIEGFIENGWEGMIDGGC (diINF-3) (SEQ ID NO: 9); GLFGALAEALAEALAEHLAEALAEALEALAAGGSC (GLF) (SEQ ID NO: 10); GLFEAIEGFIENGWEGLAEALAEALEALAAGGSC (GALA-INF3) (SEQ ID NO: 11); GLF EAI EGFI ENGW EGnI DG K GLF EAI EGFI ENGW EGnI DG (INF-5, n is norleucine) (SEQ ID NO: 12); LFEALLELLESLWELLLEA (JTS-1) (SEQ ID NO: 13); GLFKALLKLLKSLWKLLLKA (ppTG1) (SEQ ID NO: 14); GLFRALLRLLRSLWRLLLRA (ppTG20) (SEQ ID NO: 15); WEAKLAKALAKALAKHLAKALAKALKACEA (KALA) (SEQ ID NO: 16); GLFFEAIAEFIEGGWEGLIEGC (HA) (SEQ ID NO: 17); GIGAVLKVLTTGLPALISWIKRKRQQ (Melittin) (SEQ ID NO: 18); H₅WYG (SEQ ID NO: 19); and CHK₆HC (SEQ ID NO: 20).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin) (SEQ ID NO: 21); GRKKRRQRRRPPQC (Tat fragment 48-60) (SEQ ID NO: 22); GALFLGWLGAAGSTMGAWSQPKKKRKV (signal sequence based peptide) (SEQ ID NO: 23); LLIILRRRIRKQAHAHSK (PVEC) (SEQ ID NO: 24); GWTLNSAGYLLKINLKALAALAKKIL (transportan) (SEQ ID NO: 25); KLALKLALKALKAALKLA (amphiphilic model peptide) (SEQ ID NO: 26); RRRRRRRRR (Arg9) (SEQ ID NO: 27); KFFKFFKFFK (Bacterial cell wall permeating peptide) (SEQ ID NO: 28); LLGDFFRKSKEKIGKEFKRIVQRIKDFLRNLVPRTES (LL-37) (SEQ ID NO: 29); SWLSKTAKKLENSAKKRISEGIAIAIQGGPR (cecropin P1) (SEQ ID NO: 30); ACYCRIPACIAGERRYGTCIYQGRLWAFCC (α-defensin) (SEQ ID NO: 31); DHYNCVSSGGQCLYSACPIFTKIQGTCYRGKAKCCK (β-defensin) (SEQ ID NO: 32); RRRPRPPYLPRPRPPPFFPPRLPPRIPPGFPPRFPPRFPGKR-NH2 (PR-39) (SEQ ID NO: 33); ILPWKWPWWPWRR-NH2 (indolicidin) (SEQ ID NO: 34); AAVALLPAVLLALLAP (RFGF) (SEQ ID NO: 35); AALLPVLLAAP (RFGF analogue) (SEQ ID NO: 36); and RKCRIVVIRVCR (bactenecin) (SEQ ID NO: 37).

Exemplary cationic groups include, but are not limited to, protonated amino groups, derived from e.g., 0-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the compound of the invention (e.g., a compound of the invention or linker). In some embodiments, the ligand can be incorporated via coupling to a “precursor” monomer after said “precursor” monomer has been incorporated into a component of the compound of the invention (e.g., a compound of the invention or linker). For example, a monomer having, e.g., an amino-terminated tether (i.e., having no associated ligand), e.g., monomer-linker-NH₂ can be incorporated into a component of the compounds of the invention (e.g., a compound of the invention or linker). In a subsequent operation, i.e., after incorporation of the precursor monomer into a component of the compounds of the invention (e.g., a compound of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the dsRNA agent of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Exemplary carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the dsRNA agent of the inventions via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578, 718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

Evaluation of Candidate dsRNAs

One can evaluate a candidate dsRNA agent, e.g., a modified RNA, for a selected property by exposing the agent or modified molecule and a control molecule to the appropriate conditions and evaluating for the presence of the selected property. For example, resistance to a degradant can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNA compounds.

In an alternative functional assay, a candidate dssiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dssiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The siRNA compounds described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of an siRNA compound on a human, e.g., any unwanted effect, such as a toxic effect, or any positive, or desired effect.

Increasing Cellular Uptake of siRNAs

Described herein are various siRNA compositions that contain covalently attached conjugates that increase cellular uptake and/or intracellular targeting of the siRNAs.

Additionally provided are methods of the invention that include administering an siRNA compound and a drug that affects the uptake of the siRNA into the cell. The drug can be administered before, after, or at the same time that the siRNA compound is administered. The drug can be covalently or non-covalently linked to the siRNA compound. The drug can be, for example, a lipopolysaccharide, an activator of p38 MAP kinase, or an activator of NF-κB. The drug can have a transient effect on the cell. The drug can increase the uptake of the siRNA compound into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the siRNA compound into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

siRNA Production

An siRNA can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilot II reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove endsiRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9; and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

### Making Double-Stranded RNA Agents Conjugated to a Lipophilic Moiety

In some embodiments, one or more lipophilic moieties are conjugated to the dsRNA agent via a nucleobase, sugar moiety, or internucleosidic linkage.

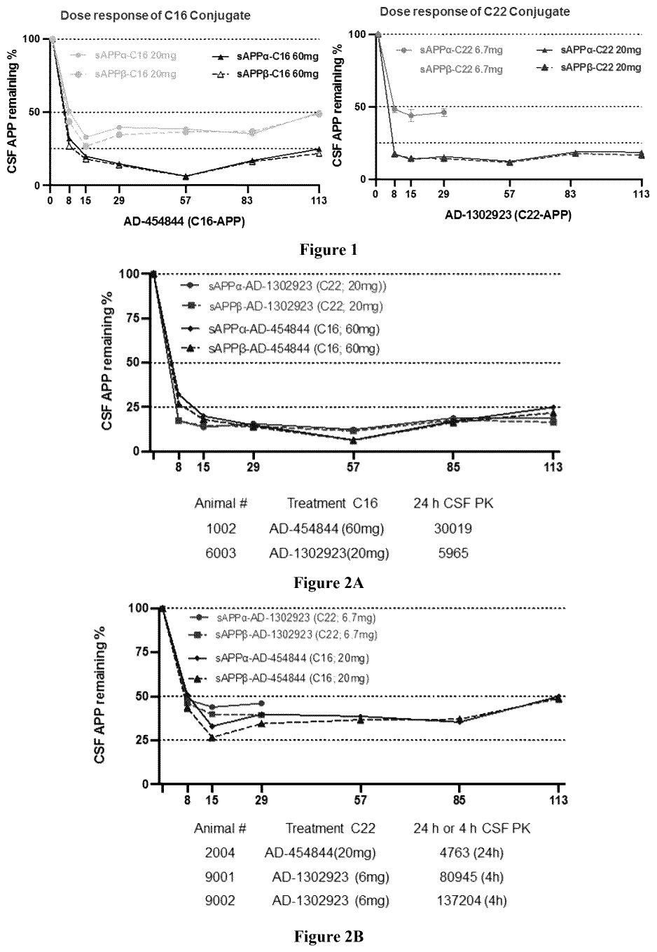

Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a lipophilic moiety is conjugated to a nucleobase, the preferred position is one that does not interfere with hybridization, i.e., does not interfere with the hydrogen bonding interactions needed for base pairing. In one embodiment, the lipophilic monomer containing a lipophilic moieties may be conjugated to a nucleobase via a linker containing an alkyl, alkenyl or amide linkage. Exemplary conjugations of the lipophilic moieties to the nucleobase are illustrated in FIG. 1 and Example 7 of WO 2019/217459, which is incorporated herein by reference in its entirety.

Conjugation to sugar moieties of nucleosides can occur at any carbon atom. Exemplary carbon atoms of a sugar moiety that a lipophilic moiety can be attached to include the 2′, 3′, and 5′ carbon atoms. A lipophilic moiety can also be attached to the 1′ position, such as in an abasic residue. In one embodiment, the lipophilic moieties may be conjugated to a sugar moiety, via a 2′-O modification, with or without a linker. Exemplary conjugations of the lipophilic moieties to the sugar moiety (via a 2′-O modification) are illustrated in FIG. 1 and Examples 1, 2, 3, and 6 of WO 2019/217459, which is incorporated herein by reference in its entirety.

Internucleosidic linkages can also bear lipophilic moieties. For phosphorus-containing linkages (e.g., phosphodiester, phosphorothioate, phosphorodithioate, phosphoroamidate, and the like), the lipophilic moiety can be attached directly to the phosphorus atom or to an O, N, or S atom bound to the phosphorus atom. For amine- or amide-containing internucleosidic linkages (e.g., PNA), the lipophilic moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant lipophilic moiety, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, a lipophilic molecule having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the lipophilic molecule has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the lipophilic molecule having the phosphoramidite group may include phosphitylation of a free hydroxyl to generate the phosphoramidite group.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### Pharmaceutical Compositions

In one aspect, the invention features a pharmaceutical composition that includes an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) including a nucleotide sequence complementary to a target RNA, e.g., substantially and/or exactly complementary. The target RNA can be a transcript of an endogenous human gene. In one embodiment, the siRNA compound (a) is 19-25 nucleotides long, for example, 21-23 nucleotides, (b) is complementary to an endogenous target RNA, and, optionally, (c) includes at least one 3′ overhang 1-5 nt long. In one embodiment, the pharmaceutical composition can be an emulsion, microemulsion, cream, jelly, or liposome.

In one aspect, the invention features a pharmaceutical composition including an siRNA compound and a delivery vehicle. In one embodiment, the siRNA compound is (a) is 19-25 nucleotides long, for example, 21-23 nucleotides, (b) is complementary to an endogenous target RNA, and, optionally, (c) includes at least one 3′ overhang 1-5 nucleotides long.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

### Treatment Methods and Routes of Delivery

Another aspect of the invention relates to a method of modulating the expression of a target gene in a CNS cell, comprising contacting said cell with the dsRNA agent of the invention. In one embodiment, the cell is an extrahepatic cell.

Another aspect of the invention relates to a method of modulating the expression of a target gene in the CNS of a subject, comprising administering to the subject the dsRNA agent of the invention.

Another aspect of the invention relates to a method of treating or preventing a CNS disorder in a subject, comprising administering to the subject a therapeutically effective amount of the double-stranded RNAi agent of the invention, thereby treating the subject by modulating the expression of the target gene in the CNS of the subject. Exemplary CNS disorders that can be treated by the method of the invention include Alzheimer, amyotrophic lateral sclerosis (ALS), frontotemporal dementia, Huntington, Parkinson, spinocerebellar, prion, and lafora.

The dsRNA agent of the invention can be delivered to a subject by a variety of routes, depending on the type of genes targeted and the type of disorders to be treated. In some embodiments, the dsRNA agent is administered extrahepatically, such as an intrathecal or intracerebroventricular administration.

In one embodiment, the dsRNA agent is administered intrathecally or intracerebroventricularly. By intrathecal or intracerebroventricular administration of the dsRNA agent, the method can reduce the expression of a target gene in a brain or spine tissue, for instance, cortex, cerebellum, cervical spine, lumbar spine, and thoracic spine.

In some embodiments, the target genes include APP, SOD1, SCN9A, HTT (HUNTINGTIN), APOE, LRRK2, PRNP, SCD5, GPR75, MAPT, SNCA, ABLIM3, ADRA2A, ATXN1, ATXN2, ATXN3, ELOVL1, FLNA, NOGO-L or NOGO-R, HIF-1α, RHO-A, NAV1.8, CD45, GSK-3, GSK3a, MIG-12, Mgat1, Mgat4, SLC35A1, SLC35A2, GNE, TMPRSS6, Complement Component C3, APCS, C9orf72, CHI3L1/YKL-40, EXT1, EXT2, NDST2, RPS25, ALK, and SCD5. In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, TTR, SCN9A, LRRK2, GPR75, APOE, SCD5, ELOVL1, FLNA, ALK, CHI3L1(YKL-40), RPS25, α2-AR, and GSK3a.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. A composition that includes a dsRNA can be delivered to a subject by a variety of routes.

The dsRNA molecules of the invention can be incorporated into pharmaceutical compositions suitable for administration. Such compositions typically include one or more species of dsRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. For example, administration may be to parenteral. Parenteral administration includes intravenous drip, subcutaneous, intraperitoneal or intramuscular injection, or intrathecal or intraventricular or intracerebroventricular administration. The route and site of administration may be chosen to enhance targeting. For example, to target muscle cells, intramuscular injection into the muscles of interest would be a logical choice.

Compositions for intrathecal or intraventricular or intracerebroventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

In one embodiment, the administration of the siRNA compound, e.g., a double-stranded siRNA compound, or ssiRNA compound, composition is parenteral, e.g., intravenous (e.g., as a bolus or as a diffusible infusion), intradermal, intraperitoneal, intramuscular, intrathecal, intraventricular, intracerebroventricular, intracranial, or subcutaneous. Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, the dsRNA agent is delivered by intrathecal injection (i.e. injection into the spinal fluid which bathes the brain and spinal cord tissue). Intrathecal injection of dsRNA agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally or intracerebroventricularly injected dsRNA agents may vary from one target gene to another target gene and the appropriate amount that has to be applied may have to be determined individually for each target gene. Typically, this amount ranges between 10 g to 2 mg, preferably 50 g to 1500 g, more preferably 100 g to 1000 g.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, trehalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention.

An aspect of the invention also relates to a method of delivering an oligonucleotide into the CNS by intrathecal or intracerebroventricular delivery,

Some embodiments relates to a method of reducing the expression of a target gene in a cell, comprising contacting said cell with an oligonucleotide having one or more lipophilic monomers containing lipophilic moieties conjugated to oligonucleotide, optionally via a linker or carrier. In one embodiment, the cell is a cell in the CNS system.

Some embodiments relates to a method of reducing the expression of a target gene in a subject, comprising administering to the subject an oligonucleotide having one or more lipophilic monomer containing lipophilic moieties conjugated to oligonucleotide, optionally via a linker or carrier. In one embodiment, the oligonucleotide conjugate is administered intrathecally or intracerebroventricularly (to reduce the expression of a target gene in a brain or spine tissue).

In some embodiments, the oligonucleotide is double-stranded. In one embodiment, the oligonucleotide is a compound comprising an antisense strand which is complementary to a target gene and a sense strand which is complementary to said antisense strand.

In some embodiments, the oligonucleotide is single-stranded. In one embodiment, the oligonucleotide is an antisense.

In some embodiments, the lipophilic monomer containing a lipophilic moiety is located on one or more internal positions on at least one strand of the oligonucleotide. In some embodiments, the lipophilic monomer containing a lipophilic moiety is located on one or more terminal positions on at least one strand of the oligonucleotide.

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples which are included merely for purposes of illustration of certain aspects and embodiments of the present invention and are not intended to limit the invention.

### Example 1: Synthesis of C22-Nucleoside Phosphoramidites for the Synthesis of dsRNA Agent Conjugates

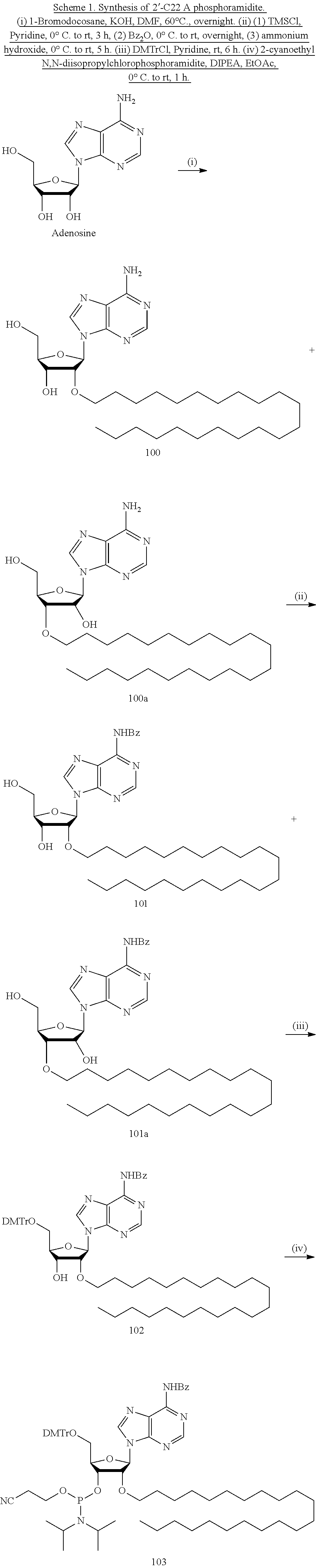

Compound 100: Adenosine (25 g, 93.6 mmol) and DMF (250 mL) were added into a 500 mL round-bottom flask, and then the suspension was warmed to 60° C. 1-Bromodocosane (54.7 g, 140 mmol) and KOH (10.5 g, 187 mmol) were added into the suspension and the reaction mixture was stirred at 60° C. overnight (16 hours). The reaction was cooled to room temperature (a lot of insoluble matters were observed) and quenched by addition of NH₄Cl (10 g). The mixture, including the insoluble matter, was poured into a 2 L separating funnel and diluted with CH₂Cl₂ and H₂O. (3 phases; organic phase, aqueous phase and emulsion phase, were observed.) The aqueous and emulsion phases were extracted with CH₂Cl₂ 3 times. TLC indicated that a major product spot was detected in the organic phase (5% MeOH in ethyl acetate, Rf=0.5). The collected organic phase was dried over anhydrous sodium sulfate and concentrated under vacuum. The crude compound was purified by column chromatography on silica gel (0-10% MeOH in CH₂Cl₂ for 60 minutes) to obtain a mixture of 2′-C22 product (compound 100) and 3′-C22 product (100a) as a white solid (28.2 g, 52%; 2′-C22/3′-C22≈9:1). ¹H NMR (600 MHz, DMSO-d₆) δ 8.38-8.33 (m, 1H), 8.13 (s, 1H), 7.35 (brs, 2H), 5.98 (d, J=6.3 Hz, 0.9H), 5.78 (d, J=6.2 Hz, 0.1H), 5.47-5.40 (m, 1H), 5.19-5.16 (m, 1H), 4.73 (q, J=5.8 Hz, 0.1H), 4.47 (dd, J=4.8, 6.4 Hz, 0.9H), 4.30-4.28 (m, 0.9H), 4.04-4.03 (m, 0.1H), 3.99-3.97 (m, 0.9H), 3.94-3.92 (m, 0.1H), 3.69-3.66 (m, 1H), 3.58-3.53 (m, 2H), 3.37-3.30 (m, 1H), 1.58-1.53 (m, 0.2H), 1.39-1.36 (m, 1.8H), 1.29-1.08 (m, 38H), 0.85 (t, J=6.6 Hz, 3H). LCMS (ESI) calculated for C₃₂H₅₈N₅O₄ [M+H]⁺ m/z=576.45, found 576.4.

Compound 101: To a suspension of compound 100 and 3′-C22 100a (mixture, 28 g, 48.6 mmol) in pyridine (40 mL) was added dropwise TMSCl (2.98 mL, 23.4 mmol) at 0° C. and the mixture was warmed to room temperature and stirred for 3 h. TLC indicated that compound 100 and 100a was consumed, and a new major spot was detected (50% ethyl acetate in hexane, Rf=0.7). The reaction mixture was cooled to 0° C. and benzoic anhydride (2.12 g, 9.38 mmol) was added. The resulting solution was wormed to room temperature and stirred overnight (14 h). TLC indicated that the protected intermediate with TMS was consumed, and a new major spot was detected (50% ethyl acetate in hexane, Rf=0.8). The reaction was cooled to 0° C. and quenched with H₂O. The resulting solution was warmed to room temperature and stirred for 5 hours. The mixture was cooled to 0° C. and 28% ammonium hydroxide solution (40 mL) was added. The resulting mixture was warmed to room temperature and stirred for 5 hours. TLC indicated that the fully protected intermediate with TMS and Bz groups was consumed, and a new major spot was detected (100% ethyl acetate, Rf=0.5). The reaction was diluted with ethyl acetate and the organic layer was washed with water, brine and dried (Na₂SO₄) and concentrated under vacuum. The crude residue was purified by column chromatography on silica gel (0-5% MeOH in CH₂Cl₂ for 30 minutes) to obtain a mixture of 2′-C22 product (compound 101) and 3′-C22 product (101a) as a white solid (24.9 g, 75%; 2′-C22/3′-C22≈9:1). ¹H NMR (600 MHz, DMSO-d₆) δ 11.22 (s, 1H), 8.75 (s, 1.8H), 8.73 (s, 0.2H), 8.06-8.04 (m, 2H), 7.66-7.63 (m, 1H), 7.57-7.54 (m, 2H), 6.14 (d, J=5.9 Hz, 0.9H), 6.04 (d, J=5.8 Hz, 0.1H), 5.53 (d, J=6.2 Hz, 0.1H), 5.23 (d, J=5.3 Hz, 0.9H), 5.17 (brs, 1H), 4.79 (q, J=5.5 Hz, 0.1H), 4.52 (dd, J=5.0, 6.1 Hz, 0.9H), 4.34 (q, J=4.2 Hz, 0.9H), 4.06 (q, J=3.9 Hz, 0.1H), 4.04-3.98 (m, 1H), 3.71-3.69 (m, 1H), 3.62-3.59 (m, 2H), 3.52-3.48 (m, 0.1H), 3.42-3.39 (m, 0.9H), 1.58-1.53 (m, 0.2H), 1.44-1.40 (m, 1.8H), 1.26-1.11 (m, 38H), 0.84 (t, J=6.8 Hz, 3H). LCMS (ESI) calculated for C₃₉H₆₂N₅O₅ [M+H]⁺ m/z=680.48, found 680.6.

Compound 102: To a solution of compound 101 and 3′-C22 product 101a (mixture, 25 g, 36.8 mmol) in pyridine (300 mL) was added 4,4′-dimethoxytriphenyl chloride (12.5 g, 36.8 mmol) and the mixture was stirred at room temperature for 6 hours. TLC indicated that compound 101 was consumed, and a new major spot was detected (90% ethyl acetate in hexane, Rf=0.8). The reaction was quenched with saturated NaHCO₃ (aqueous) and diluted with ethyl acetate. The organic layer was washed with water, brine and dried (Na₂SO₄) and concentrated under vacuum. The crude residue was purified by column chromatography on silica gel (0-50% ethyl acetate in hexane for 10 minutes and then kept 50% ethyl acetate in hexane for 10 minutes) to obtain compound 102 as a light-yellow form (28.5 g, 79%). ¹H NMR (600 MHz, DMSO-d₆) δ 11.23 (brs, 1H), 8.66 (s, 1H), 8.60 (s, 1H), 8.06-8.04 (m, 2H), 7.66-7.63 (m, 1H), 7.56-7.54 (m, 2H), 7.38-7.36 (m, 2H), 7.27-7.18 (m, 7H), 6.85-6.82 (m, 4H), 6.15 (d, J=5.3 Hz, 1H), 5.26 (d, J=5.8 Hz, 1H), 4.66 (t, J=5.1 Hz, 1H), 4.39 (q, J=5.1 Hz, 1H), 4.12 (q, J=4.5 Hz, 1H), 3.72 (s, 6H), 3.61 (dt, J=6.5, 9.7 Hz, 1H), 3.45 (dt, J=6.5, 9.7 Hz, 1H), 3.30-3.24 (m, 2H), 1.47-1.42 (m, 2H), 1.27-1.12 (m, 38H), 0.84 (t, J=6.8 Hz, 3H). LCMS (ESI) calculated for C₆₀H₈₀N₅O₇ [M+H]⁺ m/z=982.61, found 982.6.

Compound 103: To a solution of compound 102 (5 g, 5.09 mmol) in ethyl acetate (30 mL) was added dropwise 2-cyanoethyl N,N-diisopropylchlorophosphoramidite (1.48 mL, 6.62 mmol) at 0° C. and the mixture was stirred at room temperature for 1 hour. TLC indicated that compound 102 was consumed, and a new major spot was detected (40% ethyl acetate in hexane, Rf=0.4). The reaction mixture was quenched with saturated NaHCO₃ (aqueous) and then the organic layer was washed with water, brine, dried (Na₂SO₄) and concentrated under vacuum. The crude residue was purified by column chromatography on silica gel (0-40% ethyl acetate in hexane for 15 minutes and then 40-50% ethyl acetate in hexane for 20 minutes) to obtain compound 103 as a white form (4.58 g, 76%). ¹H NMR (600 MHz, CD₃CN) δ 9.36 (brs, 1H), 8.62-8.60 (m, 1H), 8.31-8.29 (m, 1H), 8.02-8.00 (m, 2H), 7.67-7.64 (m, 1H), 7.57-7.55 (m, 2H), 7.47-7.43 (m, 2H), 7.35-7.21 (m, 7H), 6.86-6.82 (m, 4H), 6.14-6.13 (m, 1H), 4.84-4.81 (m, 1H), 4.74-4.69 (m, 1H), 4.37-4.31 (m, 1H), 3.95-3.63 (m, 11H), 3.56-3.52 (m, 1H), 3.50-3.45 (m, 1H), 3.39-3.33 (m, 1H), 2.74-2.65 (m, 1H), 2.52 (t, J=6.1 Hz, 1H), 1.53-1.48 (m, 2H), 1.34-1.19 (m, 47H), 1.12 (d, J=6.8 Hz, 3H), 0.90 (t, J=6.8 Hz, 3H). ³¹P NMR (243 MHz, CD₃CN) δ 149.89, 149.85, 149.81, 149.77, 149.74, 149.70, 149.48, 149.44, 149.40, 149.36, 149.32, 149.28. LCMS (ESI) calculated for C₆₉H₉₇N₇O₈P [M+H]⁺ m/z=1182.71, found 1182.6.

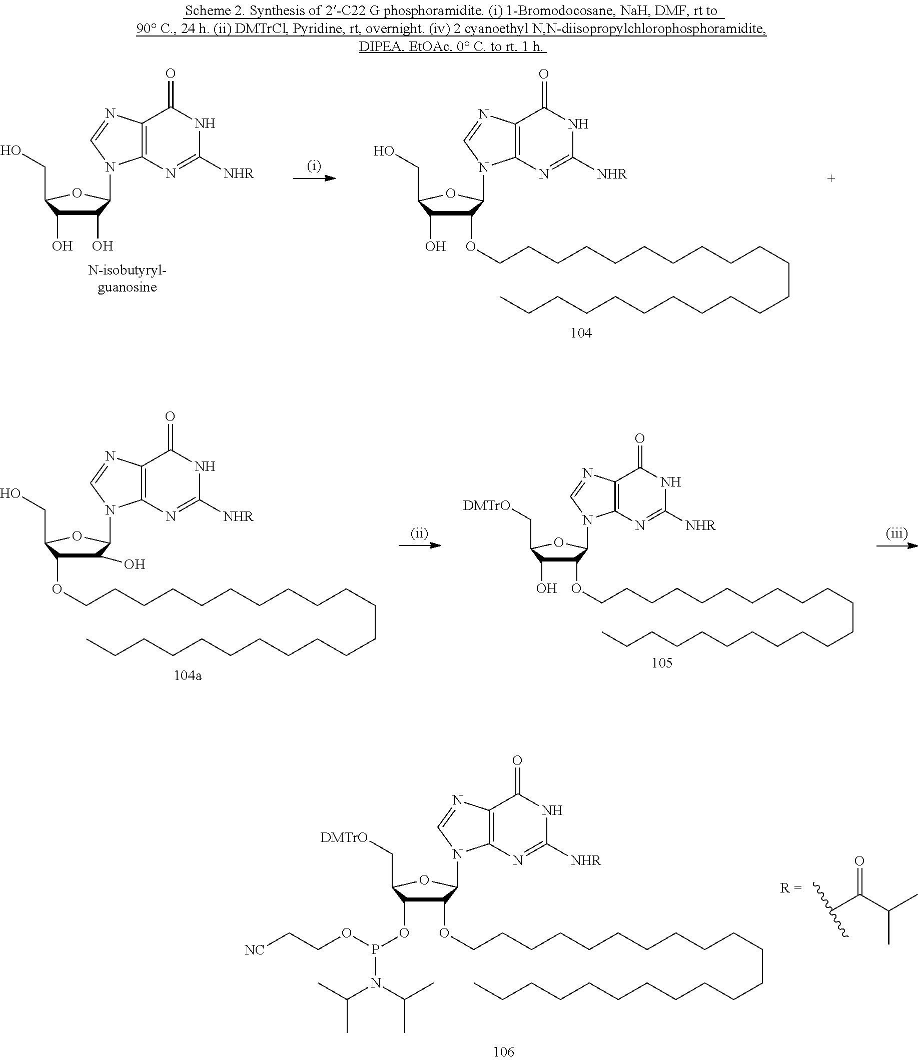

Compound 104: N-Isobutyrylguanosine (5 g, 14.2 mmol) and DMF (50 mL) were added into a 250 mL round-bottom flask, and then the solution was cooled to 0° C. NaH (60% in mineral oil; 1.42 g, 35.4 mmol) was added portion-wise into the solution and the suspension was stirred at 0° C. for 30 minutes. To the mixture was added 1-bromodocosane (8.27 g, 21.2 mmol). The suspension was wormed up to 90° C. and stirred for 24 hours. TLC indicated that N-isobutyrylguanosine remained, and a new major spot was detected (100% ethyl acetate, Rf=0.4). The reaction mixture was cooled to 0° C. and quenched by addition of NH₄Cl (a lot of insoluble matters were observed). The mixture, including the insoluble matter, was poured into a 1 L separating funnel and diluted with CH₂Cl₂ and H₂O. (3 phases; organic phase, aqueous phase and emulsion phase, were observed.) The aqueous and emulsion phases were extracted with CH₂Cl₂ 3 times. The collected organic phase was dried over anhydrous sodium sulfate (Na₂SO₄) and concentrated under vacuum. The crude compound was purified by column chromatography on silica gel (0-10% MeOH in CH₂Cl₂ for 30 min) to obtain a mixture of 2′-C22 (compound 104)/3′-C22 products (104a), contained some other impurities, as a white solid (2.10 g, 22%). LCMS (ESI) calculated for C₃₆H₆₄N₅O₆ [M+H]⁺ m/z=662.49, found 662.6. The obtained compound 104 was used for the next reaction without any further purifications.

Compound 105: To a solution of compound 104 (2.1 g, 3.17 mmol) in pyridine (30 mL) was added 4,4′-dimethoxytriphenyl chloride (1.07 g, 3.17 mmol) and the mixture was stirred at room temperature for overnight (14 hours). TLC indicated that compound 104 was consumed, and a new major spot was detected (80% ethyl acetate in hexane, Rf=0.8). The reaction was quenched with saturated NaHCO₃ (aqueous) and diluted with ethyl acetate. The organic layer was washed with water, brine and dried (Na₂SO₄) and concentrated under vacuum. The crude residue was purified by column chromatography on silica gel (0-45% ethyl acetate in hexane for 10 minutes and then kept 45% ethyl acetate in hexane for 30 min) to obtain compound 105 as a white form (2.13 g, 70%). ¹H NMR (600 MHz, DMSO-d₆) δ 12.10 (brs, 1H), 11.64 (brs, 1H), 8.12 (s, 1H), 7.36-7.34 (m, 2H), 7.27-7.19 (m, 7H), 6.85-6.81 (m, 4H), 5.93 (d, J=5.8 Hz, 1H), 5.18 (d, J=5.4 Hz, 1H), 4.40 (t, J=5.4 Hz, 1H), 4.26 (q, J=4.8 Hz, 1H), 4.05 (ddd, J=3.5, 3.5, 6.0 Hz, 1H), 3.73 (s, 6H), 3.59 (dt, J=6.4, 9.7 Hz, 1H), 3.45 (dt, J=6.4, 9.7 Hz, 1H), 3.30 (d, J=6.0, 10.5 Hz, 1H), 3.16 (d, J=3.5, 10.5 Hz, 1H), 2.76 (sept, J=6.8 Hz, 1H), 1.44-1.40 (m, 2H), 1.26-1.11 (m, 44H), 0.84 (t, J=6.8 Hz, 3H). LCMS (ESI) calculated for C₅₇H₈₂N₅O₈ [M+H]⁺ m/z=964.62, found 964.6.

Compound 106: To a solution of compound 105 (2 g, 2.07 mmol) and DIPEA (0.470 mL, 2.70 mmol) in ethyl acetate (20 mL) was added dropwise 2-cyanoethyl N,N-diisopropylchlorophosphoramidite (0.601 mL, 2.70 mmol) at 0° C. and the mixture was stirred at room temperature for 1 hour. TLC indicated that compound 105 was consumed, and a new major spot was detected (50% ethyl acetate in hexane, Rf=0.6). The reaction mixture was washed with saturated NaHCO₃ (aqueous), water, brine, dried (Na₂SO₄) and concentrated under vacuum. The crude residue was purified by column chromatography on silica gel (0-50% ethyl acetate in hexane for 30 minutes and then kept 50% ethyl acetate in hexane for 10 min) to obtain compound 106 as a white form (1.76 g, 73%). ¹H NMR (600 MHz, CD₃CN) δ 11.99 (brs, 1H), 9.16 (brs, 1H), 7.89-7.87 (m, 1H), 7.49-7.23 (m, 9H), 6.87-6.83 (m, 4H), 5.92 (t, J=6.1 Hz, 1H), 4.68-4.63 (m, 1H), 4.53-4.47 (m, 1H), 4.34-4.26 (m, 1H), 3.92-3.60 (m, 11H), 3.54-3.49 (m, 1H), 3.40 (d, J=4.0 Hz, 1H), 3.35 (d, J=4.0 Hz, 1H), 2.73-2.65 (m, 1H), 2.57-2.47 (m, 2H), 1.53-1.47 (m, 2H), 1.34-1.18 (m, 47H), 1.15-1.06 (m, 9H), 0.90 (t, J=6.7 Hz, 3H). ³¹P NMR (243 MHz, CD₃CN) δ 149.80, 149.76, 149.72, 149.68, 149.64, 149.60, 149.56, 149.51, 149.48, 149.44. LCMS (ESI) calculated for C₆₆H₉₉N₇O₉P [M+H]⁺ m/z=1164.72, found 1164.8.

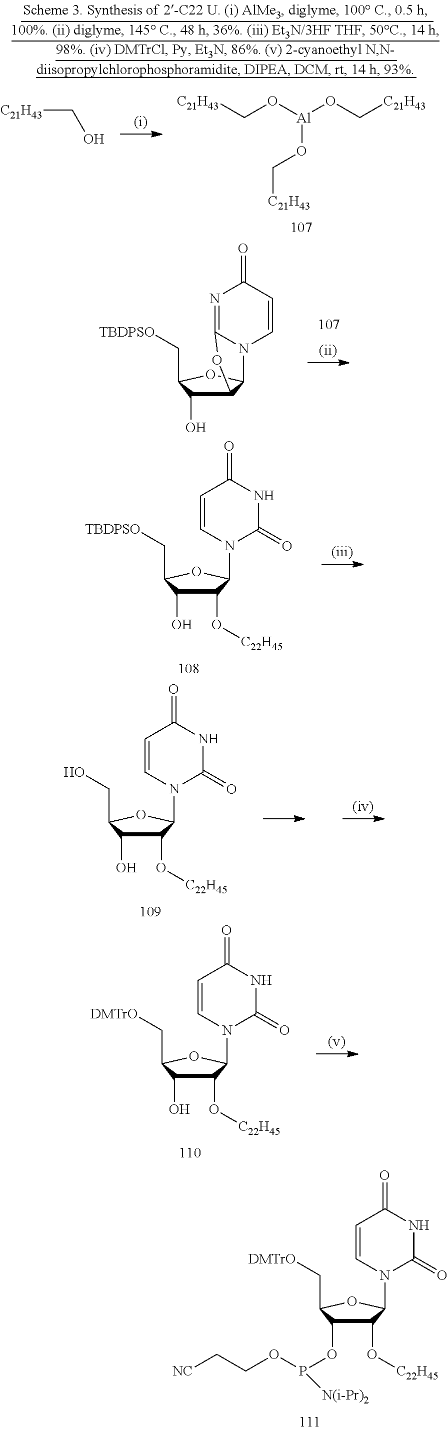

Compound 107: 2M solution of AlMe₃ (50 mL, 0.10 mol) was added slowly for about 15 minutes to a stirred suspension of 1-docosanol (108 g, 0.33 mol) in anhydrous diglyme (90 mL) under Ar atmosphere in a 2-neck 1 L flask fitted with a magnetic stirring bar, and an outlet with a gas bubbler over a reflux condenser. After completion of the addition, the mixture was heated to 100° C. until evolution of gas through the bubbler was complete (30 minutes). The mixture was cooled to 65° C. and diluted with anhydrous AcOEt (150 mL) and anhydrous ACN (150 mL). The mixture was cooled down to room temperature in the bath overnight, a white residue formed was filtered through a 600 mL glass filtering funnel under the cushion of Ar, and washed with a 1:1 mixture of anhydrous AcOEt and anhydrous ACN (400 mL×2) under the cushion of Ar. The residue was dried on the funnel in reverse flow of nitrogen, transferred to a flask and dried in high vacuum for 24 hours to afford 107.4 g of the alkoxide 107 of about 93% purity containing about 7% of 1-docosanol that was used in the next step without of further purification. The product was stored under Ar atmosphere.

Compound 108: A mixture of 5′-TBDPS-protected anhydro-uridine (18.6 g, 40 mmol), aluminum alkoxide 107 (~93%, 47.6 g, 44 mmol) and anhydrous diglyme (60 mL) was heated to 145° C. bath temperature in a flask fitted with a magnetic stirring bar and a reflux condenser under slight positive pressure of Ar using a balloon for 48 hours. The mixture was cooled down to 70° C. in the bath, diluted with AcOEt (200 mL), further cooled down 30° C. and quenched by addition of 10% H₃PO₄ (200 mL). A suspension thus formed was stirred at room temperature overnight, filtered through a 600 mL glass filtering funnel, and the solids were washed thoroughly with water (about 50 mL) and AcOEt (about 300 mL) mixture. Thoroughly compressed solid residue was dried in warm air to afford 25.4 g (55%) of recovered 1-docosanol. The filtrate was transferred to a separatory funnel, the organic layer was separated, washed with 1% NaCl (500 mL×2), saturated NaCl (200 mL) and dried over anhydrous Na₂SO₄. The solvent was removed in vacuo, the residue was co-evaporated with additional portion of AcOEt (300 mL) to afford 61.8 g of crude residue. The latter was dissolved in 190 mL of AcOEt-hexanes 1:4 mixture and liquid-loaded on a standard 330 g column of silica gel. The column was eluted with isocratic 20% AcOEt in hexanes followed by gradient of 20 to 40% of AcOEt in hexanes, the fractions containing product were pulled, evaporated in vacuum, co-evaporated twice with ACN-diethyl ether mixture, and dried in high vacuum to afford 11.4 g (36%) of pure product 108. ¹H NMR (600 MHz, Acetone-d6) δ 10.08 (s, 1H), 7.88 (d, J=7.8 Hz, 1H), 7.80-7.76 (m, 2H), 7.76-7.73 (m, 2H), 7.53-7.44 (m, 6H); 6.00 (d, J=3.0 Hz, 1H), 5.27 (d, J=8.4 Hz, 1H), 4.46 (q, J=5.4 Hz, 1H), 4.12 (dd, J=12.0, 2.4 Hz, 1H), 4.08-4.04 (m, 2H), 3.98 (dd, J=11.4, 2.4 Hz, 1H), 3.95 (d, J=7.2 Hz, 1H), 3.78 (dt, J=9.6, 6.6 Hz, 1H), 3.69 (dt, J=9.6, 6.6 Hz, 1H), 1.65-1.59 (m, 2H), 1.43-1.36 (m, 2H), 1.35-1.25 (m, 36H), 1.13 (s, 9H), 0.89 (t, J=6.6 Hz, 3H). MS (ESI+APCI), calculated for C₄₇H₇₄N₂O₆Si [M+H]⁺ exact mass m/z=791.54, found 791.7.

Compound 109: A mixture of TBDPS-protected nucleoside 108 (2.25 g, 2.8 mmol), anhydrous THE (10 mL), and triethylamine trihydrofluoride (2 mL, 12 mmol) was heated at 50° C. under Ar atmosphere for 24 h. Heptane (40 mL) followed by water (40 mL) were added, the heating bath was removed, the mixture was stirred overnight at rt, filtered, and washed thoroughly by water-heptane mixture. The solid was dried in the flow of nitrogen for 2 hours followed by warm air overnight to afford 1.52 g (98%) of 109 as a white solid. ¹H NMR (600 MHz, Acetone-d6) δ 10.01 (s, 1H), 7.88 (d, J=8.4 Hz, 1H), 5.97 (d, J=4.2 Hz, 1H), 5.60 (d, J=8.4 Hz, 1H), 4.37 (s, 1H), 4.35-4.29 (m, 1H), 4.06 (t, J=4.8 Hz, 1H), 4.00 (dt, J=5.4, 2.4 Hz, 1H), 3.93-3.87 (m, 1H), 3.84 (d, J=6.6 Hz, 1H), 3.83-3.78 (m, 1H), 3.74-3.63 (m, 2H), 1.64-1.56 (m, 2H), 1.43-1.23 (m, 38H), 0.89 (t, J=6.6 Hz, 3H). MS (ESI+APCI), calculated for C₃₁H₅₆N₂O₆ [M+H]⁺ exact mass m/z=553.42, found 553.5.

Compound 110: Triethylamine (0.77 mL, 5.5 mmol) was added to a solution of nucleoside 109 (1.49 g, 2.7 mmol) and DMTrCl (1.86 g, 5.5 mmol) in anhydrous pyridine (10 mL) under Ar atmosphere. The mixture was stirred at room temperature overnight, quenched by addition of MeOH (0.2 mL), and diluted with ACN (25 mL). The solvents were evaporated in vacuo at 25° C., the residue was co-evaporated twice with ACN at 25° C. and partitioned between AcOEt and 5% NaCl. The organic phase was separated, washed with saturated NaCl, and dried over anhydrous Na₂SO₄. The solvent was removed in vacuo to afford 3.50 g of crude product. The latter was purified by chromatography over a standard 40 g column of silicagel with isocratic 30% of AcOEt in hexanes followed by gradient of 30 to 50% of AcOEt in hexanes. Fractions contained product were pulled evaporated in vacuum, co-evaporated twice with ACN-diethyl ether mixture, and dried in high vacuum to afford 1.98 g (86%) of product 110 as a yellowish foam. ¹H NMR (600 MHz, Acetone-d6) δ 10.08 (s, 1H), 7.93 (d, J=7.8 Hz, 1H), 7.52-7.48 (m, 2H), 7.40-7.33 (m, 6H), 7.29-7.26 (m, 1H), 6.93 (d split, J=9.0 Hz, 4H), 5.96 (d, J=2.4 Hz, 1H), 5.27 (d, J=7.8 Hz, 1H), 4.52-4.48 (m, 1H), 4.12-4.07 (m, 2H), 3.94 (d, J=7.8 Hz, 1H), 3.85-3.77 (m, 1H), 3.81 (s, 6H), 3.73-3.68 (m, 1H), 3.52 (dd, J=10.8, 3.6 Hz, 1H), 3.46 (dd, J=10.8, 3.0 Hz, 1H), 1.67-1.59 (m, 2H), 1.43-1.56 (m, 2H), 1.36-1.23 (m, 36H), 0.89 (t, J=6.6 Hz, 3H). MS (ESI+APCI), calculated for C₅₂H₇₄N₂O₈ [M+H]⁺ exact mass m/z=854.54.

Compound 111: DIPEA (0.46 mL, 2.6 mmol) followed by 2-cyanoethyl N,N-diisopropylchlorophosphoramidite (0.57 mL, 2.6 mmol) were added to a solution of compound 110 (1.71 g, 2 mmol) in anhydrous DCM (10 mL) under Ar. The mixture was stirred at room temperature for 14 hours, cooled to 0° C., quenched by addition of saturated NaHCO₃ and extracted with AcOEt (25 mL). Organic phase was separated, washed with saturated NaCl, and dried over anhydrous sodium sulfate. Crude material (2.20 g) was purified over a standard 40 g flash column of silica gel that was eluted with isocratic 50% of AcOEt containing 0.3% of TEA in hexanes to afford 1.96 g (93%) of 111 as a white foam. ¹H NMR (500 MHz, CD3CN) δ 8.93 (s, 1H), 7.82-7.70 (m, 1H), 7.47-7.41 (m, 2H), 7.36-7.23 (m, 8H), 6.91-6.85 (m, 4H), 5.87-5.82 (m, 1H), 5.24-5.19 (m, 1H), 4.50-4.38 (m, 1H), 4.19-4.11 (m, 1H), 4.06-3.99 (m, 1H), 3.77 (s, 3H), 3.77 (s, 3H), 3.63-3.59 (m, 2H), 3.45-3.41 (m, 1H), 2.72-2.59 (m, 1H), 2.52 (t, J=6.0 Hz, 1H), 1.60-1.50 (m, 2H), 1.42-1.07 (m, 51H), 1.06 (d, J=6.8 Hz, 3H), 0.90-0.84 (m, 3H). C13 NMR (151 MHz, CD3CN) δ 163.47, 163.43, 159.33, 159.31, 159.30, 150.84, 145.32, 145.25, 140.54, 140.51, 135.99, 135.95, 135.85, 135.77, 130.76, 130.71, 130.69, 128.63, 128.60, 128.51, 127.57, 119.12, 119.00, 113.68, 113.67, 102.11, 102.02, 88.36, 88.23, 87.22, 87.18, 82.95, 82.93, 82.80, 82.76, 81.98, 81.25, 81.22, 71.24, 71.00, 70.76, 70.66, 70.59, 70.51, 62.45, 62.00, 59.15, 59.02, 58.78, 58.64, 55.48, 55.46, 43.58, 43.54, 43.50, 43.45, 32.21, 30.01, 29.99, 29.96, 29.92, 29.85, 29.73, 29.69, 29.65, 26.32, 26.30, 24.67, 24.62, 24.57, 24.52, 24.48, 24.46, 24.41, 22.96, 20.65, 20.60, 13.98. P31 NMR (202 MHz, CD3CN) δ 149.47, 149.08.

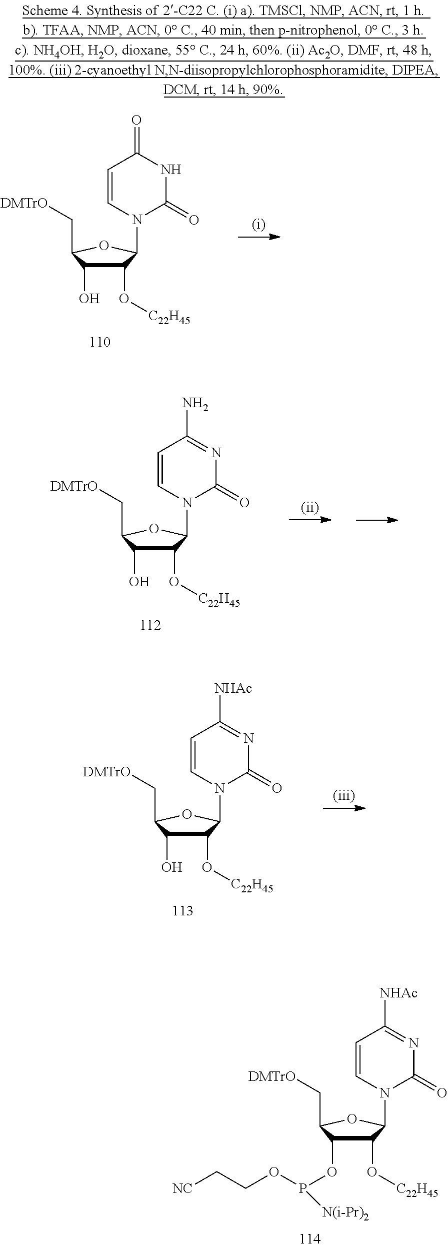

Compound 112: TMSCl (0.4 mL, 3.2 mmol) was added to a solution of 110 (1.20 g, 1.4 mmol) and NMP (1.2 mL, 11.8 mmol) in anhydrous MeCN (7 mL) under Ar atmosphere. The mixture was stirred at room temperature for 1 hour, cooled to 0° C., and TFAA (0.5 mL, 3.6 mmol) was added slowly dropwise via syringe. The mixture was stirred at 0° C. for 40 minutes, and p-nitrophenol (0.56 g, 4 mmol) was added. The mixture was stirred at 0° C. for 3 hours and quenched by addition of saturated sodium bicarbonate (15 mL). The cooling bath was removed, ethyl acetate (30 mL) was added, followed by minimal amount of water to dissolve inorganic precipitates. The organic phase was separated, washed with saturated NaCl, dried over anhydrous sodium sulfate and evaporated in vacuum to afford 1.94 g of oily residue. The latter was dissolved in dioxane (15 mL), the solution was transferred to a pressure bottle, saturated ammonium hydroxide solution (2.2 mL) was added, and the bottle was heated at 55° C. with stirring for 24 hours. The mixture was cooled to room temperature, the solvent was evaporated in vacuum and the residue (2.40 g) was chromatographed over a column of silica gel with gradient of methanol in ethyl acetate (0 to 6%). The fraction containing product were pulled, evaporated in vacuum, and the residue was treated with 5 mL of ACN that triggered extensive crystallization. The mixture was kept at 0° C. for 4 hours, filtered, the crystalline residue was washed with ACN, and air-dried to afford 0.73 g (60%) of C-22 cytidine 112 as a white solid. ¹H NMR (400 MHz, DMSO-d6) δ 7.77 (d, J=7.2 Hz, 1H), 7.42-7.35 (m, 2H), 7.31 (t, J=7.2 Hz, 2H), 7.28-7.22 (m, 5H), 7.22-7.12 (m, 2H), 6.89 (d, J=8.8 Hz, 4H), 5.80 (d, J=2.8 Hz, 1H), 5.48 (d, J=7.6 Hz, 1H), 4.98 (d, J=6.8 Hz, 1H), 4.19-4.11 (m, 1H), 3.99-3.90 (m, 1H), 3.77-3.69 (m, 1H), 3.73 (s, 6H), 3.68-3.52 (m, 2H), 3.30-3.22 (m, 2H), 1.55-1.45 (m, 2H), 1.34-1.14 (m, 38H), 0.83 (t, J=6.8 Hz, 3H). MS (ESI+APCI), calculated for C₅₂H₇₅N₃O₇ [M+H]⁺ exact mass m/z=854.57.

Compound 113: C-22-Cytidine 112 (0.68 g, 0.8 mmol) was dissolved in anhydrous DMF (4 mL) under Ar atmosphere, and acetic anhydride (0.09 mL, 0.9 mmol) was added. The mixture was stirred at room temperature for 48 hours, cooled to 0° C., quenched by addition of 5% NaCl (10 mL), and diluted with ethyl acetate (10 mL). The organic phase was separated, washed with 5% NaCl (2×20 mL), saturated sodium bicarbonate, saturated NaCl, and dried over anhydrous sodium sulfate. The solvent was removed in vacuum and the residue was co-evaporated twice with ACN-diethyl ether mixture to afford 0.72 g (100%) of C-22 N(Ac)-cytidine 113 as a white solid. ¹H NMR (400 MHz, DMSO-d6) δ 10.90 (s, 1H), 8.28 (d, J=7.6 Hz, 1H), 7.41-7.35 (m, 2H), 7.31 (t, J=7.2 Hz, 2H), 7.28-7.21 (m, 5H), 7.00 (d, J=7.2 Hz, 1H), 6.92-6.86 (m, 4H), 5.79 (d, J=1.2 Hz, 1H), 5.05 (d, J=7.2 Hz, 1H), 4.25-4.18 (m, 1H), 4.05-3.99 (m, 1H), 3.78 (dd, J=4.8, 1.2 Hz, 1H), 3.77-3.69 (m, 1H), 3.74 (s, 6H), 3.65-3.57 (m, 1H), 3.37-3.27 (m, 2H), 2.08 (s, 3H), 1.57-1.48 (m, 2H), 1.35-1.14 (m, 38H), 0.83 (t, J=6.8 Hz, 3H). MS (ESI+APCI), calculated for C₅₄H₇₇N₃O₈ [M+H]⁺ exact mass m/z=896.58.

Compound 114: DIPEA (0.16 mL, 0.9 mmol) followed by 2-cyanoethyl N,N-diisopropylchlorophosphoramidite (0.20 mL, 0.9 mmol) were added to a solution of compound 113 (0.63 g, 0.7 mmol) in anhydrous DCM (5 mL) under Ar. The mixture was stirred at room temperature for 14 hours, cooled to 0° C., quenched by addition of saturated NaHCO₃ and extracted with AcOEt (15 mL). Organic phase was separated, washed with saturated NaCl, and dried over anhydrous sodium sulfate. Crude material (0.82 g) was purified over a standard 24 g flash column of silica gel that was eluted with isocratic 70% of AcOEt containing 0.3% of TEA in hexanes followed by gradient 70 to 100% of AcOEt containing 0.3% of TEA in hexanes to afford 0.69 g (90%) of 114 as a white foam. ¹H NMR (600 MHz, DMSO) δ 10.94 (s, 1H), 8.46-8.34 (m, 1H), 7.46-7.36 (m, 2H), 7.36-7.20 (m, 7H), 6.98 (t, J=8.4 Hz, 1H), 6.89 (t, J=8.6 Hz, 4H), 5.85 (d, J=13.8 Hz, 1H), 4.55-4.34 (m, 1H), 4.20-4.11 (m, 1H), 4.06-3.83 (m, 2H), 3.75 (s, 3H), 3.74 (s, 3H), 3.68-3.64 (m, 1H), 3.50-3.44 (m, 2H), 2.77-2.69 (m, 1H), 2.66-2.59 (m, 1H), 2.10 (s, 3H), 1.56-1.49 (m, 2H), 1.26-1.16 (m, 51H), 0.96 (d, J=6.7 Hz, 3H), 0.85-0.82 (m, 3H). P31 NMR (243 MHz, DMSO) δ 149.23, 147.94.

### Example 2. Synthesis of Lipophilic Monomers

Lipophilic monomers were synthesized to introduce lipophilic ligands at various locations of siRNAs (terminal and/or internal positions) as solid support or phosphoramidites.

A variety of lipids can be conjugated via hydroxyprolinol derivatives using methods as shown in the schemes below (e.g., Schemes 5-7 for general procedures), and the resulting building block phosphoramidites can be incorporated into siRNAs.

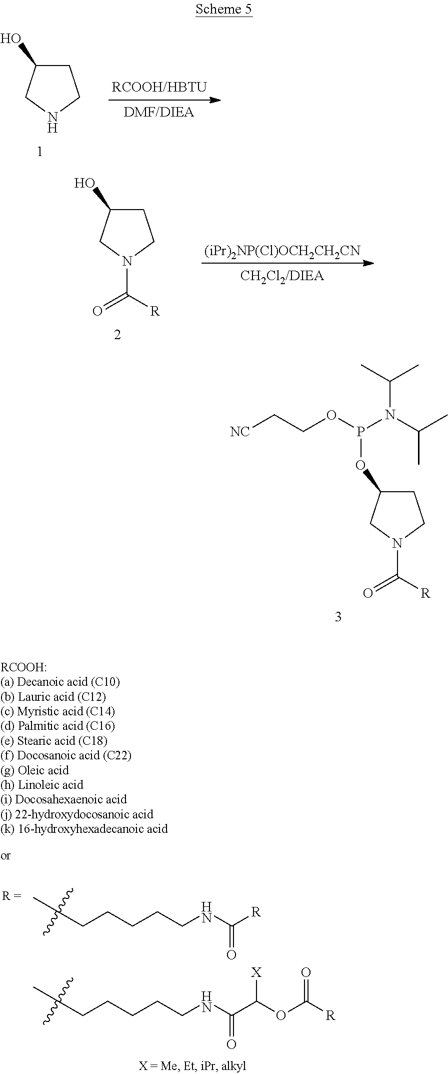

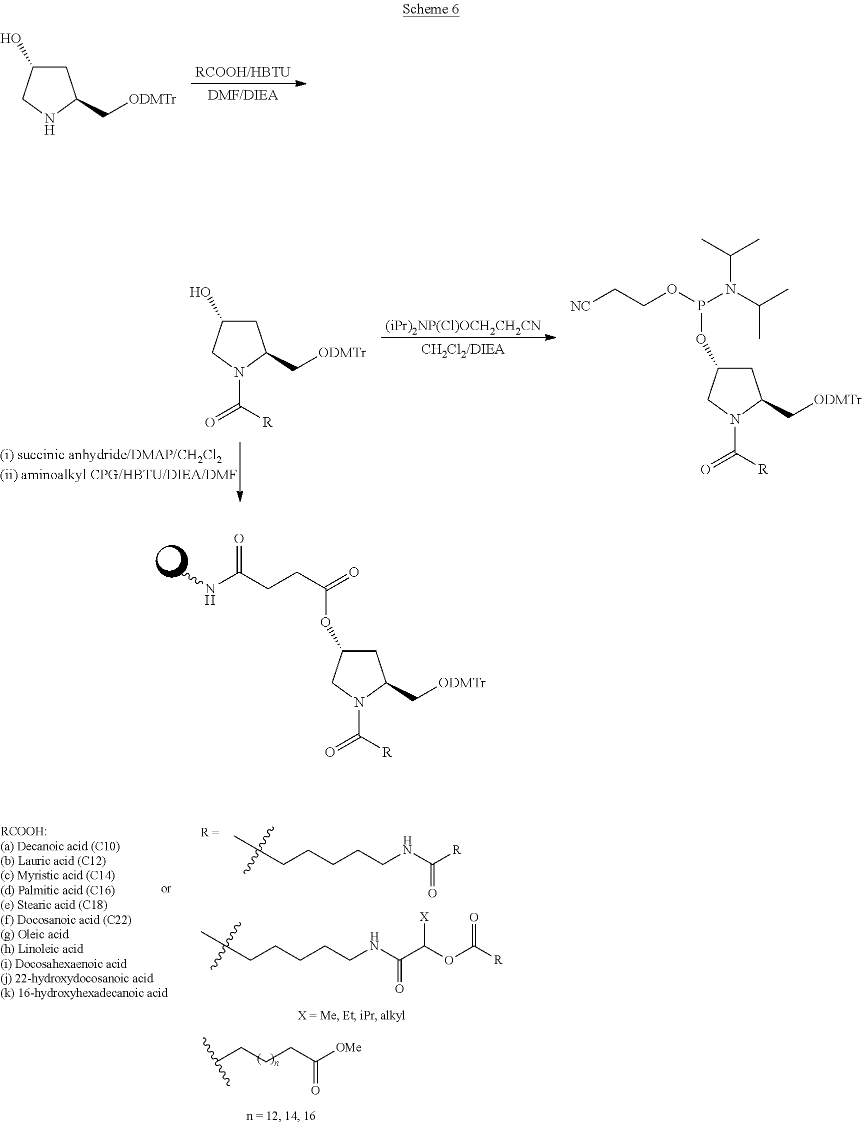

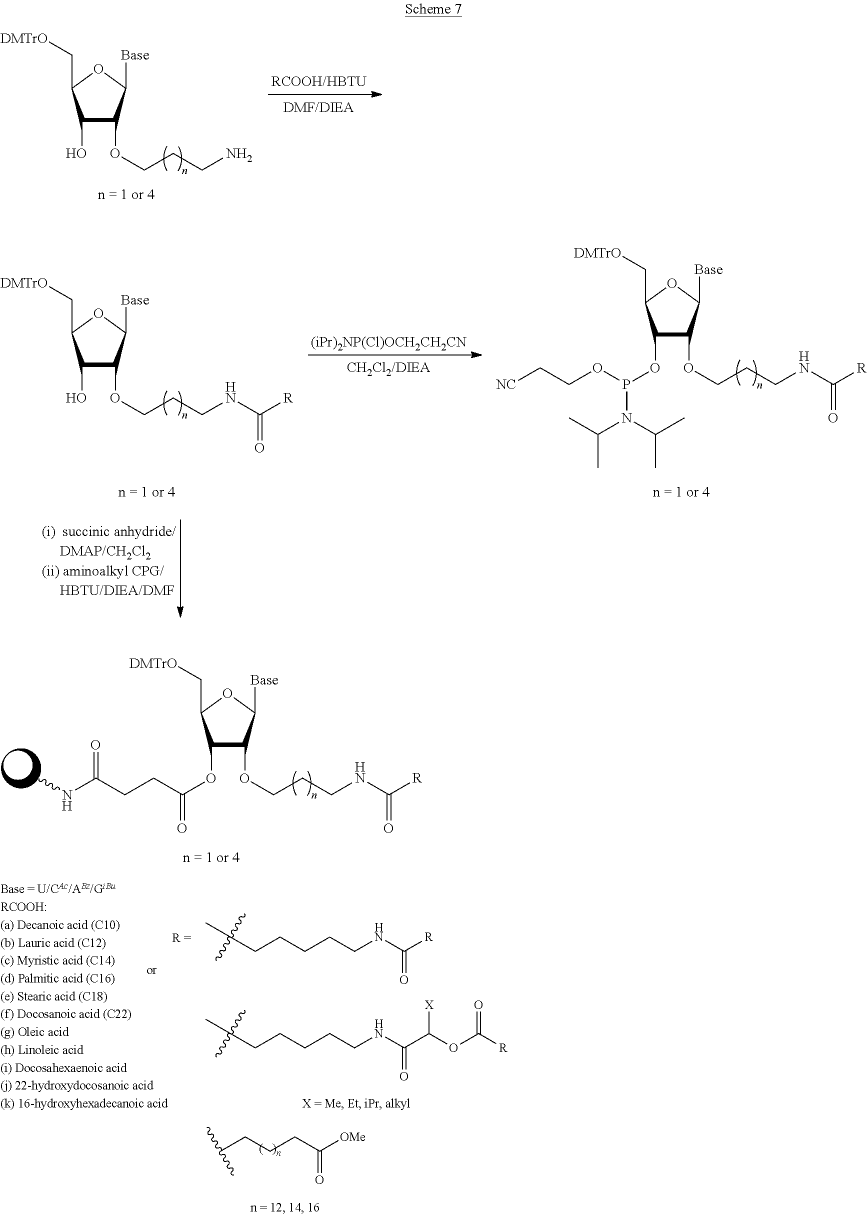

### Synthesis of Lipophilic Conjugate (Including C22) on Prolinol at 5′ End

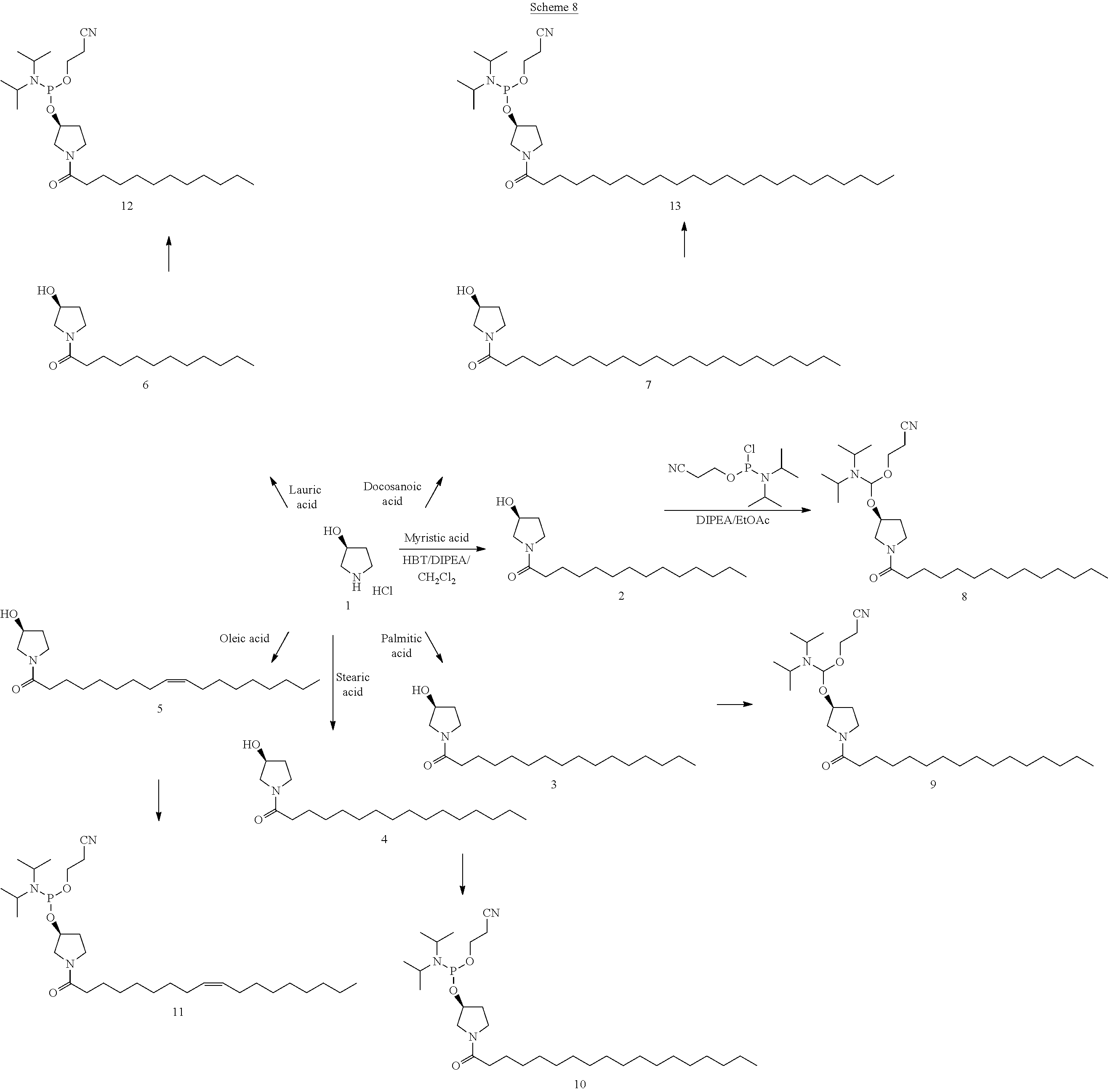

Compound 2: To a heat-oven dried 100 mL round bottle flask, added a solution of Compound 1, (3 g, 24.28 mmol, 1.0 equiv.) in anhydrous DCM (50 mL). Tetradecanoic acid 2a (6.10 g, 26.70 mmol, 1.1 eq.) was added to the solution, followed by HBTU (10.13 g, 26.70 mmol, 1.1 eq.) and DIPEA (12.68 mL, 72.53 mmol, 3 eq.). The resultant solution was stirred at room temperature under argon overnight. TLC with 80% EtOAc/hexane showed the formation of the product. The reaction mixture was quenched with brine solution, and extracted with DCM. The combined organic solution was dried over anhydrous Na₂SO₄, filtered and concentrated to an oil form residue. Purification through ISCO column chromatography with 80 g silica gel column eluted compound 2 with 0-0 EtOAc/hexane. A white oily compound was yielded (7.2 g). ¹H NMR (400 MHz, chloroform-d) δ 4.58-4.45 (m, 1H), 3.70-3.37 (m, 4H), 2.31-2.18 (m, 2H), 2.09-1.87 (m, 3H), 1.63 (t, J=7.4 Hz, 2H), 1.36-1.27 (m, 6H), 1.25 (s, 14H), 0.87 (t, J=6.8 Hz, 3H). M+1=298.3.

Compound 3: Compound 3 was obtained by using Compound 1 and palmitic acid in a procedure similar to the procedure above for synthesizing Compound 2. ¹H NMR (500 MHz, chloroform-d) δ 8.00 (s, 1H), 3.67-3.47 (m, 2H), 2.95 (s, 3H), 2.87 (s, 3H), 2.79 (s, 6H), 2.30-2.18 (m, 1H), 2.04 (h, J=3.5 Hz, 1H), 1.62 (p, J=7.2, 6.8 Hz, 2H), 1.32-1.26 (m, 4H), 1.24 (s, 11H), 0.87 (t, J=6.8 Hz, 2H). M+1=326.4.

Compound 4: Compound 4 was obtained by using Compound 1 and stearic acid in a procedure similar to the procedure above for synthesizing Compound 2. ¹H NMR (400 MHz, chloroform-d) δ 4.57-4.45 (m, 1H), 3.56 (dddd, J=31.4, 13.1, 10.0, 6.5 Hz, 4H), 2.80 (s, 3H), 2.31-2.18 (m, 3H), 2.04 (td, J=5.8, 2.9 Hz, 1H)), 1.28 (d, J=8.1 Hz, 28H), 0.87 (t, J=6.7 Hz, 3H). M+1=354.4.

Compound 5: Compound 5 was obtained by using compound 1 and oleic acid in a procedure similar to the procedure above for synthesizing Compound 2. ¹H NMR (400 MHz, chloroform-d) δ 5.40-5.27 (m, 2H), 3.67-3.46 (m, 4H), 2.80 (s, 9H), 2.36-2.16 (m, 3H), 1.36-1.21 (m, 20H), 0.91-0.83 (m, 3H). M+1=352.3.

Compound 6: Compound 6 was obtained by using compound 1 and dodecanoic acid in a procedure similar to the procedure above for synthesizing Compound 2. M+1=270.3.

Compound 7: Compound 7 was obtained by using compound 1 and docosanoic acid in a procedure similar to the procedure above for synthesizing Compound 2. ¹H NMR (400 MHz, chloroform-d) δ 4.52 (d, J=18.9 Hz, 2H), 3.69-3.15 (m, 5H), 2.32-2.18 (m, 2H), 2.03 (ddp, J=13.4, 9.0, 4.4 Hz, 2H), 1.73-1.60 (m, 3H), 1.32 (t, J=9.6 Hz, 8H), 1.25 (s, 25H), 0.88 (t, J=6.6 Hz, 3H). M+1=410.4.

Compound 8: Compound 2 (7.2 g, 24.2 mmol, 1 eq.) was dissolved in anhydrous EtOAc (120 mL). In an ice bath and under argon, DIPEA (12.65 mL, 72.61 mmol, 3 eq.) was added to the solution, followed by N,N-diisopropylaminocyanoethyl phosphonamidic-Cl (6.30 g, 26.61 mmol, 1.1 eq.). The resultant reaction mixture was stirred at room temperature overnight. TLC at 50% EtOAc/hexane showed the completion of the reaction. The reaction mixture was quenched with brine, and extracted with EtOAc. The organic layer was separated, dried over Na₂SO₄ and concentrated to a white oil. ISCO purification eluted Compound 8 with 0-50% EtOAc/hexane, with a yield of 65% (7.71 g). ¹H NMR (400 MHz, acetonitrile-d₃) δ 4.54 (dddt, J=17.4, 10.1, 5.8, 2.8 Hz, 1H), 3.88-3.34 (m, 7H), 2.66 (q, J=5.7 Hz, 2H), 2.33-2.15 (m, 3H), 2.09 (ddt, J=11.9, 7.8, 3.9 Hz, 1H), 1.62-1.51 (m, 2H), 1.38-1.25 (m, 20H), 1.25-1.13 (m, 13H), 0.95-0.87 (m, 3H). ³¹P NMR (162 MHz, CD₃CN) δ 147.33, 147.15, 146.97, 146.88.

Compound 9: Compound 9 was obtained using Compound 3 and N,N-diisopropylamino-cyanoethyl phosphonamidic-Cl in a procedure similar to the procedure above for synthesizing Compound 8. ¹H NMR (400 MHz, Acetonitrile-d₃) δ 4.61-4.43 (m, 1H), 3.87-3.70 (m, 2H), 3.70-3.34 (m, 6H), 2.67 (t, J=5.8 Hz, 2H), 2.33-2.14 (m, 3H), 2.09 (ddt, J=12.1, 7.9, 3.9 Hz, 1H), 1.30 (s, 25H), 1.25-1.14 (m, 13H), 0.97-0.87 (m, 3H). ³¹P NMR (162 MHz, CD₃CN) 6147.33, 147.15, 146.97, 146.88.

Compound 10: Compound 10 was obtained using Compound 4 and N, N-diisopropylamino-cyanoethyl phosphonamidic-Cl in a procedure similar to the procedure above for synthesizing Compound 8. ¹H NMR (400 MHz, acetonitrile-d₃) δ 4.66-4.40 (m, 1H), 3.87-3.34 (m, 8H), 2.67 (t, J=5.8 Hz, 2H), 2.30-2.16 (m, 3H), 2.15-2.02 (m, 1H), 1.30 (s, 27H), 1.29-1.16 (m, 15H), 0.95-0.87 (m, 3H). ³¹P NMR (162 MHz, CD₃CN) δ 147.32, 147.15, 146.97, 146.88.

Compound 11: Compound 11 was obtained using Compound 5 and N, N-diisopropylamino-cyanoethyl phosphonamidic-Cl in a procedure similar to the procedure above for synthesizing Compound 8. ¹H NMR (400 MHz, acetonitrile-d₃) δ 5.43-5.33 (m, 2H), 4.54 (dddd, J=20.3, 9.7, 4.8, 2.1 Hz, 1H), 3.88-3.72 (m, 2H), 3.72-3.34 (m, 6H), 2.66 (q, J=5.7 Hz, 2H), 2.33-2.16 (m, 4H), 1.42-1.28 (m, 21H), 1.28-1.14 (m, 14H), 0.95-0.87 (m, 3H). ³¹P NMR (162 MHz, CD₃CN) δ 147.34, 147.17, 147.00, 146.90.

Compound 12: Compound 12 was obtained using Compound 6 and N, N-diisopropylamino-cyanoethyl phosphonamidic-Cl in a procedure similar to the procedure above for synthesizing Compound 8. ¹H NMR (400 MHz, acetonitrile-d₃) δ 4.63-4.43 (m, 1H), 3.88-3.70 (m, 2H), 3.70-3.34 (m, 6H), 2.67 (t, J=5.8 Hz, 2H), 2.33-2.15 (m, 5H), 2.09 (ddt, J=12.3, 8.1, 3.9 Hz, 1H), 1.40-1.13 (m, 29H), 0.95-0.87 (m, 3H). ³¹P NMR (162 MHz, CD₃CN) δ 147.33, 147.15, 146.97, 146.86.

Compound 13: Compound 13 was obtained using Compound 7 and N, N-diisopropylamino-cyanoethyl phosphonamidic-Cl in a procedure similarly to the procedure above for synthesizing Compound 8. ¹H NMR (400 MHz, acetonitrile-d₃) δ 4.64-4.38 (m, 1H), 3.86-3.70 (m, 2H), 3.70-3.34 (m, 6H), 2.66 (q, J=5.7 Hz, 2H), 2.32-2.15 (m, 3H), 1.30 (s, 37H), 1.25-1.12 (m, 13H), 0.95-0.87 (m, 3H). ³¹P NMR (162 MHz, CD₃CN) δ 148.29, 147.33, 147.19, 147.01, 146.94.

Additional synthesis schemes and examples regarding conjugating lipophilic ligand to the siRNAs via hydroxyprolinol derivatives, may be found in the Examples of WO 2021/092371, which is incorporated herein by reference in its entirety.

A variety of lipids can also be conjugated to the ribose sugar moieties, e.g., at the 2′-O position, using methods described and exemplified in WO 2021/092371, which is incorporated herein by reference in its entirety, and the resulting building block phosphoramidites can be incorporated into siRNAs.

### Example 3. Post-Synthetic Conjugation of Lipophilic Moieties to siRNA

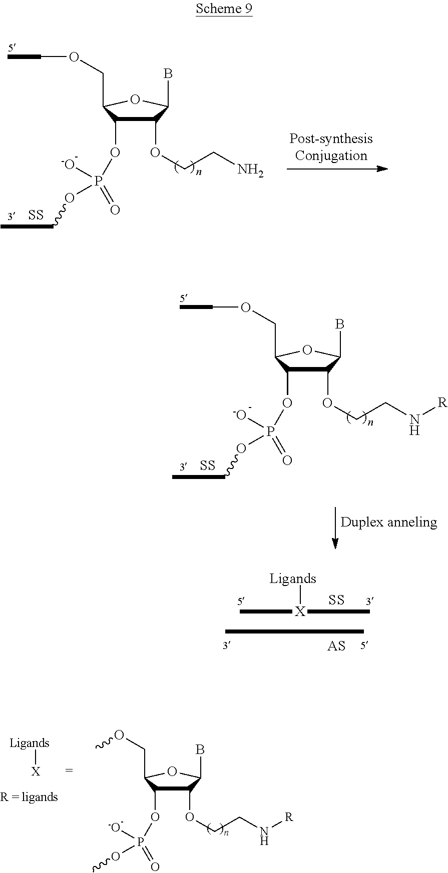

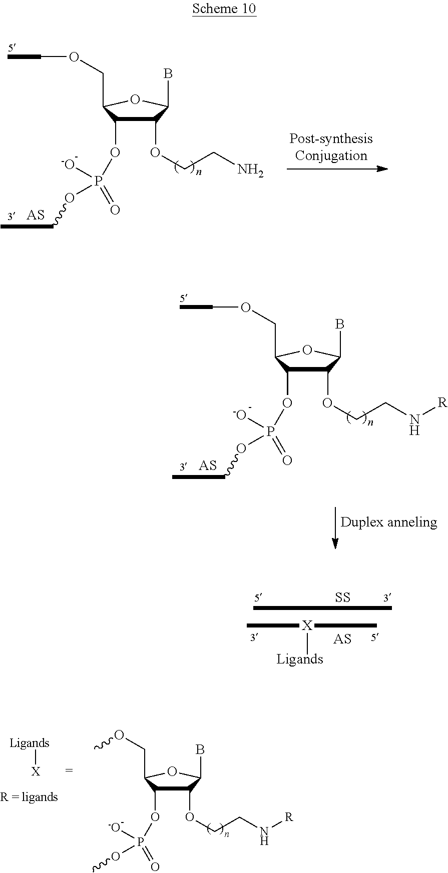

Various ligands, including various lipophilic moieties was conjugated to siRNA agents via post-synthesis conjugation methods, as shown in Schemes 9 and 10. Amino derivative of sense or antisense strand of siRNA was reacted either with NHS esters of lipophilic ligands or carboxylic acids under peptide coupling conditions. These singles strands were then purified and combined with other strands to make siRNA duplexes.

### Example 4. Synthesis of siRNA Conjugates Having Terminal Acid Functionality

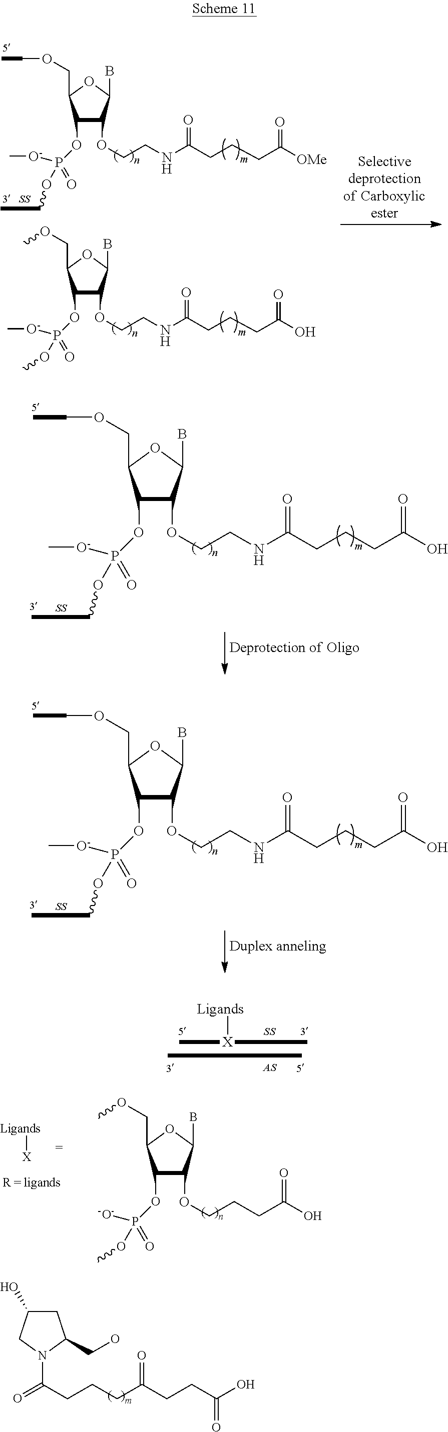

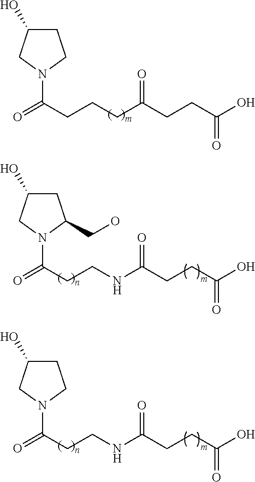

Various ligands, including various lipophilic having carboxylic moieties was conjugated to siRNA agents at terminals and internal positions via on column or post-synthetic conjugation, as shown in Scheme 11.

Solid supported single strands containing lipophilic moieties having terminal esters were first treated with 20% piperidine in water overnight followed by 2:1 NH₄OH in ethanol for 15 hours at room temperature to generate single strands having terminal carboxylic acids. These single strands were combined with corresponding antisense strands to generate siRNA duplexes for various assays.

### Example 5. Synthesis of siRNA Conjugates Having Lipophilic Groups Attached to Phosphate Backbone

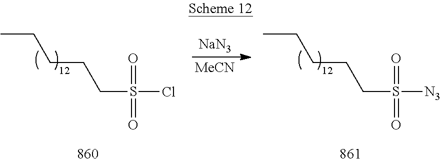

Compound 861: Sodium azide (2.57 g, 39.53 mmol) was added to a stirred solution of hexadecane-1-sulfonyl chloride (10.08 g, 30.4 mmol) in MeCN (100 mL). After stirring at room temperature for 10 hours, the reaction mixture was diluted with EtOAc (200 mL) and washed with water (50 mL). The organic phase was dried over Na₂SO₄ and evaporated to dryness. The residue was purified by ISCO automated column using 0-5% EtOAc in hexanes as eluent to give Compound 861 (7.71 g, 76%). ¹H NMR (400 MHz, chloroform-d) δ 3.33-3.28 (m, 2H), 1.96-1.87 (m, 2H), 1.51-1.41 (m, 2H), 1.33-1.23 (m, 24H), 0.92-0.86 (m, 3H).

Reaction between compound 861 and an oligonucleotide (sense or antisense strand). During the solid-phase synthesis of an oligonucleotide (Scheme 9), a solution of Compound 861 (0.5 M in acetonitrile) was used to oxidize the P(III) phosphite ester intermediate 862 to produce a sulfonyl phosphoramidite Compound 863. This oxidation step is used instead of common oxidizing reagents (I₂ or sulfurizing reagent) and can be performed at any stage of the oligonucleotide synthesis that involve oxidation of a P(III) phosphite. At the end of the synthesis, the oligo is fully deprotected using standard conditions, and cleaved from the solid support to give oligonucleotide 864 containing the sulfonylphosphoramidate.

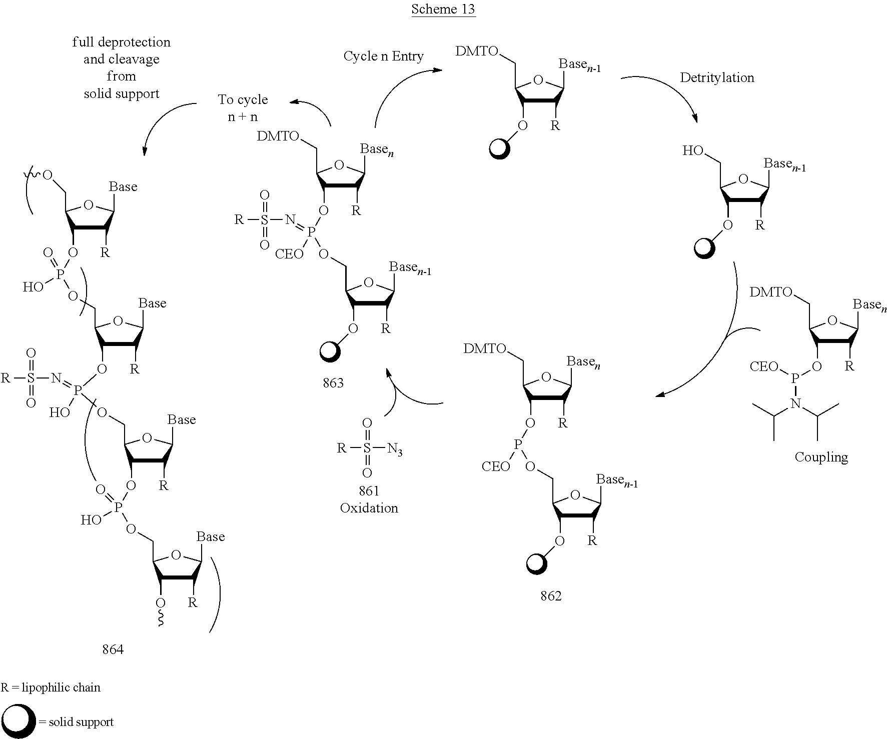

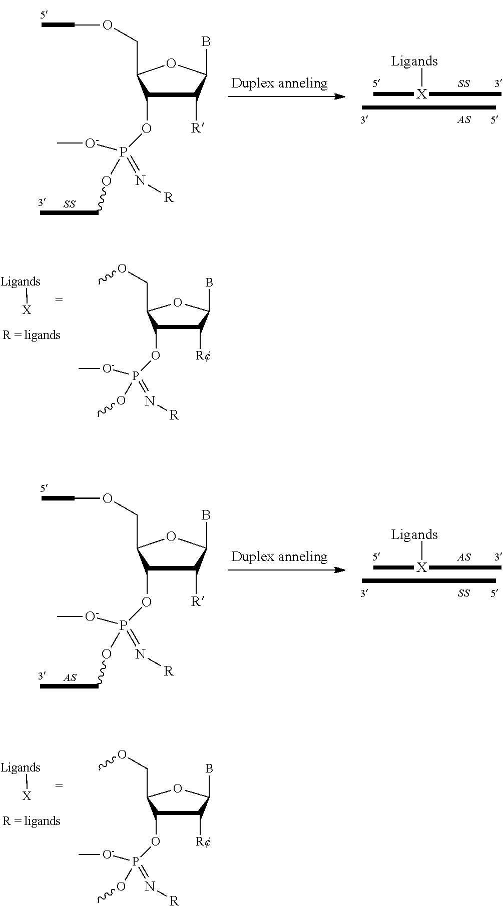

### Example 6. C22 Modification of siRNA Duplexes

TABLE 1
Abbreviations of nucleotide monomers used in nucleic acid sequence representation.
It will be understood that these monomers, when present in an oligonucleotide, are mutually
linked by 3′->5′phosphodiester bonds; and it is understood that when the nucleotide contains
a 2′-fluoro modification, then the fluoro replaces the hydroxy at that position in the parent
nucleotide (i.e., it is a 2′-deoxy-2′-fluoronucleotide). The abbreviations are understood to
omit the 3′-phosphate (i.e. they are 3′—OH) when placed at the 3′-terminal position of an
oligonucleotide.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine -3′-phosphorothioate |
| Us | uridine -3′-phosphorothioate |
| N | any nucleotide, modified or unmodified |
| a | 2′-O-methyladenosine-3′-phosphate |
| as | 2′-O-methyladenosine-3′-phosphorothioate |
| C | 2′-O-methylcytidine-3′-phosphate |
| CS | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage |
| Y34 | 2-hydroxymethyl-tetrahydrofurane-4-methoxy-3-phosphate (abasic 2′- |
| | OMe furanose) |
| | 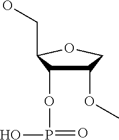 |
| Y44 | inverted abasic DNA (2-hydroxymethyl-tetrahydrofurane-5-phosphate) |
| | 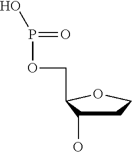 |
| L10 | N-(cholesterylcarboxamidocaproyl)-4-hydroxyprolinol (Hyp-C6-Chol) |
| | 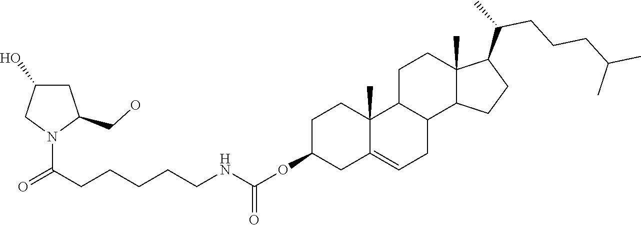 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate (e.g., 5′-E-vinyl phosphonate) |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| (Chds) | 2′-O-hexadecyl-cytidine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |
| S | Phosphorothioate |
| (Ada) | 2′-O-docosanyl-adenosine-3′-phosphate |
| | 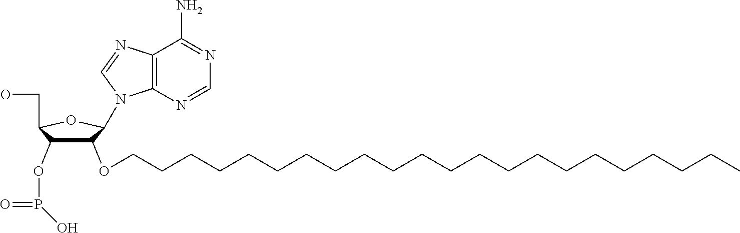 |
| | (or |
| | 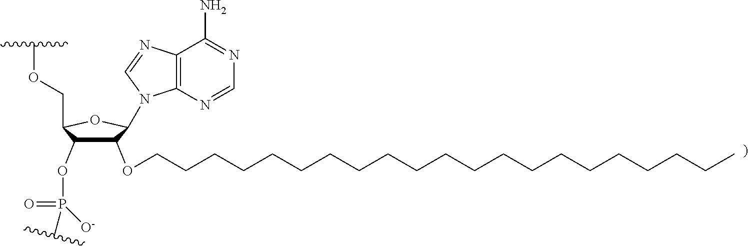 |
| (Cda) | 2′-O-docosanyl-cytidine-3′-phosphate |
| | 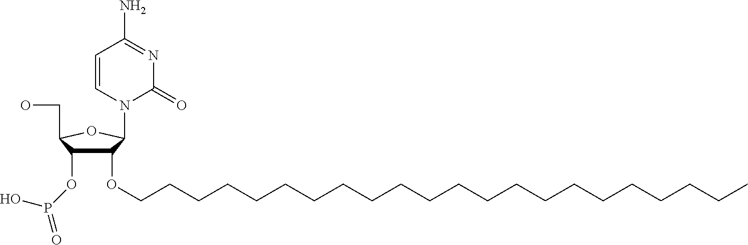 |
| | (or |
| | 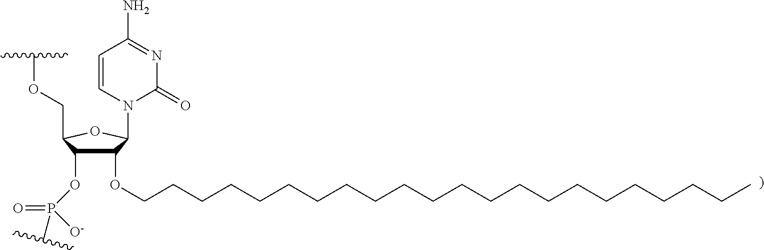 |
| (Gda) | 2′-O-docosanyl-guanosine-3′-phosphate |
| | 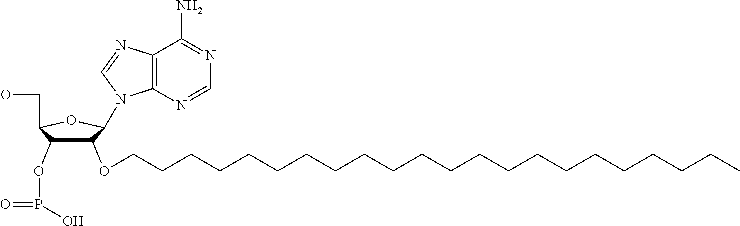 |
| | (or |
| | 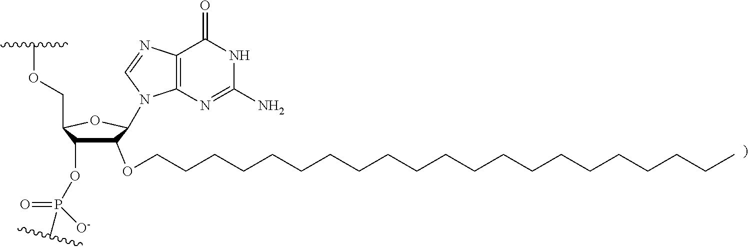 |
| (Uda) | 2′-O-docosanyl-uridine-3′-phosphate |
| | 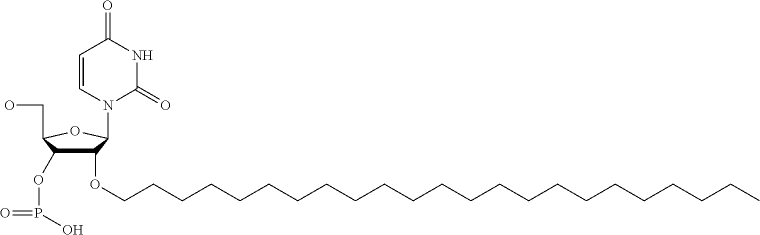 |
| | (or |
| | 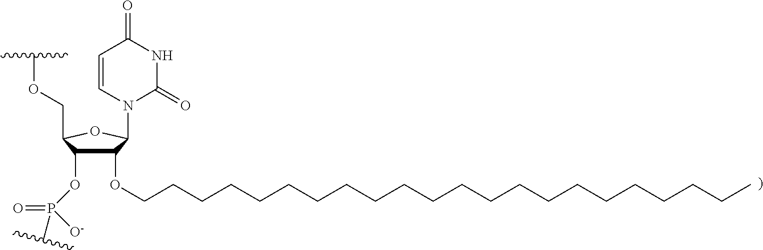 |
| (Adas) | 2′-O-docosanyl-adenosine-3′- phosphorothioate |
| (Cdas) | 2′-O-docosanyl-cytidine-3′- phosphorothioate |
| (Gdas) | 2′-O-docosanyl-guanosine-3′- phosphorothioate |
| (Udas) | 2′-O-docosanyl-uridine-3′-phosphorothioate |
| Y177 | 2′-O-methylcytidine 3′-(N-hexadecylsulfonyl) phosphorimidate |
| | 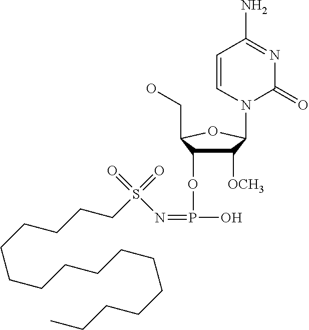 |
| Y211 | 2′-O-methyluridine 3′-(N-hexadecyl)phosphoramidate |
| | 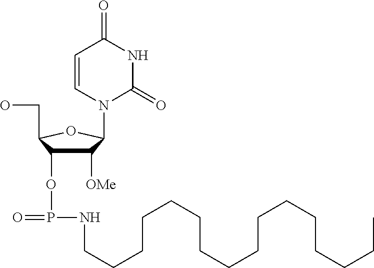 |
| Y229 | 2′-O-methyluridine 3′-(N,N-dimethyl)phosphoramidate |
| | 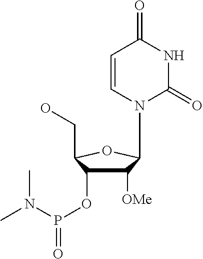 |
| Y230 | 2′-O-methyluridine 3′-(N-methyl)phosphoramidate |
| | 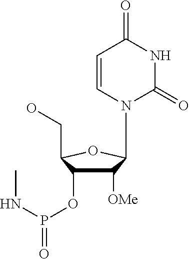 |
| Y250 | 2′-O-(2-oxo-2-(N-hexadec-1-ylamino)ethyl)uridine-3′-phosphate |
| | 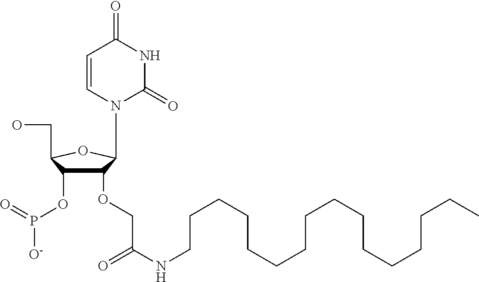 |
| Y270 | 2′-O-(2-oxo-2-(N-docosan-1-ylamino)ethyl)uridine-3′-phosphate |
| | 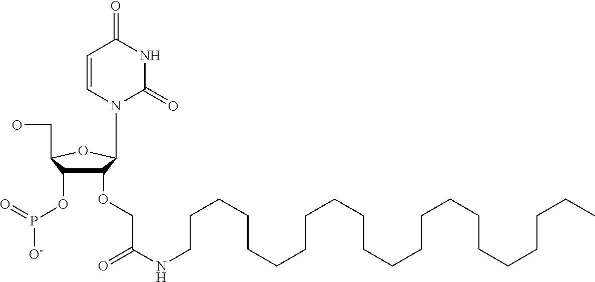 |
| Y318 | 2′-O-methyluridine 3′-(N-docosanyl)phosphoramidate |
| | 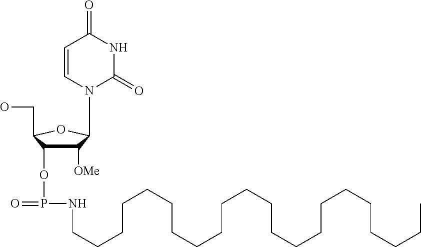 |
| Y320 | 2′-O-methyluridine 3′-(11-carboxyundec-1-yl)phosphoramidate |
| | 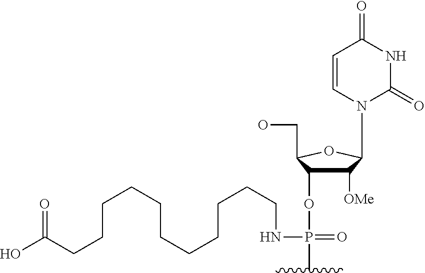 |
| Y321 | 2′-O-methyluridine 3′-(6-((3-beta-cholest-5-en-3-yl)oxcarboxamido)hexyl)phosphoramidate |
| | 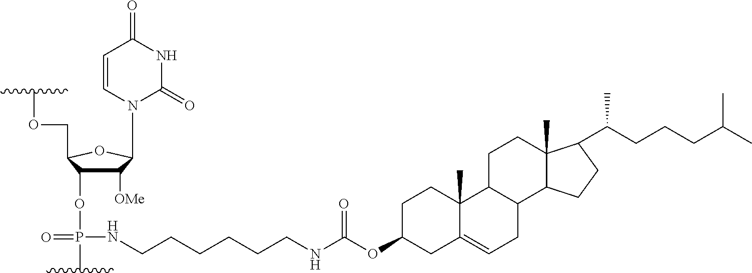 |
| Y327 | 2′-deoxy-2′-fluoroadenosine 3′-(6-aminohexyl)phosphoramidate |
| | 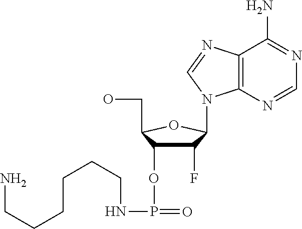 |
| Y329 | 2′-O-methyladenosine 3′-(6-aminohexyl)phosphoramidate |
| | 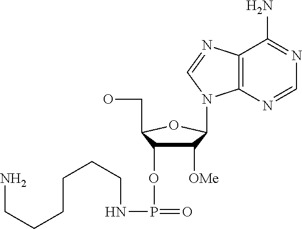 |
| Y333 | 2′-O-methyluridine 3′-(2-((2,5,7,8-tetramethyl-2-(4,8,12- |
| | trimethyltridecyl)chroman-6-yl)oxy)ethyl)phosphoramidate |
| | 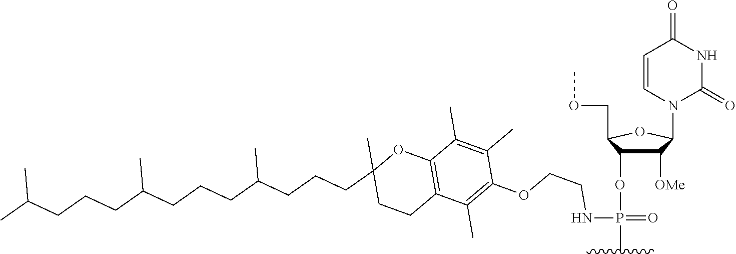 |
| Y505 | 2′-O-(6-(16-hydroxyhexadecan-1-oylamino)hex-1-yl)uridine 3′-phosphate |
| | 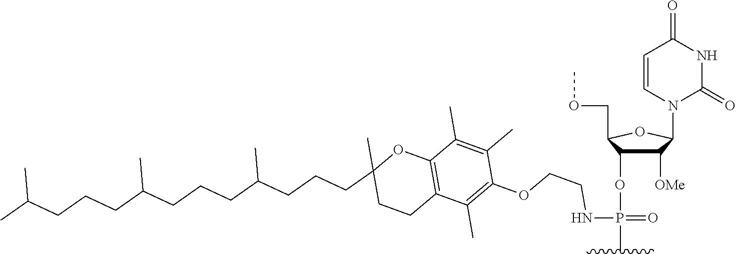 |
| | (or |
| | 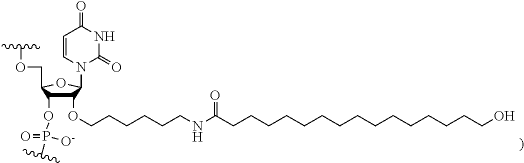 |
| Y999 | 2′-O-methyladenosine 3′-(11-carboxyundec-1-y1)phosphoramidate |
| | 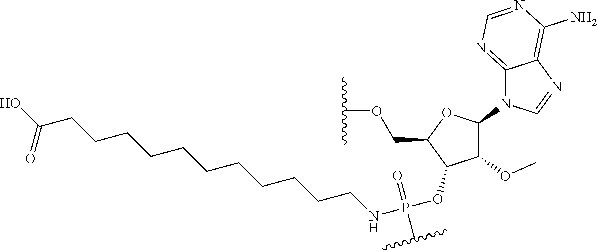 [SMILES: *OC[C@H]1O[C@@H](n2cnc3c(N)ncnc32)[C@H](OC)[C@@H]1OP(*)(=O)NCCCCCCCCCCCC(=O)O] |
| Y998 | 2′-deoxy-2′-fluoroadenosine 3′-(11-carboxyundec-1-yl)phosphoramidate |
| | 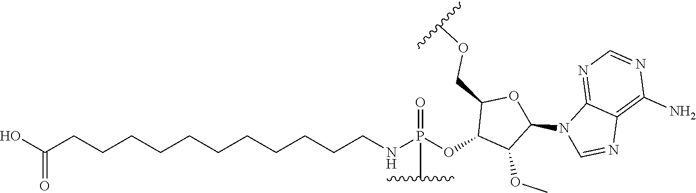 [SMILES: *OC[C@H]1O[C@@H](n2cnc3c(N)ncnc32)[C@H](OC)[C@@H]1OP(*)(=O)NCCCCCCCCCCCC(=O)O] |
| L58 | (2S,4R)-1-[6-(docosan-1-oylamino)hexan-1-oyl]-4-hydroxy-2- |
| | (hydroxymethyl)-pyrrolidine |
| | 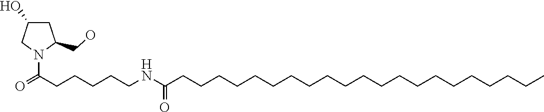 [SMILES: CCCCCCCCCCCCCCCCCCCCCC(=O)NCCCCCC(=O)N1C[C@H](O)C[C@H]1CO] |
| | (or |
| | 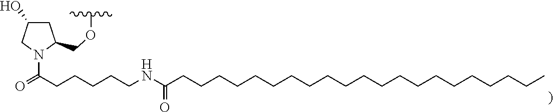 [SMILES: *OC[C@@H]1C[C@@H](O)CN1C(=O)CCCCCNC(=O)CCCCCCCCCCCCCCCCCCCCC] |
| L54 | (2S,4R)-1-[6-(hexadecan-1-oylamino)hexan-1-oy]]-4-hydroxy-2- |
| | (hydroxymethyl)-pyrrolidine |
| | 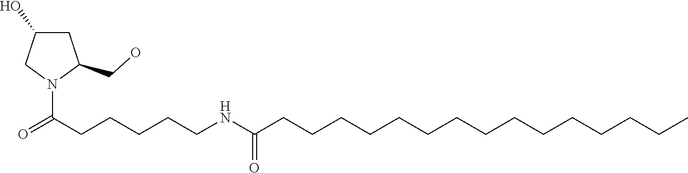 [SMILES: CCCCCCCCCCCCCCCC(=O)NCCCCCC(=O)N1C[C@H](O)C[C@H]1CO] |
| | (or |
| | 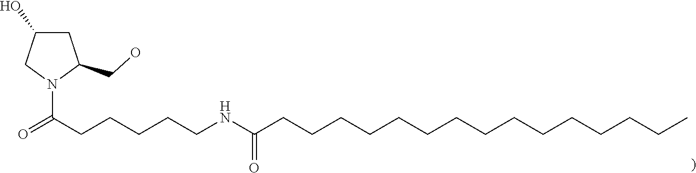 [SMILES: CCCCCCCCCCCCCCCC(=O)NCCCCCC(=O)N1C[C@H](O)C[C@H]1CO] |
| L321 | (2S,4R)-1-[hexadecan-1-oyl]-4-hydroxy-2-(hydroxymethyl)-pyrrolidine |
| | 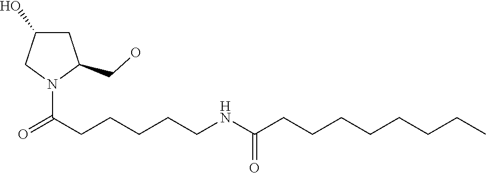 [SMILES: CCCCCCCCC(=O)NCCCCCC(=O)N1C[C@H](O)C[C@H]1CO] |
| | (or |
| | 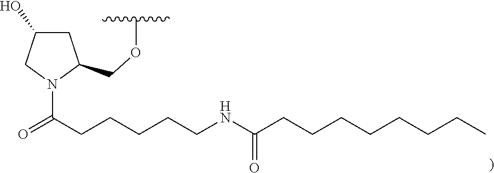 [SMILES: *OC[C@@H]1C[C@@H](O)CN1C(=O)CCCCCNC(=O)CCCCCCCC] |
| Q447 | (3S)-1-[6-(hexadecan-1-oylamino)hexan-1-oyl]-3-hydroxypyrrolidine 3- |
| | phosphate |
| | 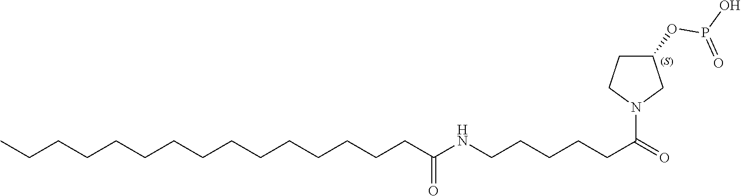 |
| | (or |
| | 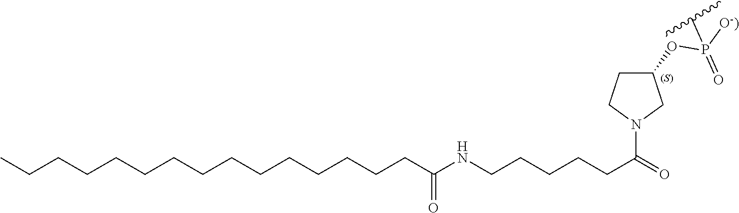 |
| Q447s | (3S)-1-[6-(hexadecan-1-oylamino)hexan-1-oyl]-3-hydroxypyrrolidine 3- |
| | phosphorothioate |
| | 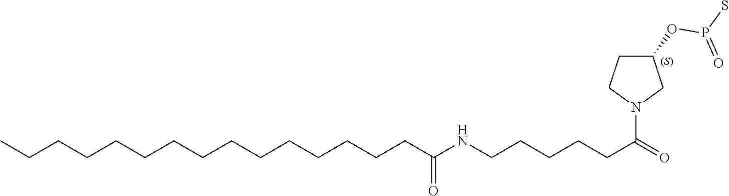 |
| | (or |
| | 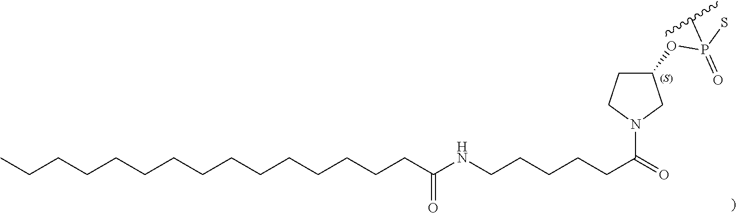 |
| Q448 | (3S)-1-[6-(docosan-1-oylamino)hexan-1-oy1]-3-hydroxypyrrolidine 3- |
| | phosphate |
| | 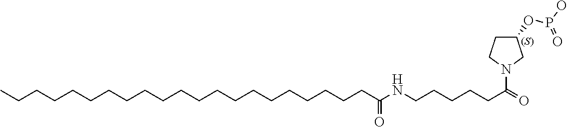 |
| | (or |
| | 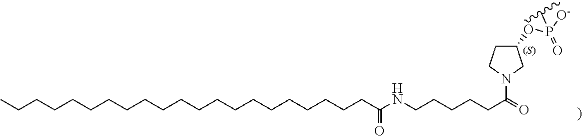 |
| Q448s | (3S)-1-[6-(docosan-1-oylamino)hexan-1-oyl]-3-hydroxypyrrolidine 3- |
| | phosphorothioate |
| | 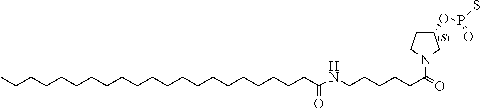 |
| | (or |
| | 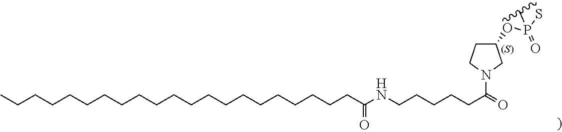 |
| Q466 | 6-palmitamidohexyl phosphate |
| | 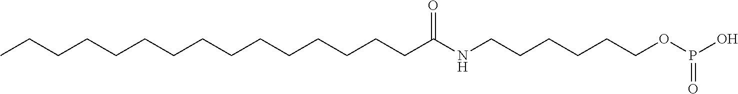 |
| | (or |
| | 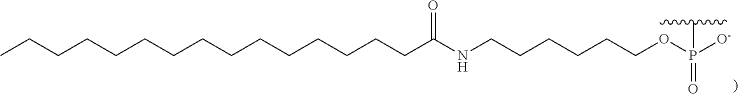 [SMILES: *P(=O)([O-])OCCCCCCNC(=O)CCCCCCCCCCCCCCC] |
| Q466s | 6-palmitamidohexyl phosphorothioate |
| | 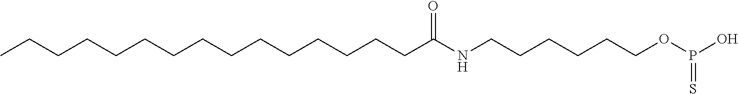 |
| | (or |
| | 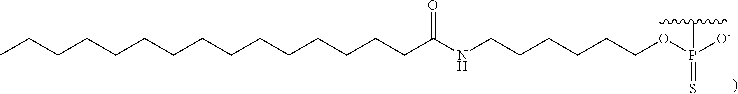 [SMILES: *P([O-])(=S)OCCCCCCNC(=O)CCCCCCCCCCCCCCC] |
| Q478 | 6-(16-hydroxyhexadecanamido)hexyl phosphate |
| | 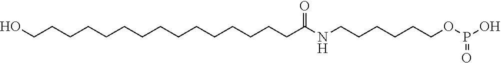 |
| | (or |
| | 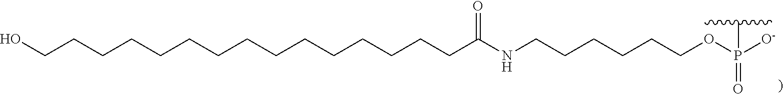 [SMILES: *P(=O)([O-])OCCCCCCNC(=O)CCCCCCCCCCCCCCCO] |
| Q478s | 6-(16-hydroxyhexadecanamido)hexyl phosphorothioate |
| | 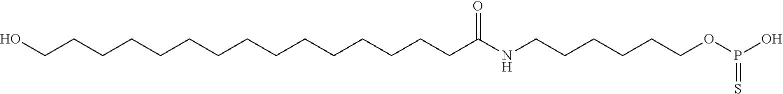 |
| | (or |
| | 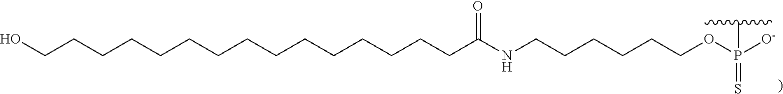 [SMILES: *P([O-])(=S)OCCCCCCNC(=O)CCCCCCCCCCCCCCCO] |
| Q483 | 6-docosanamidohexyl phosphate |
| | 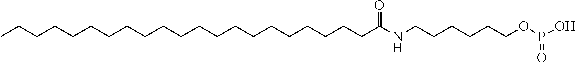 |
| | (or |
| | 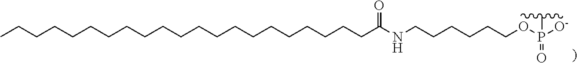 [SMILES: *P(=O)([O-])OCCCCCCNC(=O)CCCCCCCCCCCCCCCCCCCCC] |
| Q483s | 6-docosanamidohexyl phosphorothioate |
| | 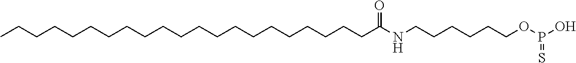 |
| | (or |
| | 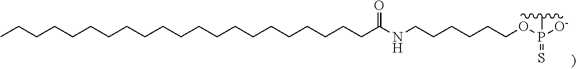 [SMILES: *P([O-])(=S)OCCCCCCNC(=O)CCCCCCCCCCCCCCCCCCCCC] |
| (Uah): Y505 | 2′-O-(6-aminohexyl)-uridine-3′-phosphate |
| precursor | |
| | 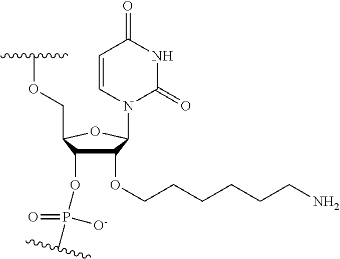 |
| Q70: Q478 | 6-aminohexyl phosphate |
| precursor | |
| | 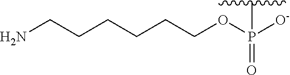 [SMILES: *P(=O)([O-])OCCCCCCN] |
| Q70s: Q478s | 6-aminohexyl phosphorothioate |
| precursor | |
| | 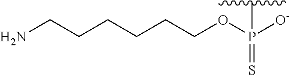 [SMILES: *P([O-])(=S)OCCCCCCN] |

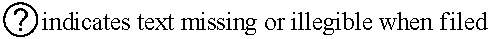

### General Oligonucleotide Synthesis and Analysis

General oligonucleotide synthesis procedures for the oligonucleotides disclosed in all the examples are described herein in this example.

Oligonucleotides were synthesized on a Bioautomation Mermade 12, Cytiva Akta Oligopilot 100, or K&AH-8 Oligo Synthesizer using commercially available RNA amidites, 5′-O-(4,4′-dimethoxytrityl)-2′-deoxy-2′-fluoro-, and 5′-O-(4,4′-dimethoxytrityl)-2′-O-methyl-3′-O-(2-cyanoethyl-N,N-diisopropyl) phosphoramidite monomers of uridine, 4-N-acetylcytidine, 6-N-benzoyladenosine, and 2-N-isobutyrylguanosine. Standard solid-phase oligonucleotide synthesis protocols were used.

The lipophilic moiety (e.g., the exemplary C22 conjugate and comparative C16 conjugate) were introduced as the lipophilic monomer phosphoramidites (prepared according to Examples 1-5 as described above). 3′-end lipid modifications were introduced as modifications bound to a CPG solid support.

Phosphorothioate linkages were introduced by oxidation of phosphite utilizing 0.1 M 3-((N,N-dimethyl-aminomethylidene)amino)-3H-1,2,4-dithiazole-5-thione (DDTT) in pyridine, 0.2 M xanthine hydride in pyridine, or 0.2 M phenylacetyl disulfide in 50/50 v/v lutidine and acetonitrile. If phosphorothioate isomers were to be separated post-synthesis, the final dimethoxytrityl protecting group was not removed after synthesis was completed.

The deprotection procedures utilized were suitable for 2′-F and 2′-OMe modified oligonucleotides. After synthesis, the solid support was treated on column with 0.5 M piperidine in acetonitrile (ACN) for 15 minutes. The column was washed with ACN and treated again with 0.5 M piperadine in ACN for an additional 15 minutes, then washed again with ACN. The support was dried on-column under vacuum, and then added to a sealable container and heated at 60° C. in aqueous ammonium hydroxide (28-30%) for 2-3 hours. The deprotection procedure was completed by shaking overnight at 30° C. Alternatively, after synthesis, the solid support was treated with 5% diethylamine in aqueous ammonia (28-30%) at 65° C. for 5 hours or 24 hours at 30° C. The oligonucleotide was then filtered to remove the support with 5× volume of water and analyzed by LC-MS and ion-exchange analysis to determine the quality of the crude as described in Nair et al. and Parmar et al. provided above.

After deprotection and crude quality confirmation, ion-exchange HPLC purification was performed. The column size for the ion-exchange HPLC purification depended on scale (total OD load). TSKgel Super Q-5PW (20) anion exchange resin from Tosoh Corporation was used for purification. Purification buffer A consisted of 20 mM sodium phosphate (pH 8.5), 15% ACN, and Buffer B was 20 mM sodium phosphate (pH 8.5), 15% ACN, 1M sodium bromide. A gradient of 15% to 45% in about 20 column volumes was sufficient, unless isomer separation post-synthesis was performed. The gradient start time was adjusted depending on the retention time of the full-length product in the ion-exchange analysis of the crude. Fractions were analyzed by the ion-exchange analysis using the Dionex DNAPac PA200 ion-exchange analytical column, 4 mm×250 mm (ThermoFisher Cat #063000) at room temperature. Buffer A was 20 mM sodium phosphate (pH 12), 15% acetonitrile, Buffer B was 20 mM sodium phosphate (pH 12), 15% acetonitrile, 1M sodium bromide. A gradient of 30% to 50% over 12 minutes at a flow rate of 1 ml/min was used to analyze the fractions.

The fractions with greater than 85% purity were pooled, dried, dissolved in water, and desalted over size exclusion columns (GE Healthcare) at a flow rate of 10 ml/min. The desalted final product was dried, resuspended in water, filtered through 0.2 μm polyethersulfone filters, and quantified analysis of absorbance at 260 nm. Samples approximately 1 OD/ml were assessed by LC-MS and ion-exchange analysis. The oligonucleotides were then frozen and lyophilized, followed by annealing of equimolar amounts of complementary strands to provide the desired siRNA duplexes by heating to 90° C. and slow cooling. The siRNA samples were analyzed by mass spectrometry and capillary gel electrophoresis and for endotoxin and osmolality as described in Nair et al. and Parmar et al. provided above.

Hydroxyl lipids (e.g., Q478 and Y505), were introduced via an amide coupling to an amino functionalized oligonucleotide. The 5′ amino group was introduced as a trifluoroacete protected amino phosphoramidite (Glen Research, product 10-1916). The internal amino group was introduced as the trifluoroacetate protected 2′ hexylamino uridine phosphoramidite, Oligosynthesis was completed as described above and the TFA amino oligonucleotides were deprotected in a 50/50 v/v solution of aqueous ammonia (28-40%) and aqueous methylamine (40%) by shaking at 25° C. for 3-4 hours. The crude amino functionalized oligonucleotides were then filtered and purified as described above.

To install the hydroxyhexadecanoyl lipid, the aminofunctionalized oligonucleotides were dissolved to a concentration of 100 mg/mL in water. Hydroxyhexadecanoic acid (3 eq. to oligonucleotide), PyAOP (7-Azabenzotriazol-1-yloxy)trispyrrolidinophosphonium hexafluorophosphate) (3.1 equivalents to the oligonucleotide), and N,N-diisopropylethylamine (9 equivalents to the oligonucleotide) were mixed in a volume of NMP (N-Methyl-2-pyrrolidone) matching the volume of oligonucleotide solution to be conjugated for 30 seconds. The two mixtures were then combined and shaken at 25° C. for 5 minutes. The crude hydroxyhexadecanoyl conjugated oligonucleotides were purified by reverse phase chromatography using 20 mM sodium acetate pH 7 buffer with a gradient of acetonitrile from 3% to 80%. The fractions were collected and desalted as described above.

### Example 7: Evaluating Translation of CNS siRNA Conjugate Delivery to Non-Human Primate (NHP)-Single Dose NHP Design

Exemplary siRNAs comprising a C₂₂ hydrocarbon chain conjugated to the sense strand at position 6, counting from the 5′-end of the sense strand, were used in this example (AD-1302923). Comparative siRNAs included the following siRNAs:
the siRNAs comprising a C₁₆ hydrocarbon chain conjugated to the sense strand at position 6, counting from the 5′-end of the sense strand (AD-454844, AD-476454 AD-890098, and AD-1350088);
the siRNA comprising a C₁₆ hydrocarbon chain conjugated to the sense strand at position 1, counting from the 5′-end of the sense strand (AD-1302922);
the siRNA comprising a C₁₆ hydrocarbon chain conjugated to the sense strand at position 6, counting from the 5′-end of the sense strand, with a modified phosphate backbone where C₁₆ was conjugated (AD-1718639).

The sequences were designed to target non-human primate (NHP) CNS genes APP, MAP2, SOD1, or MBP, with assessing the mRNA expression in frontal cortex, hippocampus, cerebellum, brain stem, striatum, or spine (cervical, lumbar, or thoracic) tissues.

A detailed list of the siRNA duplexes is shown in Table 2 below.

TABLE 2
siRNAs having lipophilic moiety conjugated via 2′- or 3′ positions

| | | | | | SEQ |
|---|---|---|---|---|---|
| | | | | | ID |
| Duplex ID | Oligo ID | Strand | Target | OligoSeq | NO: |
| AD-476454 | A-920992 | sense | MAP2 | asgsgau(Ahd)GfuUfCfAfaguagucascsa | 38 |
| | A-859918 | antisense | MAP2 | VPusGfsugaCfuAfCfuugaAfcUfauccususg | 39 |
| AD-454844 | A-882381 | sense | APP | asasaau(Chd)CfaAfCfCfuacaaguuscsa | 40 |
| | A-882382 | antisense | APP | VPusGfsaacu(Tgn)guagguUfgGfauuuuscsg | 41 |
| AD-1302923 | A-2365995 | sense | APP | asasaau(Cda)CfaAfCfCfuacaaguuscsa | 42 |
| | A-882382 | antisense | APP | VPusGfsaacu(Tgn)guagguUfgGfauuuuscsg | 41 |
| AD-1302922 | A-2365988 | sense | APP | (Ahds)asaaucCfaAfCfCfuacaaguuscsa | 43 |
| | A-882382 | antisense | APP | VPusGfsaacu(Tgn)guagguUfgGfauuuuscsg | 41 |
| AD-1718639 | A-3154600 | sense | APP | asasaauY177CfaAfCfCfuacaaguuscsa | 44 |
| | A-882382 | antisense | APP | VPusGfsaacu(Tgn)guagguUfgGfauuuuscsg | 41 |
| AD-890098 | A-1684938 | sense | SOD1 | ususggg(Chd)AfaAfGfGfuggaaaugsasa | 45 |
| | A-859296 | antisense | SOD1 | VPusUfscauUfuCfCfaccuUfuGfcccaasgsu | 46 |
| AD-1350088 | A-2514557 | sense | MBP | gscsuuc(Ahd)GfaCfCfAfuccaagaasgsa | 47 |
| | A-874305 | antisense | MBP | VPusCfsuucUfuGfGfauggUfcUfgaagcsusc | 48 |

Gene-silenting in non-human primates (NHP) was studied with siRNA conjugates listed in Table 2 above, evaluating a single target—APP—expression level in the CSF in the NHP, after a single intrathecal injection at a dose regime shown in the chart below.

| | | | | Dose | | |
|---|---|---|---|---|---|---|
| | | | Dose | Con- | # of | |
| | | Dose | Volume | tration | ani- | Time- |
| AD# | Chemistry | (mg) | (mL) | (mg/mL) | mals | points |
| AD-454844 | C16 Control | 60 | 2 | 30 | 4 | 3 |
| AD-454844 | C16 Control | 20 | 2 | 10 | 4 | months |
| AD-1302923 | C22 | 20 | 2 | 10 | 4 | |
| | | 6.7 | | 3.3 | | |
| AD-1302922 | SS1 C16 | 20 | 2 | 10 | 4 | |
| AD-1718639 | Backbone | 20 | 2 | 10 | 4 | |
| | C16 | | | | | |

APP mRNA Knockdown in NHP Striatum at Various Time Points Over 113 Days Post Dose

A single intrathecal (IT) injection, via percutaneous needle stick, of the APP siRNA of interest at a dose regime shown in the chart above, was administered in NHP on D1. The results in APP mRNA knockdown in non-human primate striatum at various time points (pre-dose at −D7 was considered as baseline) (24 hours, D8, D15, D29, D57, D83, and D113) over 113 days post dose were analyzed.

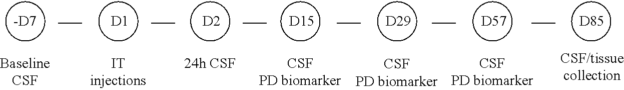

Soluble APP Alpha (sAPPα)/Soluble APP Beta (sAPPβ)

CSF levels of sAPPα and sAPPβ were determined utilizing a sandwich immunoassay MSD® 96-well MULTI-SPOT sAPPα/sAPPβ assay (Catalog no. K15120E; Meso Scale Discovery, Rockville, MD, USA) according to the manufacturer's protocol with some modifications. The standards, blanks, and non-human primate CSF samples (8× dilution) were prepared with the 1% Blocker-A/TB ST (provided in the kit). Pre-coated plate (provided in the kit) was blocked with 150 pL/well of 3% Blocker A/TB ST solution at room temperature for 1 hour with shaking. After three washes with 1×TBST, 25 pL/well of prepared standard, blanks, and CSF samples were added to the plate in two replicates and incubated for 1 hour at room temperature with shaking. Following subsequent plate washes, 50 pL/well of detection antibody prepared in 1% Blocker A/TBST (50× dilution) was added and incubated at room temperature for 1 hour with shaking. After plate washes, IX Read Buffer T was added to the plate and incubated for 10 minutes at room temperature (without shaking) before imaging and analyzing in MSD QuickPlex Imager.

Raw data were analyzed using SoftMax Pro, version 7.1 (Molecular Devices). A 5-parameter, logistic curve fitting with 1/Y2 weighing function was used to model the individual calibration curves and calculate the concentration of analytes in the samples.

### Mass Spec Method

Drug concentrations in CSF and CNS tissue samples were quantitated using a qualified LC-MS/MS method. Briefly, tissue samples were homogenized in lysis buffer, then the oligonucleotides were extracted from CSF or tissue lysate by solid phase extraction and analyzed using ion-pairing reverse phase liquid chromatography coupled with mass spectrometry under negative ionization mode. The concentration of the full-length antisense strand of the dosed duplex was measured. The drug concentrations were reported as the antisense-based duplex concentrations. The calibration range is 10-5000 ng/mL for CSF samples, and 100-50000 ng/g for CNS tissue samples. Concentrations that were calculated below the LLOQ are reported as <LLOQ. An analog duplex with different molecular weight was used as internal standard. mRNA knockdown by qPCR method.

Total RNA was isolated from rat brain and spinal cord tissue samples using the miRNeasy Mini Kit from (Qiagen, Catalog No. 217004) according to the manufacturer's instructions. Following isolation, RNA was reverse transcribed using Superscript™ IV VILO™ Reverse Transcriptase (Thermo Fisher Scientific). Quantitative PCR analysis was performed using a ViiA7 Real-Time PCR System from Thermo Fisher Scientific of Waltham MA 02451 (Catalog No. 4453537) with Taqman Fast Universal PCR Master Mix (Applied Biosystems Catalog No. 4352042), pre-validated amyloid beta precursor protein (APP) (Mf01552291_m1) and peptidylprolyl isomerase B (PPIB) (Mf02802985_m1) Taqman Gene Expression Assays (Thermo Fisher Scientific).

The relative reduction of APP mRNA was calculated using the comparative cycle threshold (Ct) method. During qPCR, the instrument sets a baseline in the exponential phase of the amplification curve and assigns a Ct value based on the intersection point of the baseline with the amplification curve. The APP mRNA reduction was normalized to the experimental untreated control group as a percentage for each respective group using the Ct values according to the following calculations:

$$Δ {Ct}_{App} = {Ct}_{App} - {Ct}_{Ppib}$$

$$ΔΔ {Ct}_{App} = Δ {Ct}_{App} - Δ {Ct}_{untreatedcontrolgroupmean}$$

$$Relative mRNA level = {2}^{- Δ Δ C t}$$

The results are shown in FIGS. 1-4.

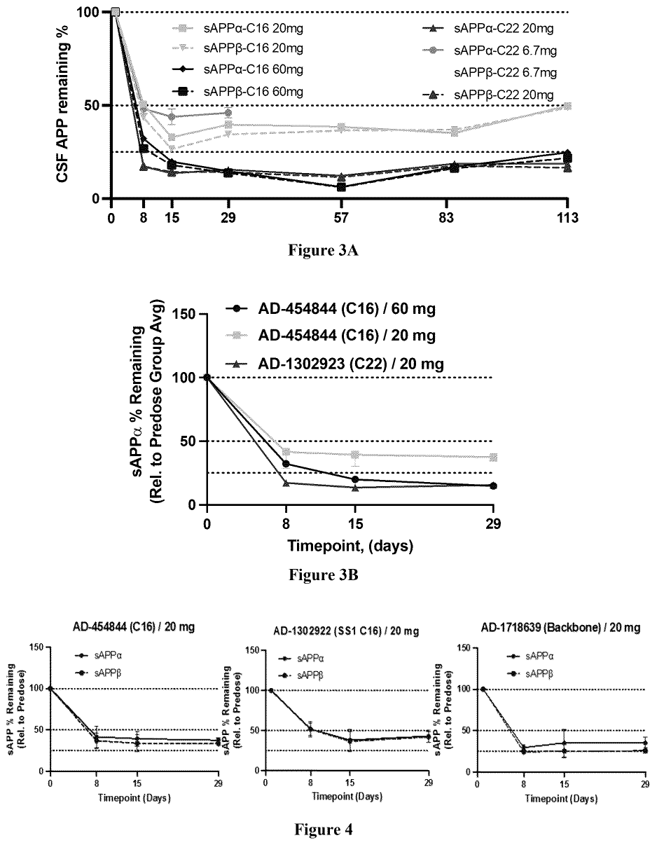

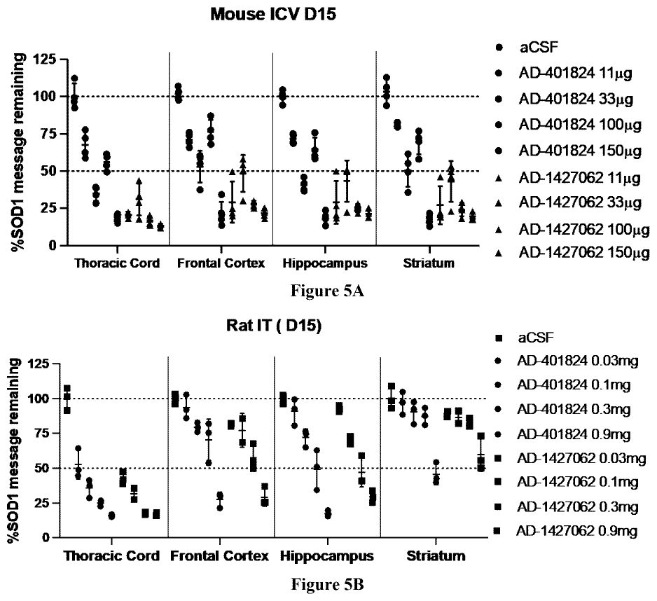

FIG. 1, FIG. 2A, FIGS. 3A-3B show that the siRNA duplexes comprising a C₂₂ conjugate at 20 mg dosage level had a silencing activity comparable to the siRNA duplexes comprising a C₁₆ conjugate at 60 mg dosage level. FIG. 1, FIG. 2B, and FIG. 3A show that the siRNA duplexes comprising a C₂₂ conjugate at 6.7 mg dosage level had a silencing activity comparable to the siRNA duplexes comprising a C₁₆ conjugate at 20 mg dosage level. These figures indicate that the siRNA duplexes comprising a C₂₂ hydrocarbon chain conjugated to the sense strand demonstrated a significant potency boost and much enhanced activity in the NHP over the siRNA duplexes comprising a C₁₆ hydrocarbon chain conjugated to the sense strand. The siRNA duplexes comprising a C₂₂ hydrocarbon chain conjugate had a three-fold PD enhancement over the siRNA duplexes comprising a C₁₆ hydrocarbon chain conjugate. Thus, with the siRNA duplexes comprising C₂₂ conjugate, a significantly reduced dosage level (⅓) is needed to reach the desired silencing activity, as compared to the siRNA duplexes comprising the C₁₆ conjugate.

Although the results in FIGS. 1-3 were based on a comparison against the siRNA duplexes comprising a C₁₆ hydrocarbon chain conjugated to N6 of the sense strand, FIG. 4 shows that the results of the siRNA duplexes with a C₁₆ hydrocarbon chain conjugated to N1 of the sense strand, and the siRNA duplexes with a C₁₆ hydrocarbon chain conjugated to N6 of the sense strand with a modified phosphate backbone are similar to the results of the siRNA duplexes comprising a C₁₆ hydrocarbon chain conjugated to N6 of the sense strand.

These results are important, because 1000 ng/ml CSF is the typical failed IT cutoff with 60 mg IT dose, and as shown in the 24-hour CSF PK data below for the siRNA duplexes comprising C₁₆ conjugate (AD-454844) and the siRNA duplexes comprising C₂₂ conjugate (AD-1302923), the siRNA duplexes comprising C₁₆ conjugate had the CSF PK significantly higher than 1000 ng/ml, whereas the siRNA duplexes comprising C₂₂ conjugate had the CSF PK mostly within 500 ng/ml.

| | AD-454844 C16 (20 mg) | | AD-1302923 (20 mg) | | | |
|---|---|---|---|---|---|---|
| | Animal ID | | | | | |
| | 2001 | 2004 | 6001 | 6002 | 6003 | 6004 |
| 24 hour | 1602 | 4763 | 433 | 164 | 5965 | 257 |
| CSF TA | (ng/ml) | (ng/ml) | (ng/ml) | (ng/ml) | (ng/ml) | (ng/ml) |
| D 8 | 32.7 | 50.7 | 72.5 | 87.7 | 17.2 | 42.8 |
| D 15 | 45.8 | 33 | 64.3 | 53.2 | 13.6 | 43.6 |
| D 29 | 35.5 | 39.8 | 66.9 | 71.4 | 15.6 | 71.4 |

### Example 8: Evaluating the Dose Dependence of C22-Conjugated siRNA Delivery to Rodent (Mouse ICV or Rat IT)

Exemplary siRNAs comprising a C₂₂ hydrocarbon chain conjugated to the sense strand at position 6, counting from the 5′-end of the sense strand (AD-1427062)(C22), were used in this example at various dosages (0.03 mg, 0.1 mg, 0.3 mg, and 0.9 mg for rat IT, and 11 μg, 33 μg, 100 μg, and 150 g for mouse ICV). Comparative siRNAs comprising a C₁₆ hydrocarbon chain conjugated to the sense strand at position 6, counting from the 5′-end of the sense strand (AD-401824)(C16), were used in this example, at the same dosages as the exemplary siRNAs. The sequences were designed to target SOD1, with assessing the mRNA expression in frontal cortex, hippocampus, cerebellum, striatum, or thoracic cord tissues. The sequences used are shown in Table 3 below.

TABLE 3
siRNAs having C22 conjugated at 6 position of sense strand (with C16 control)

| | | | | | SEQ ID |
|---|---|---|---|---|---|
| Duplex ID | Oligo ID | Strand | Target | OligoSeq | NO: |
| AD-401824 | A-637448 | sense | SOD1 | csasuuu(Uhd)AfaUfCfCfucacucuas | 49 |
| | | | | asa | |
| | A-444402 | antisense | SOD1 | VPusUfsuagAfgUfGfaggaUfuAfaa | 50 |
| | | | | augsasg | |
| AD-1427062 | A-2578434 | sense | SOD1 | csasuuu(Uda)AfaUfCfCfucacucuas | 51 |
| | | | | asa | |
| | A-444402 | antisense | SOD1 | VPusUfsuagAfgUfGfaggaUfuAfaa | 50 |
| | | | | augsasg | |

In vivo studies were performed in mice (female C57/1B16 Mouse, aged 6-8 weeks; N=4) dosed via ICV (intracerebroventricular) administration or rat (Sprague Dawley Rat, aged 6-8 weeks; N=3) dosed via IT (intrathecal administration).

Mouse ICV. The in vivo evaluation protocol and dosage regimen for Mouse ICV are shown in the chart below. The siRNAs (Table 3) formulated at various dosage level (11 μg, 33 μg, 100 pg, or 150 pg) in artificial cerebrospinal fluid (aCSF) were administered as 10 μL ICV injections in single dose, to female C57/BL6 mice (aged 6-8 weeks; N=4). The tissues from right hemisphere were collected on D15. The results of the SOD1 mRNA knockdown in brain hemisphere in mice on D15 at these various dose levels were analyzed. The controls were aCSF without siRNAs administrations.

Quantitative PCR analysis was performed using a Roche Cycler 480 ii system from Roche Diagnostics Indianapolis, IN 46256 (Catalog No. 05 015 243 001) with qPCR Master Mix (Roche, 04887301001), pre-validated superoxide dismutase 1, soluble (SOD1) (Rn00566938_m1) and primer for Mouse GAPD (GAPDH) Endogenous Control (Applied Biosystems Catalog No. 4351309) Taqman Gene Expression Assays (Thermo Fisher Scientific).

Rat IT. The in vivo evaluation protocol and dosage regime for Rat IT are shown in the chart below. The siRNAs (Table 3) formulated at various dosage level (0.03 mg, 0.1 mg, 0.3 mg, and 0.9 mg) in artificial cerebrospinal fluid (aCSF) were administered as 30 μL direct stick IT injections in single dose, to female Sprague Dawley rat (aged 6-8 weeks; N=3). The tissues from CNS (striatum, frontal cortex, cerebellum, hippocampus, whole spinal cord (e.g., cervical, lumbar, or thoracic)) were collected on D15. The results of the SOD1 mRNA knockdown in various CNS tissues in rat on D15 at these various dose levels were analyzed. The controls were aCSF without siRNAs administrations.

Quantitative PCR analysis was performed using a Roche Cycler 480 ii system from Roche Diagnostics Indianapolis, IN 46256 (Catalog No. 05 015 243 001) with qPCR Master Mix (Roche, 04887301001), pre-validated superoxide dismutase 1, soluble (SOD1) (Rn00566938_m1), rat peptidylprolyl isomerase B (Ppib) (Rn03302274_m1), and primer for Rat GAPD (GAPDH) Endogenous Control (Applied Biosystems Catalog No. 4351317) Taqman Gene Expression Assays (Thermo Fisher Scientific).

Drug concentrations in the CNS tissue samples were quantitated using a qualified LC-MS/MS method. Briefly, for each dosage level, tissue samples (for spinal cord and for frontal cortex) for all animal groups (N=3) were homogenized in lysis buffer, then the oligonucleotides were extracted from the tissue lysate by solid phase extraction and analyzed using ion-pairing reverse phase liquid chromatography coupled with mass spectrometry under negative ionization mode. The concentration of the full-length antisense strand of the dosed duplex was measured. The drug concentrations were reported as the antisense-based duplex concentrations. The calibration range was 100-50000 ng/g for CNS tissue samples. Concentrations that were calculated below the LLOQ are reported as <LLOQ.

| | | Surgical | | ICV | | | | |
|---|---|---|---|---|---|---|---|---|
| Duplex | | IT (rat)/ | | (mouse)/ | | | | |
| ID | Chemistry | Dose | | Dose | | Duration | Animals | Tissue Collection |
| AD- | C16 SS6, | 0.03 | mg | 11 | μg | D 15 | N = 3 | Rat CNS (frozen for |
| 401824 | VP | 0.1 | mg | 33 | μg | | Female | qPCR): |
| | Control | 0.3 | mg | 100 | μg | | SD Rats | Thoracic Spine |
| | | 0.9 | mg | 150 | μg | | N = 4 | Frontal cortex, |
| AD- | C22 SS6, | 0.03 | mg | 11 | μg | | Female | cerebellum, |
| 1427062 | VP | 0.1 | mg | 33 | μg | | C57B1/6 | hippocampus, |
| | | 0.3 | mg | 100 | μg | | | striatum |
| | | 0.9 | mg | 150 | μg | | | Mouse CNS (frozen |
| | | | | | | | | for qPCR): |
| | | | | | | | | Right hemisphere |
| | | | | | | | | CNS (fixed for |
| | | | | | | | | histology): |
| | | | | | | | | Left hemisphere |
| | | | | | | | | Periphery (frozen for |
| | | | | | | | | qPCR): |
| | | | | | | | | heart, liver |

The results of the inhibition of SOD1 gene expression in mouse CNS tissues ICV) and rat CNS tissues (via IT) by the C22-conjugated siRNA as compared to the results by the C16-conjugated siRNA are shown in FIGS. 5A and 5B. The results indicate that, overall, in both rodent models (mouse model via ICV administration and rat model via IT administration), the C22-conjugated siRNA had a biodistribution and silencing activity (0% knockdown) similar to or better than that of the C16-conjugated siRNA across all CNS tissues. In particular, at the lowest dosage levels, the C22-conjugated siRNA had a higher KD (0% knockdown) than the C16-conjugated siRNA in both rodent models. For instance, in rat IT, as shown in FIG. 5B, at the lowest dosage level (0.03 mg), the C22-conjugated siRNA showed at least 10% more KD (00 knockdown) than the C16-conjugated siRNA (e.g., in the thoracic spinal cord).

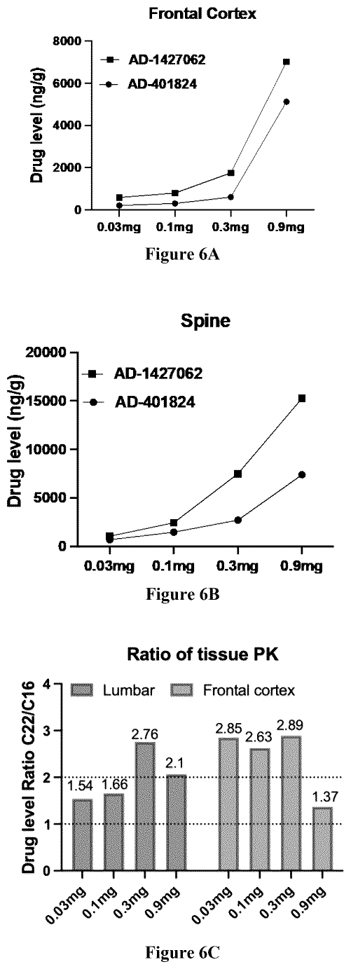

The results of the concentrations of the C22-conjugated siRNA duplex as compared to that of the C16-conjugated siRNA duplex in rat CNS tissues (frontal cortex tissue and spine), following IT administration of the siRNA duplex are shown in FIGS. 6A-6C. The results indicate that C22-conjugated siRNA duplexes were observed in higher concentrations than the C16-conjugated siRNA duplexes in both the thoracic cord and frontal cortex tissues, in a dose response manner.

Overall these results provided evidence for improved CNS tissue uptake and distributions of the C22-conjugated siRNA duplex in rodents as compared to the C16-conjugated siRNA duplexes, even though the absolute KD (% knockdown) for the C22-conjugated siRNA duplex could appear comparable to that of the C16-conjugated siRNA duplex in the short term.

### Example 9: Duration Evaluating of C22-Conjugated siRNA Delivery to Rodent (Rat IT)

Exemplary siRNAs comprising a C₂₂ hydrocarbon chain conjugated to the sense strand at position 6, counting from the 5′-end of the sense strand (AD-1427062), were used in this example at various dosages (0.1 mg, 0.3 mg, and 0.6 mg), at various time points over a duration of 90 days. Comparative siRNAs comprising a C₁₆ hydrocarbon chain conjugated to the sense strand at position 6, counting from the 5′-end of the sense strand (AD-401824), were used in this example, at higher dosages (0.3 mg, 0.6 mg, 0.9 mg) than the exemplary siRNAs, at the same time points over a duration of 90 days. The sequences were designed to target SOD1, with assessing the mRNA expression in frontal cortex, hippocampus, cerebellum, striatum, or thoracic cord tissues. The sequences used in this example are the same as those listed in Table 3 above.

In vivo studies were performed in rat (Sprague Dawley Rat, aged 6-8 weeks; N=3) dosed via IT (intrathecal administration). The in vivo evaluation protocol and dosage regimen for Rat IT are shown in the chart below. The siRNAs (Table 3) formulated at various dosage levels in artificial cerebrospinal fluid (aCSF), according to the chart below, were administered as 30 μL surgical IT injections in single dose, to female Sprague Dawley rat (aged 6-8 weeks; N=3). The tissues from CNS (striatum, frontal cortex, cerebellum, hippocampus) and spine (thoracic cord) were collected on D30 and D90. The results of the SOD1 mRNA knockdown in various CNS tissues in rat at these various dose levels and various time points were analyzed. The controls were aCSF without siRNAs administrations.

Quantitative PCR analysis was performed using a Roche Cycler 480 ii system from Roche Diagnostics Indianapolis, IN 46256 (Catalog No. 05 015 243 001) with qPCR Master Mix (Roche, 04887301001), pre-validated superoxide dismutase 1, soluble (SOD1) (Rn00566938_m1), rat peptidylprolyl isomerase B (Ppib) (Rn03302274_m1), and primer for Rat GAPD (GAPDH) Endogenous Control (Applied Biosystems Catalog No. 4351317) Taqman.

Gene Expression Assays (Thermo Fisher Scientific).

| | | | Dose | | | | |
|---|---|---|---|---|---|---|---|
| Group | Duplex | | (mg in | Route of | | | |
| No. | ID | Chemistry | 30 μl) | Admin | Duration | Animal | Tissues/Analysis |
| 1 | aCSF | — | — | D0 | D30 | Female | CNS (frozen for qPCR): |
| | | | | Single | | SD Rat | Thoracic Spine |
| 2 | AD- | C16 SS6, | 0.3 | Surgical | | | Frontal cortex, |
| 3 | 401824 | VP | 0.6 | IT Dose | | | cerebellum, |
| 4 | | | 0.9 | | | | hippocampus, |
| 5 | AD- | C22 SS6, | 0.1 | | | | striatum |
| 6 | 1427062 | VP | 0.3 | | | | CNS (fixed for |
| 7 | | | 0.6 | | | | histology): |
| 8 | aCSF | — | — | | D90 | | Left hemisphere |
| 9 | AD- | C16 SS6, | 0.3 | | | | Periphery (frozen |
| 10 | 401824 | VP | 0.6 | | | | for qPCR): |
| 11 | | | 0.9 | | | | heart, liver |
| 12 | AD- | C22 SS6, | 0.3 | | | | |
| 13 | 1427062 | VP | 0.6 | | | | |

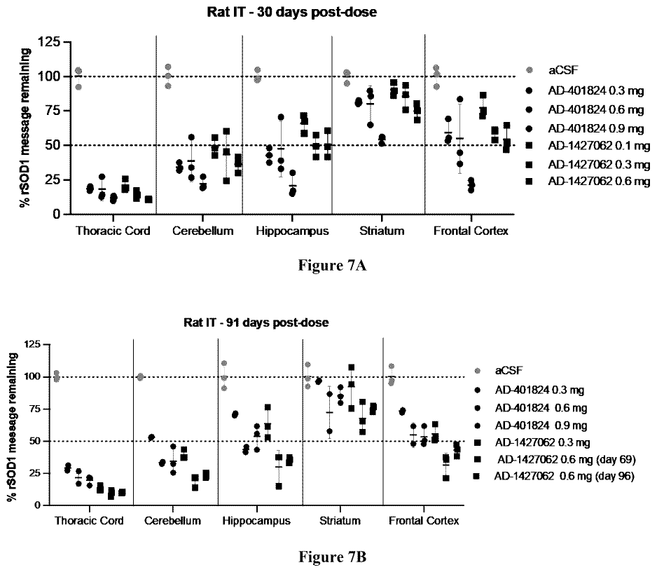

The results of the inhibition of SOD1 gene expression in rat CNS tissues (via IT) by the C22-conjugated siRNA at low to medium doses as compared to the results by the C16-conjugated siRNA at higher doses at various time points are shown in FIGS. 7A (D30) and 7B (D90).

The results indicate that, in a shorter term (e.g., 30 days), at equal doses, the C22-conjugated siRNAs resulted in a SOD1 knockdown comparable to those of the C16-conjugated siRNAs in all brain tissues tested. For instance, as shown in FIG. 7A, at day 30, the C22-conjugated siRNAs resulted in an approximately 50% SOD1 knockdown in the hippocampus and frontal cortex, at a dosage level of 0.6 mg, which was comparable to that of the C16-conjugated siRNAs in the hippocampus and frontal cortex, at dosage level of 0.6 mg.

At three months, the C22-conjugated siRNAs exhibited a better activity (i.e., more knockdown) as compared to the C16-conjugated siRNAs at equal doses. For instance, as shown in FIG. 7B3, at day 30, the C22-conjugated siRNAs resulted in an approximately 3500 rSOD1 remaining in the hippocampus (vs 440% rSOD1 remaining in the hippocampus for the C16-conjugated siRNAs) and 43% rSOD1 remaining in the frontal cortex (versus 55% remaining in the frontal cortex for the C16-conjugated siRNAs), at a dosage level of 0.6 mg.

Overall these results provided evidence for improved durability of silencing activity of the C22-conjugated siRNA duplex in the CNS tissues in rodents as compared to the C16-conjugated siRNA duplexes.

### Example 10: Evaluating of C22-Conjugated siRNA in Cell Specific CNS Delivery to Rodent (Rat IT)

Exemplary siRNAs comprising a C₂₂ hydrocarbon chain conjugated to the sense strand at position 6, counting from the 5′-end of the sense strand, targeting various CNS targets (MAP2, GFAP, MBP, Aif1), were used in this example at 0.6 mg. Comparative siRNAs comprising a C₁₆ hydrocarbon chain conjugated to the sense strand at position 6, counting from the 5′-end of the sense strand, targeting the same CNS targets, were used in this example at the same dosage level as the exemplary siRNAs. The sequences were designed to target MAP2 (microtubule-associated protein 2), GFAP (glial fibrillary acidic protein), MBP (myelin basic protein), and Aif1 (allograft inflammatory factor 1), respectively, with assessing the mRNA expression in frontal cortex, hippocampus, cerebellum, striatum, or thoracic cord tissues. The sequences are listed in Table 4 below.

TABLE 4
siRNAs having C22 conjugated at N6 position of sense strand (with C16 controls)

| | | | | | SEQ ID | |
|---|---|---|---|---|---|---|
| Duplex ID | Oligo ID | strand | target | oligoSeq | NO: | Chemistry |
| AD- | A-3448237.2 | sense | Map2 | asgsgau(Ada)GfuUfCfAfaguaguca | 52 | SS6 C22 |
| 1942522.2 | | | | scsa | | |
| | A-859918.4 | antis | Map2 | VPusGfsugaCfuAfCfuugaAfcUfau | 39 | |
| | | | | ccususg | | |
| AD- | A-920992.3 | sense | Map2 | asgsgau(Ahd)GfuUfCfAfaguaguca | 38 | SS6 C16 |
| 476454.14 | | | | scsa | | |
| | A-859918.4 | antis | Map2 | VPusGfsugaCfuAfCfuugaAfcUfau | 39 | |
| | | | | ccususg | | |
| AD- | A-3448238.2 | sense | Gfap | csasgau(Ada)GfuCfUfUfguuugcua | 53 | SS6 C22 |
| 1942524.2 | | | | sgsa | | |
| | A-871673.6 | antis | Gfap | VPusCfsuagCfaAfAfcaagAfcUfau | 54 | |
| | | | | cugsasg | | |
| AD- | A-921034.4 | sense | Gfap | csasgau(Ahd)GfuCfUfUfguuugcua | 55 | SS6 C16 |
| 476496.7 | | | | sgsa | | |
| | A-871673.4 | antis | Gfap | VPusCfsuagCfaAfAfcaagAfcUfau | 54 | |
| | | | | cugsasg | | |
| AD- | A-3448243.2 | sense | Mbp | gscsuuc(Ada)GfaCfCfAfuccaagaas | 56 | SS6 C22 |
| 1942527.2 | | | | gsa | | |
| | A-874305.3 | antis | Mbp | VPusCfsuucUfuGfGfauggUfcUfga | 48 | |
| | | | | agcsusc | | |
| AD- | A-2514557.3 | sense | MBP | gscsuuc(Ahd)GfaCfCfAfuccaagaas | 47 | SS6 C16 |
| 1350088.9 | | | | gsa | | |
| | A-874305.5 | antis | Mbp | VPusCfsuucUfuGfGfauggUfcUfga | 48 | |
| | | | | agcsusc | | |
| AD- | A-3448239.3 | sense | Aifl | usgsaga(Ada)UfgAfUfUfcugaugua | 57 | SS6 C22 |
| 1942526.3 | | | | susa | | |
| | A-876505.6 | antis | Aifl | VPusAfsuacAfuCfAfgaauCfaUfuc | 58 | |
| | | | | ucasasg | | |
| AD- | A-1036803.4 | sense | Aifl | usgsaga(Ahd)UfgAfUfUfcugaugua | 59 | SS6 C16 |
| 538824.12 | | | | susa | | |
| | A-876505.7 | antis | Aifl | VPusAfsuacAfuCfAfgaauCfaUfuc | 58 | |
| | | | | ucasasg | | |

In vivo studies were performed in rat (Sprague Dawley Rat, aged 6-8 weeks; N=3) dosed via IT (intrathecal administration). The in vivo evaluation protocol and dosage regime for Rat IT are shown in the chart below. The siRNAs (Table 4) formulated at 0.6 mg per dose in artificial cerebrospinal fluid (aCSF), according to the chart below, were administered as 30 μL surgical IT injections in single dose, to female Sprague Dawley rat (aged 6-8 weeks; N=3). The tissues from CNS (striatum, frontal cortex, cerebellum, and hippocampus) and spine (thoracic cord) were collected on D15. The results of the mRNA knockdown for these various targets in rat in various tissues were analyzed. The controls were aCSF without siRNAs administrations.

Quantitative PCR analysis was performed using a Roche Cycler 480 ii system from Roche Diagnostics Indianapolis, IN 46256 (Catalog No. 05 015 243 001) with qPCR Master Mix (Roche, 04887301001), pre-validated superoxide dismutase 1, soluble (SOD1) (Rn00566938_m1), rat peptidylprolyl isomerase B (Ppib) (Rn03302274_m1), and primer for Rat GAPD (GAPDH) Endogenous Control (Applied Biosystems Catalog No. 4351317) Taqman

Gene Expression Assays (Thermo Fisher Scientific).

| | Duplex | | | | Route of | | | Tissue |
|---|---|---|---|---|---|---|---|---|
| Group | ID | Target | Chemistry | Dose | Admin | Duration | Animals | Collection |
| 1 | aCSF | | n/a | — | Surgical | D15 | SD Rat | CNS (frozen |
| | | | | | IT | | Female | for qPCR): |
| 2 | AD- | MAP2 | SS6 C16 | 0.6 | | | 6-8 wks | Thoracic |
| | 476454.14 | | | mg | | | | spine, frontal |
| 3 | AD- | | SS6 C22 | | | | | cortex, |
| | 1942522.2 | | | | | | | cerebellum, |
| 5 | AD- | GFAP | SS6 C16 | | | | | hippocampus, |
| | 476496.7* | | | | | | | striatum |
| 6 | AD- | | SS6 C22 | | | | | CNS (fixed for |
| | 1942524.2 | | | | | | | histology): |
| 8 | AD- | MBP | SS6 C16 | | | | | Left hemisphere |
| | 1350088.9 | | | | | | | Periphery (frozen |
| 9 | AD- | | SS6 C22 | | | | | for qPCR): |
| | 1942527.2 | | | | | | | heart, liver |
| 11 | AD- | Aif1 | SS6 C16 | | | | | |
| | 538824.12 | | | | | | | |
| 12 | AD- | | SS6 C22 | | | | | |
| | 1942526.3 | | | | | | | |

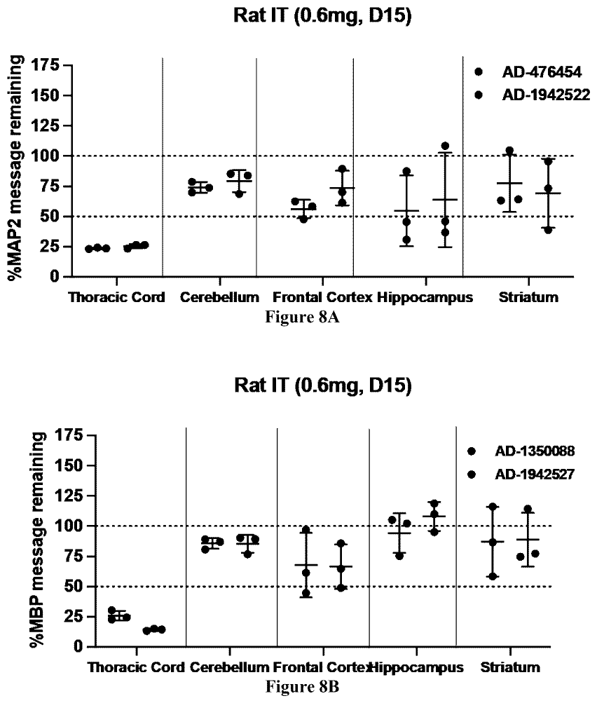

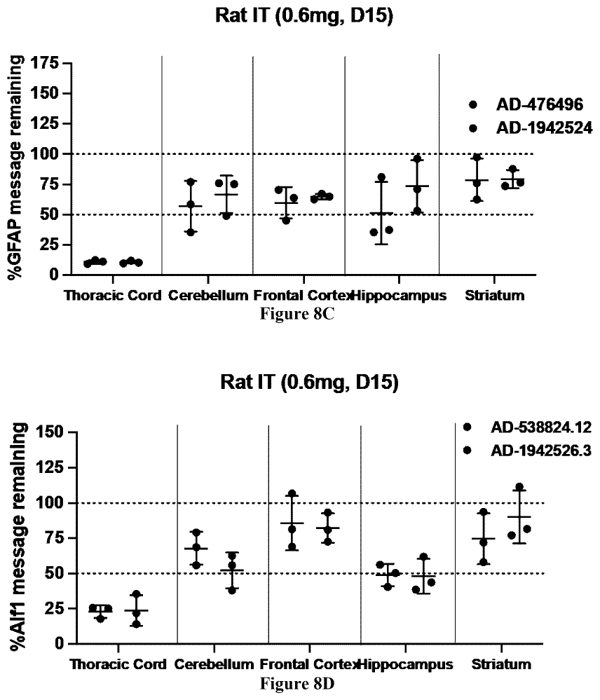

The results of the inhibition of various CNS target genes (MAP2, GFAP, MBP, Aif1) in rat CNS tissues (via IT) by the C22-conjugated siRNA as compared to the results by the C16-conjugated siRNA are shown in FIGS. 8A-8D. The results in FIGS. 8A, 8C, and 8D indicate that the mRNA silencing activities of MAP2, GFAP, and Aif1 in the rat CNS by the C22-conjugated siRNAs were comparable to those by the C16-conjugated siRNAs. FIG. 8B shows that the C22-conjugated siRNAs resulted in an increase in rMBP (myelin basic protein) silencing activity (% knockdown) in the thoracic cord tissue with C22 as compared to that of the C16-conjugated siRNAs in the thoracic cord tissue.

### Example 11: Evaluating Potency and Duration of C22-Conjugated siRNA Delivery to Non-Human Primate (NHP)

Exemplary siRNAs comprising a C₂₂ hydrocarbon chain conjugated to the sense strand at position 6, counting from the 5′-end of the sense strand, targeting various regions of APP transcript, respectively, were used in this example at various dosages (20 mg and 60 mg). Comparative siRNAs comprising a C₁₆ hydrocarbon chain conjugated to the sense strand at position 6, counting from the 5′-end of the sense strand, targeting the same CNS targets, were used in this example at the same dosage levels as the exemplary siRNAs. The sequences were designed to target APP, with assessing the mRNA expression in prefrontal cortex, hippocampus, cerebellum, caudate, putamen, striatum, or spine (e.g., lumbar spine) tissues. The siRNA duplexes sequences are listed in Table 5 below.

TABLE 5
siRNAs having C22 conjugated at N6 position of sense strand (with C16 controls)
target oligoSeq

| | | | | | SEQ | | Molecular |
|---|---|---|---|---|---|---|---|
| Duplex | | | | | ID | Molecular | Weight |
| Id | Oligo Id | Strand | target | oligoSeq | NO: | Weight | found |
| AD- | A- | sense | APP | usasgug(Cda)AfuGfAfAfuag | 60 | 7287.256 | 7283.389 |
| 2034769 | 3654750 | | | auucuscsa | | | |
| | A- | antis | APP | VPusGfsagaa(Tgn)cuauucAf | 61 | 7662.09 | 7658.152 |
| | 882380 | | | uGfcacuasgsu | | | |
| AD- | A- | sense | APP | ususugu(Gda)UfaCfUfGfuaa | 62 | 7289.228 | 7285.357 |
| 1956470 | 3476740 | | | agaaususa | | | |
| | A- | antis | APP | VPusAfsauuc(Tgn)uuacagUf | 63 | 7613.108 | 7609.183 |
| | 882378 | | | aCfacaaasasc | | | |
| AD- | A- | sense | APP | usasuga(Ada)GfuUfCfAfuca | 64 | 7278.296 | 7274.426 |
| 2034768 | 3654749 | | | ucaaasasa | | | |
| | A- | antis | APP | VPusUfsuuug(Agn)ugaugaA | 65 | 7586.97 | 7583.071 |
| | 882514 | | | fcUfucauasusc | | | |
| AD- | A- | sense | APP | usasgug(Chd)AfuGfAfAfua | 66 | 7203.082 | 7199.295 |
| 454843 | 882379 | | | gauucuscsa | | | |
| | A- | antis | APP | VPusGfsagaa(Tgn)cuauucAf | 61 | 7662.09 | 7658.152 |
| | 882380 | | | uGfcacuasgsu | | | |
| AD- | A- | sense | APP | ususugu(Ghd)UfaCfUfGfuaa | 67 | 7205.068 | 7201.265 |
| 454842 | 882377 | | | agaaususa | | | |
| | A- | antis | APP | VPusAfsauuc(Tgn)uuacagUf | 63 | 7613.108 | 7609.183 |
| | 882378 | | | aCfacaaasasc | | | |
| AD- | A- | sense | APP | usasuga(Ahd)GfuUfCfAfuca | 68 | 7194.139 | 7190.329 |
| 454972 | 882363 | | | ucaaasasa | | | |
| | A- | antis | APP | VPusUfsuuug(Agn)ugaugaA | 65 | 7586.97 | 7583.071 |
| | 882514 | | | fcUfucauasusc | | | |

Gene-silencing activities and siRNA concentrations (PK) in non-human primates (NHP) was studied with siRNA conjugates listed in Table 5 above, evaluating the APP target gene expression level in the CSF in the NHP, after a single intrathecal injection at a dose regimen shown in the chart below.

| Group | | | Route of | Dose | # of |
|---|---|---|---|---|---|
| No | Treatment | Target | Amin | (mg) | NHP |
| 1 | aCSF | — | IT | — | 4 |
| 3 | AD- | APP | IT | 20 | 4 |
| | 2034769 | (Parent: AD- | | | |
| 4 | AD- | 454843, C16 | IT | 60 | 4 |
| | 2034769 | SS6 VP control) | | | |
| 5 | AD- | APP siRNA 2 | IT | 20 | 4 |
| | 1956470 | (Parent: AD- | | | |
| 6 | AD- | 454842, C16 | IT | 60 | 4 |
| | 1956470 | SS6 VP control) | | | |
| 7 | AD- | APP siRNA 3 | IT | 20 | 4 |
| | 2034768 | (Parent: AD- | | | |
| 8 | AD- | 454972, C16 | IT | 60 | 4 |
| | 2034768 | SS6 VP control) | | | |

Soluble APP alpha (sAPPα)/soluble APP Beta (sAPPβ)

CSF Levels of sAPPα and sAPPβ were Determined Utilizing a Sandwich immunoassay MSD® 96-well MULTI-SPOT sAPPα/sAPPβ assay (Catalog no. K15120E; Meso Scale Discovery, Rockville, MD, USA) according to the manufacturer's protocol with some modifications. The standards, blanks, and non-human primate CSF samples (8× dilution) were prepared with the 1% Blocker-A/TB ST (provided in the kit). Pre-coated plate (provided in the kit) was blocked with 150 pL/well of 3% Blocker A/TB ST solution at room temperature for 1 hour with shaking. After three washes with 1×TBST, 25 pL/well of prepared standard, blanks, and CSF samples were added to the plate in two replicates and incubated for 1 hour at room temperature with shaking. Following subsequent plate washes, 50 pL/well of detection antibody prepared in 1% Blocker A/TBST (50× dilution) was added and incubated at room temperature for 1 hour with shaking. After plate washes, IX Read Buffer T was added to the plate and incubated for 10 minutes at room temperature (without shaking) before imaging and analyzing in MSD QuickPlex Imager.

Raw data were analyzed using SoftMax Pro, version 7.1 (Molecular Devices). A 5-parameter, logistic curve fitting with 1/Y2 weighing function was used to model the individual calibration curves and calculate the concentration of analytes in the samples.

### Mass Spec Method

Drug concentrations in CSF, and CNS tissue samples were quantitated using a qualified LC-MS/MS method. Briefly, for each dosage level, tissue samples for all animal groups were homogenized in lysis buffer, then the oligonucleotides were extracted from CSF or tissue lysate by solid phase extraction and analyzed using ion-pairing reverse phase liquid chromatography coupled with mass spectrometry under negative ionization mode. The concentration of the full-length antisense strand of the dosed duplex was measured. The drug concentrations were reported as the antisense-based duplex concentrations. The calibration range is 10-5000 ng/mL for CSF samples, and 100-50000 ng/g for CNS tissue samples. Concentrations that were calculated below the LLOQ are reported as <LLOQ. An analog duplex with different molecular weight was used as internal standard. mRNA knockdown by qPCR method.

Total RNA was isolated from rat brain and spinal cord tissue samples using the miRNeasy Mini Kit from (Qiagen, Catalog No. 217004) according to the manufacturer's instructions. Following isolation, RNA was reverse transcribed using Superscript™ IV VILO™ Reverse Transcriptase (Thermo Fisher Scientific). Quantitative PCR analysis was performed using a ViiA7 Real-Time PCR System from Thermo Fisher Scientific of Waltham MA 02451 (Catalog No. 4453537) with Taqman Fast Universal PCR Master Mix (Applied Biosystems Catalog No. 4352042), pre-validated amyloid beta precursor protein (APP) (Mf01552291_m1) and peptidylprolyl isomerase B (PPIB) (Mf02802985_m1) Taqman Gene Expression Assays (Thermo Fisher Scientific).

The relative reduction of APP mRNA was calculated using the comparative cycle threshold (Ct) method. During qPCR, the instrument sets a baseline in the exponential phase of the amplification curve and assigns a Ct value based on the intersection point of the baseline with the amplification curve. The APP mRNA reduction was normalized to the experimental untreated control group as a percentage for each respective group using the Ct values according to the following calculations:

$$Δ {Ct}_{App} = {Ct}_{App} - {Ct}_{Ppib}$$

$$ΔΔ {Ct}_{App} = Δ {Ct}_{App} - Δ {Ct}_{untreatedcontrolgroupmean}$$

$$Relative mRNA level = {2}^{- Δ Δ C t}$$

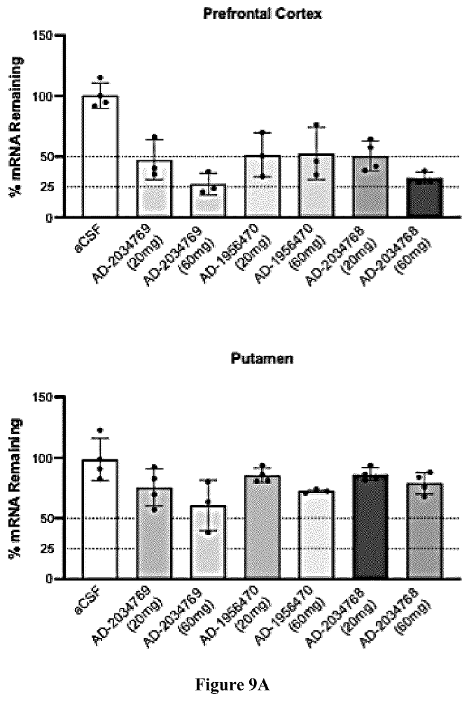

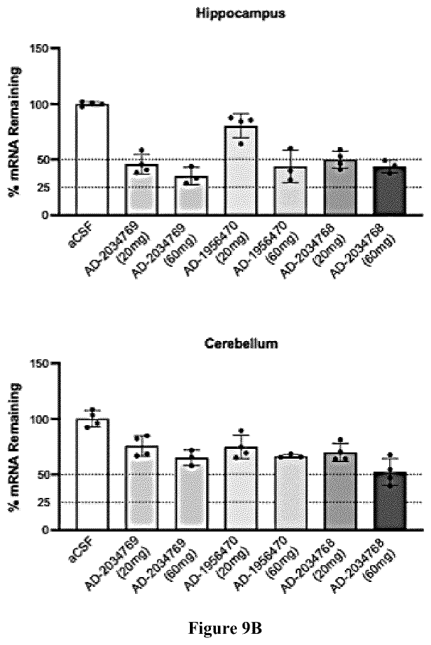

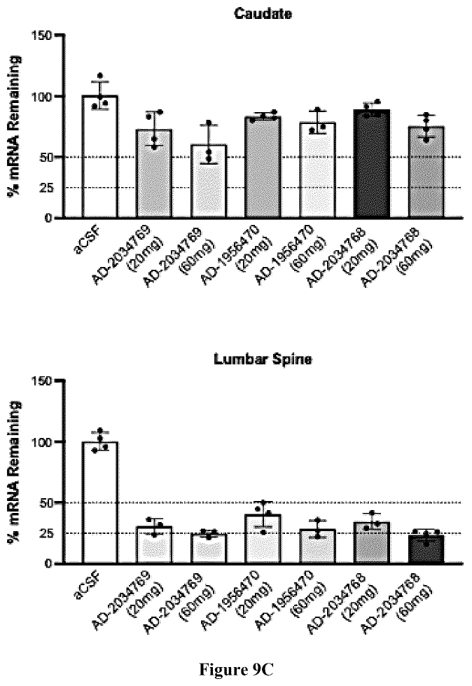

The results of the inhibition of CSF biomarker APP gene expression after intrathecal (IT) injection of the C22-conjugated siRNA duplexes (dosed at 20 mg and 60 mg at DO; and redosed approximately D29) in NHP CNS tissues (prefrontal cortex, putamen, hippocampus, cerebellum, caudate, and lumbar spine tissues) at D106 are shown in FIGS. 9A-9C. These figures indicate that the siRNA duplexes comprising a C₂₂ hydrocarbon chain conjugated to the sense strand demonstrated a significant and durable knockdown in the NHP CNS tissues in a dose dependent manner, particularly in prefrontal cortex, hippocampus, and lumbar spine tissues after a single IT dose. The magnitude of knockdown is driven by inherent potency of the siRNA sequences as shown by NHP tissue IC50 (shown in the graph and table in FIG. 11).

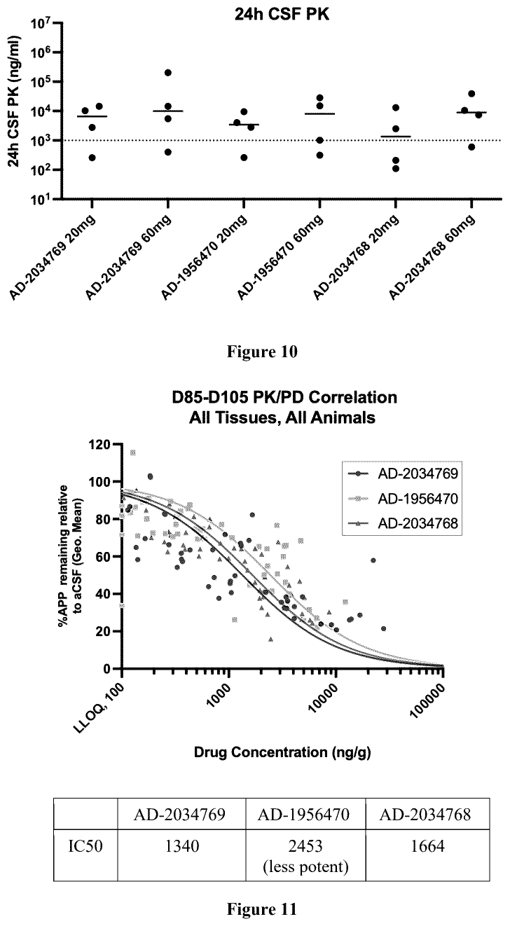

The 24-hour CSF PK data for the C22-conjugated siRNA duplexes (AD-2034769, AD-1956470, and AD-2034768) are shown in FIG. 10, which indicates that these C22-conjugated siRNA duplexes (at both 20 mg and 60 mg) had the CSF PK mostly within 10000 ng/ml. The C22-conjugated siRNA duplexes at 20 mg dosage level had the CSF PK close to or within 3000 ng/ml.

FIG. 11 shows the results of the D85-D105 PK/PD correlation in NHP for all tissues following IT administration of a C22-conjugated siRNA duplex (AD-2034769, AD-1956470, and AD-2034768, respectively). The X axis shows the concentrations of the C22-conjugated siRNA duplexes in NHP CNS (all tissues) following IT administration of the siRNA duplex D85-D105. The Y axis shows the % cyno APP remaining in NHP CNS (all tissues) following IT administration of the siRNA duplex D85-D105. The results indicate that the amount of the C22-conjugated siRNA duplexes in the CNS tissues of the NHP had a strong correlation with the amount of the target gene (APP) knockdown observed, that is to say, more tissue drug accumulation correlates with higher degree of target mRNA knockdown.

The levels of CSF soluble APP proteins (a and R) after intrathecal (IT) injection of the C22-conjugated siRNA duplexes (AD-2034769, AD-1956470, and AD-2034768) (dosed at 20 mg and 60 mg) at various time points (−D37 (baseline), DO (baseline), D8, D15, D29, D57, and D78) in the cynomolgus monkeys were compared to the levels of CSF soluble APP proteins (sAPPα/β) after intrathecal (IT) injection of the C16-conjugated siRNA duplexes (AD-454972, AD-454842, and AD-454843) (dosed at 72 mg) at various time points (DO, D8, D15, D29, D43, D57, and D78) in the cynomolgus monkeys. A single IT injection, via percutaneous needle stick, of the desired dosage (as discussed above) of an APP siRNA of interest was administered in cynomolgus monkeys between L2/L3 or L4/L5 in the lumbar cistern. CSF levels of sAPPa and sAPP and drug concentrations in CSF and CNS tissue were measured using the methods as discussed above. The sequences of the C22-conjugated siRNA duplexes (AD-2034769, AD-1956470, and AD-2034768) and the comparable C16-conjugated siRNA duplexes (AD-454972, AD-454842, and AD-454843) are shown in Table 5 above.

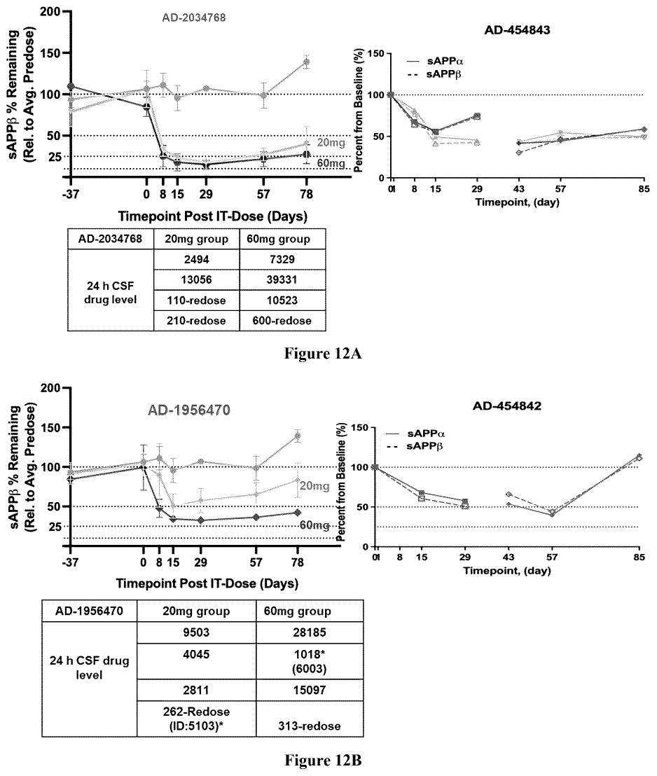

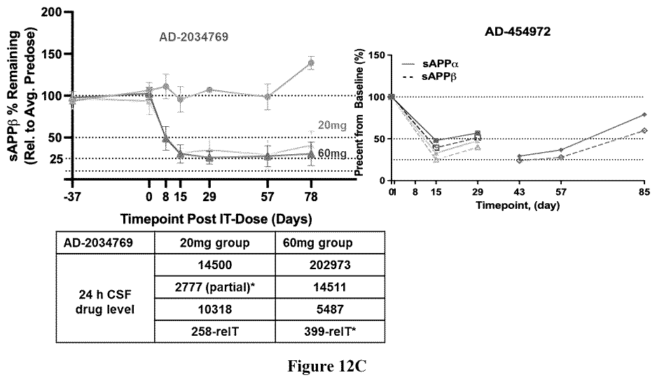

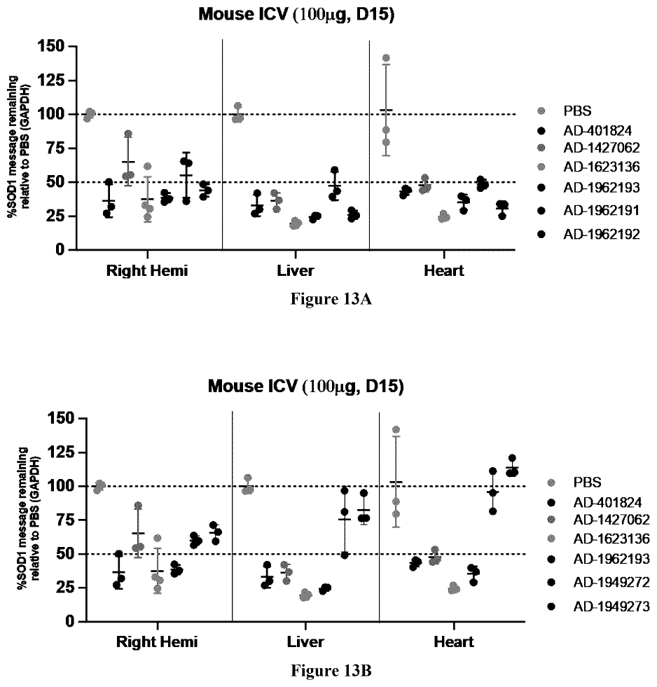

The results of the comparison are shown in FIGS. 12A-12C. Although the dosage levels for the C22-conjugated siRNA duplexes (20 mg and 60 mg) were not the same as that of the C16-conjugated siRNA duplexes (72 mg), the results nevertheless indicate that, for each duplex having a C22 conjugate versus a C16 conjugate, the C22-conjugated siRNA duplexes resulted in an enhanced potency, as evidenced by the comparable or enhanced knockdown at a lower dosage level (20 mg or 60 mg for the C22-conjugated siRNA vs. 72 mg for the C16-conjugated siRNA). This trend was even more apparent at longer duration, e.g., post dosage D57 and longer (e.g., D78 for C22-conjugated siRNA vs D85 for the C16-conjugated siRNA), indicating a better durability of the C22-conjugated siRNA duplexes as compared to the C16-conjugated siRNA duplexes.

### Example 12: SAR Evaluation of C22-Conjugated siRNAs (with Various Modifications on the Lipophilic Moiety) in Mice

The structure activity relationships of siRNAs conjugated with a lipophilic moiety containing a C₂₂ hydrocarbon chain, with various chemical modifications on the lipophilic moiety, were studied in mice. The sequences in this example are shown in Table 6 below.

TABLE 6
siRNAs sequences targeting SOD1

| Duplex | | | SEQ ID | |
|---|---|---|---|---|
| Name | Strand | OligoSeq (5′-3′) | NO: | Chemistry |
| AD-401824 | sense | csasuuu(Uhd)AfaUfCfCfucacucuasasa | 49 | C16 (Uhd), VP |
| | antis | VPusUfsuagAfgUfGfaggaUfuAfaaaugsasg | 50 | |
| AD-1623136 | sense | csasuuY211UfAfaUfCfCfucacucuasasa | 69 | C16 PN backbone |
| | antis | VPusUfsuagAfgUfGfaggaUfuAfaaaugsasg | 70 | (Y211), VP |
| AD-1427062 | sense | csasuuu(Uda)AfaUfCfCfucacucuasasa | 51 | C22 (Uda), VP |
| | antis | VPusUfsuagAfgUfGfaggaUfuAfaaaugsasg | 50 | |
| AD-1962193 | sense | csasuuY318UfAfaUfCfCfucacucuasasa | 71 | C22 PN backbone |
| | antis | VPusUfsuagAfgUfGfaggaUfuAfaaaugsasg | 72 | (Y318), VP |
| AD-1949272 | sense | csasuuY229UfAfaUfCfCfucacucuasasa | 73 | Dimethyl PN backbone |
| | antis | VPusUfsuagAfgUfGfaggaUfuAfaaaugsasg | 74 | (Y229), VP |
| AD-1949273 | sense | csasuuY230UfAfaUfCfCfucacucuasasa | 75 | Methyl PN backbone |
| | antis | VPusUfsuagAfgUfGfaggaUfuAfaaaugsasg | 76 | (Y230), VP |
| AD-1962191 | sense | csasuuuY250AfaUfCfCfucacucuasasa | 77 | C16 (Y250) |
| | antis | VPusUfsuagAfgUfGfaggaUfuAfaaaugsasg | 78 | NMA, VP |
| AD-1962192 | sense | csasuuuY270AfaUfCfCfucacucuasasa | 79 | C22 (Y270) |
| | antis | VPusUfsuagAfgUfGfaggaUfuAfaaaugsasg | 80 | NMA, VP |

AD-401824 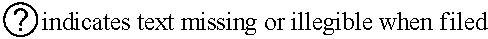
AD-1623136 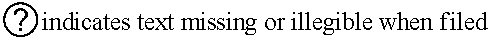
AD-1427062 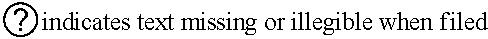
AD-1962193 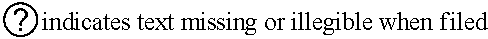
AD-1949272 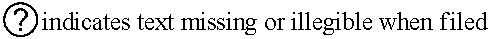
AD-1949273 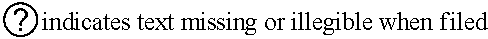
AD-1962191 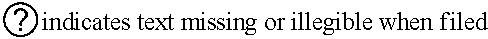
AD-1962192

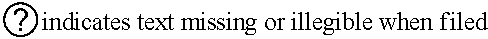

In vivo gene-silenting studies were performed in mice with the siRNA conjugates listed in Table 6 above, dosed via ICV (intracerebroventricular) administration. The in vivo evaluation protocol and dosage regimen for Mouse JCV are shown in the chart below. The siRNAs formulated at 100 g in PBS were administered as 10 μL ICV injections in single dose, to female C57/B16 mice (aged 6-8 weeks; N=4). The tissues from right hemisphere, heart, and liver were collected on D15. The results of the SOD1 mRNA knockdown in brain right hemisphere, liver, and heart in mice on D15 were analyzed. The controls were PBS without siRNAs administrations.

| | | Route of | | | |
|---|---|---|---|---|---|
| | Dose | Adminis- | Dura- | | |
| Duplex ID | (ug) | tration | tion | Animals | Tissue Collection |
| PBS | — | — | D 15 | C57/B16 | CNS (frozen for |
| AD-401824 | 100 | ICV | | Female | qPCR): |
| AD-1427062 | | | | | Right hemisphere - |
| AD-1623136 | | | | | olfactory bulbs, |
| AD-1962193 | | | | | brainstem, |
| AD-1949272 | | | | | cerebellum removed |
| AD-1949273 | | | | | Periphery (frozen |
| AD-1962191 | | | | | for qPCR): |
| AD-1962192 | | | | | heart, liver |

FIGS. 13A-13B show the inhibition of SOD1 gene expression in mouse CNS tissue (right hemisphere) and periphery tissues (heart, liver) following ICV administration (dosed at 100 pg) of an siRNA duplex, with a lipophilic moiety containing a C₂₂ hydrocarbon chain, with various chemical modifications on the lipophilic moiety, on D15. As shown in Table 6 above, for the lipophilic moiety containing a C16 conjugate or a C22 conjugate, C16 or C22 was conjugated to either at the 2′-position, with the C16 or C22 modifying 2′-0 or modifying 2′-O—N-methylacetamido (2′-O-NMA), or at the 3′-position, with the C16 or C22 modifying N-alkylphosphoramidate. The lipophilic moiety containing a methyl modifying N-alkylphosphoramidate (PN methyl) and a dimethyl modifying N-alkylphosphoramidate (PN dimethyl) were used as additional controls. As shown in FIG. 13A, the SOD1 knockdown ability for the siRNAs with C22 lipophilic conjugations in the CNS tissue as well as in the liver and heart tissues have been improved when using a lipophilic moiety containing C22 modifying 2′-O—N-methylacetamido (2′-O-NMA) and lipophilic moiety containing C22 at the 3′-position, modifying N-alkylphosphoramidate, as compared to a lipophilic moiety containing C22 modifying 2′-0. As shown in FIG. 13B, the SOD1 knockdown ability for the siRNAs with PN-C22 modification as well as PN-C16 modification in the CNS tissue as well as in the liver and heart tissues were better compared to the siRNAs with a PN methyl modification and PN dimethyl modification.

Thus, the siRNA duplexes with PN-lipid modification (e.g., C22 Y318 PN or C22 Y270 NMA) demonstrated at least equivalent or superior pharmacology than the siRNA duplexes with lipid modification itself (2′ C22) in the CNS tissue (e.g., by ICV administration). The siRNA duplexes with PN-lipid modification also demonstrated silencing in heart and liver at the same time. In addition, the siRNA duplexes with PN-C22 modification performed superior to the siRNA duplexes with PN-C16 modification in the CNS tissue (e.g., by ICV administration).

### Example 13. Evaluating the Positional Impact of C22-Conjugation Across the siRNA Sequences In Vitro

Cell Culture and Transfection: siRNA was transfected in Neuro2A (ATCC Cat #CCL-131) or Be2C (ATCC Cat #CRL-2268) cells at 10, 1, 0.1 nM by adding 0.1 μl of RNAiMAX (Invitrogen, 13778) in 4.9 μl of Opti-MEM and 5 μl of siRNA in a 384-well plate. After a 15-minute room temperature incubation, 40 μl of media containing ~5×103 cells was added to the wells. Cells were incubated for 24 hours.

RNA Isolation: RNA was isolated using an automated protocol on a BioTek-EL406 platform using Dynabeads™ mRNA DIRECT™ Purification Kit (Invitrogen™ Catalog No. 61012). Briefly, 70 μL of Lysis/Binding Buffer and shake for 15 minutes. Ten L of lysis buffer containing 3 μL of magnetic beads were then added to the plates. Plates were incubated on an electromagnetic shaker for 10 minutes at room temperature and then magnetic beads were captured and the supernatant was removed. Bead-bound RNA was then washed 2 times with 90 μL Wash Buffer A and once with 90 μL Wash Buffer B. Beads were then washed with 90 μL Elution Buffer, re-captured, and supernatant was removed.

Complementary DNA Synthesis: Complementary DNA (cDNA) was synthesized using High-Capacity cDNA Reverse Transcription Kit with RNase Inhibitor (Applied Biosystems™, Catalog No. 4374967) according to the manufacturer's recommendations. A master mix containing 1 μL 10× Buffer, 0.4 μL 25× deoxyribonucleotide triphosphate, 1 μL 10× Random primers, 0.5 μL Reverse Transcriptase, 0.5 μL RNase inhibitor, and 6.6 μL of water per reaction was added to RNA isolated above. The plates were sealed, mixed, and incubated on a shaker for 10 minutes at room temperature, followed by 2 hours incubation at 37° C.

Quantitation of SOD1 Messenger RNA by RT-qPCR: SOD1 mRNA levels were quantified by performing RT-qPCR analysis. Two L of cDNA prepared above were added to a RT-qPCR master mix containing 0.5 μL of 20× human or mouse GAPDH TaqMan probe, 0.5 μL 20× human SOD1 probe (ThermoFisher Scientific 4331182, HsOO533490_m1) or 0.5 μL 20× mouse SOD1 probe (ThermoFisher Scientific 4331182, Mm01344233_g1) and L LightCycler 480 Probes Master mix per well in 384 well plates. The RT-qPCR assay was carried out in a LightCycler480 Real Time PCR system (Roche) using the TaqMan gene expression assay.

Quantitation of APP Messenger RNA by RT-qPCR: APP mRNA levels were quantified by performing RT-qPCR analysis. Quantitative PCR analysis was performed using a ViiA7 Real-Time PCR System from Thermo Fisher Scientific of Waltham MA 02451 (Catalog No. 4453537) with Taqman Fast Universal PCR Master Mix (Applied Biosystems Catalog No. 4352042), pre-validated amyloid beta precursor protein (APP) (Mf01552291_m1) and peptidylprolyl isomerase B (PPIB) (Mf02802985_m1) Taqman Gene Expression Assays (Thermo Fisher Scientific).

Data Analysis: To calculate relative fold change, real-time data were analyzed using the Delta-Delta Threshold Cycle (Relative Quantification) (ΔΔCt[RQ]) method [Schmittgen and Livak 2008] and normalized to control assays performed using cells transfected with 10 nmol/L AD1955 (nontarget control). For all samples, SOD1 mRNA levels were first normalized to GAPDH as a reference gene. Data are expressed as percent of SOD1 mRNA remaining and error is expressed as standard deviation (SD), derived from the 4 transfection replicates. Means and SDs were calculated using Microsoft® Office Excel (Microsoft Corporation [Redmond, WA]).

### Sense Strand

The effect of the conjugation position of a lipophilic moiety containing a C₂₂ hydrocarbon chain across the entire siRNA sequence on the sense strand were evaluated using exemplary siRNAs that target SOD1, comprising a C₂₂ hydrocarbon chain, at various dosages (0.1 nM, 1 nM, and 10 nM, respectively) in rodent Neuro2a cells as well as in human Be2C cells. Cells were incubated with each siRNA conjugate at the indicated dosage level with transfection reagent RNAiMAX and SOD1 mRNA was measured after 24 hours. Control siRNAs include a parent siRNA duplex without a lipophilic conjugate (unconj.), as well as the siRNA duplex comprising a C₁₆ hydrocarbon chain conjugated to the sense strand at position 6, counting from the 5′-end of the sense strand (S6-C16), were used in this example, at the same dosages as the exemplary siRNAs. The in vitro mRNA expression for each sample was assessed in respective rodent Neuro2a cells and human Be2C cells.

The sequences used are shown in Table 7 below.

TABLE 7
siRNAs sequences targeting SOD1, used in in vitro evaluation of positional impact
of C22-Conjugation across the sense strand

| | | | | SEQ | Molecular | Molecular | |
|---|---|---|---|---|---|---|---|
| Duplex | | | | ID | Weight | Weight | |
| ID | Strand | Oligo ID | Oligo Seq | NO | (cal) | (Found) | Notes |
| AD- | Sense | A-1684938 | ususggg(Chd)AfaAfGfGfuggaaa | 45 | 7384.248 | 7380.379 | S6-C16 |
| 890098 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-3507231 | ususgggcAfaAfGfGfuggaaaugsa | 81 | 7173.854 | 7170.144 | Parent |
| 1964624 | | | sa | | | | No |
| | Antis | A-859296 | VPusUfscauUfuCfCfaccuUfuGf | 46 | 7532.881 | 7529.035 | Lipophile |
| | | | cccaasgsu | | | | |
| AD- | Sense | A-3507248 | (Udas)usgggcAfaAfGfGfuggaaa | 82 | 7468.419 | 7464.473 | S1 |
| 2026257 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235755 | us(Udas)gggcAfaAfGfGfuggaaa | 83 | 7468.419 | 7464.473 | S2 |
| 2273356 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235756 | usus(Gda)ggcAfaAfGfGfuggaaa | 84 | 7468.422 | 7464.473 | S3 |
| 2273357 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235757 | ususg(Gda)gcAfaAfGfGfuggaaa | 85 | 7468.422 | 7464.473 | S4 |
| 2273358 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235758 | ususgg(Gda)cAfaAfGfGfuggaaa | 86 | 7468.422 | 7464.473 | S5 |
| 2273359 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-3340763 | ususggg(Cda)AfaAfGfGfuggaaa | 87 | 7468.423 | 7464.473 | S6 |
| 1812376 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235759 | ususgggc(Ada)aAfGfGfuggaaau | 88 | 7480.457 | 7476.493 | S7 |
| 2273360 | | | gsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235760 | ususgggcAf(Ada)AfGfGfuggaaa | 89 | 7468.421 | 7464.473 | S8 |
| 2273361 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235761 | ususgggcAfa(Ada)GfGfuggaaau | 90 | 7480.457 | 7476.493 | S9 |
| 2273362 | | | gsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235762 | ususgggcAfaAf(Gda)Gfuggaaau | 91 | 7480.458 | 7476.493 | S10 |
| 2273363 | | | gsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235763 | ususgggcAfaAfGf(Gda)uggaaau | 92 | 7480.458 | 7476.493 | S11 |
| 2273364 | | | gsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235764 | ususgggcAfaAfGfGf(Uda)ggaaa | 93 | 7468.412 | 7464.473 | S12 |
| 2273365 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235765 | ususgggcAfaAfGfGfu(Gda)gaaa | 94 | 7468.422 | 7464.473 | S13 |
| 2273366 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235766 | ususgggcAfaAfGfGfug(Gda)aaa | 95 | 7468.422 | 7464.473 | S14 |
| 2273367 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235767 | ususgggcAfaAfGfGfugg(Ada)aa | 96 | 7468.421 | 7464.473 | S15 |
| 2273368 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235768 | ususgggcAfaAfGfGfugga(Ada)a | 97 | 7468.421 | 7464.473 | S16 |
| 2273369 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235769 | ususgggcAfaAfGfGfuggaa(Ada) | 98 | 7468.421 | 7464.473 | S17 |
| 2273370 | | | ugsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235770 | ususgggcAfaAfGfGfuggaaa | 99 | 7468.412 | 7464.473 | S18 |
| 2273371 | | | (Uda)gsasa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235771 | ususgggcAfaAfGfGfuggaaau | 100 | 7468.421 | 7464.473 | S19 |
| 2273372 | | | (Gdas)asa | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235772 | ususgggcAfaAfGfGfuggaaaugs | 101 | 7468.421 | 7464.473 | S20 |
| 2273373 | | | (Adas)a | | | | |
| | Antis | A-859296 | VPusUfscauUfuCfCfaccuUfuGf | 46 | 7532.881 | 7529.035 | |
| | | | cccaasgsu | | | | |
| AD- | Sense | A-4235773 | ususgggcAfaAfGfGfuggaaaugsa | 102 | 7468.421 | 7464.473 | S21 |
| 2273374 | | | s(Ada) | | | | |
| | Antis | A-859296 | VPusUfscauUfuCfCfaccuUfuGf | 46 | 7532.881 | 7529.035 | |
| | | | cccaasgsu | | | | |

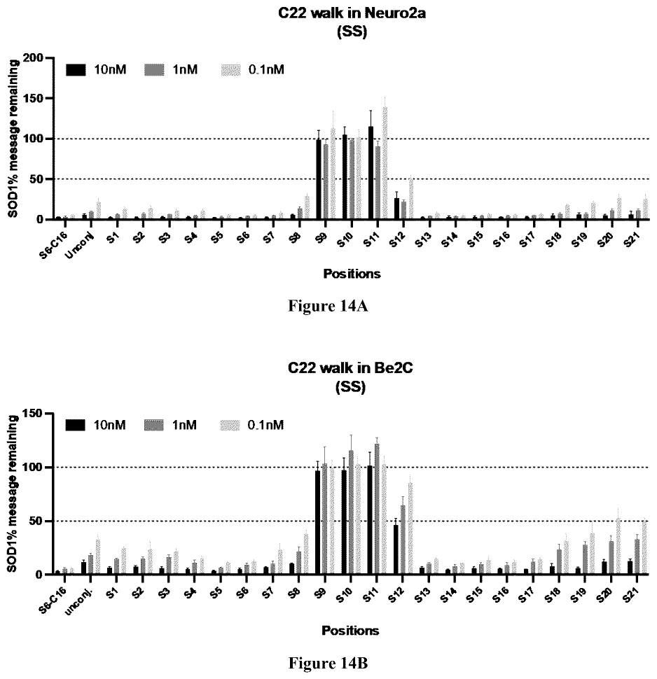

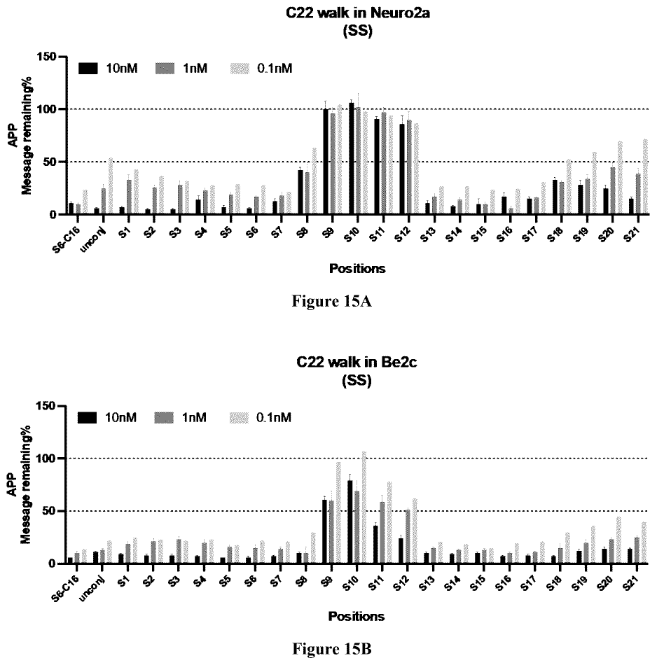

The results from in vitro silencing studies in rodent Neuro2a cells are summarized in FIG. 14A, and the results from in vitro silencing studies in human Be2C cells are summarized in FIG. 14B. As shown in FIGS. 14A-14B, the C22 conjugation to the sense strand at positions other than positions 9, 10, and 11 generally maintain an activity similar to the S6-C16 control and similar or even better than the parent siRNA duplex without a lipophilic conjugate (unconj.). The C22 conjugation to the sense strand at a position close to positions 9, 10, and 11 (e.g., S8 and S12) did not provide an activity as good as those provided by C22 conjugation at a position that is not close to positions 9, 10, and 11.

The effect of the conjugation position of a lipophilic moiety containing a C₂₂ hydrocarbon chain across the entire siRNA sequence on the sense strand were also evaluated using exemplary siRNAs that target APP, comprising a C₂₂ hydrocarbon chain, at various dosages (0.1 nM, 1 nM, and 10 nM, respectively) in rodent Neuro2a cells as well as in human Be2C cells. Cells were incubated with each siRNA conjugate at the indicated dosage level with transfection reagent RNAiMAX and APP mRNA was measured after 24 hours. Control siRNAs include a parent siRNA duplex without a lipophilic conjugate (unconj.), as well as the siRNA duplex comprising a C₁₆ hydrocarbon chain conjugated to the sense strand at position 6, counting from the 5′-end of the sense strand (S6-C16), were used in this example, at the same dosages as the exemplary siRNAs. The in vitro mRNA expression for each sample was assessed in respective rodent Neuro2a cells and human Be2C cells.

The sequences used are shown in Table 8 below.

TABLE 8
siRNAs sequences targeting APP, used in in vitro evaluation of positional impact of
C22-Conjugation across the sense strand

| | | | | SEQ | Molecular | Molecular | |
|---|---|---|---|---|---|---|---|
| Duplex | | | | ID | Weight | Weight | Pos- |
| ID | Strand | Oligo ID | Sense Oligo Seq | NO | (cal) | (Found) | ition |
| AD- | Sense | A-3476807 | (Adas)asaaucCfaAfCfCfuacaagu | 103 | 7236.301 | 7232.452 | S1 |
| 2273314 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235714 | as(Adas)aaucCfaAfCfCfuacaagu | 104 | 7236.301 | 7232.452 | S2 |
| 2273315 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235715 | asas(Ada)aucCfaAfCfCfuacaagu | 105 | 7236.301 | 7232.452 | S3 |
| 2273316 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235716 | asasa(Ada)ucCfaAfCfCfuacaagu | 106 | 7236.301 | 7232.452 | IS4 |
| 2273317 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235717 | asasaa(Uda)cCfaAfCfCfuacaagu | 107 | 7236.292 | 7232.452 | IS5 |
| 2273318 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-2365995 | asasaau(Cda)CfaAfCfCfuacaagu | 42 | 7236.303 | 7232.452 | S6 |
| 1302923 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235718 | asasaauc(Cda)aAfCfCfuacaaguu | 108 | 7248.339 | 7244.472 | S7 |
| 2273319 | | | scsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235719 | asasaaucCf(Ada)AfCfCfuacaagu | 109 | 7236.301 | 7232.452 | S8 |
| 2273320 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235720 | asasaaucCfa(Ada)CfCfuacaaguu | 110 | 7248.337 | 7244.472 | S9 |
| 2273321 | | | scsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235721 | asasaaucCfaAf(Cda)Cfuacaaguu | 111 | 7248.339 | 7244.472 | S10 |
| 2273322 | | | scsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235722 | asasaaucCfaAfCf(Cda)uacaaguu | 112 | 7248.339 | 7244.472 | S11 |
| 2273323 | | | scsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235723 | asasaaucCfaAfCfCf(Uda)acaagu | 113 | 7236.292 | 7232.452 | S12 |
| 2273324 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235724 | asasaaucCfaAfCfCfu(Ada)caagu | 114 | 7236.301 | 7232.452 | S13 |
| 2273325 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235725 | asasaaucCfaAfCfCfua(Cda)aagu | 115 | 7236.303 | 7232.452 | S14 |
| 2273326 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235726 | asasaaucCfaAfCfCfuac(Ada)agu | 116 | 7236.301 | 7232.452 | S15 |
| 2273327 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235727 | asasaaucCfaAfCfCfuaca(Ada)gu | 117 | 7236.301 | 7232.452 | S16 |
| 2273328 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235728 | asasaaucCfaAfCfCfuacaa(Gda)u | 118 | 7236.302 | 7232.452 | S17 |
| 2273329 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235729 | asasaaucCfaAfCfCfuacaag(Uda) | 119 | 7236.292 | 7232.452 | S18 |
| 2273330 | | | uscsa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235730 | asasaaucCfaAfCfCfuacaagu | 120 | 7236.299 | 7232.452 | S19 |
| 2273331 | | | (Udas)csa | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235731 | asasaaucCfaAfCfCfuacaaguus | 121 | 7236.301 | 7232.452 | S20 |
| 2273332 | | | (Cdas)a | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-4235732 | asasaaucCfaAfCfCfuacaaguuscs | 122 | 7236.301 | 7232.452 | S21 |
| 2273333 | | | (Ada) | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-1690014 | asasaaucCfaAfCfCfuacaaguuscs | 123 | 6941.738 | 6938.124 | unconj |
| 960500 | | | a | | | | |
| | Antis | A-882382 | VPusGfsaacu(Tgn)guagguUfgGf | 41 | 7712.057 | 7708.105 | |
| | | | auuuuscsg | | | | |
| AD- | Sense | A-882381 | asasaau(Chd)CfaAfCfCfuacaagu | 40 | 7152.13 | 7148.358 | S6-C16 |
| 454844 | | | uscsa | | | | |
| | Antis | A-882382 | VPusGfsaacu(Tgn)guagguUfgGf | 41 | 7712.057 | 7708.105 | |
| | | | auuuuscsg | | | | |

The results from in vitro silencing studies in rodent Neuro2a cells are summarized in FIG. 15A, and the results from in vitro silencing studies in human Be2C cells are summarized in FIG. 15B. Similar to the results from the siRNA duplexes targeting SOD1, shown in FIGS. 14A-14B, the results in FIGS. 15A-15B also show that the C22 conjugation to the sense strand at positions other than positions 9, 10, and 11 generally maintain an activity similar to the S6-C16 control and similar or even better than the parent siRNA duplex without a lipophilic conjugate (unconj.); and the C22 conjugation to the sense strand at a position close to positions 9, 10, and 11 (e.g., S8 and S12) did not provide an activity as good as those provided by C22 conjugation at a position that is not close to positions 9, 10, and 11.

### Antisense Strand

The effect of the conjugation position of a lipophilic moiety containing a C₂₂ hydrocarbon chain across the entire siRNA sequence on the antisense strand were evaluated using exemplary siRNAs that target SOD1, comprising a C₂₂ hydrocarbon chain, at various dosages (0.1M, 1 nM, and 10 nM, respectively) in rodent Neuro2a cells as well as in human Be2C cells. Cells were incubated with each siRNA conjugate at the indicated dosage level with transfection reagent RNAiMAX and SOD1 mRNA was measured after 24 hours. Control siRNAs include a parent siRNA duplex without a lipophilic conjugate (unconj. in Table 7), as well as the siRNA duplex comprising a C₁₆ hydrocarbon chain conjugated to the sense strand at position 6, counting from the 5′-end of the sense strand (S6-C16 in Table 7), were used in this example, at the same dosages as the exemplary siRNAs. The in vitro mRNA expression for each sample was assessed in respective rodent Neuro2a cells and human Be2C cells.

The exemplary sequences used are shown in Table 9 below.

TABLE 9
siRNAs sequences targeting SOD1, used in in vitro evaluation of positional impact
of C22-Conjugation across the antisense strand

| | | | | SEQ | Molecular | Molecular | AS |
|---|---|---|---|---|---|---|---|
| Duplex | | | | ID | Weight | Weight | pos- |
| ID | Strand | Oligo ID | Oligo Seq | NO | (cal) | (Found) | ition |
| AD- | Sense | A-3507231 | ususgggcAfaAfGfGfuggaaaug | 81 | 7173.854 | 7170.144 | AS2 |
| 2273375 | | | sasa | | | | |
| | Antis | A-4235774 | VPus(Udas)cauUfuCfCfaccuU | 124 | 7839.479 | 7835.384 | |
| | | | fuGfcccaasgsu | | | | |
| AD- | Sense | A-3507231 | ususgggcAfaAfGfGfuggaaaug | 81 | 7173.854 | 7170.144 | AS3 |
| 2273376 | | | sasa | | | | |
| | Antis | A-4235775 | VPusUfs(Cda)cauUfuCfCfacc | 125 | 8146.656 | 8142.421 | |
| | | | uUfuGfcccaasgsu | | | | |
| AD- | Sense | A-3507231 | ususgggcAfaAfGfGfuggaaaug | 81 | 7173.854 | 7170.144 | AS4 |
| 2273377 | | | sasa | | | | |
| | Antis | A-4235776 | VPusUfsc(Ada)uUfuCfCfaccu | 126 | 7827.446 | 7823.364 | |
| | | | UfuGfcccaasgsu | | | | |
| AD- | Sense | A-3507231 | ususgggcAfaAfGfGfuggaaaug | 81 | 7173.854 | 7170.144 | AS5 |
| 2273378 | | | sasa | | | | |
| | Antis | A-4235777 | VPusUfsca(Uda)UfuCfCfaccu | 127 | 7827.437 | 7823.364 | |
| | | | UfuGfcccaasgsu | | | | |
| AD- | Sense | A-3507231 | ususgggcAfaAfGfGfuggaaaug | 81 | 7173.854 | 7170.144 | AS6 |
| 2273379 | | | sasa | | | | |
| | Antis | A-4235778 | VPusUfscau(Uda)uCfCfaccuU | 128 | 7839.473 | 7835.384 | |
| | | | fuGfcccaasgsu | | | | |
| AD- | Sense | A-3507231 | ususgggcAfaAfGfGfuggaaaug | 81 | 7173.854 | 7170.144 | AS7 |
| 2273380 | | | sasa | | | | |
| | Antis | A-4235779 | VPusUfscauUf(Uda)CfCfaccu | 129 | 7827.437 | 7823.364 | |
| | | | UfuGfcccaasgsu | | | | |
| AD- | Sense | A-3507231 | ususgggcAfaAfGfGfuggaaaug | 81 | 7173.854 | 7170.144 | AS8 |
| 2273381 | | | sasa | | | | |
| | Antis | A-4235780 | VPusUfscauUfu(Cda)CfaccuU | 130 | 7839.484 | 7835.384 | |
| | | | fuGfcccaasgsu | | | | |
| AD- | Sense | A-3507231 | ususgggcAfaAfGfGfuggaaaug | 81 | 7173.854 | 7170.144 | AS9 |
| 2273382 | | | sasa | | | | |
| | Antis | A-4235781 | VPusUfscauUfuCf(Cda)accuU | 131 | 7839.484 | 7835.384 | |
| | | | fuGfcccaasgsu | | | | |
| AD- | Sense | A-3507231 | ususgggcAfaAfGfGfuggaaaug | 81 | 7173.854 | 7170.144 | AS10 |
| 2273383 | | | sasa | | | | |
| | Antis | A-4235782 | VPusUfscauUfuCfCf(Ada)ccu | 132 | 7827.446 | 7823.364 | |
| | | | UfuGfcccaasgsu | | | | |
| AD- | Sense | A-3507231 | ususgggcAfaAfGfGfuggaaaug | 81 | 7173.854 | 7170.144 | AS11 |
| 2273384 | | | sasa | | | | |
| | Antis | A-4235783 | VPusUfscauUfuCfCfa(Cda)cu | 133 | 7827.448 | 7823.364 | |
| | | | UfuGfcccaasgsu | | | | |
| AD- | Sense | A-3507231 | ususgggcAfaAfGfGfuggaaaug | 81 | 7173.854 | 7170.144 | AS12 |
| 2273385 | | | sasa | | | | |
| | Antis | A-4235784 | VPusUfscauUfuCfCfac(Cda)u | 134 | 7827.448 | 7823.364 | |
| | | | UfuGfcccaasgsu | | | | |
| AD- | Sense | A-3507231 | ususgggcAfaAfGfGfuggaaaug | 81 | 7173.854 | 7170.144 | AS13 |
| 2273386 | | | sasa | | | | |
| | Antis | A-4235785 | VPusUfscauUfuCfCfacc(Uda) | 135 | 7827.437 | 7823.364 | |
| | | | UfuGfcccaasgsu | | | | |
| AD- | Sense | A-3507231 | ususgggcAfaAfGfGfuggaaaug | 81 | 7173.854 | 7170.144 | AS14 |
| 2273387 | | | sasa | | | | |
| | Antis | A-4235786 | VPusUfscauUfuCfCfaccu | 136 | 7839.473 | 7835.384 | |
| | | | (Uda)uGfcccaasgsu | | | | |
| AD- | Sense | A-3507231 | ususgggcAfaAfGfGfuggaaaug | 81 | 7173.854 | 7170.144 | AS15 |
| 2273388 | | | sasa | | | | |
| | Antis | A-4235787 | VPusUfscauUfuCfCfaccuUf | 137 | 7827.437 | 7823.364 | |
| | | | (Uda)Gfcccaasgsu | | | | |
| AD- | Sense | A-3507231 | ususgggcAfaAfGfGfuggaaaug | 81 | 7173.854 | 7170.144 | AS16 |
| 2273389 | | | sasa | | | | |
| | Antis | A-4235788 | VPusUfscauUfuCfCfaccuUfu | 138 | 7839.483 | 7835.384 | |
| | | | (Gda)cccaasgsu | | | | |
| AD- | Sense | A-3507231 | ususgggcAfaAfGfGfuggaaaug | 81 | 7173.854 | 7170.144 | AS17 |
| 2273390 | | | sasa | | | | |
| | Antis | A-4235789 | VPusUfscauUfuCfCfaccuUfu | 139 | 7827.448 | 7823.364 | |
| | | | Gf(Cda)ccaasgsu | | | | |
| AD- | Sense | A-3507231 | ususgggcAfaAfGfGfuggaaaug | 81 | 7173.854 | 7170.144 | AS18 |
| 2273391 | | | sasa | | | | |
| | Antis | A-4235790 | VPusUfscauUfuCfCfaccuUfu | 140 | 7827.448 | 7823.364 | |
| | | | Gfc(Cda)caasgsu | | | | |
| AD- | Sense | A-3507231 | ususgggcAfaAfGfGfuggaaaug | 81 | 7173.854 | 7170.144 | AS19 |
| 2273392 | | | sasa | | | | |
| | Antis | A-4235791 | VPusUfscauUfuCfCfaccuUfu | 141 | 7827.448 | 7823.364 | |
| | | | Gfcc(Cda)aasgsu | | | | |
| AD- | Sense | A-3507231 | ususgggcAfaAfGfGfuggaaaug | 81 | 7173.854 | 7170.144 | AS20 |
| 2273393 | | | sasa | | | | |
| | Antis | A-4235792 | VPusUfscauUfuCfCfaccuUfu | 142 | 7827.446 | 7823.364 | |
| | | | Gfccc(Ada)asgsu | | | | |
| AD- | Sense | A-3507231 | ususgggcAfaAfGfGfuggaaaug | 81 | 7173.854 | 7170.144 | AS21 |
| 2273394 | | | sasa | | | | |
| | Antis | A-4235793 | VPusUfscauUfuCfCfaccuUfu | 143 | 7827.446 | 7823.364 | |
| | | | Gfccca(Adas)gsu | | | | |
| AD- | Sense | A-3507231 | ususgggcAfaAfGfGfuggaaaug | 81 | 7173.854 | 7170.144 | AS22 |
| 2273395 | | | sasa | | | | |
| | Antis | A-4235794 | VPusUfscauUfuCfCfaccuUfu | 144 | 7827.446 | 7823.364 | |
| | | | Gfcccaas(Gdas)u | | | | |
| AD- | Sense | A-3507231 | ususgggcAfaAfGfGfuggaaaug | 81 | 7173.854 | 7170.144 | AS23 |
| 2273396 | | | sasa | | | | |
| | Antis | A-4235795 | VPusUfscauUfuCfCfaccuUfu | 145 | 7827.437 | 7823.364 | |
| | | | Gfcccaasgs(Uda) | | | | |

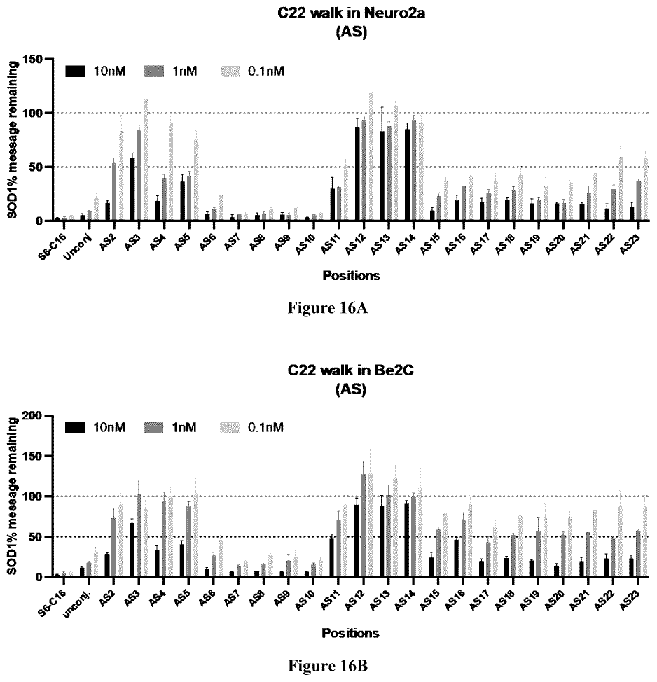

The results from in vitro silencing studies in rodent Neuro2a cells are summarized in FIG. 16A, and the results from in vitro silencing studies in human Be2C cells are summarized in FIG. 16B. As shown in FIGS. 16A-16B, the C22 conjugation to the antisense strand at positions other than positions 2-5 and other than positions 12-14 generally maintain an activity similar to the S6-C16 control and similar or even better than the parent siRNA duplex without a lipophilic conjugate (unconj.). The C22 conjugation to the antisense strand at a position close to positions 12-14 (e.g., AS11) did not provide an activity as good as those provided by C22 conjugation at a position that is not close to positions 12-14. Overall, C22 conjugation to one of positions 6-10 provided the best silencing activities.

The effect of the conjugation position of the a lipophilic moiety containing a C₂₂ hydrocarbon chain across the entire siRNA sequence on the antisense strand were also evaluated using exemplary siRNAs that target APP, comprising a C₂₂ hydrocarbon chain, at various dosages (0.1 nM, 1 nM, and 10 nM, respectively) in rodent Neuro2a cells as well as in human Be2C cells. Cells were incubated with each siRNA conjugate at the indicated dosage level with transfection reagent RNAiMAX and APP mRNA was measured after 24 hours. Control siRNAs include a parent siRNA duplex without a lipophilic conjugate (unconj. in Table 8), as well as the siRNA duplex comprising a C₁₆ hydrocarbon chain conjugated to the sense strand at position 6, counting from the 5′-end of the sense strand (S6-C16 in Table 8), were used in this example, at the same dosages as the exemplary siRNAs. The in vitro mRNA expression for each sample was assessed in respective rodent Neuro2a cells and human Be2C cells.

The exemplary sequences used are shown in Table 10 below.

TABLE 10
siRNAs sequences targeting APP, used in in vitro evaluation of positional impact
of C22-Conjugation across the antisense strand

| | | | | SEQ | Weight | Molecular | AS |
|---|---|---|---|---|---|---|---|
| Duplex | | | | ID | Molecular | Weight | pos- |
| ID | Strand | Oligo ID | Oligo Seq | NO | (cal) | (Found) | ition |
| AD- | Sense | A-1690014 | asasaaucCfaAfCfCfuacaaguuscsa | 123 | 6941.738 | 6938.124 | AS2 |
| 2273334 | Antis | A-4235733 | VPus(Gdas)aacu(Tgn)guagguUfg | 146 | 8018.656 | 8014.453 | |
| | | | Gfauuuuscsg | | | | |
| AD- | Sense | A-1690014 | asasaaucCfaAfCfCfuacaaguuscsa | 123 | 6941.738 | 6938.124 | AS3 |
| 2273335 | Antis | A-4235734 | VPusGfs(Ada)acu(Tgn)guagguUf | 147 | 8006.622 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS4 |
| 2273336 | Antis | A-4235735 | VPusGfsa(Ada)cu(Tgn)guagguUf | 148 | 8006.622 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS5 |
| 2273337 | Antis | A-4235736 | VPusGfsaa(Cda)u(Tgn)guagguUf | 149 | 8006.624 | 8002.434 | |
| | | | gGfauuuuscsg | | | | |
| AD- | Sense | A-1690014 | asasaaucCfaAfCfCfuacaaguuscsa | 123 | 6941.738 | 6938.124 | AS6 |
| 2273338 | Antis | A-4235737 | VPusGfsaac(Uda)(Tgn)guagguUf | 150 | 8006.613 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS7 |
| 2273339 | Antis | A-4235738 | VPusGfsaacu(Uda)guagguUfgGf | 151 | 8064.65 | 8060.439 | |
| | | | auuuuscsg | | | | |
| AD- | Sense | A-1690014 | asasaaucCfaAfCfCfuacaaguuscsa | 123 | 6941.738 | 6938.124 | AS8 |
| 2273340 | Antis | A-4235739 | VPusGfsaacu(Tgn)(Gda)uagguUf | 152 | 8006.623 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS9 |
| 2273341 | Antis | A-4235740 | VPusGfsaacu(Tgn)g(Uda)agguUf | 153 | 8006.613 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS10 |
| 2273342 | Antis | A-4235741 | VPusGfsaacu(Tgn)gu(Ada)gguUf | 154 | 8006.622 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS11 |
| 2273343 | Antis | A-4235742 | VPusGfsaacu(Tgn)gua(Gda)guUf | 155 | 8006.623 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS12 |
| 2273344 | Antis | A-4235743 | VPusGfsaacu(Tgn)guag(Gda)uUf | 156 | 8006.623 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS13 |
| 2273345 | Antis | A-4235744 | VPusGfsaacu(Tgn)guagg(Uda)Uf | 157 | 8006.613 | 8002.433 | |
| | | | gGfauuuuscsg | | | | |
| AD- | Sense | A-1690014 | asasaaucCfaAfCfCfuacaaguuscsa | 123 | 6941.738 | 6938.124 | AS14 |
| 2273346 | Antis | A-4235745 | VPusGfsaacu(Tgn)guaggu(Uda)g | 158 | 8018.649 | 8014.453 | |
| | | | Gfauuuuscsg | | | | |
| AD- | Sense | A-1690014 | asasaaucCfaAfCfCfuacaaguuscsa | 123 | 6941.738 | 6938.124 | AS15 |
| 2273347 | Antis | A-4235746 | VPusGfsaacu(Tgn)guagguUf(Gda) | 159 | 8006.623 | 8002.433 | |
| | | | Gfauuuuscsg | | | | |
| AD- | Sense | A-1690014 | asasaaucCfaAfCfCfuacaaguuscsa | 123 | 6941.738 | 6938.124 | AS16 |
| 2273348 | Antis | A-4235747 | VPusGfsaacu(Tgn)guagguUfg(Gda) | 160 | 8018.659 | 8014.453 | |
| | | | auuuuscsg | | | | |
| AD- | Sense | A-1690014 | asasaaucCfaAfCfCfuacaaguuscsa | 123 | 6941.738 | 6938.124 | AS17 |
| 2273349 | Antis | A-4235748 | VPusGfsaacu(Tgn)guagguUfgGf | 161 | 8006.622 | 8002.433 | |
| | | | (Ada)uuuuscsg | | | | |
| AD- | Sense | A-1690014 | asasaaucCfaAfCfCfuacaaguuscsa | 123 | 6941.738 | 6938.124 | AS18 |
| 2273350 | Antis | A-4235749 | VPusGfsaacu(Tgn)guagguUfgGfa | 162 | 8006.613 | 8002.433 | |
| | | | (Uda)uuuscsg | | | | |
| AD- | Sense | A-1690014 | asasaaucCfaAfCfCfuacaaguuscsa | 123 | 6941.738 | 6938.124 | AS19 |
| 2273351 | Antis | A-4235750 | VPusGfsaacu(Tgn)guagguUfgGfa | 163 | 8006.613 | 8002.433 | |
| | | | u(Uda)uuscsg | | | | |
| AD- | Sense | A-1690014 | asasaaucCfaAfCfCfuacaaguuscsa | 123 | 6941.738 | 6938.124 | AS20 |
| 2273352 | Antis | A-4235751 | VPusGfsaacu(Tgn)guagguUfgGfa | 164 | 8006.613 | 8002.433 | |
| | | | uu(Uda)uscsg | | | | |
| AD- | Sense | A-1690014 | asasaaucCfaAfCfCfuacaaguuscsa | 123 | 6941.738 | 6938.124 | AS21 |
| 2273353 | Antis | A-4235752 | VPusGfsaacu(Tgn)guagguUfgGfa | 165 | 8006.62 | 8002.434 | |
| | | | uuu(Udas)csg | | | | |
| AD- | Sense | A-1690014 | asasaaucCfaAfCfCfuacaaguuscsa | 123 | 6941.738 | 6938.124 | AS22 |
| 2273354 | Antis | A-4235753 | VPusGfsaacu(Tgn)guagguUfgGfa | 166 | 8006.622 | 8002.433 | |
| | | | uuuus(Cdas)g | | | | |
| AD- | Sense | A-1690014 | asasaaucCfaAfCfCfuacaaguuscsa | 123 | 6941.738 | 6938.124 | AS23 |
| 2273355 | Antis | A-4235754 | VPusGfsaacu(Tgn)guagguUfgGfa | 167 | 8006.623 | 8002.433 | |
| | | | uuuuscs(Gda) | | | | |

The results from in vitro silencing studies in rodent Neuro2a cells are summarized in FIG. 17A, and the results from in vitro silencing studies in human Be2C cells are summarized in FIG. 17B. The results from in vitro silencing studies in human Be2C cells are very similar to the results from the siRNA duplexes targeting SOD1, shown in FIGS. 16A-16B. As shown in FIG. 17B, the C22 conjugation to the antisense strand at positions other than positions 2-5 and other than positions 12-14 generally maintain an activity similar to the S6-C16 control and similar or even better than the parent siRNA duplex without a lipophilic conjugate (unconj.). The C22 conjugation to the antisense strand at a position close to positions 12-14 (e.g., AS11) did not provide an activity as good as those provided by C22 conjugation at a position that is not close to positions 12-14. Overall, C22 conjugation to one of positions 6-10 provided the best silencing activities.

### Example 14. In Vivo Evaluation of the Positional Impact of C22-Conjugation-Mouse ICV

The effect of the conjugation position of a lipophilic moiety containing a C₂₂ hydrocarbon chain were also evaluated using exemplary siRNAs that target SOD1, comprising a C₂₂ hydrocarbon chain, at dosage level of 150 pg (in aCSF) administered in a single dose at 10 μL ICV injection to female C57/BL6 mice

Mouse ICV. The in vivo evaluation protocol and dosage regimen for Mouse ICV are shown in the chart below. The siRNAs formulated at dosage level of 150 μg in artificial cerebrospinal fluid (aCSF) were administered as 10 μL ICV injections in single dose to female C57/BL6 mice (aged 6-8 weeks; N=4). The tissues from right hemisphere were collected on D7. The results of the SOD1 mRNA knockdown in brain hemisphere in mice on D7 were analyzed. The controls were aCSF without siRNAs administrations.

| | | | | Route | | | |
|---|---|---|---|---|---|---|---|
| | | | | of | | | Tissue |
| Group | Duplex ID | Target | Dose | Admin. | Duration | Animals | Collection |
| 1 | aCSF | | — | ICV | D7 | C57BL/6J | CNS (frozen for |
| 2 | AD-890098 | SOD1 | 150 ug | | | | qPCR): Right |
| 3 | AD-1964624 | | | | | | brain hemisphere |
| 4 | AD-1812376 | | | | | | |
| 5 | AD-2273364 | | | | | | |
| 6 | AD-2273368 | | | | | | |
| 7 | AD-2273369 | | | | | | |
| 8 | AD-2273370 | | | | | | |
| 9 | AD-2273383 | | | | | | |

RNA extraction: Total RNA was isolated from mouse brain tissue samples using the miRNeasy Mini Kit from (Qiagen, Catalog No. 217004) according to the manufacturer's instructions. Following isolation, RNA was reverse transcribed using Superscript™ IV VILO™ Reverse Transcriptase (Thermo Fisher Scientific). Quantitative PCR analysis was performed using a ViiA7 Real-Time PCR System from Thermo Fisher Scientific of Waltham MA 02451 (Catalog No. 4453537) with Taqman Fast Universal PCR Master Mix (Applied Biosystems Catalog No. 4352042), soluble (SOD1) (Rn00566938_m1) and primer for Mouse GAPD (GAPDH) Endogenous Control (Applied Biosystems Catalog No. 4351309) Taqman Gene Expression Assays (Thermo Fisher Scientific).

The relative reduction of SOD1 mRNA was calculated using the comparative cycle threshold (Ct) method. During qPCR, the instrument sets a baseline in the exponential phase of the amplification curve and assigns a Ct value based on the intersection point of the baseline with the amplification curve. The SOD1 mRNA reduction was normalized to the experimental untreated control group as a percentage for each respective group using the Ct values according to the following calculations:

$$Δ {Ct}_{App} = {Ct}_{SOD} - {Ct}_{Ppib}$$

$$ΔΔ {Ct}_{App} = Δ {Ct}_{SOD} - Δ {Ct}_{untreatedcontrolgroupmean}$$

$$Relative mRNA level = {2}^{- Δ Δ C t}$$

The sequences used are shown in Table 11 below.

TABLE 11
siRNAs sequences, targeting SOD1 (having a lipophilic moiety), used in in vivo
evaluation in mouse after a single intracerebroventricular injection

| | | | | SEQ | Molecular | Molecular | Lipid |
|---|---|---|---|---|---|---|---|
| Duplex | Oligo | | | ID | (cal) | Weight | Chem- |
| ID | ID | Strand | Oligonucleotide Sequence | NO: | Weight | (Found) | istry |
| AD- | A- | sense | ususggg(Chd)AfaAfGfGfuggaa | 45 | 7384.248 | 7380.379 | SS6 C16 |
| 890098 | 1684938 | | augsasa | | | | |
| | A- | antis | VPusUfscauUfuCfCfaccuUfuGf | 46 | 7532.881 | 7529.035 | |
| | 859296 | | cccaasgsu | | | | |
| AD- | A- | sense | ususgggcAfaAfGfGfuggaaaugsa | 81 | 7173.854 | 7170.144 | Unconj. |
| 1964624 | 3507231 | | sa | | | | |
| | A- | antis | VPusUfscauUfuCfCfaccuUfuGf | 46 | 7532.881 | 7529.035 | |
| | 859296 | | cccaasgsu | | | | |
| AD- | A- | sense | ususggg(Cda)AfaAfGfGfuggaaa | 87 | 7468.423 | 7464.473 | SS6 C22 |
| 1812376 | 3340763 | | ugsasa | | | | |
| | A- | antis | VPusUfscauUfuCfCfaccuUfuGf | 46 | 7532.881 | 7529.035 | |
| | 859296 | | cccaasgsu | | | | |
| AD- | A- | sense | ususgggcAfaAfGf(Gda)uggaaau | 92 | 7480.458 | 7476.493 | SS11 |
| 2273364 | 4235763 | | gsasa | | | | C22 |
| | A- | antis | VPusUfscauUfuCfCfaccuUfuGf | 46 | 7532.881 | 7529.035 | |
| | 859296 | | cccaasgsu | | | | |
| AD- | A- | sense | ususgggcAfaAfGfGfugg(Ada)a | 96 | 7468.421 | 7464.473 | SS15 |
| 2273368 | 4235767 | | augsasa | | | | C22 |
| | A- | antis | VPusUfscauUfuCfCfaccuUfuGf | 46 | 7532.881 | 7529.035 | |
| | 859296 | | cccaasgsu | | | | |
| AD- | A- | sense | ususgggcAfaAfGfGfugga(Ada) | 97 | 7468.421 | 7464.473 | SS16 |
| 2273369 | 4235768 | | augsasa | | | | C22 |
| | A- | antis | VPusUfscauUfuCfCfaccuUfuGf | 46 | 7532.881 | 7529.035 | |
| | 859296 | | cccaasgsu | | | | |
| AD- | A- | sense | ususgggcAfaAfGfGfuggaa | 98 | 7468.421 | 7464.473 | SS17 |
| 2273370 | 4235769 | | (Ada)ugsasa | | | | C22 |
| | A- | antis | VPusUfscauUfuCfCfaccuUfuGf | 46 | 7532.881 | 7529.035 | |
| | 859296 | | cccaasgsu | | | | |
| AD- | A- | sense | ususgggcAfaAfGfGfuggaaaugsa | 81 | 7173.854 | 7170.144 | |
| 2273383 | 3507231 | | sa | | | | |
| | A- | antis | VPusUfscauUfuCfCf(Ada)ccuU | 132 | 7827.446 | 7823.364 | AS10 |
| | 4235782 | | fuGfcccaasgsu | | | | C22 |
| AD- | A- | sense | (Udas)usgggcAfaAfGfGfuggaaa | 82 | 7468.419 | 7464.473 | |
| 2026257 | 3507248 | | ugsasa | | | | |
| | A-859296 | antis | VPusUfscauUfuCfCfaccuUfuGf | 46 | 7532.881 | 7529.035 | |
| | | | cccaasgsu | | | | |
| AD- | A- | sense | ususgg(Gda)cAfaAfGfGfuggaaa | 86 | 7468.422 | 7464.473 | |
| 2273359 | 4235758 | | ugsasa | | | | |
| | A- | antis | VPusUfscauUfuCfCfaccuUfuGf | 46 | 7532.881 | 7529.035 | |
| | 859296 | | cccaasgsu | | | | |
| AD- | A- | sense | ususgggc(Ada)aAfGfGfuggaaau | 88 | 7480.457 | 7476.493 | |
| 2273360 | 4235759 | | gsasa | | | | |
| | A-859296 | antis | VPusUfscauUfuCfCfaccuUfuGf | 46 | 7532.881 | 7529.035 | |
| | | | cccaasgsu | | | | |

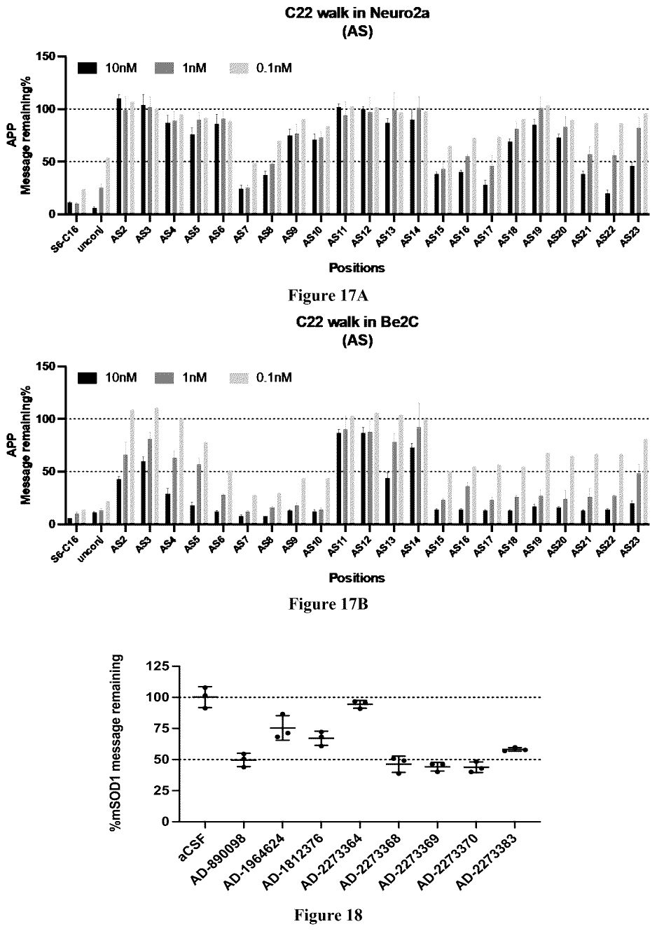

The results from in vivo silencing studies via mouse ICV are summarized in FIG. 18. As shown in FIG. 18, the C22 conjugation to positions 6, 15, 16, or 17 of the sense strand or position 10 of the antisense strand resulted in an activity similar to or better than the SS6-C16 control, all of which were better than the parent siRNA duplex without a lipophilic conjugate (unconj.).

### Example 15: Evaluation of siRNA Containing Internal Lipid Conjugation (Rat IT)

An SAR of internal conjugation of a lipophilic moiety containing a C₂₂ hydrocarbon chain to an siRNA (position 6 of the sense strand), targeting SOD1 was evaluated in CNS, liver, and heart via rat IT at a mid-range dose.

In vivo studies were performed in rat (Sprague Dawley Rat, aged 6-8 weeks; N=3) dosed via IT (intrathecal administration). The in vivo evaluation protocol and dosage regime for Rat IT are shown in the chart below. The siRNAs formulated at 0.6 mg per dose in artificial cerebrospinal fluid (aCSF), according to the chart below, were administered as 30 μL surgical IT injections in single dose, to female Sprague Dawley rat (aged 6-8 weeks; N=3). The tissues from CNS (striatum, frontal cortex, cerebellum, and hippocampus) and spine (thoracic cord) were collected on D15. The results of the mRNA knockdown for these various targets in rat in various tissues were analyzed. The controls were aCSF without siRNAs administrations.

| | | | Route | Du- | | |
|---|---|---|---|---|---|---|
| | | | of | ra- | | Tissue |
| Group | Duplex ID | Dose | Admin | tion | Animals | Collection |
| 1 | aCSF | — | D0 | D14 | SD Rat | Flash frozen |
| 2 | AD-401824 | | IT | | Female | (qPCR) |
| 3 | AD-1427062 | | dose | | 6-8 wks | CNS: Brain |
| 4 | AD-2700143 | | | | | (from right |
| | | | | | | hemisphere): |
| | | | | | | Frontal cortex, |
| | | | | | | cerebellum, |
| | | | | | | hippocampus, |
| | | | | | | striatum |
| | | | | | | Thoracic cord |
| | | | | | | Periphery: |
| | | | | | | liver, heart |

Quantitative PCR analysis was performed using a Roche Cycler 480 ii system from Roche Diagnostics Indianapolis, IN 46256 (Catalog No. 05 015 243 001) with qPCR Master Mix (Roche, 04887301001), pre-validated superoxide dismutase 1, soluble (SOD1) (Rn00566938_m1), rat peptidylprolyl isomerase B (Ppib) (Rn03302274_m1), and primer for Rat GAPD (GAPDH) Endogenous Control (Applied Biosystems Catalog No. 4351317) Taqman Gene Expression Assays (Thermo Fisher Scientific).

An exemplary siRNA comprising a C₂₂ hydrocarbon chain conjugated to the sense strand at position 6, counting from the 5′-end of the sense strand, targeting SOD1, was used in this example at 0.6 mg. Comparative siRNAs comprising a C₁₆ hydrocarbon chain (SS6 C16, Uhd; and SS6 C₆-C₁₆—OH, Y505) conjugated to the sense strand at position 6, counting from the 5′-end of the sense strand, targeting the same CNS targets, were used in this example at the same dosage level as the exemplary siRNAs. The sequences used are listed in Table 12 below.

TABLE 12
siRNAs sequences targeting SOD1 (having a lipophilic moiety), used in in vivo
evaluation in rats after a single intrathecal injection

| | | | | SEQ | Molecular | Molecular | Lipid |
|---|---|---|---|---|---|---|---|
| Duplex | Oligo | | | ID | Weight | Weight | Chem- |
| ID | ID | Strand | Oligonucleotide Sequence | NO: | (cal) | (Found) | istry |
| AD- | A- | sense | csasuuu(Uhd)AfaUfCfCfucacu | 49 | 7043.976 | 7040.254 | SS6 C16 |
| 401824 | 637448 | | cuasasa | | | | |
| | A- | antis | VPusUfsuagAfgUfGfaggaUfu | 50 | 7851.156 | 7847.154 | |
| | 444402 | | Afaaaugsasg | | | | |
| AD- | A- | sense | csasuuu(Uda)AfaUfCfCfucacu | 51 | 7128.132 | 7124.348 | SS6 C22 |
| 1427062 | 2578434 | | cuasasa | | | | |
| | A- | antis | VPusUfsuagAfgUfGfaggaUfu | 50 | 7851.156 | 7847.154 | |
| | 444402 | | Afaaaugsasg | | | | |
| AD- | A- | sense | csasuuuY505AfaUfCfCfucacu | 168 | 7173.139 | 7169.333 | SS6 C6- |
| 2700143 | 4985613 | | cuasasa | | | | C16-OH |
| | A- | antis | VPusUfsuagAfgUfGfaggaUfu | 50 | 7851.156 | 7847.154 | |
| | 444402 | | Afaaaugsasg | | | | |

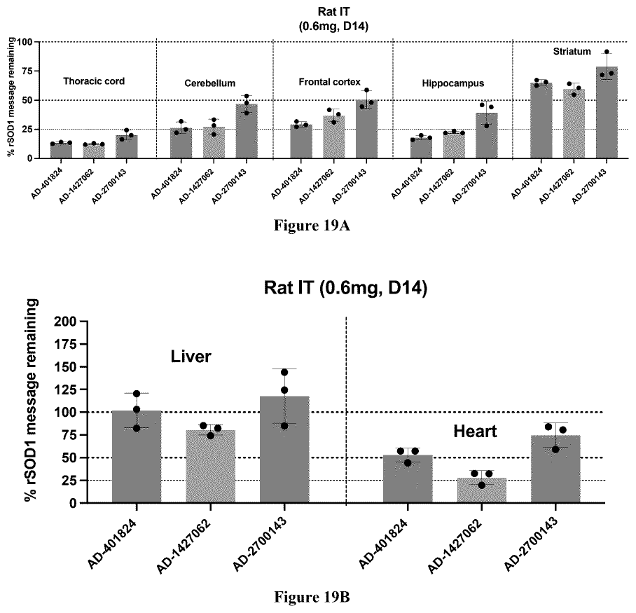

The results from in vivo silencing studies via rat IT are summarized in FIGS. 19A-19B. As shown in the figures, similar silencing activities in CNS tissue were observed for the siRNA duplexes having an internal lipid conjugation; C22-conjugated siRNA duplexes (SS6 C22) exhibited activities comparable to the C16-conjugated siRNA (SS6-C16). The siRNA duplexes having an internal conjugation of C16-OH (SS6 C₆-C₁₆—OH) had the least activities across CNS tissue and periphery tissues. C22-conjugated siRNA duplexes at an internal position exhibited an increased activities as compared to C16-conjugated siRNA duplexes in the periphery tissues, especially in heart. For instance, the siRNA duplexes having an internal conjugation of C22 showed 75% KD in the rat heart 14 days post 0.6 mg IT dose.

### Example 16: Evaluation of siRNA Containing Terminal Lipid Conjugation (Rat IT)

An SAR of 3′-terminal and 5′-terminal conjugation of a lipophilic moiety (C16 or C22) to an siRNA, targeting SOD1, was evaluated in CNS, liver, and heart via rat IT at a mid-range dose.

In vivo studies were performed in rat (Sprague Dawley Rat, aged 6-8 weeks; N=3) dosed via IT (intrathecal administration). The in vivo evaluation protocol and dosage regime for Rat IT are shown in the chart below. The siRNAs formulated at 0.6 mg per dose in artificial cerebrospinal fluid (aCSF), according to the chart below, were administered as 30 μL surgical IT injections in single dose, to female Sprague Dawley rat (aged 6-8 weeks; N=3). The tissues from CNS (striatum, frontal cortex, cerebellum, and hippocampus) and spine (thoracic cord) were collected on D15. The results of the mRNA knockdown for these various targets in rat in various tissues were analyzed. The controls were aCSF without siRNAs administrations.

| | Duplex | | | Route of | | | Tissue |
|---|---|---|---|---|---|---|---|
| Group | ID | Lipid Chemistry | Dose | Admin | Duration | Animals | Collection |
| 1 | aCSF | | — | D0 | D14 | SD Rat | Flash frozen |
| 2 | AD- | | 0.6 mg in | IT dose | | Female | (qPCR) |
| | 2700130 | | 30 ul (20 | | | 6-8 wks | CNS: Brain |
| 3 | AD- | 3′ C6 + C16 − | mg/ml) | | | | (from right |
| | 2700131 | L54 | | | | | hemisphere): |
| 4 | AD-953561 | 3′ C16 − L321 | | | | | Frontal cortex, |
| | | | | | | | cerebellum, |
| 5 | AD- | 5′ C16 (Alny) − | | | | | hippocampus, |
| | 2700132 | Q447 | | | | | striatum |
| 6 | AD- | 5′ C22 (Alny) − | | | | | Thoracic cord |
| | 2700133 | Q448 | | | | | Periphery: |
| 7 | AD- | 5′ aminolinker + | | | | | liver, heart |
| | 2700134 | C16 − Q466 | | | | | |
| 8 | AD- | 5′ aminolinker + | | | | | |
| | 2700135 | C16OH − | | | | | |
| | | Q478 | | | | | |
| 9 | AD- | 5′ aminolinker + | | | | | |
| | 2700136 | C22 − Q483 | | | | | |
| 10 | AD-401824 | Internal C16 | | | | | |
| | | Control | | | | | |

Quantitative PCR analysis was performed using a Roche Cycler 480 ii system from Roche Diagnostics Indianapolis, IN 46256 (Catalog No. 05 015 243 001) with qPCR Master Mix (Roche, 04887301001), pre-validated superoxide dismutase 1, soluble (SOD1) (Rn00566938_m1), rat peptidylprolyl isomerase B (Ppib) (Rn03302274_m1), and primer for Rat GAPD (GAPDH) Endogenous Control (Applied Biosystems Catalog No. 4351317) Taqman Gene Expression Assays (Thermo Fisher Scientific).

An exemplary siRNA comprising a C₂₂ hydrocarbon chain conjugated to the sense strand at position 6, counting from the 5′-end of the sense strand, targeting SOD C, was used in this example at 0.6 mg. Comparative siRNAs comprising a C₁₆ hydrocarbon chain (SS6 C16, Uhd; SS6 C₆-C₁₆—OH, Y505) as well as siRNAs comprising a C3′, C₁₇-C₂₁, and C23-C24) hydrocarbon chain conjugated to the sense strand at position 6, counting from the 5′-end of the sense strand, targeting the same CNS targets, were used in this example at the same dosage level as the exemplary siRNAs. The sequences used are listed in Table 13 below.

TABLE 13
siRNAs sequences targeting SOD1 (having a lipophilic moiety), used in in vivo
evaluation in rats after a single intrathecal injection

| | | | | SEQ | Molecular | Molecular | Lipid |
|---|---|---|---|---|---|---|---|
| Duplex | Oligo | | | ID | Weight | Weight | Chem- |
| ID | ID | Strand | Oligonucleotide Sequence | NO: | (cal) | (Found) | istry |
| AD- | A- | sense | csasuuuuAfaUfCfCfucacu | 169 | 7448.411 | 7444.462 | 3′ Hyp- |
| 2700130 | 4985616 | | cuaasasL58 | | | | C6-C22 |
| | A- | antis | VPusUfsuagAfgUfGfagga | 50 | 7851.156 | 7847.154 | |
| | 444402 | | UfuAfaaaugsasg | | | | |
| AD- | A- | sense | csasuuuuAfaUfCfCfucacu | 170 | 7364.251 | 7360.368 | 3′ Hyp- |
| 2700131 | 4985676 | | cuaasasL54 | | | | C6-C16 |
| | A- | antis | VPusUfsuagAfgUfGfagga | 50 | 7851.156 | 7847.154 | |
| | 444402 | | UfuAfaaaugsasg | | | | |
| AD- | A- | sense | csasuuuuAfaUfCfCfucacu | 171 | 7251.109 | 7247.284 | 3′ Hyp- |
| 953561 | 1700503 | | cuaasasL321 | | | | C16 |
| | A- | antis | VPusUfsuagAfgUfGfagga | 50 | 7851.156 | 7847.154 | |
| | 444402 | | UfuAfaaaugsasg | | | | |
| AD- | A- | sense | Q447scsauuuuAfaUfCfCfu | 172 | 7334.232 | 7330.358 | 5′ p-C6- |
| 2700132 | 4985617 | | cacucuasasa | | | | C16 |
| | A- | | VPusUfsuagAfgUfGfagga | 50 | 7851.156 | 7847.154 | |
| | 444402 | antis | UfuAfaaaugsasg | | | | |
| AD- | A- | sense | Q448scsauuuuAfaUfCfCfu | 173 | 7418.394 | 7414.452 | 5′ p-C6- |
| 2700133 | 4985618 | | cacucuasasa | | | | C22 |
| | A- | antis | VPusUfsuagAfgUfGfagga | 50 | 7851.156 | 7847.154 | |
| | 444402 | | UfuAfaaaugsasg | | | | |
| AD- | A- | sense | Q466scsauuuuAfaUfCfCfu | 174 | 7252.15 | 7248.328 | 5′ C6-C16 |
| 2700134 | 4962135 | | cacucuasasa | | | | |
| | A- | antis | VPusUfsuagAfgUfGfagga | 50 | 7851.156 | 7847.154 | |
| | 444402 | | UfuAfaaaugsasg | | | | |
| AD- | A- | sense | Q478scsauuuuAfaUfCfCfu | 175 | 7268.149 | 7264.323 | 5′ C6- |
| 2700135 | 4962137 | | cacucuasasa | | | | C16-OH |
| | A- | antis | VPusUfsuagAfgUfGfagga | 50 | 7851.156 | 7847.154 | |
| | 444402 | | UfuAfaaaugsasg | | | | |
| AD- | A- | sense | Q483scsauuuuAfaUfCfCfu | 176 | 7336.312 | 7332.422 | 5′ C6-C22 |
| 2700136 | 4985614 | | cacucuasasa | | | | |
| | A- | antis | VPusUfsuagAfgUfGfagga | 50 | 7851.156 | 7847.154 | |
| | 444402 | | UfuAfaaaugsasg | | | | |
| AD- | A- | sense | csasuuu(Uhd)AfaUfCfCfu | 49 | 7043.976 | 7040.254 | SS6 C16 |
| 401824 | 637448 | | cacucuasasa | | | | |
| | A- | antis | VPusUfsuagAfgUfGfagga | 50 | 7851.156 | 7847.154 | |
| | 444402 | | UfuAfaaaugsasg | | | | |

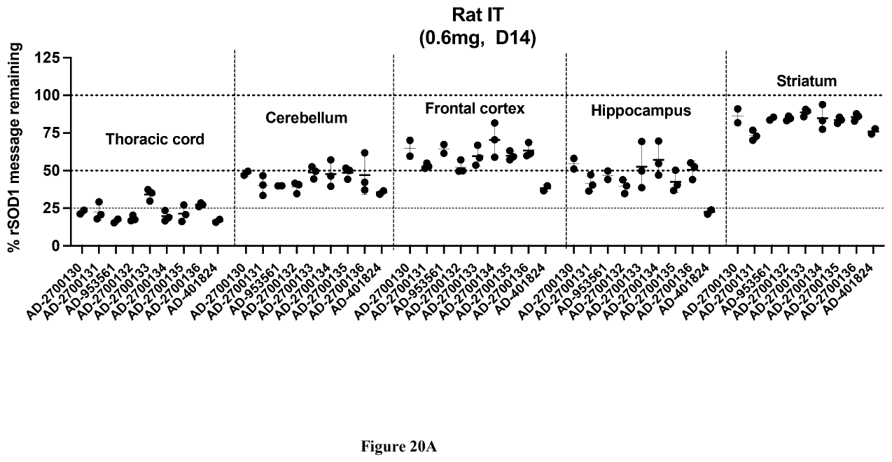

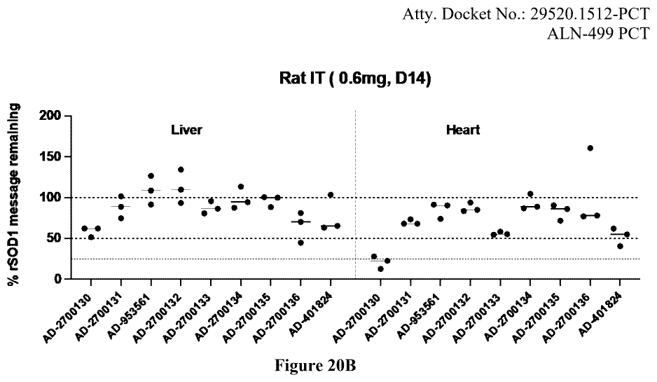

The results from in vivo silencing studies via rat IT are summarized in FIGS. 20A-20B. As shown in the figures, similar silencing activities in CNS tissue were observed for most of the siRNA duplexes having a terminal lipid conjugation of C16 or C22. In periphery tissues, the siRNA duplex having a 3′-terminal conjugation of a lipophilic moiety containing C22 (3′ C6+C22−L58) exhibited robust activity in the heart (>75% KD) and also had the most activity in liver (42% KD) 14 days post 0.6 mg IT dose. The siRNA duplex having a 5′-terminal conjugation of a lipophilic moiety containing C22 (5′ C22 (Alny)-Q448) exhibited robust activity in the rat heart (~50% KD) 14 days post 0.6 mg IT dose.

### Example 17. PN-Lipid Modification of siRNA Duplexes

Oligonucleotides containing one of the PN linkages, phosphorodiamidate, have been well-studied. Oligonucleotides containing other types of PN-linkage chemistries have been reported. See Letsinger et al., “Cationic oligonucleotides,” J Am Chem Soc. 1988; 110(13):4470-1; Maier et al., “Synthesis of Chimeric Oligonucleotides Containing Phosphodiester, Phosphorothioate, and Phosphoramidate Linkages,” Org Lett. 2000; 2(13):1819-22; Vlaho et al., “Structural Studies and Gene Silencing Activity of siRNAs Containing Cationic Phosphoramidate Linkages,” Nucleic Acid Ther. 2018; 28(1):34-43, which are incorporated by reference in their entirety.

For instance, the PN-lipids can be conjugated to the siRNA duplex via H-phosphonate chemistry illustrated in the scheme shown below.

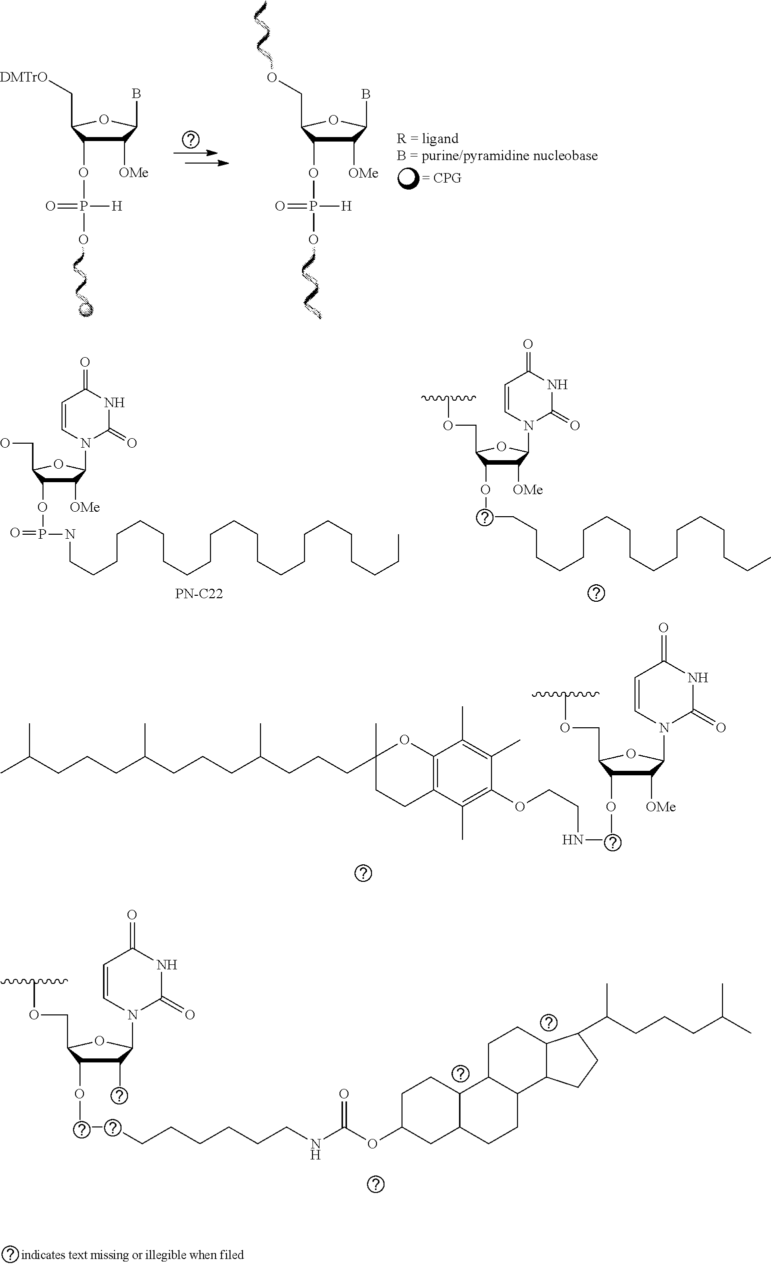

Synthesis of PN Linkages—Dimer Approach (UU Dimers with PN Linkages)

Synthesis oligonucleotides containing PN linkages by a dimer approach can be found in Asseline et al., “Synthesis and properties of oligo-2′-deoxyribonucleotides containing internucleotidic phosphoramidate linkages modified with pendant groups ending with either two amino or two hydroxyl functions,” Bioorg Med Chem. 2003; 11(16):3499-511; Seela et al., “Diastereomerically pure Rp and Sp dinucleoside H-phosphonates: the stereochemical course of their conversion into P-methylphosphonates, phosphorothioates, and [oxygen-18] chiral phosphates,” The Journal of Organic Chemistry. 1991; 56(12):3861-9, which are incorporated by reference in their entirety.

These approaches can be used to prepare racemic PN linkages and chiral PN linkages discussed below.

### Racemic PN Linkages and Chiral PN Linkages

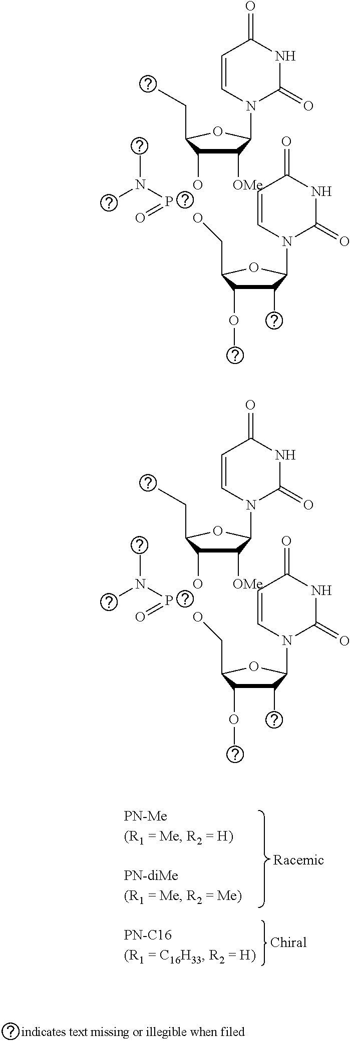

In the above scheme, PN-C22 racemic or chiral forms can be similarly prepared based on the methods described herein when R₁═C₂₂H₄₅, R₂═H.

Variations of R₁ and/or R₂ in the above PN-linkages may lead to improvement of RNAi potency and biodistribution for extrahepatic application.

Synthesis of alkylamines (e.g., MeNH₂, Me₂NH and C₁₆H₃₃NH₂) are illustrated in the following reactions.

### Synthesis of PN-C16

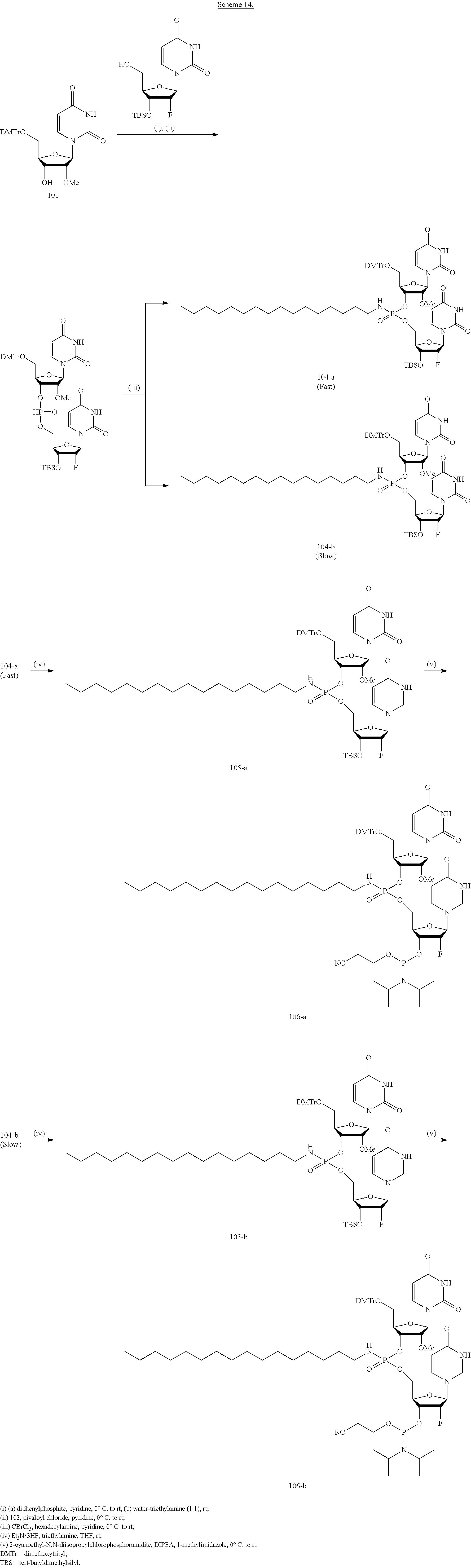

Synthesis of compound 103. (a) To a solution of compound 101 (2.5 g, 4.46 mmol) in pyridine (40 mL) was added dropwise diphenylphosphate (2.57 mL, 13.4 mL) at 0° C. The mixture was stirred at room temperature for 1 hour, and then was quenched by addition of the mixture of water-triethylamine (1:1 v/v, 20 mL) and was left standing for 15 minutes. The solvent was evaporated under vacuum and the residue was partitioned between CH₂Cl₂ and 5% aqueous NaHCO₃ and the aqueous layer was extracted with CH₂C2. The combined organic layer was washed with brine and dried over Na₂SO₄ and concentrated under vacuum. The crude residue was purified by column chromatography on silica gel (0-50% MeOH in CH₂Cl₂) to obtain the H-phosphonate intermediate as a Et₃N salt. (b) The obtained fractions were collected and concentrated under vacuum. The resulting residue and compound 102 (1.61 g, 4.46 mmol) were dissolved in pyridine (40 mL) and the mixture was cooled to 0° C. Pivaloyl chloride (0.819 mL, 6.69 mmol) was added dropwise to the mixture and the mixture was stirred at rt for 1 h. The solvent was evaporated under vacuum and the residue was partitioned between ethyl acetate and saturated aqueous NaHCO₃ and the organic layer was washed with water and brine and dried with Na₂SO₄ and concentrated under vacuum. The crude residue was purified by column chromatography on silica gel (50-90% ethyl acetate (5% MeOH) in hexane) to obtain a diastereomeric mixture of compound 103 as a white form (2.86 g, 66%). ¹H NMR (600 MHz, CD₃CN) δ 9.53-9.51 (m, 2H), 7.66-7.60 (m, 1H), 7.46-7.23 (m, 10H), 6.88-6.87 (m, 4H), 5.92-5.89 (m, 1H), 5.85-5.76 (m, 1H), 5.66-5.59 (m, 1H), 5.37-5.36 (m, 1H), 5.12-4.98 (m, 2H), 4.47-4.06 (m, 7H), 3.76 (s, 6H), 3.49 (s, 3H), 3.43-3.36 (m, 2H), 0.91-0.89 (m, 9H) 0.13-0.11 (m, 6H) ppm. ¹³C NMR (151 MHz, CD₃CN) δ 163.74, 163.72, 163.59, 163.55, 159.38, 159.37, 151.11, 151.07, 150.81, 150.80, 150.79, 145.15, 145.13, 141.92, 141.47, 140.53, 135.93, 135.89, 135.77, 135.74, 130.69, 130.66, 128.60, 128.57, 127.65, 113.79, 113.77, 102.82, 102.65, 102.62, 102.60, 102.52, 93.62, 93.54, 92.37, 92.29, 90.87, 90.64, 90.28, 90.04, 87.60, 87.48, 87.46, 87.18, 82.50, 82.46, 82.30, 82.29, 82.25, 82.24, 82.19, 82.14, 81.54, 81.50, 81.47, 81.42, 74.29, 74.25, 72.98, 72.94, 69.74, 69.71, 69.65, 69.63, 69.60, 64.24, 64.20, 63.65, 63.61, 62.57, 62.23, 58.98, 58.71, 55.51, 25.56, 18.19, −5.01, −5.03, −5.31 ppm. ¹⁹F NMR (565 MHz, CD₃CN) δ −200.51, −200.54, −200.58, −200.60, −200.64, −200.67, −201.26, −201.29, −201.33, −201.35, −201.39, −201.42. ³¹P NMR (243 MHz, CD₃CN) δ 11.22, 11.19, 11.15, 11.12, 11.09, 9.72, 9.69, 9.66, 9.62, 9.41, 9.38, 9.34, 9.31, 8.26, 8.22, 8.19, 8.15, 8.12, 6.70, 6.66, 6.63, 6.60, 6.41, 6.38, 6.34, 6.31. HRMS calc. for C₄₆H₅₆FN₄NaO₁₄ [M+Na]⁺ 989.3182, found 989.3163.

Synthesis of compound 104-a and 104-b. To a vigorously stirred suspension of compound 3 (3.5 g, 3.62 mmol) and hexadecylamine (6.12 g, 25.3 mmol) in pyridine (18 mL) was added CBrCl₃ (18 mL) at 0° C. and the mixture was stirred at room temperature for 3 hours. The reaction mixture was concentrated under vacuum and the residue was partitioned between ethyl acetate and saturated aqueous NH₄Cl and filtered through a short silica gel pad to remove insoluble matter. The organic layer was washed with water and dried over Na₂SO₄ and concentrated under vacuum. The crude residue was purified by column chromatography on silica gel (35-80% ethyl acetate (5% MeOH) in hexane) to obtain fast eluting compound 104-a as a light brawn form (600 mg, 13.8%), slow eluting compound 104-b as a light brawn form (1.25 g, 28.6%) and a mixture of compounds 104-a and 104-b (961 mg, 22%) as a light brawn form.

Compound 104-a: ¹H NMR (400 MHz, DMSO-d₆) δ 11.45-11.43 (m, 2H), 7.72 (d, J=8.1 Hz, 1H), 7.58 (d, J=8.1 Hz, 1H), 7.39-7.20 (m, 9H), 6.88-6.86 (m, 4H), 5.85-5.79 (m, 2H), 5.56 (dd, J=8.1, 2.2 Hz, 1H), 5.28 (dd, J=8.1, 2.2 Hz, 1H), 5.23-5.08 (m, 2H), 4.87-4.83 (m, 1H), 4.34-4.26 (m, 1H), 4.22-4.19 (m, 1H), 4.09-3.96 (m, 4H), 3.72 (s, 6H), 3.40 (s, 3H), 3.38-3.34 (m, 1H), 3.32-3.28 (m, 1H), 2.77-2.69 (m, 2H), 1.39-1.19 (m, 29H), 0.85-0.81 (m, 12H) 0.08-0.08 (m, 6H) ppm. ¹³C NMR (126 MHz, DMSO-d₆) δ 163.05, 162.82, 158.16, 150.28, 150.13, 144.40, 140.85, 139.93, 135.12, 134.86, 129.80, 129.78, 127.83, 127.73, 126.77, 113.17, 101.81, 92.73, 91.24, 88.82, 88.54, 86.34, 86.15, 81.40, 81.36, 80.89, 80.83, 80.59, 80.56, 71.62, 71.58, 69.29, 69.17, 64.23, 61.97, 57.74, 54.96, 54.95, 54.85, 40.75, 31.27, 31.22, 31.17, 29.03, 28.98, 28.97, 28.73, 28.68, 26.16, 25.45, 22.06, 17.61, 13.88, −5.04, −5.31 ppm. ¹⁹F NMR (376 MHz, DMSO-d₆) δ −200.75, −200.80, −200.85, −200.89, −200.94, −200.99. ³¹P NMR (162 MHz, DMSO-d₆) δ 10.77. HRMS calc. for C₆₂H₈₉FN₅NaO₁₄ Psi [M+Na]⁺ 1228.5795, found 1228.5850.

Compound 104-b: ¹H NMR (600 MHz, DMSO-d₆) δ 11.44 (brs, 2H), 7.69 (d, J=8.1 Hz, 1H), 7.65 (d, J=8.1 Hz, 1H), 7.38-7.22 (m, 9H), 6.89-6.87 (m, 4H), 5.88-5.84 (m, 2H), 5.59 (d, J=8.1 Hz, 1H), 5.36 (d, J=8.1 Hz, 1H), 5.23-5.12 (m, 2H), 4.80-4.77 (m, 1H), 4.33-4.22 (m, 3H), 4.12-4.02 (m, 3H), 3.73 (s, 6H), 3.40 (s, 3H), 3.35-3.32 (m, 1H), 3.30-3.27 (m, 1H), 2.63-2.58 (m, 2H), 1.28-1.14 (m, 29H), 0.86-0.83 (m, 12H) 0.09 (s, 6H) ppm. ¹³C NMR (151 MHz, DMSO-d₆) δ 163.62, 163.30, 158.65, 150.84, 150.64, 144.92, 141.08, 140.61, 135.62, 135.43, 130.28, 130.27, 128.34, 128.22, 127.30, 113.66, 102.43, 102.21, 93.20, 91.96, 89.12, 88.89, 86.86, 86.55, 82.05, 82.01, 81.20, 81.14, 80.89, 80.87, 72.06, 72.03, 69.39, 69.28, 64.15, 63.13, 58.26, 55.47, 41.19, 31.76, 31.67, 31.63, 29.52, 29.51, 29.48, 29.46, 29.43, 29.18, 29.15, 26.56, 25.95, 22.57, 18.13, 14.41, −4.55, −4.86 ppm. ¹⁹F NMR (565 MHz, DMSO-d₆) δ −201.09, −201.12, −201.16, −201.18, −201.22, −201.25. ³¹P NMR (243 MHz, DMSO-d₆) δ 10.01. HRMS calc. for C₆₂H₈₉FN₅NaO₁₄ Psi [M+Na]⁺ 1228.5795, found 1228.5793.

Synthesis of compound 105-a. To a solution of compound 104-a (600 mg, 0.497 mmol) and triethylamine (0.208 mL, 1.49 mmol) in THE (5 mL) was added dropwise triethylamine trihydrofluoride (0.243 mL, 1.49 mmol). The mixture was stirred at room temperature overnight and concentrated under vacuum. The crude residue was purified by column chromatography on silica gel (0-5% MeOH in ethyl acetate) to obtain compound 105-a as a white form (392 mg, 72%). ¹H NMR (600 MHz, DMSO-d₆) δ 11.46 (brs, 1H), 11.43 (brs, 1H), 7.72 (d, J=8.1 Hz, 1H), 7.58 (d, J=8.1 Hz, 1H), 7.39-7.23 (m, 9H), 6.90-6.88 (m, 4H), 5.85-5.82 (m, 2H), 5.79 (d, J=6.4 Hz, 1H), 5.55 (dd, J=8.1, 1.9 Hz, 1H), 5.30 (dd, J=8.1, 1.9 Hz, 1H), 5.18-5.04 (m, 2H), 4.86-4.83 (m, 1H), 4.23-3.96 (m, 6H), 3.74 (s, 6H), 3.41 (s, 3H), 3.37-3.34 (m, 1H), 3.31-3.29 (m, 1H), 2.76-2.70 (m, 2H), 1.39-1.35 (m, 2H), 1.27-1.19 (m, 27H), 0.86-0.84 (m, 3H) ppm. ¹³C NMR (151 MHz, DMSO-d₆) δ 163.51, 163.34, 158.64, 158.62, 150.78, 150.59, 144.90, 141.27, 140.48, 135.61, 135.36, 130.27, 128.37, 128.22, 127.29, 113.70, 102.28, 94.02, 92.79, 89.08, 88.85, 86.86, 86.62, 81.88, 81.84, 81.24, 81.19, 81.07, 81.05, 72.03, 71.99, 68.59, 68.48, 65.26, 65.23, 62.44, 58.22, 55.48, 55.47, 41.22, 31.77, 31.66, 31.62, 29.53, 29.49, 29.47, 29.22, 29.19, 26.65, 22.57, 14.42 ppm. ¹⁹F NMR (565 MHz, DMSO-d₆) δ −200.47, −200.51, −200.54, −200.56, −200.60, −200.64. ³¹P NMR (243 MHz, DMSO-d₆) δ 10.40. HRMS calc. for C₅₆H₇₅FN₅NaO₁₄P [M+Na]⁺ 1114.4930, found 1114.4968.

Synthesis of compound 105-b. To a solution of compound 104-b (1 g, 0.829 mmol) and triethylamine (0.347 mL, 2.49 mmol) in THE (8 mL) was added dropwise triethylamine trihydrofluoride (0.405 mL, 2.49 mmol). The mixture was stirred at room temperature overnight and concentrated under vacuum. The crude residue was purified by column chromatography on silica gel (0-5% MeOH in ethyl acetate) to obtain compound 105-b as a white form (634 mg, 70%). ¹H NMR (400 MHz, DMSO-d₆) δ 11.46-11.43 (m, 2H), 7.69-7.62 (m, 2H), 7.38-7.22 (m, 9H), 6.90-6.88 (m, 4H), 5.91-5.85 (m, 2H), 5.79 (d, J=6.4 Hz, 1H), 5.57 (dd, J=8.1, 2.2 Hz, 1H), 5.36 (dd, J=8.1, 2.2 Hz, 1H), 5.19-5.02 (m, 2H), 4.83-4.79 (m, 1H), 4.26-4.03 (m, 6H), 3.73 (s, 6H), 3.40 (s, 3H), 3.34-3.27 (m, 2H), 2.66-2.58 (m, 2H), 1.28-1.14 (m, 29H), 0.86-0.83 (m, 3H) ppm. ¹³C NMR (126 MHz, DMSO-d₆) δ 163.23, 163.02, 158.38, 158.36, 150.46, 150.27, 144.56, 140.74, 140.40, 135.30, 135.13, 129.98, 128.08, 127.92, 127.05, 113.40, 102.13, 102.03, 93.81, 92.33, 88.53, 88.25, 86.60, 86.35, 81.78, 81.73, 80.96, 80.90, 80.68, 71.96, 68.10, 67.97, 64.78, 62.70, 57.98, 55.19, 40.72, 31.44, 31.31, 31.26, 29.19, 29.17, 29.15, 29.12, 29.10, 28.85, 28.82, 26.20, 22.25, 14.08 ppm. 19F NMR (376 MHz, DMSO-d₆) δ −200.99, −201.05, −201.10, −201.14, −201.19, −201.24. ³¹P NMR (162 MHz, DMSO-d₆) δ 10.63. HRMS calc. for C₅₆H₇₅FN₅NaO₁₄P [M+Na]⁺ 1114.4930, found 1114.4972.

Synthesis of compound 106-a. To a solution of compound 105-a (500 mg, 0.458 mmol), 1-methylimidazole (3.65 μL, 45.8 umol) and DIPEA (0.239 mL, 1.37 mmol) in CH₂Cl₂ (5 mL) was added dropwise 2-cyanoethyl N,N-diisopropylchlorophosphoramidite (0.123 mL, 0.549 mmol) at 0° C. and the mixture was stirred at 0° C. for 3 hours. The reaction mixture was diluted with ethyl acetate and washed with saturated NaHCO₃ (aq.), water, brine, dried (Na₂SO₄) and concentrated under vacuum. The crude residue was purified by column chromatography on silica gel (100% ethyl acetate in hexane) to obtain compound 106-a as a white form (418 mg, 71%). ¹H NMR (400 MHz, CD₃CN) δ 9.43 (brs, 2H), 7.75-7.73 (m, 1H), 7.45-7.42 (m, 2H), 7.37-7.22 (m, 8H), 6.89-6.86 (m, 4H), 5.88-5.87 (m, 1H), 5.73-5.68 (m, 1H). 5.53-5.50 (m, 1H), 5.24-5.22 (m, 1H), 5.20-5.03 (m, 1H), 5.02-4.96 (m, 1H), 4.50-4.31 (m, 1H), 4.27-4.02 (m, 5H), 3.82-3.61 (m, 10H), 3.50-3.40 (m, 5H), 2.91-2.85 (m, 2H), 2.66-2.62 (m, 2H), 1.46-1.44 (m, 2H), 1.30-1.21 (m, 27H), 1.18-1.15 (m, 12H), 0.89-0.85 (m, 3H) ppm. ¹³C NMR (126 MHz, CD₃CN) δ 164.11, 164.09, 164.08, 159.88, 159.85, 159.83, 151.56, 151.20, 151.18, 145.71, 141.98, 141.91, 140.94, 136.43, 136.30, 131.30, 131.23, 129.21, 129.06, 128.12, 119.60, 114.29, 114.26, 103.06, 103.00, 102.97, 94.48, 93.94, 92.99, 92.41, 91.32, 91.13, 91.04, 90.84, 87.99, 87.95, 87.92, 87.90, 82.95, 82.92, 82.76, 82.71, 81.59, 81.20, 73.08, 73.04, 70.83, 70.77, 70.70, 70.65, 70.56, 65.44, 65.27, 62.66, 62.51, 59.94, 59.79, 59.64, 59.30, 59.25, 59.08, 59.05, 56.03, 47.32, 47.25, 46.70, 46.66, 46.11, 46.06, 44.35, 44.26, 42.32, 42.30, 42.20, 32.73, 32.63, 32.60, 32.58, 32.55, 30.49, 30.45, 30.44, 30.17, 27.55, 27.53, 25.15, 25.09, 25.05, 25.02, 24.99, 24.96, 24.93, 23.87, 23.48, 23.27, 23.25, 23.21, 23.19, 23.05, 22.91, 22.73, 22.66, 21.06, 21.01, 20.70, 20.65, 20.44, 14.50 ppm. ¹⁹F NMR (376 MHz, CD₃CN) δ −198.61, −198.64, −198.66, −198.69, −198.72, −198.75, −198.78, −198.80, −198.83, −198.86, −198.88, −199.37, −199.38, −199.42, −199.44, −199.47, −199.49, −199.51, −199.53, −199.56, −199.58, −199.61, −199.63. ³¹P NMR (162 MHz, CD₃CN) δ 151.48, 151.44, 151.27, 151.22, 10.60, 10.51. HRMS calc. for C₆₅H₉₂FN₇NaO₁₅P₂ [M+Na]⁺ 1314.6008, found 1314.6055.

Synthesis of compound 106-b. To a solution of compound 105-b (500 mg, 0.458 mmol), 1-methylimidazole (3.65 μL, 45.8 umol) and DIPEA (0.239 mL, 1.37 mmol) in CH₂Cl₂ (5 mL) was added dropwise 2-cyanoethyl N,N-diisopropylchlorophosphoramidite (0.123 mL, 0.549 mmol) at 0° C. and the mixture was stirred at 0° C. for 3 hours. The reaction mixture was diluted with ethyl acetate and washed with saturated NaHCO₃ (aq.), water, brine, dried (Na₂SO₄) and concentrated under vacuum. The crude residue was purified by column chromatography on silica gel (100% ethyl acetate in hexane) to obtain compound 106-b as a white form (422 mg, 71%). ¹H NMR (400 MHz, CD₃CN) δ 9.95 (brs, 2H), 7.76-7.72 (m, 1H), 7.61-7.58 (m, 1H), 7.43-7.23 (m, 9H), 6.87-6.85 (m, 4H), 5.92-5.87 (m, 2H), 5.63-5.60 (m, 1H), 5.24-5.04 (m, 2H), 4.98-4.91 (m, 1H), 4.45-4.39 (m, 2H), 4.28-4.18 (m, 2H), 4.07-4.04 (m, 1H), 3.96-3.91 (m, 1H), 3.83-3.72 (m, 8H), 3.66-3.60 (m, 2H), 3.52-3.49 (m, 4H), 3.44-3.39 (m, 1H), 2.71-2.62 (m, 4H), 1.34-1.15 (m, 41H), 0.88-0.84 (m, 3H) ppm. ¹³C NMR (101 MHz, CD₃CN) δ 164.40, 164.37, 164.34, 159.87, 159.85, 151.49, 151.44, 151.37, 151.33, 145.64, 141.57, 141.35, 141.02, 136.33, 136.31, 136.21, 131.29, 131.24, 129.21, 129.03, 128.15, 119.60, 119.57, 114.22, 103.15, 103.01, 102.93, 102.87, 94.65, 94.08, 92.78, 92.18, 90.51, 90.33, 90.16, 89.99, 88.47, 88.37, 87.92, 87.90, 83.19, 82.49, 82.41, 82.31, 81.73, 81.24, 72.68, 72.52, 70.84, 70.68, 70.45, 65.30, 64.97, 62.80, 62.73, 59.91, 59.77, 59.72, 59.58, 59.22, 56.01, 46.08, 46.01, 44.35, 44.23, 42.08, 32.73, 30.49, 30.46, 30.43, 30.41, 30.17, 30.10, 27.40, 25.18, 25.11, 25.06, 25.03, 25.01, 24.99, 24.96, 24.93, 23.48, 21.07, 21.00, 14.54 ppm. ¹⁹F NMR (376 MHz, CD₃CN) δ −199.98, −200.01, −200.04, −200.06, −200.09, −200.11, −200.12, −200.15, −200.18, −200.20, −200.23, −200.25, −200.76, −200.78, −200.81, −200.83, −200.86, −200.88, −200.90, −200.92, −200.95, −200.97, −201.00, −201.02. ³¹P NMR (162 MHz, CD₃CN) δ 151.42, 151.37, 151.31, 10.08, 9.94. HRMS calc. for C₆₅H₉₂FN₇NaO₁₅P₂ [M+Na]⁺ 1314.6008, found 1314.6024.

PN-C22,

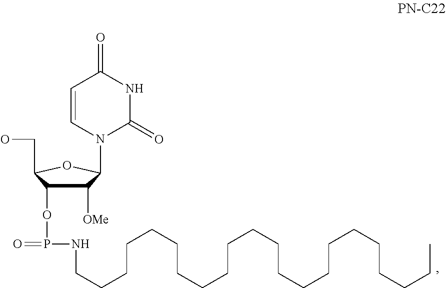

can be synthesized using the same method as discussed above used to synthesize PN-C16, substituting nC₂₂H₄₅NH₂ for C₁₆H₃₃NH₂.

### Synthesis of PN-Me

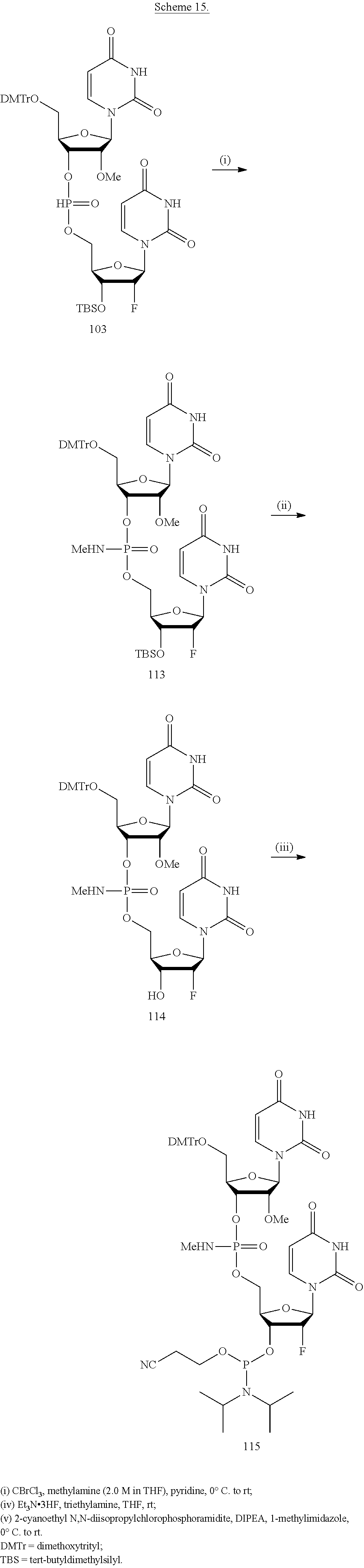

Synthesis of compound 113. To a solution of compound 103 (2 g, 2.07 mmol) and methylamine (2.0 M in THF; 5.2 mL, 10.3 mmol) in pyridine (10 mL) was added CBrCl₃ (10 mL) at 0° C. and the mixture was stirred at room temperature for 1 hour. The reaction mixture was concentrated under vacuum and the residue was partitioned between ethyl acetate and saturated aqueous NaHCO₃ and filtered to remove insoluble matter. The organic layer was washed with water and dried over Na₂SO₄ and concentrated under vacuum. The crude residue was purified by column chromatography on silica gel (50-90% ethyl acetate (5% MeOH) in hexane) to obtain a diastereomeric mixture of compound 113 as a white form (1.75 g, 85%). ¹H NMR (500 MHz, DMSO-d₆) δ 11.43-11.40 (m, 2H), 7.72-7.58 (m, 2H), 7.38-7.22 (m, 9H), 6.90-6.87 (m, 4H), 5.89-5.80 (m, 2H), 5.63-5.56 (m, 1H), 5.34-5.28 (m, 1H), 5.23-5.04 (m, 2H), 4.85-4.76 (m, 1H), 4.35-4.28 (m, 1H), 4.25-3.96 (m, 5H), 3.73-3.72 (m, 6H), 3.41-3.40 (m, 3H), 3.36-3.28 (m, 2H), 2.43-2.39 (m, 2H), 2.26-2.22 (m, 1H), 0.86-0.85 (m, 9H), 0.10-0.07 (m, 6H) ppm. ¹³C NMR (126 MHz, DMSO-d₆) δ 163.18, 163.14, 163.12, 162.85, 158.20, 158.17, 150.31, 150.28, 150.19, 150.17, 150.15, 144.40, 144.35, 140.86, 140.83, 140.60, 140.20, 139.99, 135.14, 135.11, 134.92, 134.86, 129.82, 129.79, 127.88, 127.79, 127.75, 126.86, 126.80, 113.22, 101.87, 101.84, 101.80, 101.77, 101.66, 92.89, 92.85, 92.76, 92.73, 91.40, 91.36, 91.27, 88.87, 88.81, 88.74, 88.59, 88.53, 88.46, 86.89, 86.45, 86.16, 86.11, 81.40, 81.36, 81.28, 81.23, 81.01, 80.95, 80.84, 80.77, 80.69, 80.67, 80.63, 80.57, 80.55, 71.47, 71.43, 71.38, 69.17, 69.14, 69.04, 68.92, 68.78, 68.65, 63.96, 63.92, 63.59, 63.44, 63.40, 62.38, 61.91, 57.91, 57.72, 55.02, 54.99, 26.73, 26.71, 25.48, 17.64, −4.97, −5.00, −5.02, −5.28, −5.33, −5.35 ppm. ¹⁹F NMR (471 MHz, DMSO-d₆) δ −201.06, −201.10, −201.14, −201.17, −201.21, −201.24, −201.26, −201.28, −201.32, −201.35, −201.39, −201.43. ³¹P NMR (202 MHz, DMSO-d₆) δ 9.54, 8.95. HRMS calc. for C₄₇H₅₉FN₅NaO₁₄ Psi [M+Na]⁺ 1018.3447, found 1018.3420.

Synthesis of compound 114. To a solution of compound 113 (1.5 g, 1.51 mmol) and triethylamine (0.630 mL, 4.52 mmol) in THE (15 mL) was added dropwise triethylamine trihydrofluoride (0.736 mL, 4.52 mmol). The mixture was stirred at room temperature for 24 hours and concentrated under vacuum. The crude residue was purified by column chromatography on silica gel (0-10% MeOH in CH₂Cl₂) to obtain a diastereomeric mixture of compounds 114 as a white form (945 mg, 71%). ¹H NMR (500 MHz, DMSO-d₆) δ 11.43-11.39 (m, 2H), 7.72-7.69 (m, 2H), 7.62 (d, J=9.7 Hz, 0.8H), 7.57 (d, J=9.7 Hz, 0.2H), 7.37-7.23 (m, 9H), 6.90-6.88 (m, 4H), 5.89-5.76 (m, 3H), 5.59-5.53 (m, 1H), 5.34-5.28 (m, 1H), 5.15-5.03 (m, 2H), 4.82-4.78 (m, 1H), 4.26-4.01 (m, 6H), 3.73 (s, 6H), 3.40 (s, 3H), 3.32-3.31 (m, 2H), 2.41 (dd, J=15.4, 6.6 Hz, 0.5H), 2.28 (dd, J=15.4, 6.6 Hz, 2.5H) ppm. ¹³C NMR (101 MHz, DMSO-d₆) δ 163.12, 163.09, 162.88, 158.21, 158.17, 150.35, 150.31, 150.18, 150.14, 144.39, 140.63, 140.26, 135.15, 134.94, 134.89, 129.83, 127.93, 127.80, 126.88, 113.27, 101.92, 101.80, 93.88, 92.04, 88.54, 88.19, 86.74, 86.16, 81.40, 81.34, 80.78, 80.71, 80.59, 71.55, 68.02, 67.86, 64.55, 62.35, 57.88, 57.73, 55.05, 55.02, 26.72 ppm. ¹⁹F NMR (471 MHz, DMSO-d₆) δ −200.82, −200.86, −200.90, −200.93, −200.97, −201.08, −201.13, −201.17, −201.20, −201.24, −201.28. ³¹P NMR (202 MHz, DMSO-d₆) δ 13.18, 12.84. HRMS calc. for C₄₁H₄₅FN₅NaO₁₄P [M+Na]⁺ 904.2582, found 904.2570.

Synthesis of compound 115. To a solution of compound 114 (800 mg, 0.907 mmol), 1-methylimidazole (7.23 μL, 90.7 mol) and DIPEA (0.474 mL, 2.72 mmol) in CH₂Cl₂ (9 mL) was added dropwise 2-cyanoethyl N,N-diisopropylchlorophosphoramidite (0.243 mL, 1.09 mmol) at 0° C. and the mixture was stirred at 0° C. for 3 hours. The reaction mixture was diluted with ethyl acetate and washed with saturated NaHCO₃ (aq.), water, brine, dried (Na₂SO₄) and concentrated under vacuum. The crude residue was purified by column chromatography on silica gel (100% ethyl acetate) to obtain a diastereomeric mixture of compound 115 as a white form (639 mg, 65%). ¹H NMR (500 MHz, CD₃CN) δ 9.55 (brs, 2H), 7.74-7.71 (m, 1H), 7.58 (t, J=16.1 Hz, 0.6H), 7.45-7.38 (m, 2.4H), 7.34-7.24 (m, 7H), 6.89-6.86 (m, 4H), 5.91-5.85 (m, 1.6H), 5.77-5.73 (m, 0.4H), 5.60 (dd, J=8.2, 2.4 Hz, 0.6H), 5.53 (d, J=8.2 Hz, 0.4H), 5.26-5.23 (m, 1H), 5.20-5.17 (m, 0.5H), 5.10-5.06 (m, 0.5H), 5.01-4.97 (m, 0.4H), 4.95-4.89 (m, 0.6H), 4.52-4.03 (m, 6H), 3.83-3.73 (m, 8H), 3.66-3.57 (m, 3H), 3.51-3.39 (m, 5H), 2.68-2.62 (m, 2H), 2.58-2.53 (m, 1.2H), 2.39-2.33 (m, 1.8H), 1.18-1.15 (m, 12H) ppm. ¹³C NMR (101 MHz, CD₃CN) δ 164.25, 164.21, 164.18, 164.15, 159.92, 159.91, 159.87, 159.84, 151.54, 151.48, 151.44, 151.32, 151.29, 151.21, 151.19, 145.70, 145.62, 141.93, 141.85, 141.62, 141.39, 141.06, 140.96, 136.43, 136.40, 136.37, 136.27, 136.24, 131.30, 131.28, 131.23, 129.24, 129.19, 129.06, 128.18, 128.11, 119.65, 114.28, 114.25, 103.05, 102.96, 102.91, 94.69, 94.11, 92.82, 92.25, 91.25, 91.10, 90.75, 90.64, 90.54, 90.28, 90.19, 88.63, 88.52, 87.99, 87.90, 87.87, 83.15, 82.86, 82.79, 82.73, 82.49, 82.42, 82.31, 81.68, 81.24, 72.95, 72.72, 72.68, 72.55, 70.71, 70.54, 70.38, 70.22, 65.12, 64.77, 62.79, 62.72, 62.49, 59.93, 59.74, 59.61, 59.23, 59.00, 56.04, 56.02, 44.35, 44.23, 27.84, 25.14, 25.10, 25.06, 25.04, 25.02, 24.97, 24.92, 24.90, 21.07, 21.00 ppm. ¹⁹F NMR (471 MHz, CD₃CN) δ −199.04, −199.06, −199.08, −199.10, −199.13, −199.15, −199.17, −199.19, −199.22, −199.24, −199.26, −199.76, −199.77, −199.80, −199.81, −199.84, −199.85, −199.87, −199.88, −199.91, −199.92, −199.95, −199.97, −200.23, −200.25, −200.27, −200.29, −200.31, −200.34, −200.36, −200.38, −200.40, −200.43, −200.45, −200.86, −200.87, −200.90, −200.91, −200.94, −200.96, −200.97, −200.99, −201.01, −201.03, −201.05, −201.07. ³¹P NMR (202 MHz, CD₃CN) δ 152.79, 152.77, 152.76, 152.73, 152.62, 152.58, 152.52, 152.47, 12.63, 12.53, 12.13, 11.94. HRMS calc. for C₅₀H₆₂FN₇NaO₁₅P₂ [M+Na]⁺ 1104.3661, found 1104.3656.

### Synthesis of PN-diMe

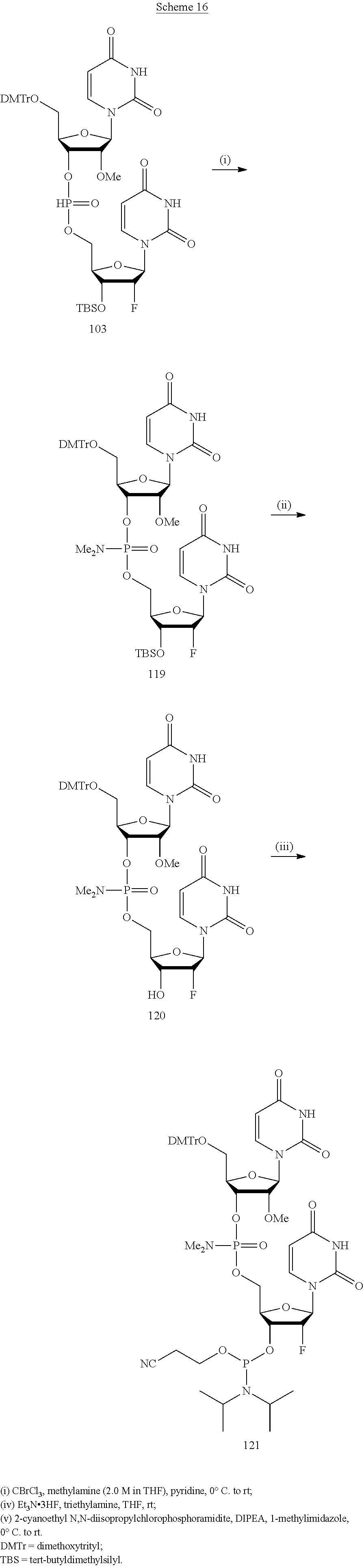

Synthesis of compound 119. To a solution of compound 103 (500 mg, 0.517 mmol) and dimethylamine (2.0 M in THF; 1.30 mL, 2.59 mmol) in pyridine (3 mL) was added CBrCl₃ (3 mL) at 0° C. and the mixture was stirred at room temperature for 1 hour. The reaction mixture was concentrated under vacuum and the residue was partitioned between ethyl acetate and saturated aqueous NaHCO₃ and filtered to remove insoluble matter. The organic layer was washed with water and dried over Na₂SO₄ and concentrated under vacuum. The crude residue was purified by column chromatography on silica gel (50-80% ethyl acetate (5% MeOH) in hexane) to obtain a diastereomeric mixture of compound 119 as a white form (927 mg, 74%). ¹H NMR (600 MHz, DMSO-d₆) δ 11.46-11.44 (m, 2H), 7.75-7.58 (m, 2H), 7.39-7.23 (m, 9H), 6.91-6.88 (m, 4H), 5.89-5.80 (m, 2H), 5.64-5.58 (m, 1H), 5.39-5.30 (m, 0.8H), 5.27-5.14 (m, 1.2H), 4.85-4.74 (m, 0.8H), 4.40-4.30 (m, 1.2H), 4.24-3.94 (m, 5H), 3.74-3.73 (m, 6H), 3.42-3.41 (m, 3H), 3.33-3.30 (m, 2H), 2.61-2.59 (m, 3H), 2.45-2.43 (m, 3H), 0.87-0.86 (m, 9H), 0.11-0.07 (m, 6H) ppm. ¹³C NMR (151 MHz, DMSO-d₆) δ 163.67, 163.64, 163.40, 158.70, 158.64, 150.77, 150.75, 150.64, 150.61, 144.90, 144.82, 141.70, 141.49, 141.46, 141.27, 141.02, 140.69, 135.60, 135.41, 135.34, 130.31, 130.29, 128.38, 128.27, 128.22, 127.38, 127.30, 113.70, 102.39, 102.31, 102.29, 102.21, 102.18, 102.12, 93.24, 93.18, 93.09, 92.00, 91.94, 91.85, 89.68, 89.63, 89.50, 89.44, 89.39, 89.26, 87.94, 87.19, 86.62, 86.59, 86.53, 81.71, 81.67, 81.49, 81.45, 81.27, 81.22, 81.09, 81.07, 80.99, 80.94, 80.90, 80.84, 72.27, 72.13, 72.11, 69.51, 69.49, 69.39, 69.28, 69.17, 69.11, 69.00, 64.68, 64.53, 64.18, 63.96, 63.93, 62.89, 62.30, 58.49, 58.28, 58.15, 55.53, 55.48, 55.47, 36.56, 36.54, 36.46, 36.44, 25.99, 25.96, 18.14, −4.46, −4.51, −4.82, −4.84, −4.87, −4.89 ppm. ¹⁹F NMR (565 MHz, DMSO-d₆) δ −200.26, −200.29, −200.33, −200.35, −200.39, −200.42, −200.45, −200.46, −200.48, −200.50, −200.52, −200.55, −200.59. ³¹P NMR (243 MHz, DMSO-d₆) δ 11.73, 11.32, 11.06, 10.51. HRMS calc. for C₄₈H₆₁FN₅NaO₁₄ Psi [M+Na]⁺ 1032.3604, found 1032.3575.

Synthesis of compound 120. To a solution of compound 119 (2.1 g, 2.08 mmol) and triethylamine (0.869 mL, 6.24 mmol) in THE (20 mL) was added dropwise triethylamine trihydrofluoride (1.02 mL, 6.24 mmol). The mixture was stirred at room temperature for 24 hours and concentrated under vacuum. The crude residue was purified by column chromatography on silica gel (0-10% MeOH in ethylacetate) to obtain a diastereomeric mixture of compounds 120 as a white form (1.42 g, 76%). ¹H NMR (400 MHz, DMSO-d₆) δ 11.45-11.42 (m, 2H), 7.75-7.72 (m, 1H), 7.62-7.56 (m, 1H), 7.40-7.23 (m, 9H), 6.92-6.88 (m, 4H), 5.91-5.78 (m, 3H), 5.60-5.55 (m, 1H), 5.39-5.32 (m, 1H), 5.19-5.04 (m, 1H), 4.86-4.77 (m, 1H), 4.22-3.96 (m, 6H), 3.74 (s, 6H), 3.42-3.41 (m, 3H), 3.34-3.31 (m, 2H), 2.60 (d, J=10.3 Hz, 4H), 2.45 (d, J=10.3 Hz, 2H) ppm. ¹³C NMR (126 MHz, DMSO-d₆) δ 163.05, 162.88, 158.21, 158.15, 150.27, 150.12, 150.08, 144.39, 144.33, 140.78, 140.75, 140.55, 140.20, 135.13, 134.93, 134.91, 129.78, 127.89, 127.78, 127.73, 126.87, 126.80, 113.23, 101.92, 101.85, 101.79, 101.74, 93.64, 93.61, 92.17, 92.14, 88.85, 88.69, 88.57, 88.41, 87.35, 86.68, 86.15, 86.09, 81.29, 81.24, 81.09, 81.04, 80.58, 80.54, 80.48, 80.46, 80.43, 71.84, 71.80, 71.75, 71.71, 67.97, 67.95, 67.84, 67.82, 64.73, 64.68, 64.64, 62.38, 61.91, 57.96, 57.81, 55.04, 55.00, 36.07, 36.04, 35.98, 35.95 ppm. ¹⁹F NMR (471 MHz, DMSO-d₆) δ −200.37, −200.41, −200.46, −200.48, −200.52, −200.57, −200.64, −200.68, −200.73, −200.75, −200.80, −200.84. ³¹P NMR (202 MHz, DMSO-d₆) δ 12.10, 11.68. HRMS calc. for C₄₂H₄₇FN₅NaO₁₄P [M+Na]⁺ 918.2739, found 918.2700.

Synthesis of compound 121. To a solution of compound 120 (1.4 g, 1.56 mmol), 1-methylimidazole (12.5 μL, 156 mol) and DIPEA (0.817 mL, 4.69 mmol) in CH₂Cl₂ (15 mL) was added dropwise 2-cyanoethyl-N,N-diisopropylchlorophosphoramidite (0.419 mL, 1.88 mmol) at 0° C. and the mixture was stirred at 0° C. for 3 hours. The reaction mixture was diluted with ethyl acetate and washed with saturated NaHCO₃ (aq.), water, brine, dried (Na₂SO₄) and concentrated under vacuum. The crude residue was purified by column chromatography on silica gel (100% ethyl acetate) to obtain a diastereomeric mixture of compound 121 as a white form (1.31 g, 77%). ¹H NMR (500 MHz, CD₃CN) δ 9.22 (brs, 2H), 7.72 (d, J=8.2 Hz, 0.4H), 7.66 (dd, J=8.2, 3.2 Hz, 0.6H), 7.55 (dd, J=8.2, 2.7 Hz, 0.6H), 7.46-7.41 (m, 2H), 7.35-7.25 (m, 7.4H), 6.89-6.87 (m, 4H), 5.91-5.84 (m, 1.6H), 5.74-5.70 (m, 0.4H), 5.59 (dd, J=8.2, 6.2 Hz, 0.6H), 5.52 (d, J=8.2 Hz, 0.4H), 5.29-5.24 (m, 1H), 5.20-5.16 (m, 0.5H), 5.10-5.06 (m, 0.5H), 4.95-4.91 (m, 0.4H), 4.87-4.81 (m, 0.6H), 4.51-4.00 (m, 6H), 3.83-3.77 (m, 8H), 3.67-3.61 (m, 2H), 3.50-3.34 (m, 5H), 2.70-2.63 (m, 5H), 2.53-2.50 (m, 3H), 1.18-1.16 (m, 12H) ppm. ¹³C NMR (151 MHz, CD₃CN) δ 163.61, 163.59, 163.56, 163.54, 163.52, 163.49, 163.47, 159.42, 159.41, 159.36, 159.33, 150.97, 150.95, 150.93, 150.76, 150.72, 150.62, 150.60, 145.23, 145.21, 145.09, 141.44, 141.33, 141.07, 140.88, 140.60, 140.45, 135.91, 135.79, 135.76, 130.81, 130.78, 130.76, 128.73, 128.69, 128.55, 127.68, 127.61, 119.15, 119.12, 113.74, 102.56, 102.53, 102.49, 102.46, 102.40, 93.88, 93.31, 92.64, 92.06, 90.82, 90.70, 90.58, 90.46, 90.15, 90.03, 89.92, 89.80, 88.29, 88.20, 87.48, 87.39, 87.33, 87.30, 82.58, 82.57, 82.37, 82.25, 82.20, 81.98, 81.93, 81.89, 81.84, 81.15, 81.12, 80.89, 80.73, 80.68, 80.64, 80.51, 80.46, 72.44, 72.41, 72.30, 72.27, 72.22, 72.19, 70.07, 70.02, 69.97, 69.90, 69.86, 69.80, 69.76, 64.63, 64.60, 64.51, 64.47, 64.44, 64.41, 64.25, 64.22, 62.50, 62.44, 61.92, 59.42, 59.39, 59.30, 59.26, 59.23, 59.10, 58.73, 58.53, 58.51, 55.53, 55.51, 43.81, 43.79, 43.73, 43.71, 36.57, 36.55, 36.52, 36.51, 36.49, 36.46, 24.63, 24.58, 24.54, 24.50, 24.49, 24.45, 24.42, 24.40, 20.55, 20.51 ppm. ¹⁹F NMR (471 MHz, CD₃CN) δ −198.75, −198.77, −198.79, −198.82, −198.84, −198.86, −198.88, −198.91, −198.93, −198.97, −199.40, −199.42, −199.45, −199.46, −199.49, −199.51, −199.52, −199.53, −199.56, −199.58, −199.60, −199.62, −200.08, −200.10, −200.12, −200.14, −200.16, −200.19, −200.21, −200.23, −200.25, −200.27, −200.30, −200.75, −200.76, −200.79, −200.80, −200.83, −200.84, −200.86, −200.87, −200.90, −200.92, −200.94, −200.96. ³¹P NMR (202 MHz, CD₃CN) δ 152.81, 152.78, 152.76, 152.57, 152.53, 152.48, 12.96, 12.94, 12.40, 12.26. HRMS calc. for C₅₁H₆₄FN₇NaO₁₅P₂ [M+Na]⁺ 1118.3817, found 1118.3838.

### Synthesis of PN Linkages—Wada Approach

Synthesis oligonucleotides containing PN linkages by Wada approach can be found in 6. Hara et al., “Stereocontrolled Synthesis of Boranophosphate DNA by an Oxazaphospholidine Approach and Evaluation of Its Properties,” The Journal of Organic Chemistry. 2019; 84(12):7971-83; Maier et al., “Synthesis of Chimeric Oligonucleotides Containing Phosphodiester, Phosphorothioate, and Phosphoramidate Linkages,” Organic Letters. 2000; 2(13):1819-22; which are incorporated by reference in their entirety.

Additional synthesis of PN linkages are shown in Schemes 17-19.

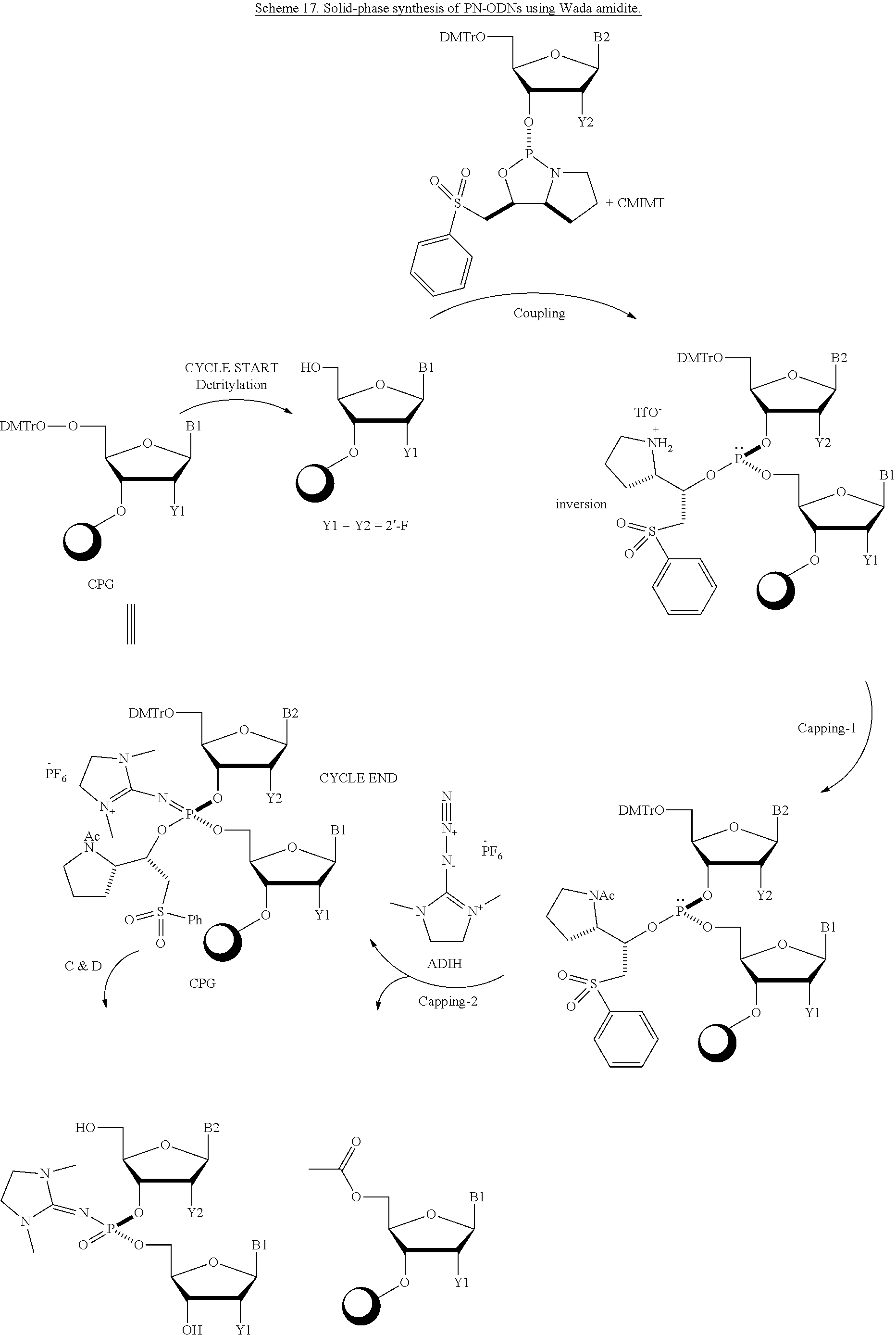

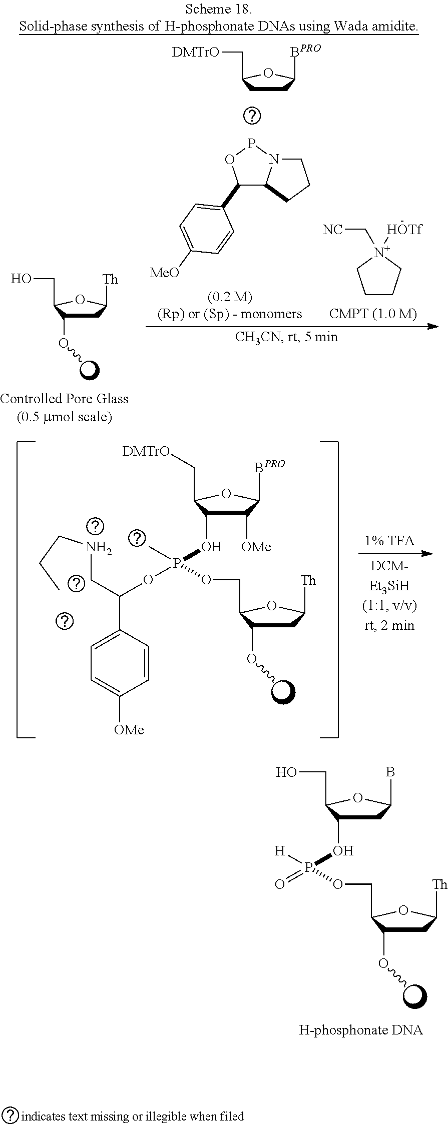

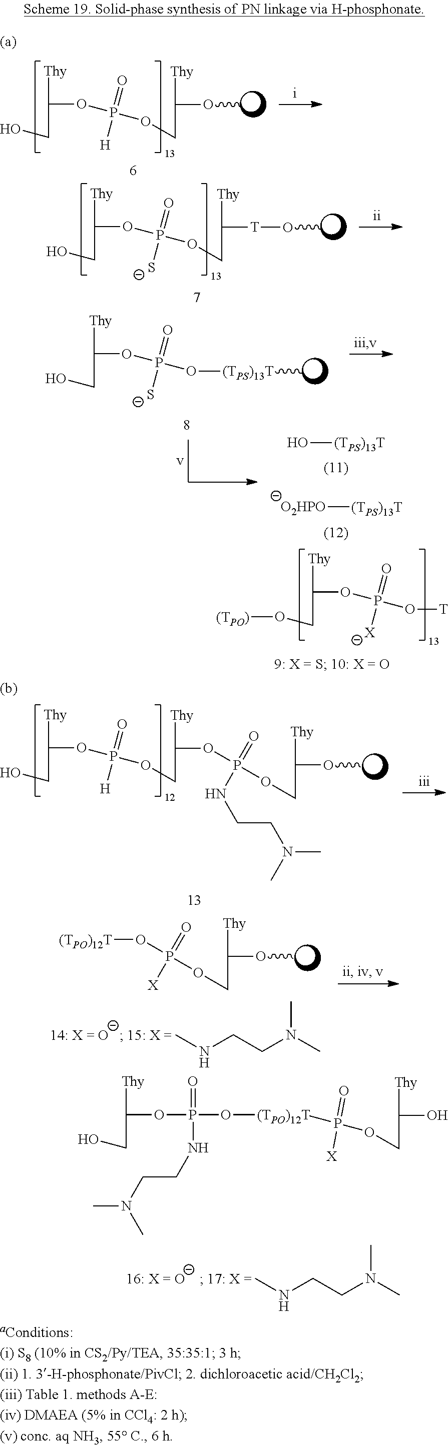

### Synthesis of Additional Dimers Containing PN Lipids

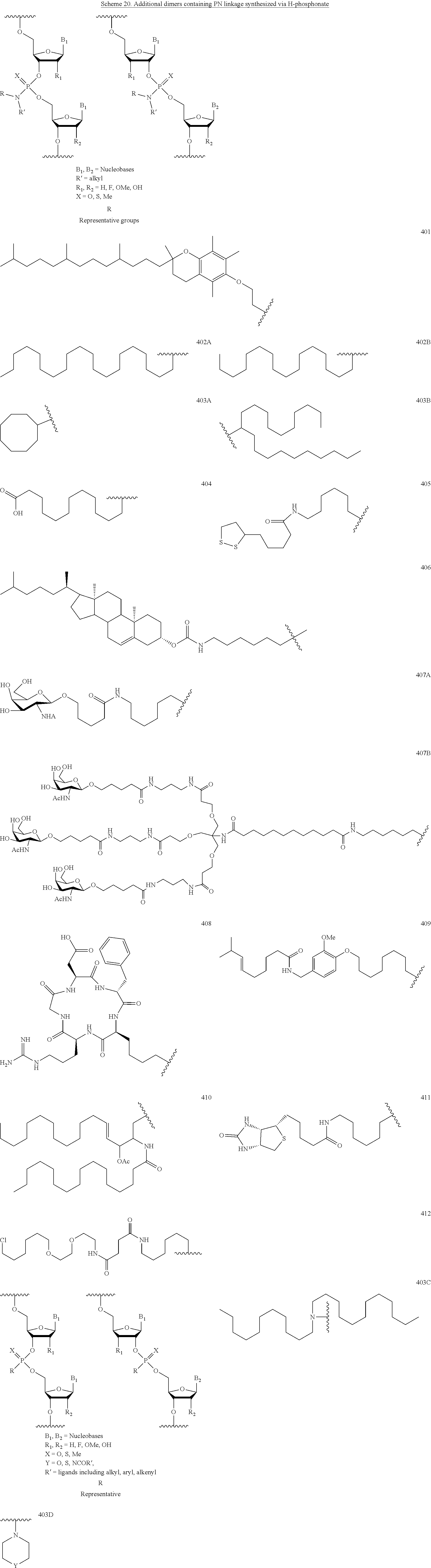

In Scheme 20, the ligand R is Tocopherol (401), alkyl (402), cyclic and branched-alkyl (403), flip-C12 (404), Lipoic acid (405), Cholesterol (406), GalNAc (407), cRGD (408), Capsaicin (409), Sphingolipid (410), Biotin (411), Chloroalkane (412) and Peptide ligands (413) viz., Apo-E, Apo-A, TfR peptide, RGD mimics, respectively.

See Braddock et al., “Conformationally Specific Enhancement of Receptor-Mediated LDL Binding and Internalization by Peptide Models of a Conserved Anionic N-Terminal Domain of Human Apolipoprotein E.,” Biochemistry. 1996; 35(44):13975-84, which is incorporated herein by reference in its entirety, for ligand R being Apo-E.

See Devlin et al., “An Apolipoprotein(a) Peptide Delays Chylomicron Remnant Clearance and Increases Plasma Remnant Lipoproteins and Atherosclerosis In Vivo,” Arteriosclerosis, Thrombosis, and Vascular Biology. 2005; 25(8):1704-10, which is incorporated herein by reference in its entirety, for ligand R being Apo-A.

See Dai et al., “TfR binding peptide screened by phage display technology—characterization to target cancer cells,” Trop J Pharm Res. 2014; 13(3):331-8, 8; Gomes et al., “Tailoring Lipid and Polymeric Nanoparticles as siRNA Carriers towards the Blood-Brain Barrier—from Targeting to Safe Administration,” J Neuroimmune Pharmacol. 2017; 12(1):107-19, which are incorporated herein by reference in their entirety, for ligand R being TfR peptide.

See Ruoslahti, “RGD and other recognition sequences for integrins,” Annu Rev Cell Dev Biol. 1996; 12:697-715; Abraham et al., “Arginine-glycine-aspartic acid mimics can identify a transitional activation state of recombinant αIIbβ3 in human embryonic kidney 293 cells,” Mol Pharmacol. 1997; 52(2):227-36; Fisher et al., “Non-Peptide RGD Surrogates Which Mimic a Gly-Asp j-Turn: Potent Antagonists of Platelet Glycoprotein IIb-IIIa,” J Med Chem. 1997; 40(13):2085-101; Fisher et al., “Dihydroisoquinolone RGD mimics. Exploration of the aspartate isostere,” Bioorg Med Chem Lett. 1997; 7(19):2537-42; Nagarajan et al., “R-Isomers of Arg-Gly-Asp (RGD) mimics as potent αvβ inhibitors,” Bioorg Med Chem. 2007; 15(11):3783-800; Narasimhan et al., “Enhanced cell adhesion and mature intracellular structure promoted by squaramide-based RGD mimics on bioinert surfaces,” Bioorg Med Chem. 2013; 21(8):2210-6; Higuchi et al., “Design and synthesis of non-peptide RGD mimics for evaluation of their utility as anti-platelet agents,” Chem Pharm Bull. 2016; 64(12):1726-38; Vasile et al., “Insight to the binding mode of triazole RGD-peptidomimetics to integrin-rich cancer cells by NMR and molecular modeling,” Bioorg Med Chem. 2016; 24(5):989-94; Piras et al., “High-Affinity “Click” RGD Peptidomimetics as Radiolabeled Probes for Imaging αvβ Integrin,” Chem Med Chem. 2017; 12(14):1142-51, which are incorporated herein by reference in their entirety, for ligand R being RGD mimics.

Oligomer Synthesis with Lipophilic P—N Linkages

Oligonucleotides were synthesized on an ABI-394 DNA/RNA synthesizer using modified synthesis cycles based on those provided with the instrument. A solution of 0.25 M 5-(S-ethylthio)-1H-tetrazole in acetonitrile was used as the activator. The phosphoramidite solutions were 0.15 M in anhydrous acetonitrile. The oxidizing reagent was 0.02 M I₂ in THF/pyridine/H₂O. N,N-Dimethyl-N′-(3-thioxo-3H-1,2,4-dithiazol-5-yl)methanimidamide (DDTT), 0.1 M in pyridine, was used as the sulfurizing reagent. The detritylation reagent was 3% dichloroacetic acid (DCA) in DCM. P—N conjugation was done using the H-phosphonate chemistry followed by amidate oxidation with 0.1 M amine in CCl₄/CH₂Cl₂ solution (1:1 v/v). See Maier et al., “Synthesis of Chimeric Oligonucleotides Containing Phosphodiester, Phosphorothioate, and Phosphoramidate Linkages,” Organic Letters. 2000; 2(13):1819-22; Vlaho et al., “Synthesis of Chimeric Oligonucleotides Having Modified Internucleotide Linkages via an Automated H-Phosphonate/Phosphoramidite Approach,” Current Protocols in Nucleic Acid Chemistry. 2018; 73(1):e53; Danielle, “Structural Studies and Gene Silencing Activity of siRNAs Containing Cationic Phosphoramidate Linkages,” Nucleic Acid Therapeutics. 2018; 28(1):34-43; Kostov et al., “Solid-Phase Synthesis of Phosphorothioate/Phosphonothioate and Phosphoramidate/Phosphonamidate Oligonucleotides,” Molecules. 2019; 24(10):1872, all of which are incorporated herein by reference in their entirety.

After completion of the automated synthesis, the oligonucleotide was then manually released from support by ammonia treatment for 5 hours at 60° C. For the carboxylic acid protected oligonucleotide, CPG was first treated with 10-15% piperidine aqueous solution at 37° C. for 24 hours followed by ammonia treatment.

Crude oligonucleotides were purified by preparative HPLC custom packed with TSKGel SuperQ-5PW(20) (Sigma) using an appropriate gradient of mobile phase (buffer A: 20 mM sodium phosphate, 15% ACN, pH 8.5; buffer B: 1 M NaBr, 20 mM sodium phosphate, 15% ACN, pH 8.5) and desalted using size-exclusion chromatography using a custom packed with Sephadex G25 (GE Healthcare) and water as an eluent.

Alternatively, crude oligonucleotides were purified by reverse phase C18 column preparative HPLC (prep RP-HPLC, Agilent, ZORBAX 300SB-C18 5 m 9.4×250 mm) using an appropriate gradient of mobile phase (buffer A: 50 mM TEAA, 3% CH₃CN; buffer B 50 mM TEAA, 80% CH₃CN) and desalted using size-exclusion chromatography using a custom packed with Sephadex G25 (GE Healthcare) and water as an eluent. Triethyl ammonium cation was displaced by Na with an excess of 0.1M AcONa solution, and a second desalting process.

Oligonucleotide concentrations were calculated based on absorbance at 260 nm and the following extinction coefficients: A/2,6-diaminoA, 13.86 M⁻¹cm⁻¹; T/U, 7.92 M⁻¹cm⁻¹; C, 6.57 M⁻¹cm⁻¹; and G, 10.53M⁻¹cm⁻¹. The purity and identity of oligonucleotides were verified by analytical anion exchange chromatography and mass spectrometry.

TABLE 14
Sequences and mass spectroscopy
characterization of conjugated SOD1 sense strands

| | | | | SEQ | Mass (m/z) | | LCMS |
|---|---|---|---|---|---|---|---|
| | Oligo | | | ID | Negative mode | | purity |
| Strand | ID | Reference | Sequence 5′-3′ | NO: | Calc. | Obs. | (%) |
| Sense | ON1 | A-2686439 | csasuuY211UfAf | 177 | 7044.987 | 7044.31 | 85 |
| | | | aUfCfCfucacucu | | | | |
| | | | asasa | | | | |
| Sense | ON2 | A-3476774 | csasuuY318UfAf | 178 | 7129.149 | 7128.79 | 79 |
| | | | aUfCfCfucacucu | | | | |
| | | | asasa | | | | |
| Sense | ON3 | A-3507227 | csasuuY320UfAf | 179 | 7018.861 | — | — |
| | | | aUfCfCfucacucu | | | | |
| | | | asasa | | | | |
| Sense | ON4 | A-3507258 | csasuuY321UfAf | 180 | 7332.390 | 7331.90 | 89 |
| | | | aUfCfCfucacucu | | | | |
| | | | asasa | | | | |
| Sense | ON5 | A-3507228 | csasuuY333UfAf | 181 | 7277.310 | 7275.59 | 85 |
| | | | aUfCfCfucacucu | | | | |
| | | | asasa | | | | |

TABLE 15
Sequences and mass spectroscopy characterization of conjugated SOD1 sense
strands

| | | | | | Mass |
|---|---|---|---|---|---|
| | | | | | (m/z) |
| | | | | SEQ | Negative |
| | PN | | | ID | mode |
| Strand | position | # | Sequence 5′-3′ | NO: | Calc. |
| Antisense | 3/4 | ON1 | usUfsY999UfaGfaGfcAfagaAfcAfcUfgUfususu | 182 | |
| Antisense | 3/4 | ON2 | usUfsY329UfaGfaGfcAfagaAfcAfcUfgUfususu | 183 | 7694.13 |
| Antisense | 5/6 | ON3 | usUfsaUfY999GfaGfcAfagaAfcAfcUfgUfususu | 184 | |
| Antisense | 5/6 | ON4 | usUfsaUfY329GfaGfcAfagaAfcAfcUfgUfususu | 185 | 7694.13 |
| Antisense | 7/8 | ON5 | usUfsaUfaGfY999GfcAfagaAfcAfcUfgUfususu | 186 | |
| Antisense | 7/8 | ON6 | usUfsaUfaGfY329GfcAfagaAfcAfcUfgUfususu | 187 | 7694.13 |
| Antisense | 10/11 | ON7 | usUfsaUfaGfaGfcY998agaAfcAfcUfgUfususu | 188 | |
| Antisense | 10/11 | ON8 | usUfsaUfaGfaGfcY327agaAfcAfcUfgUfususu | 189 | 7694.13 |
| Antisense | 14/15 | ON9 | usUfsaUfaGfaGfcAfagaY998cAfcUfgUfususu | 190 | |
| Antisense | 14/15 | ON10 | usUfsaUfaGfaGfcAfagaY327cAfcUfgUfususu | 191 | 7694.13 |

1. A double-stranded RNA (dsRNA) agent for modulating the expression of a target gene in the central nervous system (CNS), comprising
an antisense strand which is complementary to the target gene in the CNS;
a sense strand which is complementary to the antisense strand; and
one or more lipophilic moieties containing one or more saturated or unsaturated C₂₂ hydrocarbon chains conjugated to at least one strand, optionally via a linker or carrier.

2. The dsRNA agent of claim 1, wherein at least one C₂₂ hydrocarbon chain is a saturated or unsaturated, linear or branched C₂₂ hydrocarbon chain.

3. The dsRNA agent of claim 1, wherein the one or more C₂₂ hydrocarbon chains contains a functional group selected from the group consisting of hydroxyl, amine, carboxylic acid, carboxylic acid ester, sulfonate, ether, phosphate, thiol, azide, alkyne, cycloalkyne, trans-cyclooctenyl, N-maleimidyl, and 1,2,4,5-tetrazin-3-yl.

4-8. (canceled)

9. The dsRNA agent of claim 1, wherein the lipophilic moiety is a lipophilic monomer selected from the group consisting of: 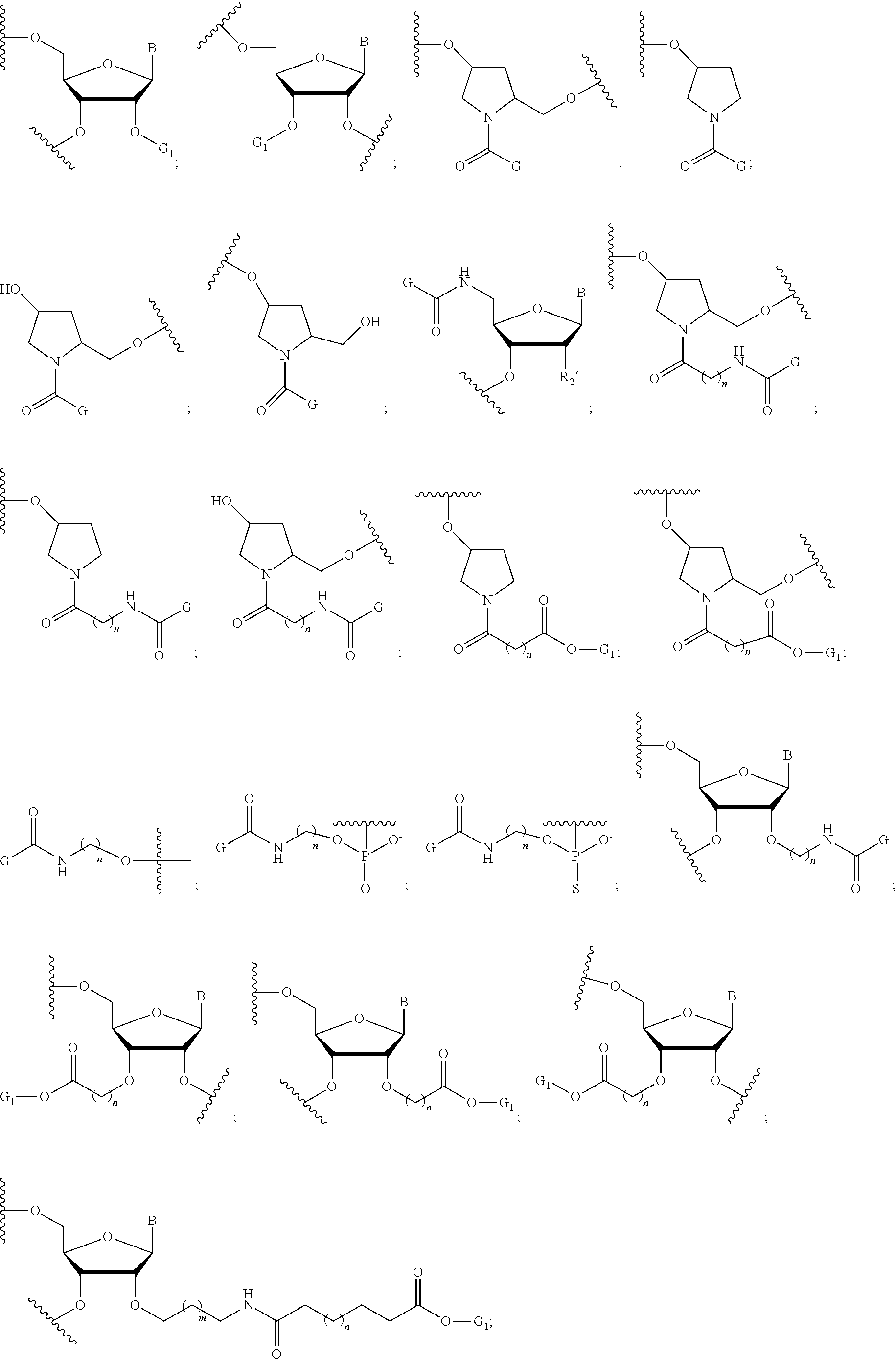 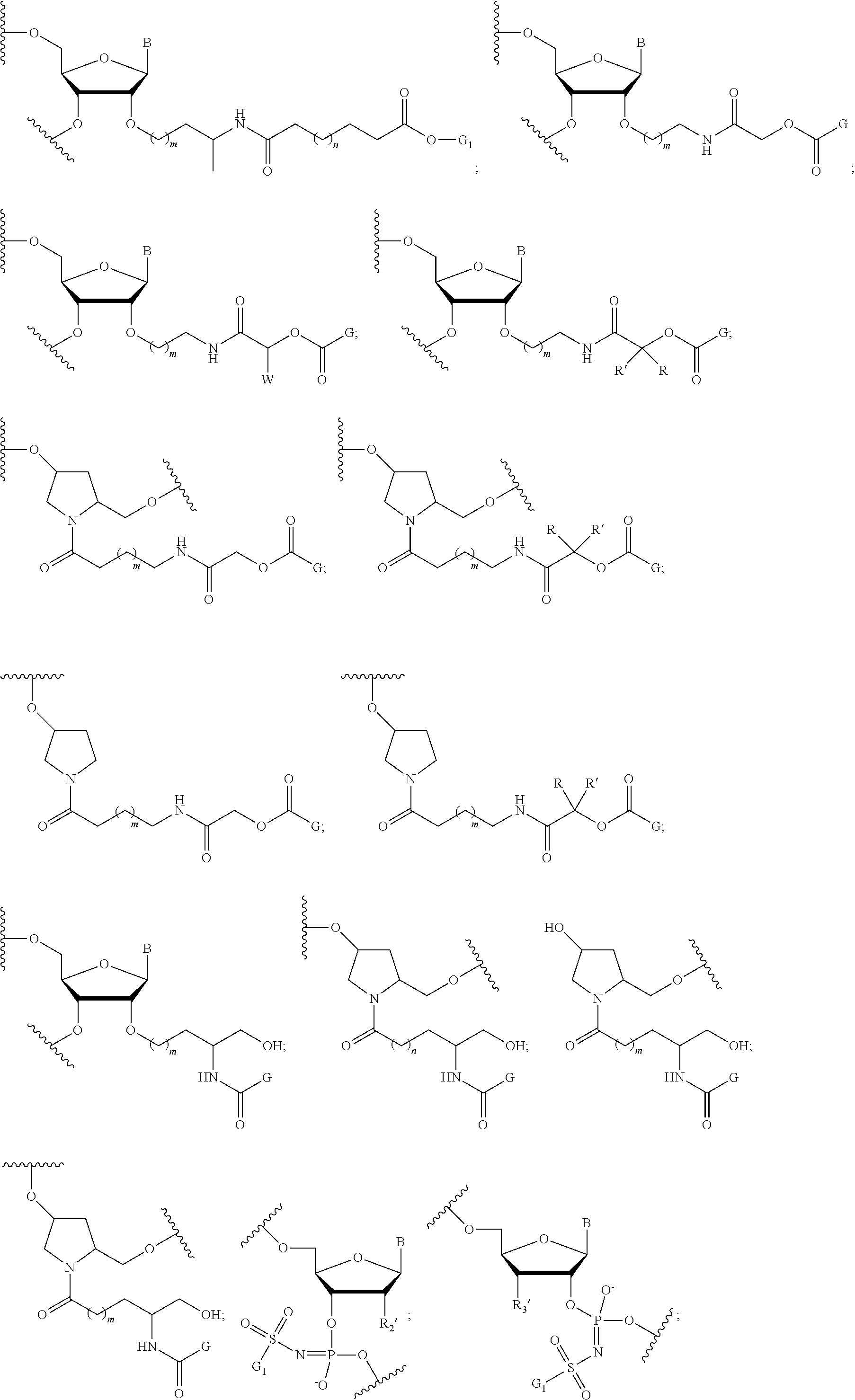 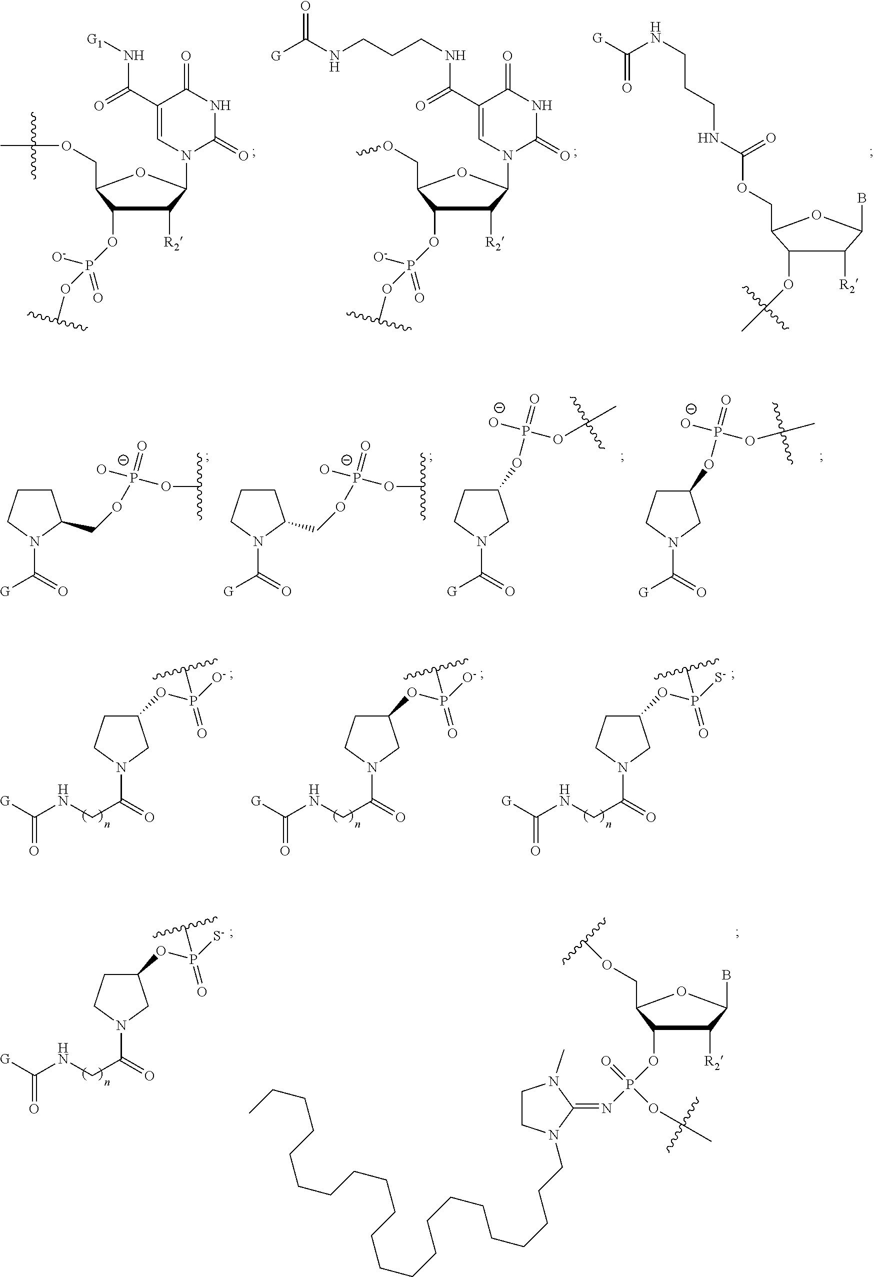 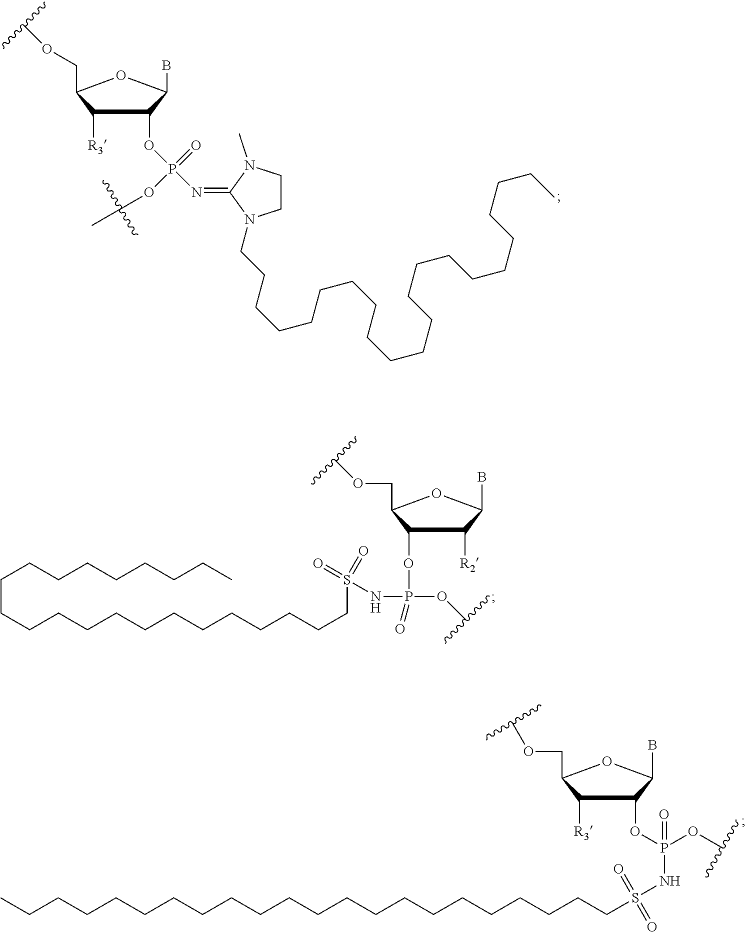 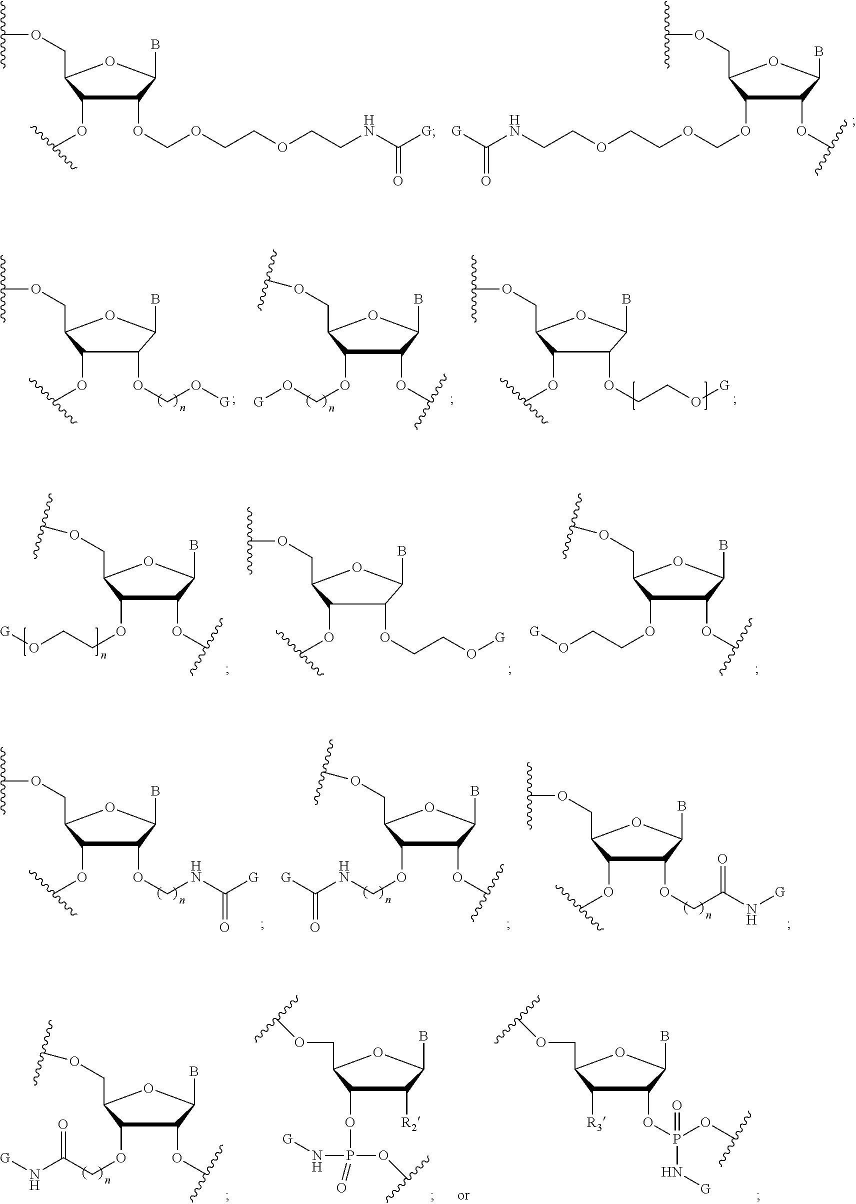
wherein:
G is G₁ or a saturated or unsaturated C₂₁ hydrocarbon chain, wherein G is optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆ alkyl;
G₁ is a saturated or unsaturated C₂₂ hydrocarbon chain, wherein G¹ is optionally substituted with one or two groups selected from the group consisting of halogen, —OR^{G1}, SR^{G1}, —N(R^{G1})₂, —C(O)OR^{G1}, —OC(O)R^{G1}, —C(O)N(R^{G1})₂, —N(R^{G1})C(O)R^{G1}, —N(R^{G1})C(O)OR^{G1}, —N(R^{G1})SO₂(R^{G1}), or —SO₂N(R^{G1})₂, wherein each R^{G1} is independently hydrogen or C₁-C₆ alkyl;
m is an integer of 0-8;
n is an integer of 1-21;
R₂′ and R₃′ are each independently H, OH, F, OMe, O-methoxyalkyl, O-allyl, O—N-methylacetamido, O-dimethylaminoethoxyethyl, or O-aminopropyl;
B is a modified or unmodified nucleobase;
W is an alkyl group; and
R and R′ are each independently H or an alkyl group.

10. The dsRNA agent of claim 1, wherein the lipophilic moiety is a lipophilic monomer selected from one of the members of group (i), group (ii), and group (iii): 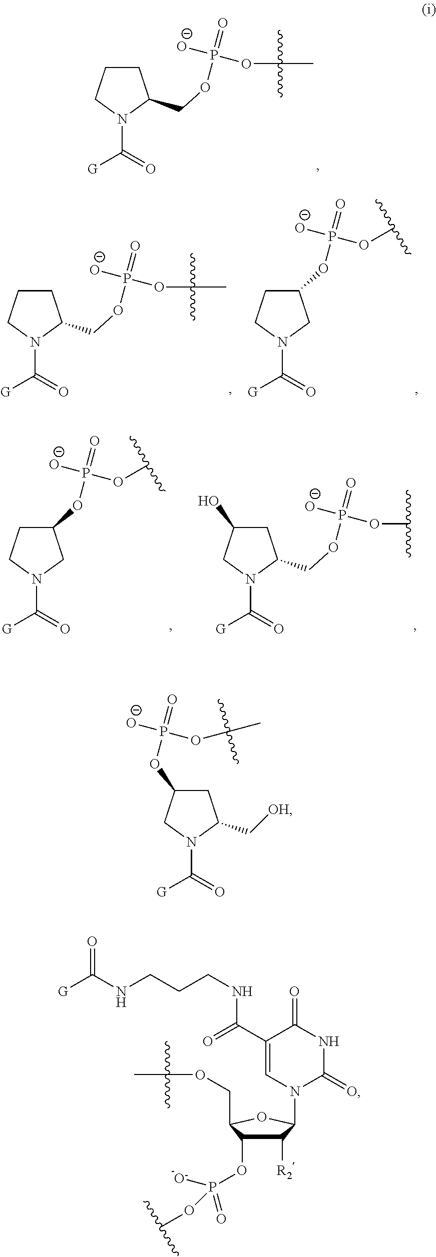 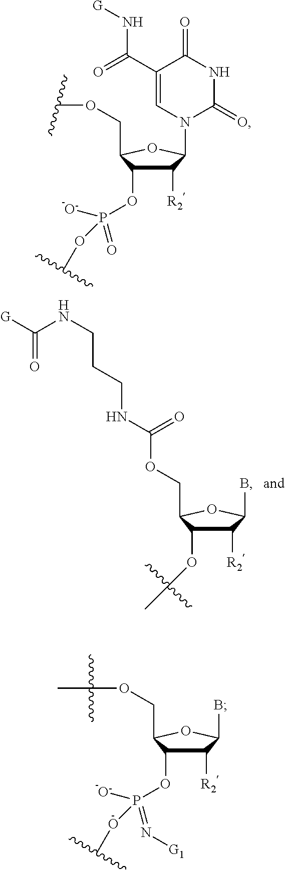 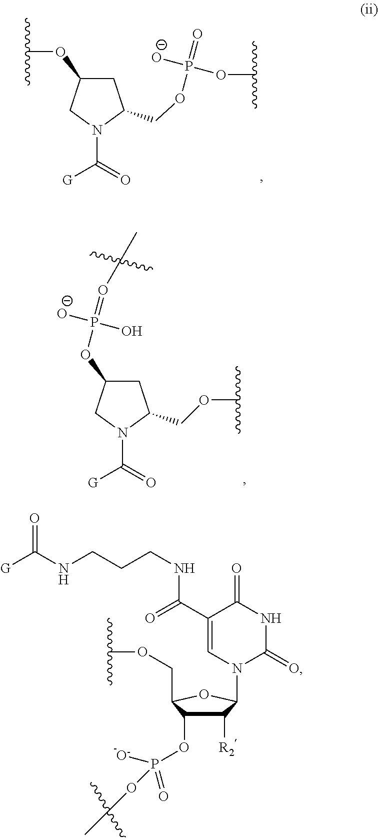 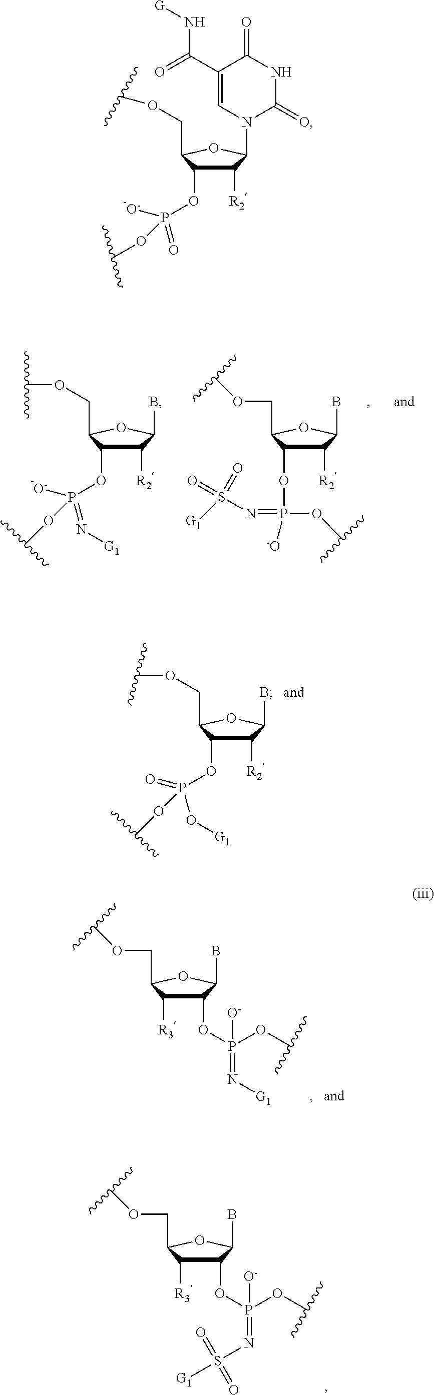
wherein:
G is G₁ or a saturated or unsaturated C₂₁ hydrocarbon chain, wherein G is optionally substituted with one or two groups selected from the group consisting of halogen, —OR^{G}, —SR^{G}, —N(R^{G})₂, —C(O)OR^{G}, —OC(O)R^{G}, —C(O)N(R^{G})₂, —N(R^{G})C(O)R^{G}, —N(R^{G})C(O)OR^{G}, —N(R^{G})SO₂(R^{G}), or —SO₂N(R^{G})₂, wherein each R^{G} is independently hydrogen or C₁-C₆alkyl;
G₁ is a saturated or unsaturated C₂₂ hydrocarbon chain, wherein G¹ is optionally substituted with one or two groups selected from the group consisting of halogen, —OR^{G1}, —SR^{G1}, —N(R^{G1})₂, —C(O)OR^{G1}, —OC(O)R^{G1}, —C(O)N(R^{G1})₂, —N(R^{G1})C(O)R^{G1}, —N(R^{G1})C(O)OR^{G1}, —N(R^{G1})SO₂(R^{G1}), or —SO₂N(R^{G1})₂, wherein each R^{G1} is independently hydrogen or C₁-C₆alkyl;
R₂′ and R₃′ are each independently H, OH, F, OMe, O-methoxyalkyl, O-allyl, O—N-methylacetamido, O-dimethylaminoethoxyethyl, or O-aminopropyl; and
B is a modified or unmodified nucleobase.

11. The dsRNA agent of claim 1, wherein the lipophilic monomer is 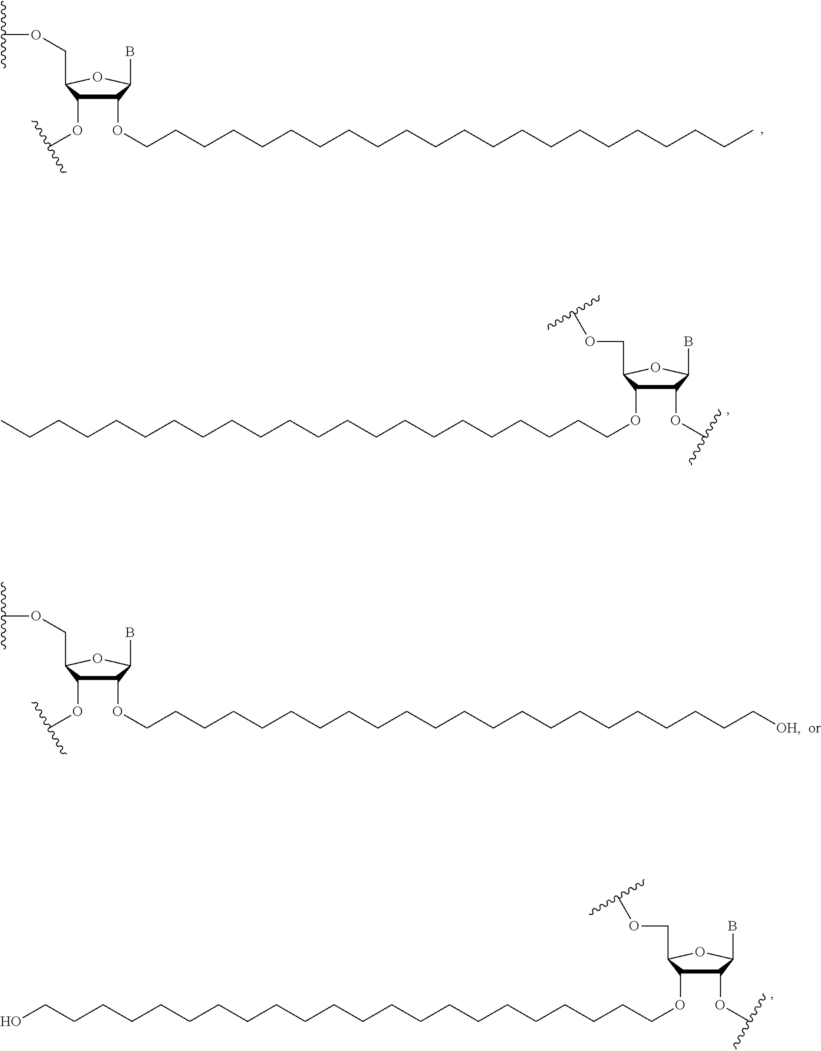
wherein B is a modified or unmodified nucleobase.

12. The dsRNA agent of claim 1, wherein the lipophilic monomer is 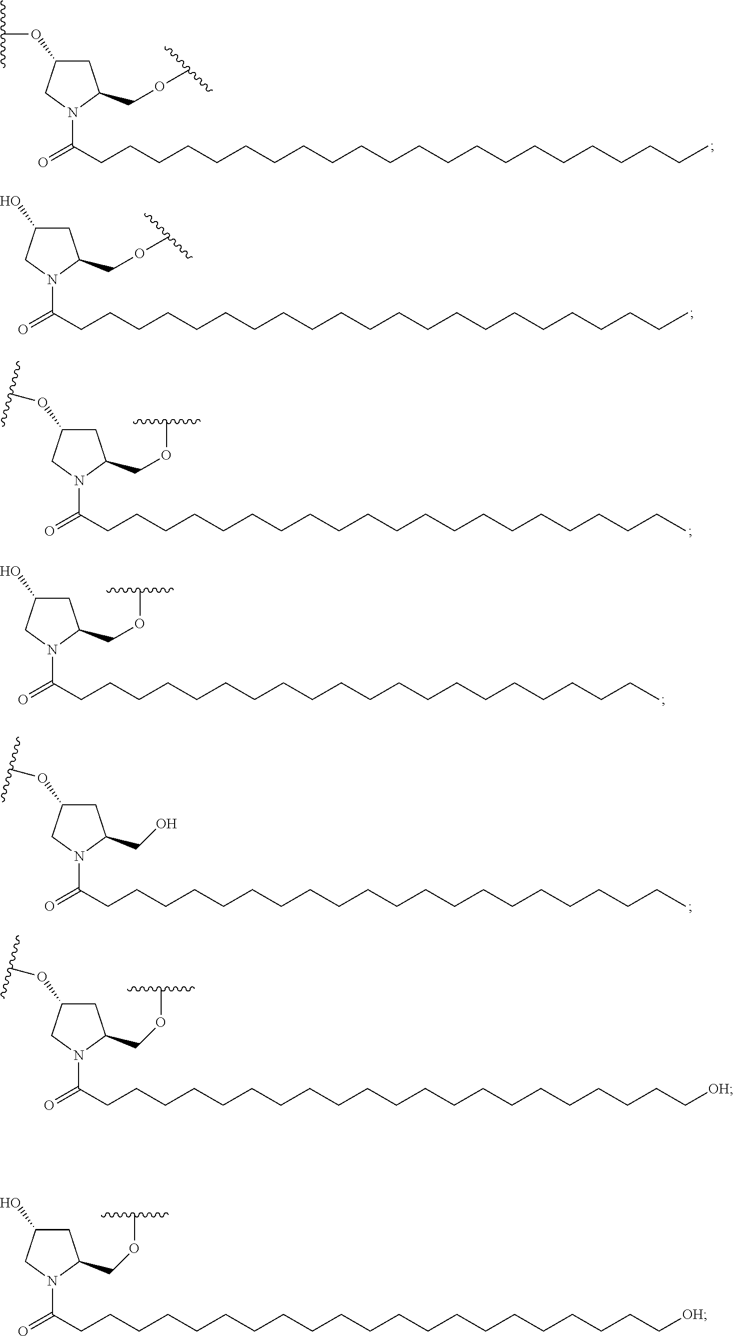 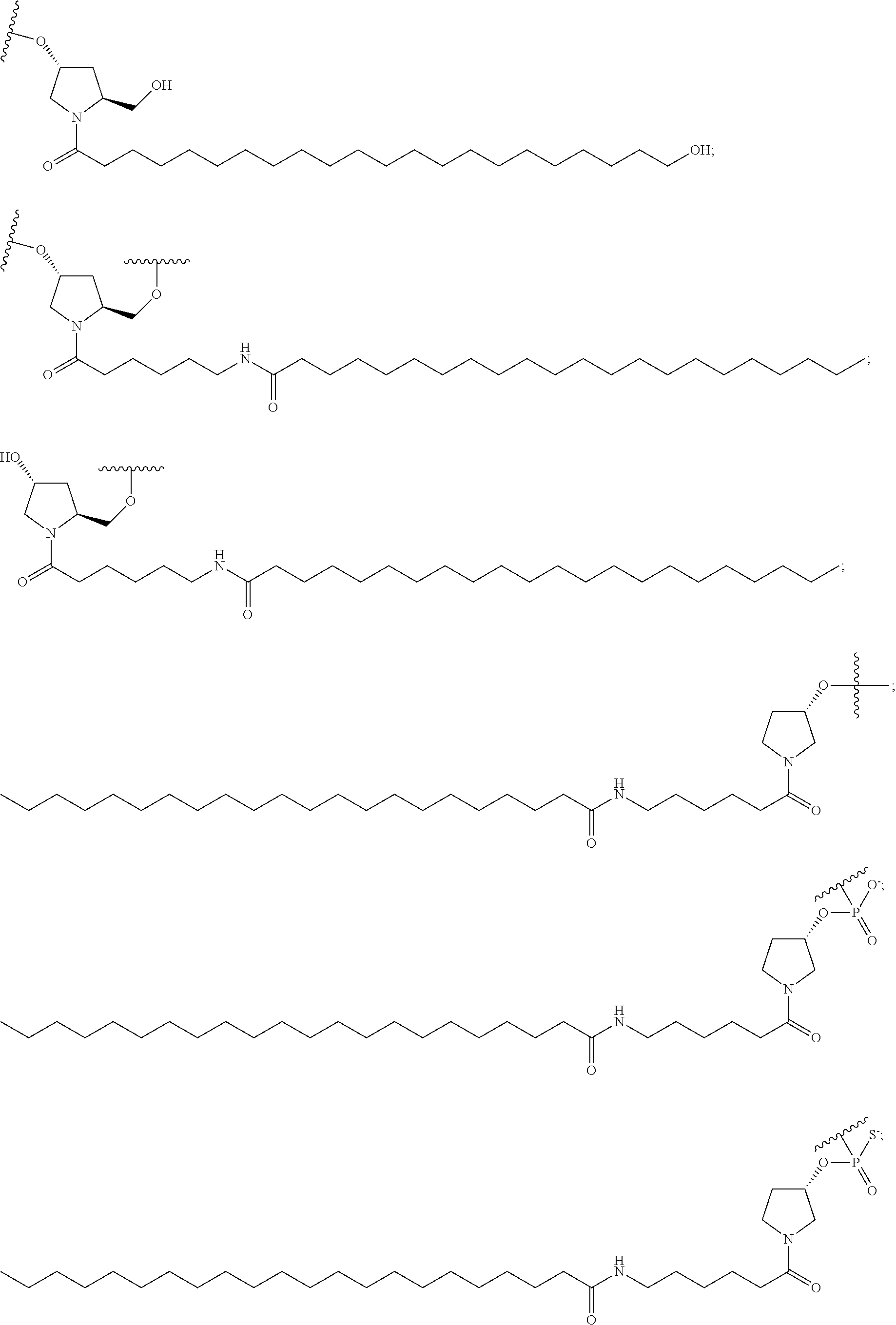 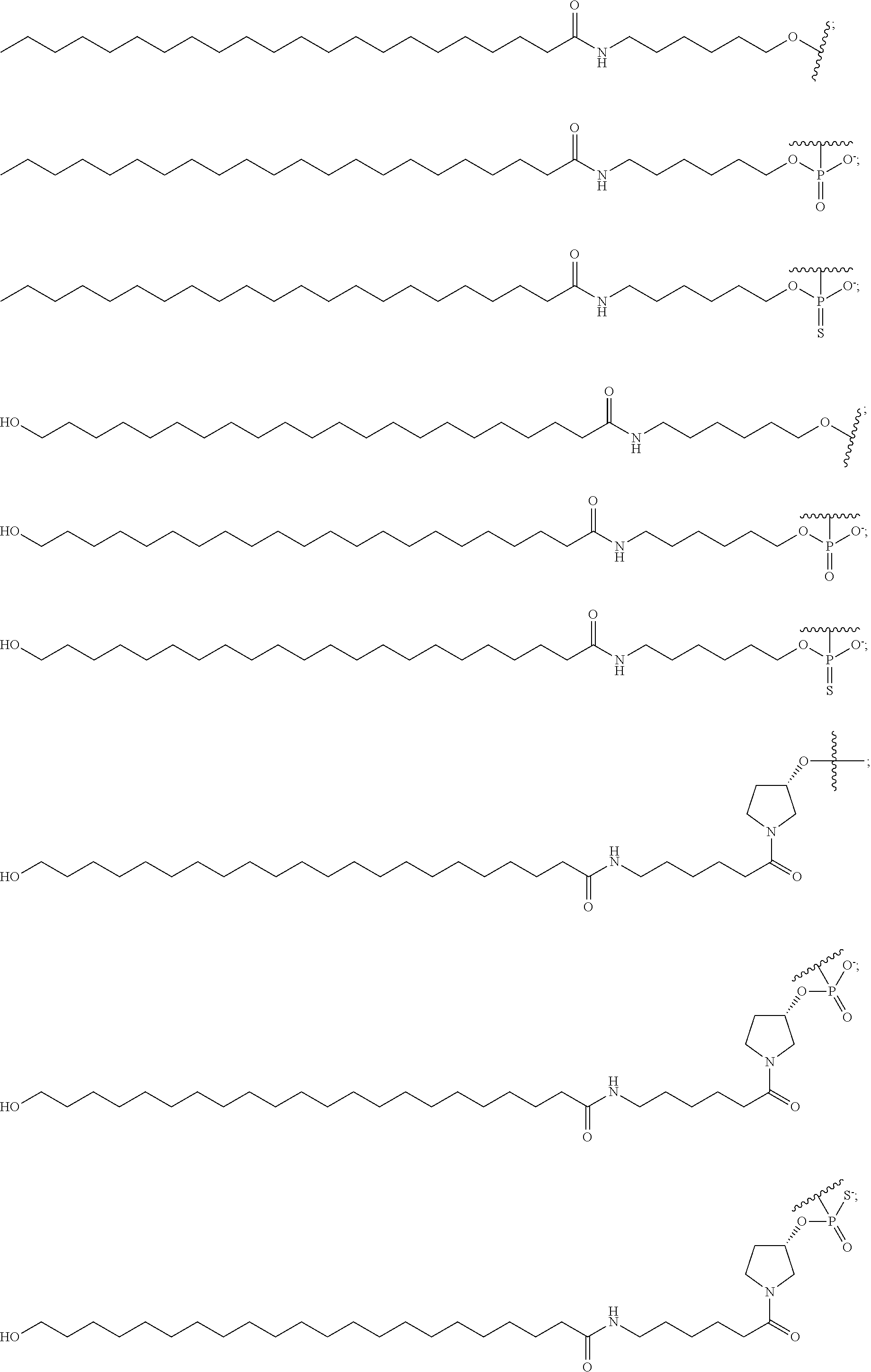 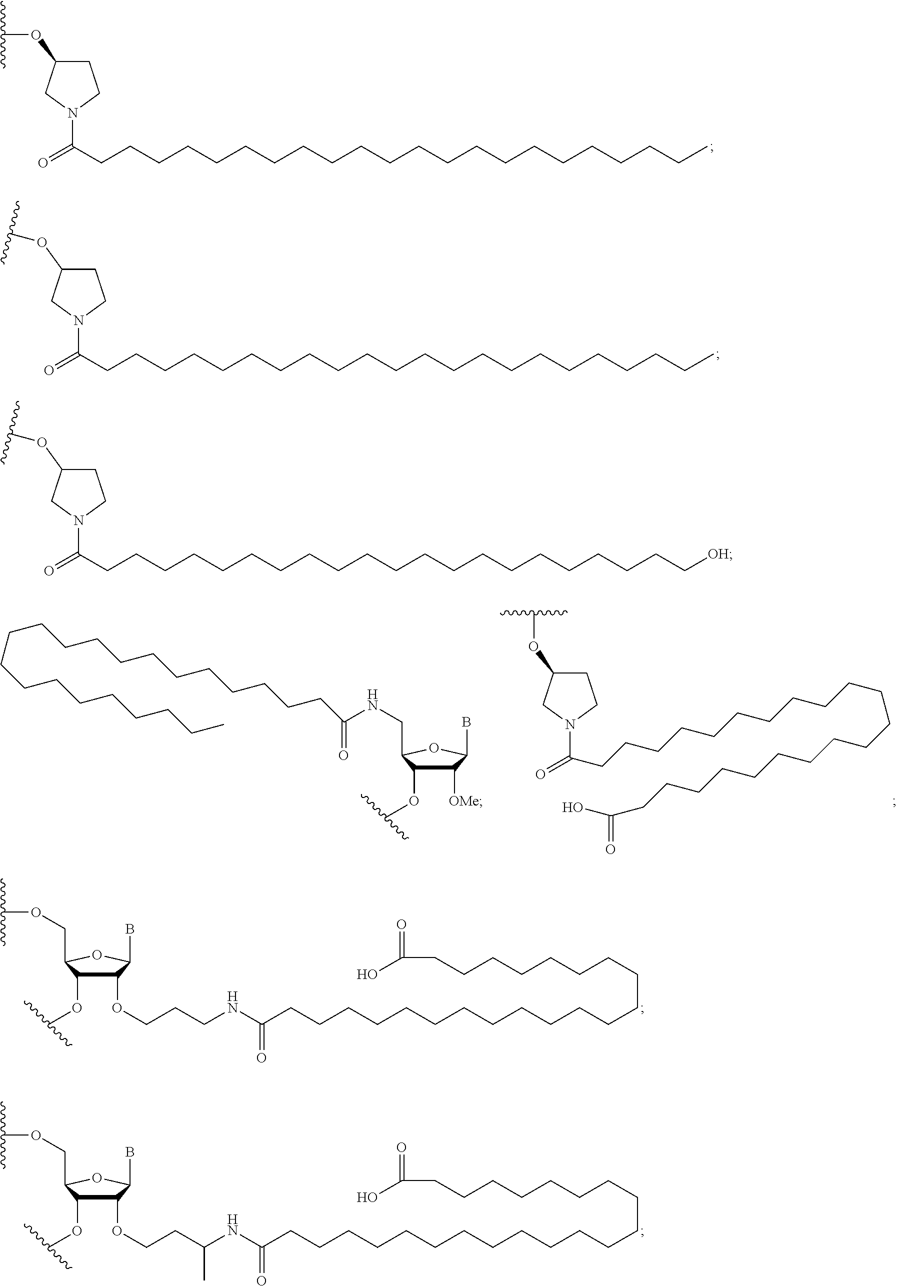 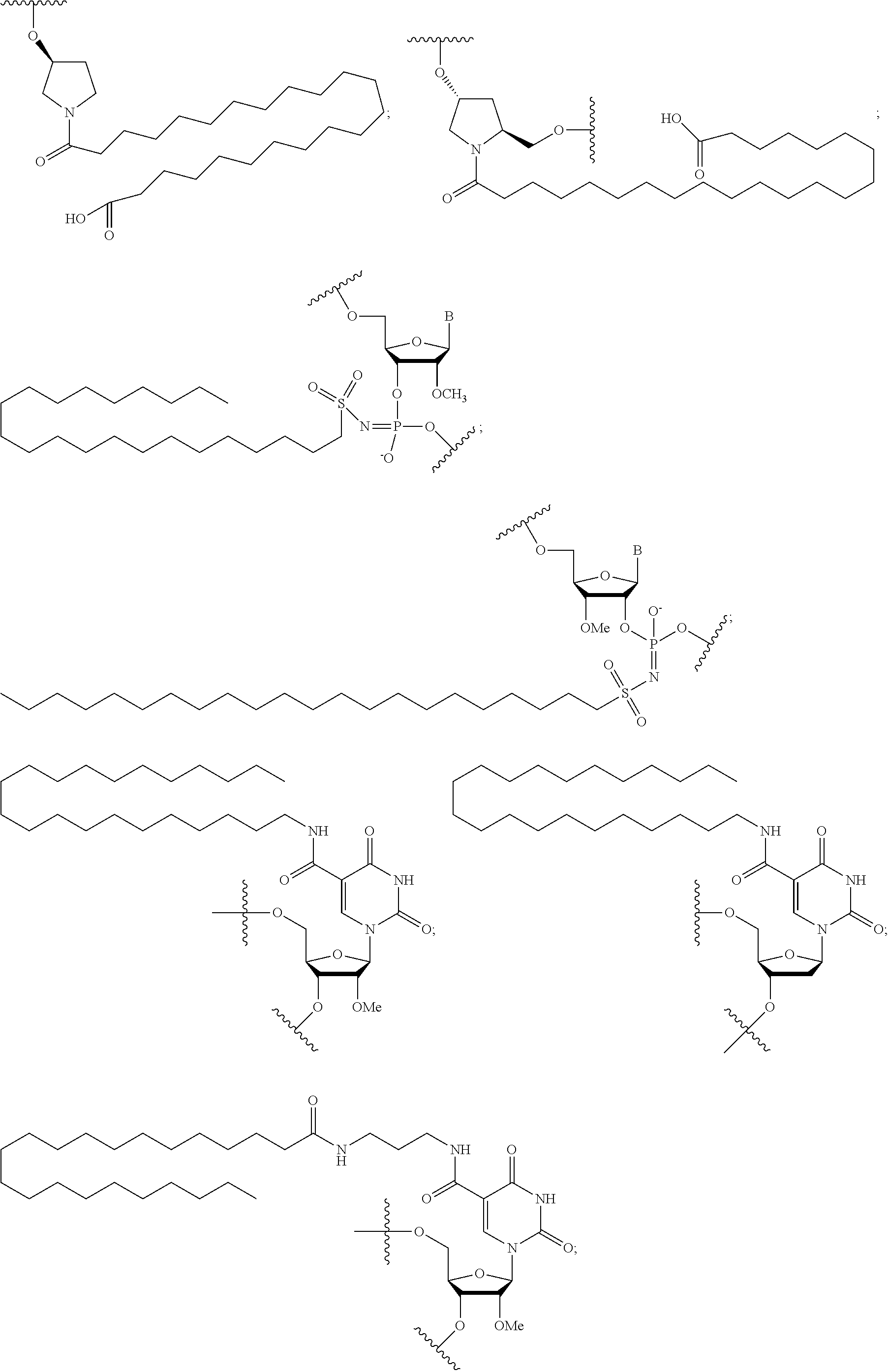 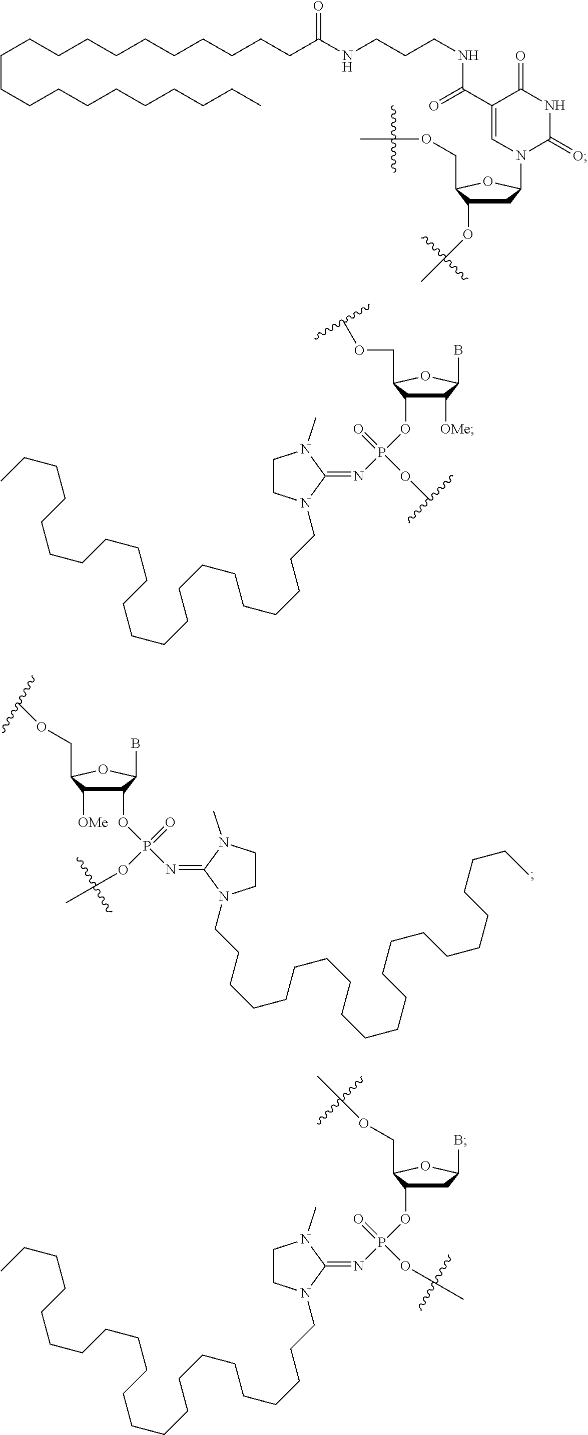 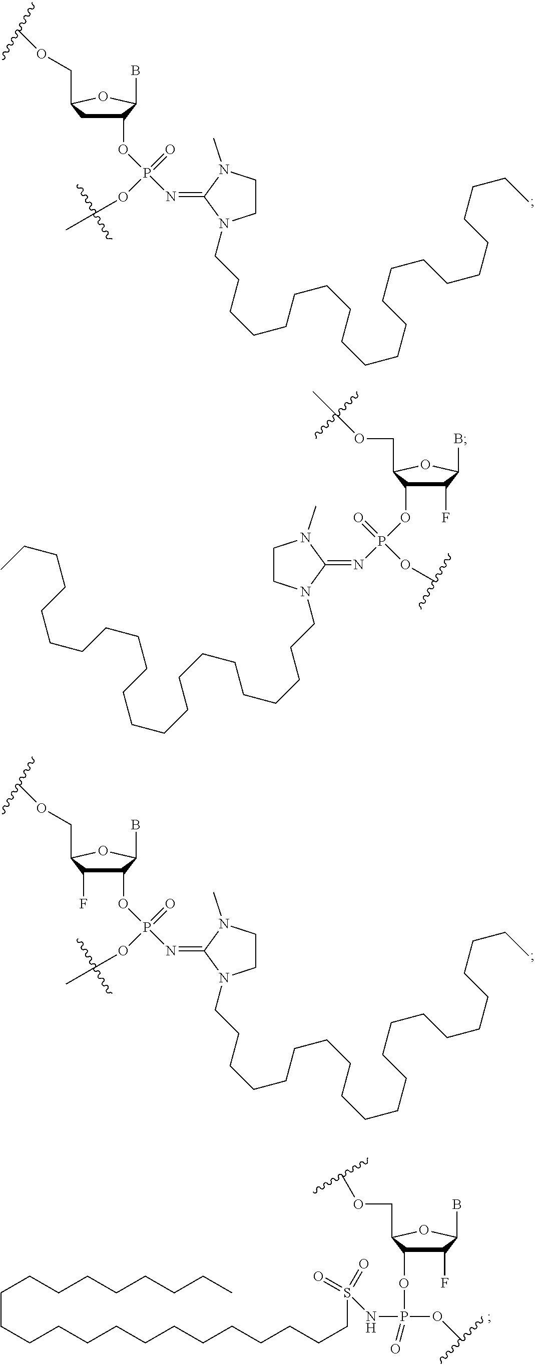 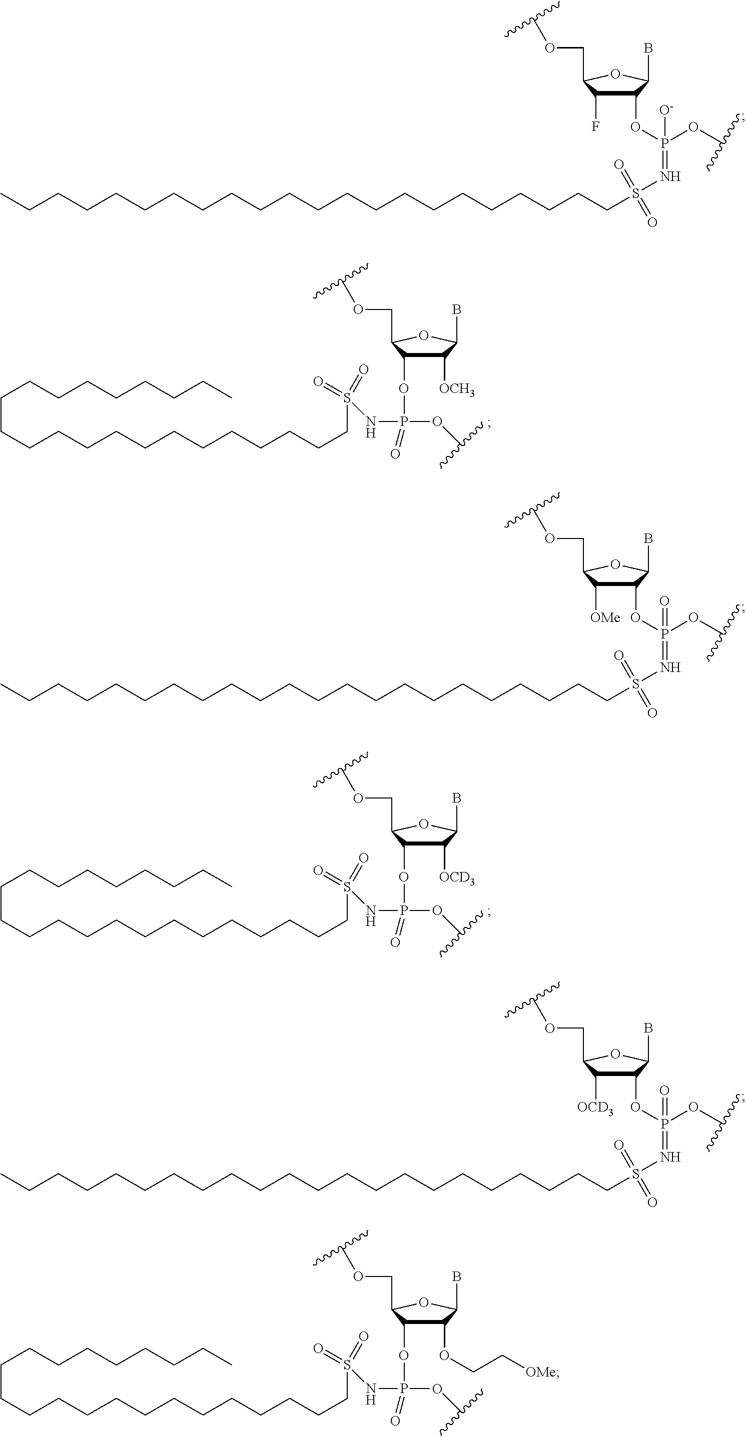 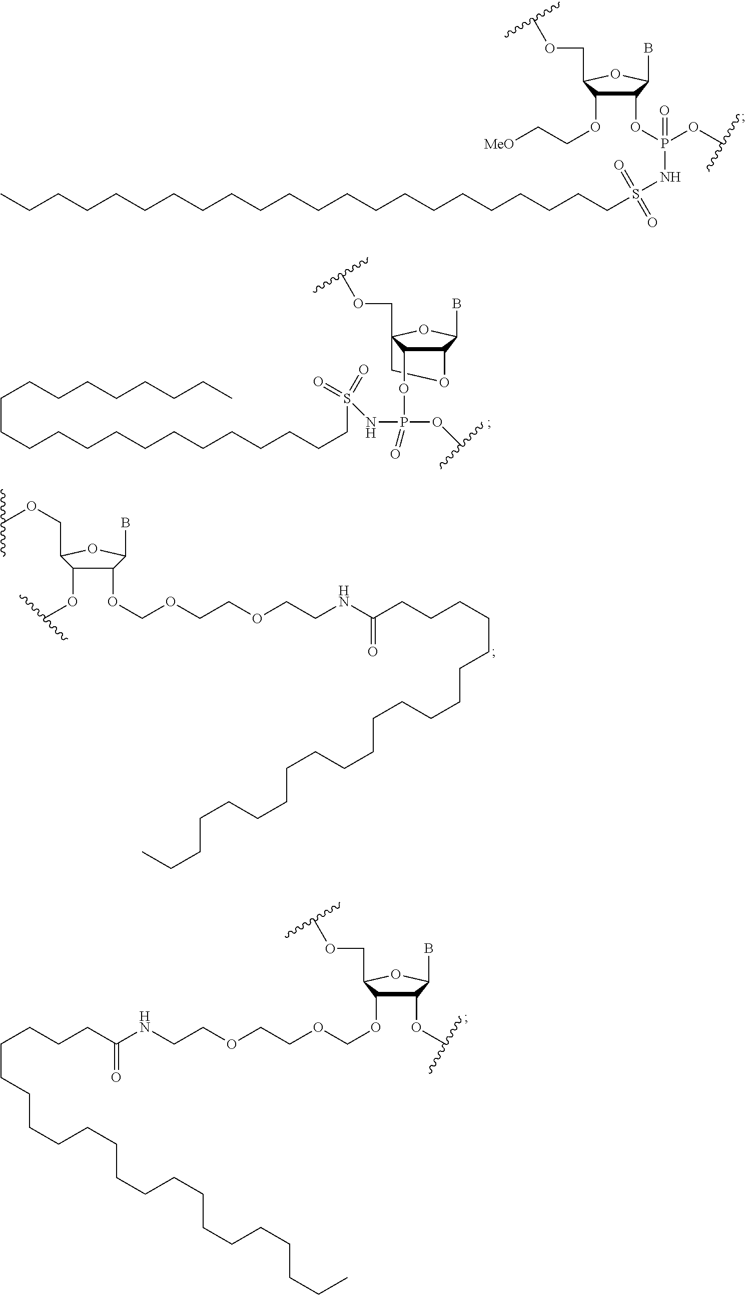 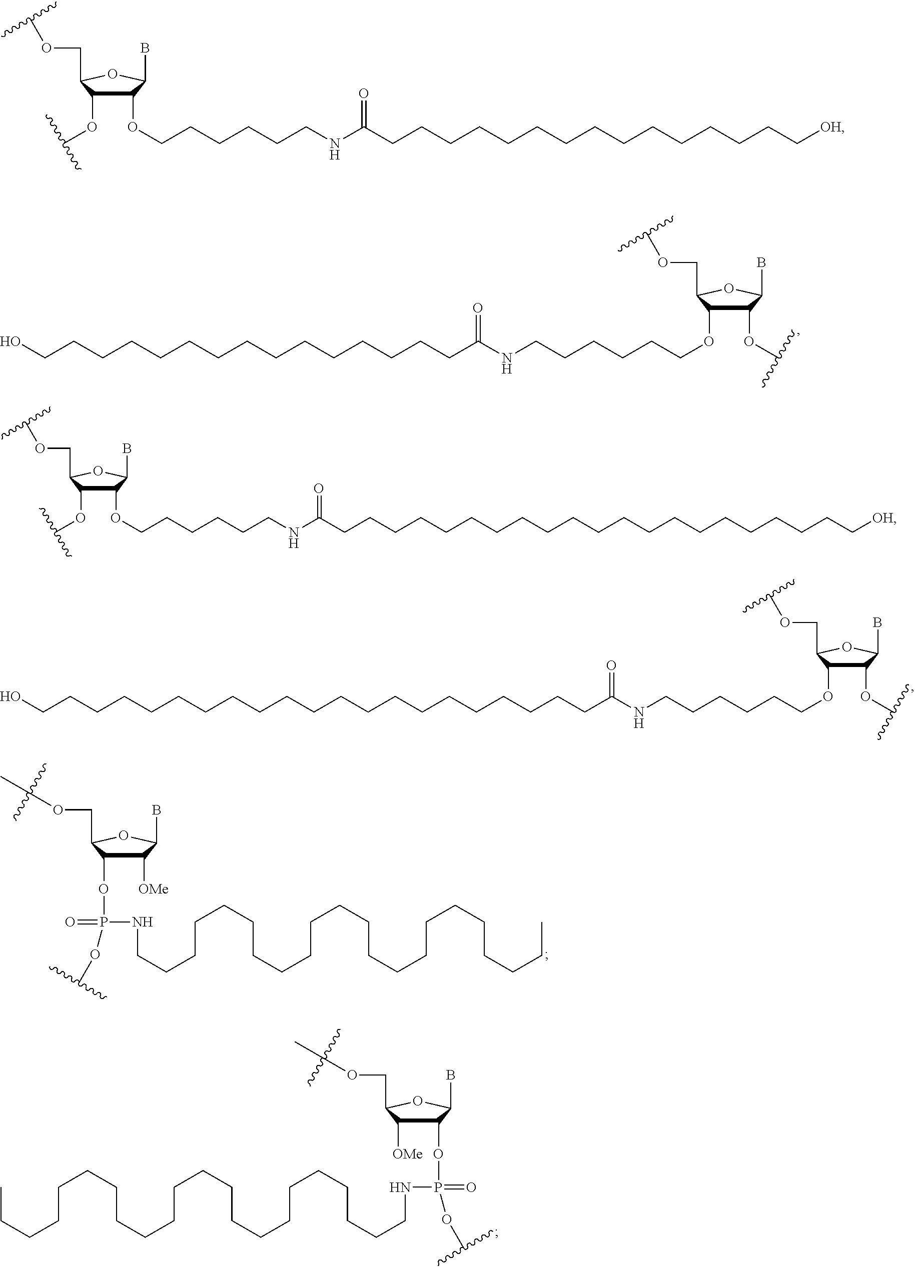 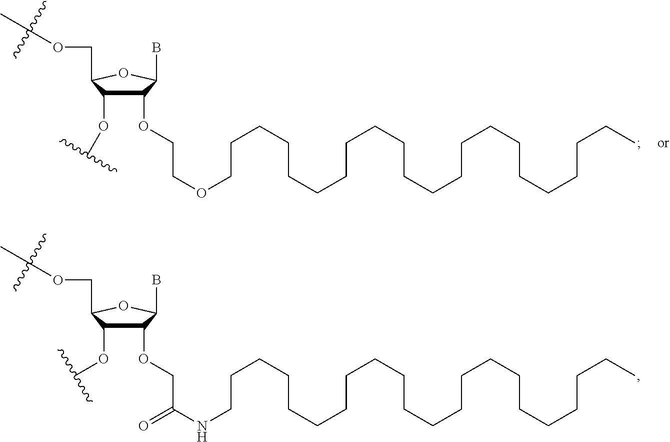
wherein B is a modified or unmodified nucleobase.

13. The dsRNA agent of claim 1, wherein the dsRNA agent comprises a double-stranded region formed between the sense and antisense strands and optionally one or two single-stranded non-loop overhang, and wherein the one or more lipophilic moieties are conjugated to either the double-stranded region or the non-loop overhang.

14. The dsRNA agent of claim 1, wherein the one or more lipophilic moieties are conjugated to one or more internal positions on at least one strand.

15-19. (canceled)

20. The dsRNA agent of claim 14, wherein the one or more lipophilic moieties are conjugated to one or more of the following internal positions: positions 4-8 and 13-18 on the sense strand, and positions 6-10 and 15-18 on the antisense strand, counting from the 5′ end of each strand.

21. The dsRNA agent of claim 20, wherein the one or more lipophilic moieties are conjugated to one or more of the following internal positions: positions 5, 6, 7, 15, 16, and 17 on the sense strand, and positions 10, 15, 16, and 17 on the antisense strand, counting from the 5′-end of each strand.

22. The dsRNA agent of claim 21, wherein the one or more lipophilic moieties are conjugated to position 6 on the sense strand, counting from the 5′-end of the sense strand.

23. The dsRNA agent of claim 1, wherein the one or more lipophilic moieties are conjugated to one or more terminal positions: position 1, 2, or 3 on the sense or antisense strand, counting from the 5′ end or 3′ end of each strand.

24. The dsRNA agent of claim 23, wherein at least one lipophilic moiety is conjugated to position 1 on the sense or antisense strand, counting from the 5′ end of each strand, by modification of the 2′-position, 5′-position, or 4′-position on the sugar moiety, or by modification of the nucleobase of the nucleotide at position 1.

25. The dsRNA agent of claim 1, wherein the dsRNA agent comprises at least one single-stranded overhang on at least one of the termini.

26. (canceled)

27. The dsRNA agent of claim 1, wherein the sense and antisense strands are each independently 15-30 nucleotides in length, 19 to 25 nucleotides in length, or 21 to 23 nucleotides in length.

28. The dsRNA agent of claim 27, wherein:
the sense strand is 21 nucleotides in length, and the antisense strand is 23 nucleotides in length, wherein the strands form a double-stranded region of 21 consecutive base pairs having a 2-nucleotide long single-stranded overhangs at the 3′-end; or
the dsRNA agent has two blunt ends at both ends of the strands, wherein the strands form a double-stranded region of 19-23 consecutive base pairs.

29. (canceled)

30. The dsRNA agent of claim 1, wherein:
the sense strand is 12 to 40 nucleotides in length, and forms a duplex region with the antisense strand;
the sense strand comprises a stem-loop at the 3′ end of the sense strand set forth as: S₁-L-S₂, wherein S1 is complementary to S2, and wherein L forms a loop; and
at least one lipophilic moiety is conjugated to a nucleotide of S₁-L-S₂.

31-37. (canceled)

38. The dsRNA agent of claim 1, wherein the antisense strand comprises a phosphate or phosphate mimic at the 5′-end.

39. The dsRNA agent of claim 38, wherein the phosphate mimic is a 5′-vinyl phosphonate (VP).

40. The dsRNA agent of claim 1, wherein the antisense strand comprises at least one GNA in the seed region.

41-45. (canceled)

46. A cell containing the dsRNA agent of claim 1.

47. A pharmaceutical composition comprising the dsRNA agent of claim 1.

48. A method of modulating the expression of a target gene in a CNS cell, comprising administering to the cell the dsRNA agent of claim 1.

49. (canceled)

50. A method of treating or preventing a CNS disorder in a subject, comprising: administering to the subject a therapeutically effective amount of the dsRNA agent of claim 1, thereby treating the subject by modulating the expression of the target gene in the CNS of the subject.

51-55. (canceled)


SEQUENCE LISTING (191 sequences)

SEQ ID NO: 1 - RNA, 16 nt, synthetic construct
  source 1..16: mol_type = other RNA
gcagccgaaaggctgc

SEQ ID NO: 2 - RNA, 16 nt, synthetic construct
  source 1..16: mol_type = other RNA
gcagccgaaaggctgc

SEQ ID NO: 3 - protein, 29 aa, unidentified
AALEALAEALEALAEALEALAEAAAAGGC

SEQ ID NO: 4 - protein, 30 aa, unidentified
AALAEALAEALAEALAEALAEALAAAAGGC

SEQ ID NO: 5 - protein, 15 aa, unidentified
ALEALAEALEALAEA

SEQ ID NO: 6 - protein, 22 aa, unidentified
GLFEAIEGFIENGWEGMIWDYG

SEQ ID NO: 7 - protein, 23 aa, unidentified
GLFGAIAGFIENGWEGMIDGWYG

SEQ ID NO: 8 - protein, 43 aa, unidentified
GLFEAIEGFIENGWEGMIDGWYGCGLFEAIEGFIENGWEGMID

SEQ ID NO: 9 - protein, 44 aa, unidentified
GLFEAIEGFIENGWEGMIDGGCGLFEAIEGFIENGWEGMIDGGC

SEQ ID NO: 10 - protein, 35 aa, unidentified
GLFGALAEALAEALAEHLAEALAEALEALAAGGSC

SEQ ID NO: 11 - protein, 34 aa, unidentified
GLFEAIEGFIENGWEGLAEALAEALEALAAGGSC

SEQ ID NO: 12 - protein, 41 aa, unidentified
  SITE 17: note = Norleucine
  SITE 38: note = Norleucine
GLFEAIEGFIENGWEGXIDGKGLFEAIEGFIENGWEGXIDG

SEQ ID NO: 13 - protein, 19 aa, unidentified
LFEALLELLESLWELLLEA

SEQ ID NO: 14 - protein, 20 aa, unidentified
GLFKALLKLLKSLWKLLLKA

SEQ ID NO: 15 - protein, 20 aa, unidentified
GLFRALLRLLRSLWRLLLRA

SEQ ID NO: 16 - protein, 30 aa, unidentified
WEAKLAKALAKALAKHLAKALAKALKACEA

SEQ ID NO: 17 - protein, 22 aa, unidentified
GLFFEAIAEFIEGGWEGLIEGC

SEQ ID NO: 18 - protein, 26 aa, unidentified
GIGAVLKVLTTGLPALISWIKRKRQQ

SEQ ID NO: 19 - protein, 8 aa, unidentified
HHHHHWYG

SEQ ID NO: 20 - protein, 10 aa, unidentified
CHKKKKKKHC

SEQ ID NO: 21 - protein, 16 aa, unidentified
RQIKIWFQNRRMKWKK

SEQ ID NO: 22 - protein, 14 aa, unidentified
GRKKRRQRRRPPQC

SEQ ID NO: 23 - protein, 27 aa, unidentified
GALFLGWLGAAGSTMGAWSQPKKKRKV

SEQ ID NO: 24 - protein, 18 aa, unidentified
LLIILRRRIRKQAHAHSK

SEQ ID NO: 25 - protein, 26 aa, unidentified
GWTLNSAGYLLKINLKALAALAKKIL

SEQ ID NO: 26 - protein, 18 aa, unidentified
KLALKLALKALKAALKLA

SEQ ID NO: 27 - protein, 9 aa, unidentified
RRRRRRRRR

SEQ ID NO: 28 - protein, 10 aa, unidentified
KFFKFFKFFK

SEQ ID NO: 29 - protein, 37 aa, unidentified
LLGDFFRKSKEKIGKEFKRIVQRIKDFLRNLVPRTES

SEQ ID NO: 30 - protein, 31 aa, unidentified
SWLSKTAKKLENSAKKRISEGIAIAIQGGPR

SEQ ID NO: 31 - protein, 30 aa, unidentified
ACYCRIPACIAGERRYGTCIYQGRLWAFCC

SEQ ID NO: 32 - protein, 36 aa, unidentified
DHYNCVSSGGQCLYSACPIFTKIQGTCYRGKAKCCK

SEQ ID NO: 33 - protein, 42 aa, unidentified
  SITE 42: note = C-terminal amide
RRRPRPPYLPRPRPPPFFPPRLPPRIPPGFPPRFPPRFPGKR

SEQ ID NO: 34 - protein, 13 aa, unidentified
  SITE 13: note = C-terminal amide
ILPWKWPWWPWRR

SEQ ID NO: 35 - protein, 16 aa, unidentified
AAVALLPAVLLALLAP

SEQ ID NO: 36 - protein, 11 aa, unidentified
AALLPVLLAAP

SEQ ID NO: 37 - protein, 12 aa, unidentified
RKCRIVVIRVCR

SEQ ID NO: 38 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyladenosine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aggatagttcaagtagtcaca

SEQ ID NO: 39 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-deoxy-2'-fluorocytidine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluorocytidine-3'-phosphate
  modified_base 10: mod_base = OTHER; note = 2'-O-methyluridine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluorouridine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methylguanosine
tgtgactacttgaactatccttg

SEQ ID NO: 40 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 41 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 42 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-docosanyl-cytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 43 - RNA, 21 nt, synthetic construct
  source 1..21: mol_type = other RNA
  modified_base 1: mod_base = OTHER; note = 2'-O-hexadecyl-adenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 44 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine 3'-(N-hexadecylsulfonyl) phosphorimidate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 45 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 46 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 47 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methylcytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylcytidine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
gcttcagaccatccaagaaga

SEQ ID NO: 48 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylcytidine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluorouridine-3'-phosphate
  modified_base 17: mod_base = OTHER; note = 2'-O-methylguanosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methylcytidine-3'-phosphorothioate
  modified_base 22: mod_base = OTHER; note = 2'-O-methyluridine-3'-phosphorothioate
  modified_base 23: mod_base = OTHER; note = 2'-O-methylcytidine
tcttcttggatggtctgaagctc

SEQ ID NO: 49 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 50 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 51 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-docosanyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 52 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-docosanyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyladenosine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aggatagttcaagtagtcaca

SEQ ID NO: 53 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-docosanyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyluridine-3'-phosphate
  modified_base 16: mod_base = OTHER; note = 2'-O-methylguanosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
cagatagtcttgtttgctaga

SEQ ID NO: 54 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluorocytidine-3'-phosphate
  modified_base 7: mod_base = OTHER; note = 2'-O-methyladenosine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluorouridine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tctagcaaacaagactatctgag

SEQ ID NO: 55 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyluridine-3'-phosphate
  modified_base 16: mod_base = OTHER; note = 2'-O-methylguanosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
cagatagtcttgtttgctaga

SEQ ID NO: 56 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methylcytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methylcytidine-3'-phosphate
  modified_base 6: mod_base = OTHER; note = 2'-O-docosanyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
gcttcagaccatccaagaaga

SEQ ID NO: 57 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-docosanyl-adenosine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyluridine-3'-phosphorothioate
  modified_base 21: mod_base = OTHER; note = 2'-O-methyladenosine
tgagaatgattctgatgtata

SEQ ID NO: 58 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluoroadenosine-3'-phosphate
  modified_base 10: mod_base = OTHER; note = 2'-O-methylguanosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methyluridine-3'-phosphate
  modified_base 14: mod_base = OTHER; note = 2'-deoxy-2'-fluorocytidine-3'-phosphate
  modified_base 15: mod_base = OTHER; note = 2'-O-methyladenosine-3'-phosphate
  modified_base 16: mod_base = OTHER; note = 2'-deoxy-2'-fluorouridine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tatacatcagaatcattctcaag

SEQ ID NO: 59 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyluridine-3'-phosphorothioate
  modified_base 21: mod_base = OTHER; note = 2'-O-methyladenosine
tgagaatgattctgatgtata

SEQ ID NO: 60 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-docosanyl-cytidine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
tagtgcatgaatagattctca

SEQ ID NO: 61 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methyladenosine-3'-phosphate
  modified_base 7: mod_base = OTHER; note = Thymidine-glycol nucleic acid (GNA), S-Isomer
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
tgagaatctattcatgcactagt

SEQ ID NO: 62 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-docosanyl-guanosine-3'-phosphate
  modified_base 7: mod_base = OTHER; note = 2'-deoxy-2'-fluorouridine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyluridine-3'-phosphorothioate
  modified_base 21: mod_base = OTHER; note = 2'-O-methyladenosine
tttgtgtactgtaaagaatta

SEQ ID NO: 63 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = Thymidine-glycol nucleic acid (GNA), S-Isomer
  modified_base 8: mod_base = OTHER; note = 2'-O-methyluridine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methyladenosine-3'-phosphate
  modified_base 16: mod_base = OTHER; note = 2'-deoxy-2'-fluorocytidine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methyladenosine-3'-phosphorothioate
  modified_base 23: mod_base = OTHER; note = 2'-O-methylcytidine
taattctttacagtacacaaaac

SEQ ID NO: 64 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-docosanyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
tatgaagttcatcatcaaaaa

SEQ ID NO: 65 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methylguanosine-3'-phosphate
  modified_base 7: mod_base = OTHER; note = Adenosine-glycol nucleic acid (GNA), S-isomer
  modified_base 8: mod_base = OTHER; note = 2'-O-methyluridine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluorouridine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methylcytidine
tttttgatgatgaacttcatatc

SEQ ID NO: 66 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
tagtgcatgaatagattctca

SEQ ID NO: 67 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-hexadecyl-guanosine-3'-phosphate
  modified_base 7: mod_base = OTHER; note = 2'-deoxy-2'-fluorouridine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyluridine-3'-phosphorothioate
  modified_base 21: mod_base = OTHER; note = 2'-O-methyladenosine
tttgtgtactgtaaagaatta

SEQ ID NO: 68 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
tatgaagttcatcatcaaaaa

SEQ ID NO: 69 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(N-hexadecyl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 70 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 71 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(N-docosanyl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 72 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 73 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(N,N-dimethyl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 74 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 75 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(N-methyl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 76 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 77 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-oxo-2-(N-hexadec-1-ylamino)ethyl)uridine-3' -phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 78 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 79 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(2-oxo-2-(N-docosan-1-ylamino)ethyl)uridine-3' -phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 80 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 81 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 82 - RNA, 21 nt, synthetic construct
  source 1..21: mol_type = other RNA
  modified_base 1: mod_base = OTHER; note = 2'-O-docosanyl-uridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 83 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-docosanyl-uridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 84 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-docosanyl-guanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 85 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-docosanyl-guanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 86 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-docosanyl-guanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 87 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-docosanyl-cytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 88 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-O-docosanyl-adenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 89 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-docosanyl-adenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 90 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-O-docosanyl-adenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 91 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-O-docosanyl-guanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 92 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-O-docosanyl-guanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 93 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-docosanyl-uridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 94 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-docosanyl-guanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 95 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-docosanyl-guanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 96 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-docosanyl-adenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 97 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-docosanyl-adenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 98 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-docosanyl-adenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 99 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-docosanyl-uridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 100 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-docosanyl-guanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttgggcaaaggtggaaatgaa

SEQ ID NO: 101 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-docosanyl-adenosine-3'-phosphorothioate
  modified_base 21: mod_base = OTHER; note = 2'-O-methyladenosine
ttgggcaaaggtggaaatgaa

SEQ ID NO: 102 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-docosanyl-adenosine
ttgggcaaaggtggaaatgaa

SEQ ID NO: 103 - RNA, 21 nt, synthetic construct
  source 1..21: mol_type = other RNA
  modified_base 1: mod_base = OTHER; note = 2'-O-docosanyl-adenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 104 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-docosanyl-adenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 105 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-docosanyl-adenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 106 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-docosanyl-adenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 107 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-docosanyl-uridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 108 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-docosanyl-cytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 109 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-docosanyl-adenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 110 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-O-docosanyl-adenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 111 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-O-docosanyl-cytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 112 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-O-docosanyl-cytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 113 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-docosanyl-uridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 114 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-docosanyl-adenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 115 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-docosanyl-cytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 116 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-docosanyl-adenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 117 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-docosanyl-adenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 118 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-docosanyl-guanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 119 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-docosanyl-uridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 120 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-docosanyl-uridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 121 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-docosanyl-cytidine-3'-phosphorothioate
  modified_base 21: mod_base = OTHER; note = 2'-O-methyladenosine
aaaatccaacctacaagttca

SEQ ID NO: 122 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-docosanyl-adenosine
aaaatccaacctacaagttca

SEQ ID NO: 123 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 124 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-O-docosanyl-uridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 125 - RNA, 24 nt, synthetic construct
  source 1..24: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-docosanyl-cytidine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = 2'-deoxy-2'-fluorouridine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-O-methyladenosine-3'-phosphate
  modified_base 12: mod_base = OTHER; note = 2'-O-methylcytidine-3'-phosphate
  modified_base 13: mod_base = OTHER; note = 2'-O-methylcytidine-3'-phosphate
  modified_base 14: mod_base = OTHER; note = 2'-O-methyluridine-3'-phosphate
  modified_base 15: mod_base = OTHER; note = 2'-deoxy-2'-fluorouridine-3'-phosphate
  modified_base 16: mod_base = OTHER; note = 2'-O-methyluridine-3'-phosphate
  modified_base 17: mod_base = OTHER; note = 2'-deoxy-2'-fluoroguanosine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphate
  modified_base 22: mod_base = OTHER; note = 2'-O-methyladenosine-3'-phosphorothioate
  modified_base 23: mod_base = OTHER; note = 2'-O-methylguanosine-3'-phosphorothioate
  modified_base 24: mod_base = OTHER; note = 2'-O-methyluridine
ttccatttccacctttgcccaagt

SEQ ID NO: 126 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-docosanyl-adenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 127 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-docosanyl-uridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 128 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-docosanyl-uridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 129 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-docosanyl-uridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 130 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-O-docosanyl-cytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 131 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-O-docosanyl-cytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 132 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-docosanyl-adenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 133 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-docosanyl-cytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 134 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-docosanyl-cytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 135 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-docosanyl-uridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 136 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-docosanyl-uridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 137 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-docosanyl-uridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 138 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-O-docosanyl-guanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 139 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-docosanyl-cytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 140 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-docosanyl-cytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 141 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-docosanyl-cytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 142 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-docosanyl-adenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 143 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-docosanyl-adenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttcatttccacctttgcccaagt

SEQ ID NO: 144 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-docosanyl-guanosine-3'-phosphorothioate
  modified_base 23: mod_base = OTHER; note = 2'-O-methyluridine
ttcatttccacctttgcccaagt

SEQ ID NO: 145 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methyluridine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-docosanyl-uridine
ttcatttccacctttgcccaagt

SEQ ID NO: 146 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-O-docosanyl-guanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 147 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-docosanyl-adenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 148 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-docosanyl-adenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 149 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-docosanyl-cytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 150 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-docosanyl-uridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 151 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = 2'-O-docosanyl-uridine-3'-phosphate
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 152 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-docosanyl-guanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 153 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-docosanyl-uridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 154 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-docosanyl-adenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 155 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-docosanyl-guanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 156 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-docosanyl-guanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 157 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-docosanyl-uridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 158 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-O-docosanyl-uridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 159 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-docosanyl-guanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 160 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-O-docosanyl-guanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 161 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-docosanyl-adenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 162 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-docosanyl-uridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 163 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-docosanyl-uridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 164 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-docosanyl-uridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 165 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-docosanyl-uridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 166 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-docosanyl-cytidine-3'-phosphorothioate
  modified_base 23: mod_base = OTHER; note = 2'-O-methylguanosine
tgaacttgtaggttggattttcg

SEQ ID NO: 167 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-docosanyl-guanosine
tgaacttgtaggttggattttcg

SEQ ID NO: 168 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-(6-(16-hydroxyhexadecan-1-oylamino)hex-1-yl)ur idine 3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 169 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphorothioate
  modified_base 21: mod_base = OTHER; note = 2'-O-methyladenosine-3'-phosphorothioate ((2S,4R)-1 -[6-(docosan-1-oylamino)hexan-1-oyl]-4-hydroxy-2-pyrrolidi nyl)methyl ester
cattttaatcctcactctaaa

SEQ ID NO: 170 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphorothioate
  modified_base 21: mod_base = OTHER; note = 2'-O-methyladenosine-3'-phosphorothioate ((2S,4R)-1 -[6-(hexadecan-1-oylamino)hexan-1-oyl]-4-hydroxy-2-pyrroli dinyl)methyl ester
cattttaatcctcactctaaa

SEQ ID NO: 171 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphorothioate
  modified_base 21: mod_base = OTHER; note = 2'-O-methyladenosine-3'-phosphorothioate ((2S,4R)-1 -[hexadecan-1-oyl]-4-hydroxy-2-pyrrolidinyl)methyl ester
cattttaatcctcactctaaa

SEQ ID NO: 172 - RNA, 21 nt, synthetic construct
  source 1..21: mol_type = other RNA
  modified_base 1: mod_base = OTHER; note = 2'-O-methylcytidine-3'-phosphorothioate 5'-phosphorothioate ((3S)-1-[6-( hexadecan-1-oylamino)hexan-1-oyl]-3-hydroxypyrrolidine) ester
  modified_base 2: mod_base = OTHER; note = 2'-O-methyladenosine-3'-phosphate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 173 - RNA, 21 nt, synthetic construct
  source 1..21: mol_type = other RNA
  modified_base 1: mod_base = OTHER; note = 2'-O-methylcytidine-3'-phosphorothioate 5'-phosphorothioate ((3S)-1-[6-(docosan-1-oylamino)hexan-1 -oyl]-3-hydroxypyrrolidine) ester
  modified_base 2: mod_base = OTHER; note = 2'-O-methyladenosine-3'-phosphate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 174 - RNA, 21 nt, synthetic construct
  source 1..21: mol_type = other RNA
  modified_base 1: mod_base = OTHER; note = 2'-O-methylcytidine-3'-phosphorothioate 5'-phosphorothioate 6-palmitamidohexyl ester
  modified_base 2: mod_base = OTHER; note = 2'-O-methyladenosine-3'-phosphate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 175 - RNA, 21 nt, synthetic construct
  source 1..21: mol_type = other RNA
  modified_base 1: mod_base = OTHER; note = 2'-O-methylcytidine-3'-phosphorothioate 5'-phosphorothioate 6-(16-hydroxyhexadecanamido)hexyl ester
  modified_base 2: mod_base = OTHER; note = 2'-O-methyladenosine-3'-phosphate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 176 - RNA, 21 nt, synthetic construct
  source 1..21: mol_type = other RNA
  modified_base 1: mod_base = OTHER; note = 2'-O-methylcytidine-3'-phosphorothioate 5'-phosphorothioate 6-docosanamidohexyl ester
  modified_base 2: mod_base = OTHER; note = 2'-O-methyladenosine-3'-phosphate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-methyluridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 177 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(N-hexadecyl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 178 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(N-docosanyl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 179 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(11-carboxyundec-1-yl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 180 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(6-((3-beta-cholest-5-en-3-yl )oxcarboxamido)hexyl)phosphoramidate
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 181 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine 3'-(2-((2,5,7,8-tetramethyl-2-(4 ,8,12-trimethyltridecyl)chroman-6-yl)oxy)ethyl)phosphorami date
  modified_base 6: mod_base = OTHER; note = 2'-deoxy-2'-fluorouridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 182 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine 3'-(11-carboxyundec-1-yl)phosphoramidate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 183 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine 3'-(6-aminohexyl)phosphoramidate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 184 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine 3'-(11-carboxyundec-1-yl)phosphoramidate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 185 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine 3'-(6-aminohexyl)phosphoramidate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 186 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine 3'-(11-carboxyundec-1-yl)phosphoramidate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 187 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine 3'-(6-aminohexyl)phosphoramidate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 188 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine 3'-(11-carboxyundec-1-yl)phosphoramidate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 189 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine 3'-(6-aminohexyl)phosphoramidate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 190 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine 3'-(11-carboxyundec-1-yl)phosphoramidate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 191 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine 3'-(6-aminohexyl)phosphoramidate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt